(19) United States
(12) Patent Application Publication
(10) Pub. No.: US 2007/0293573 A1
(43) Pub. Date: Dec. 20, 2007

(54) GLU2 RECEPTOR MODULATORS

(76) Inventors: ERIC GOUAUX, Portland, OR (US); Langzhou SONG, Freehold, NJ (US); Yuli XIE, New York, NY (US); Donald W. LANDRY, New York, NY (US)

Correspondence Address: BAKER BOTTS L.L.P., 30 ROCKEFELLER PLAZA, 44TH FLOOR, NEW YORK, NY, 10112-4498, US

(21) Appl. No.: 11/678,396

(22) Filed: Feb. 23, 2007

Related U.S. Application Data
(60) Provisional application No. 60/776,708, filed on Feb. 24, 2006.

(51) Int. Cl. A61K 31/195; A61K 31/10; A61P 25/24; C07C 233/00; C07C 317/00; C07C 311/00; C07C 229/00; A61K 31/145

(52) U.S. Cl. 514/561; 514/605; 514/616; 514/709; 562/433; 564/123; 564/82; 568/34

(57) ABSTRACT

The present invention provides for compositions and methods for modulating the GluR2 receptor. It is based, at least in part, on the discovery, by X-ray crystallography, that a known GluR2 agonist binds to the receptor in two different orientations, thereby diminishing its potency. The present invention provides for structural alternatives in which alternative binding possibilities are substantially eliminated.

### PRIORITY CLAIM

This application claims priority to U.S. Provisional Application No. 60/776,708, filed Feb. 24, 2006, incorporated by reference in its entirety herein.

### GRANT INFORMATION

The subject matter of this application was developed, at least in part, using National Institutes of Health Grant No. 5 RO1NS38631-04, so that the United States Government has certain rights herein.

### 1. INTRODUCTION

The present invention provides for compositions and methods for modulating the GluR2 receptor. It is based, at least in part, on the discovery, by X-ray crystallography, that LY451646, a known GluR2 modulator, binds to the receptor in two different orientations, thereby diminishing its potency. The present invention provides for structural alternatives in which alternative binding possibilities are substantially eliminated.

### 2. BACKGROUND OF THE INVENTION

Communication between cells of the mammalian central nervous system (CNS) is controlled by the interactions between neurotransmitters, produced by a sending (presynaptic) cell, and their receptors in the membrane of a receiving (postsynaptic) cell. These interactions can have excitatory or inhibitory affects on the postsynaptic cell. The major excitatory pathways in the mammalian CNS are mediated by the amino acid neurotransmitter glutamate (Watkins and Evans, 1981, Annu Rev Pharmacol Toxicol. 21: 165-204; Monaghan et al., 1989, Annu Rev Pharmacol Toxicol. 29: 365-402; Watkins et al., 1990, Trends Pharmacol Sci. 11: 25-33).

The excitatory amino acid receptors that bind glutamate are classified into two different receptor types, “ionotropic” and “metabotropic.” Metabotropic glutamate receptors are slower acting than the ionotropic receptors, but are active for a longer time period upon neurotransmitter binding. The metabotropic glutamate receptors are coupled to G-protein second messenger pathways. Activation of these receptors results in changes in cytoplasmic cAMP levels which in turn alters the functionality of membrane bound ion channels. The metabotropic glutamate receptors are composed of subunits encoded for by the genes mGlu1-8 (Schoepp and Conn, 1993, Trends Pharmacol Sci. 14: 13-20).

Ionotropic receptors are directly coupled to ion channels in the plasma membranes of postsynaptic cells. Ionotropic glutamate receptors are fast acting. Binding of a neurotransmitter to an inactive receptor induces the activation of the receptor and opening of the ion channel, allowing the passage of ions across the cell membrane. Three different types of ionotropic glutamate receptors have been identified which are defined by the excitatory actions of their major activators: N-methyl-D-aspartate (NMDA), α-amino-3-hydroxy-5-methylisoxazole-4-propionic acid (AMPA), and kainic acid (Kainate); although AMPA and Kainate can activate each others' receptor types (Bleakman et al., 1996, Neuropharmacology 35: 1689-702).

Functional AMPA receptors are composed of subunits encoded by the genes GluR1-4. Functional Kainate receptors may be composed of the high affinity subunits encoded by the genes KA1 and KA2, and the low affinity subunits encoded by the genes GluR5-7. Each Kainate and AMPA receptor is composed of a heteromeric or homomeric cluster of four receptor subunits. The subunits that compose functional NMDA receptors are encoded by the genes NR1 and NR2. All of the genes that encode the various subunits for the three ionotropic glutamate receptors can undergo alternative splicing, generating a large diversity of potential subunit compositions.

The biophysical and pharmacological properties of the ionotropic glutamate receptors are dependent on their subunit composition (Hollmann and Heinemann, 1994, Annu Rev Neurosci 17:31-108). The GluR2 subunit has been shown to play a major role in modulating ionic currents, susceptibility to excitotoxicity, and ion permeability. Specifically, the presence of the GluR2 subunit in AMPA receptors decreases receptor permeability to Ca²⁺ (Hume et al. 1991, Science 253:1028-1031; Burnashev et al., 1992, Neuron 8:189-198). Furthermore, the C-terminal cytoplasmic tail of GluR2 is important for mediating interactions with various cytoplasmic proteins that may influence membrane receptor turnover (Man et al., 2000, Neuron 25:649-662), synaptic transmission, efficacy, and plasticity (Jia et al., 1996, Neuron 17:945-956), and receptor clustering (Matsuda et al., 2000, EMBO J 19:2765-2774).

Current models propose that the ionotropic receptor subunits, including GluR2, consist of three transmembrane spanning domains and a reentrant loop (Paas, 1998, Trends Neurosci. 21: 117-125; Wo and Oswald, 1995, Trends Neurosci. 18: 161-168). The ligand binding domain is composed of a clamshell-like dimer formed by the S1 region located before the first transmembrane domain, and the S2 region located between the second and third transmembrane domains (Armstrong and Gouaux, 2000, Neuron 28: 165-181; Armstrong et al., 1998, Nature 395: 913-917). The dimer is formed by two structural domains, one of which (“D1”) is composed mostly of amino acid residues from S1, and the other (“D2”), which is composed mostly of amino acid residues from S2 (as discussed in Valentine and Palmer, 2005, Biochem. 44:3410-3417). When the receptor is activated (see FIG. 1) by binding of an activator, the cleft between the two domains closes (hence the term, “clamshell”) and the ion channel opens. If the receptor is desensitized, the “clamshell” is held in a closed position, the activator is bound tightly, yet the ion channel is closed (FIG. 1).

While the ionotropic glutamate receptors are tetrameric complexes comprised of “dimers-of-dimers” (Sun et al., 2002, Nature 417:245-253), study of the conformational effects of binding of various modulators has been facilitated by the creation of a soluble ligand-binding core of GluR2, referred to as GluR2-S1S2J (Armstrong and Gouaux, 2000, Neuron 28: 165-181). GluR2-S1S2J comprises the two domains, S1 and S2which, rather than being bound to transmembrane domains, are joined by a single linker (see FIG. 2). GluR2-S1S2J can be crystallized, as can be complexes between it and various modulators. High resolution X-ray structures of GluR2-S1S2J with various activators and at least one inactivator have revealed different degrees of cleft closure between D1 and D2 (Lunn et al., 2003, J. Med. Chem. 2003, 46:872-875; Jin et al., 2005, J. Neurosci. 25(39):9027-9036; Valentine and Palmer III, 2005, Biochemistry 44:3410-3417; Kubo and Ito, 2004, Proteins 56:411-419; Jin and Gouaux, 2003, Biochemistry 42:5201-5213; Hogner et al., 2003, J. Med. Chem. 46:214-221; Armstrong et al., 2003, Proc. Natl. Acad. Sci. U.S.A. 100(10):5736-5741; Carcache et al., 2003, Bioorganic & Med. Chem. 11:551-559; Jin et al., 2002, Biochem. 41:15635-15643; Hogner et al., 2002, J. Mol. Biol. 322: 93-109).

Inappropriate stimulation of the glutamate receptors can lead to neurological disorders and neurodegeneration. For example, excessive activation of the ionotropic receptors can result in a condition known as excitotoxicity in which overactivation results in the death of the glutaminergic cells. Additional conditions such as stroke, cerebral ischemia, Alzheimer's Disease, amyotrophic lateral sclerosis, Huntington's disease, AIDS-induced dementia, depression, and anxiety related disorders can result from the inappropriate activation of glutamate receptors (see U.S. Pat. Nos. 5,446,051 and 5,670,516).

The development of compounds that can modulate the activity of the glutamate receptors when they bind neurotransmitter is essential to alleviating these neurological conditions (Weiser, 2005, Curr Drug Targets CNS Neurol Disord. 4:153-9). Currently, many candidate anti-depressant and anti-anxiety drugs are tested for their ability to reduce “despair” in mice and rats during forced swim tests (FST) and tail suspension tests (TST). 87%-94% of the compounds that successfully reduce despair in mice and rats are clinically effective at reducing depression and anxiety in humans (Porsolt and Lenerge, 1992, Behavioral models of depression. In: Elliott, J. M., Heal, D. J., and Marsden, C. A. editors. Experimental approaches to anxiety and depression. London. pp. 73-85; Borsini and Meli, 1988, Psychopharmacology 94: 147-60).

LY451646, an active isomer of LY404187 (R,S—N-2-(4-(4-Cyanophenyl)phenyl)propyl 2-propanesulfonamide) and an allosteric AMPA receptor potentiator (Miu et al., 2001, Neuropharmacology 40: 976-83), has been observed to reduce “despair” in the FST and TST (Bai et al., 2001, Pharmacology, Biochemistry, and Behavior 70: 187-192), and may therefore have potential as a candidate anti-depressant or anti-anxiety drug. It has also been found to increase hippocampal cell proliferation in rats (Bai et al., 2003, Neuropharmacol. 44(8):1013-1021). Additionally, its isomer LY404187 increases glutamate dependent AMPA stimulation of the rat brain as evident by increased glucose utilization and c-fos expression in the hippocampus, neocortex, dorsal raphe nucleus, lateral habenula, and locus coeruleus; areas associated with memory processing and cognition (Fowler et al., 2004, J Cereb Blood Flow Metab. 24: 1098-109). LY404187 interacts functionally with GluR2's Flip/Flop splice domain which is adjacent to the S1S2 dimer. Interacting with the Flip splice variant, LY404187 can potentiate the receptor's response to glutamate by decreasing the receptor's desensitization to prolonged glutamate exposure (Quirk and Nisenbaum, 2003, Journal of Neuroscience. 23: 10953-10962). LY404187 has also been found to exhibit a neurotrophic effect in a rat model of Parkinson's disease (O'Neil et al., 2004, Eur. J. Pharmacol. 486(2):163-174). Understanding glutamate receptor structure and allosteric binding sites can facilitate the development of candidate compounds that may bind the receptor and modulate its response to the neurotransmitter glutamate.

### 3. SUMMARY OF THE INVENTION

The present invention provides for methods and compositions for modulating the activity of the GluR2 receptor. It is based, at least in part, on the discovery that the GluR2 agonist, LY451646, bound to GluR2-S1S2J, equally occupies two different orientations, thereby diminishing its potency.

The present invention provides for new compounds that are designed to bind to GluR2 in essentially one structural orientation, and for methods of identifying further modulator compounds. In particular, non-limiting embodiments, the present invention provides for molecules which exhibit 2-fold molecular symmetry and which comprise a napthalene core or a bi-aryl core.

In various embodiments, the present invention encompasses agonists as well as antagonists of GluR2 activity, where an agonist may enhance, and an antagonist may inhibit, the effect of an endogenous activator of the GluR2 receptor, such as glutamate. Agonists may be used for the treatment of depression, anxiety, and cognitive disorders, and to facilitate learning. Antagonists may be used for the treatment of ischemic, infectious, and/or degenerative neurological diseases.

In further embodiments, the present invention provides for a crystal structure of GluR2-S1S2J bound to ligand LY451646, as set forth herein as atomic coordinates in Appendix A. In related embodiments, the present invention provides for a Fo-Fc electron density map of LY451646, as bound to GluR2(S1S2J), and for a virtual form of said map as embodied in a computer simulation of said map.

### 4. BRIEF DESCRIPTION OF THE FIGURES

FIG. 1. Schematic drawing showing the mechanisms of ionotropic glutamine receptor activation and desensitization.

FIG. 2. Schematic drawing showing the design of GluR2-S1S2J, wherein the connection between S1 and S2 and transmembrane domains 1 and 2 is severed and replaced by a linker molecule, and S2 is separated from transmembrane domain S3.

FIG. 3. LY451646 potentiates glutamate currents from Delta ATD GluR2 as measured by TEVC in oocytes. The top tracing represents application of glutamate alone, the bottom tracing glutamate plus LY451646.

FIG. 4A-B. LY451646 promotes dimerization of GluR2 S1S2. (A) GluR2 alone; (B) GluR2S1S2 in the presence of LY451646.

FIG. 5. Data collection and refinement statistics for LY451646 bound to GluR2-S1S2J.

FIG. 6. Schematic drawing, showing ribbon structures of GluR2-S1S2J, depicting one molecule of LY451646 binding to the dimer interface.

FIG. 7. Schematic drawing, showing ribbon structures of GluR2-S1S2J, depicting LY451646 binding near the “hinge” of the GluR2-S1S2J “clamshell.”

FIG. 8. Schematic drawing, showing a closeup of the LY451646 at its binding site in GluR2-S1S2J.

FIG. 9A-C. (A) Schematic drawing of LY451646 bound to GluR2-S1S2J in a first orientation. (B) Schematic drawing of LY451646 bound to GluR2-S1S2J bound to GluR2-S1S2J in a second orientation. (C) Schematic drawing of LY404187.

FIG. 10A-B. Fo-Fc electron density for LY451646, as bound to GluR2-S1S2J. (A) View parallel to the 2-fold axis of symmetry. (B) View perpendicular to the 2-fold axis of symmetry.

FIG. 11. Fo-Fc electron density resulting from the two binding orientations of LY451646, with their associated molecular structures superimposed.

FIG. 12. Structure and Fo-Fc electron density of SYM1, having formula

FIG. 13. SYM1 fits within the Fo-Fc electron density of LY451646, in this view parallel to the 2-fold axis.

FIG. 14A-C. Depiction of SYM1 in the binding cleft of GluR2-S1S2J. (A) Two-fold disordered molecules; (B) two-fold symmetric SYM1; (C)Schematic drawing of SYM1 bound to GluR2-S1S2J.

FIG. 15. Depiction of LY404187 and its interactions with the GluR2 S1S2 binding site.

FIG. 16A-E. Electrophysiologic studies comparing GR341 with LY451646. A. Microamperes of current over various periods of time in a GluR2 expressing oocyte (Xenopus lacevis oocyte with microinjected rat GluR2 receptor-encoding mRNA), exposed to various concentrations (0.001 μM, 0.01 μM, 0.1 μM and 1 μM) of GR341 (formula 5). B. Microamperes of current over various periods of time in a GluR2 expressing oocyte exposed to various concentrations (0.001 μM, 0.01 μM, 0.1 μM, 1 μM and 10 μM) of LY451646. C. In an oocyte expressing GluR2, ratio of current/maximal current (I/Iₘₐₓ) versus concentration of GR341. D. In an oocyte expressing GluR2, ratio of current/maximal current (I/Iₘₐₓ) versus concentration of LY451646. E. GR341.

FIG. 17. Effect of GR341 on locomotor activity in the context of various doses of amphetamine.

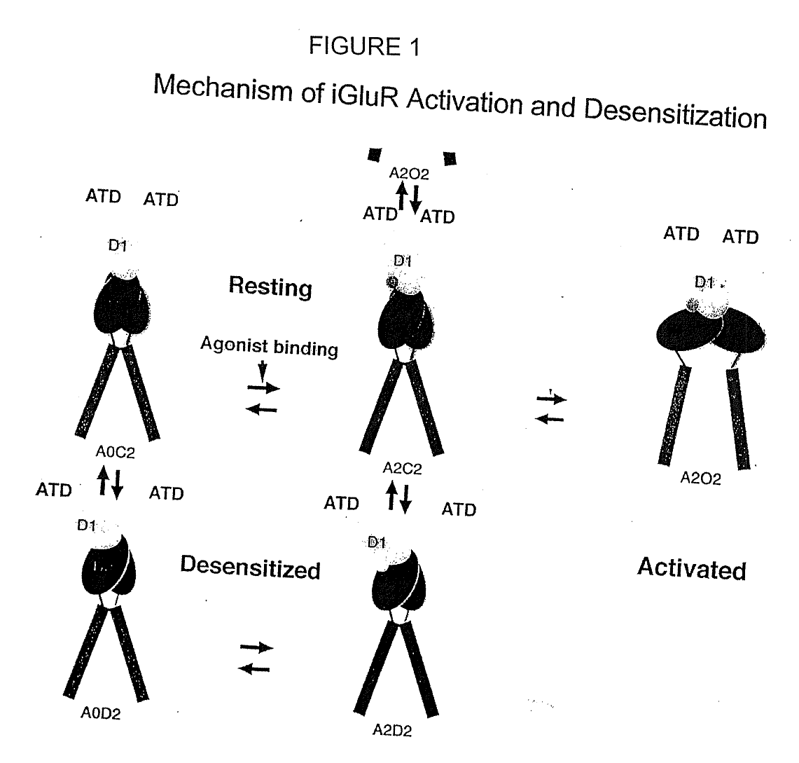

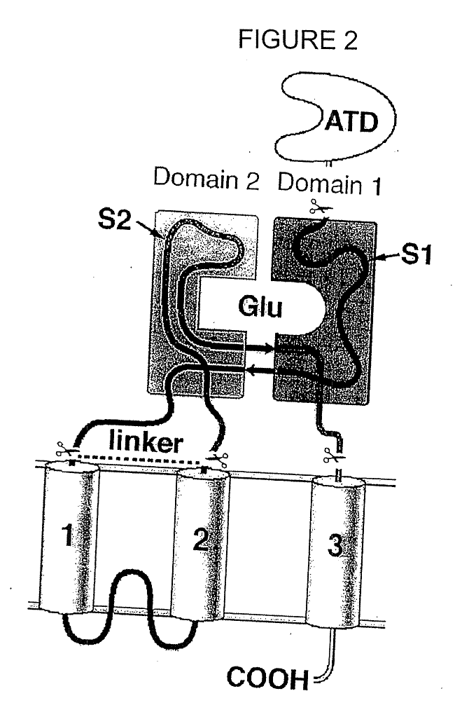

### 5. DETAILED DESCRIPTION OF THE INVENTION

For clarity, and not by way of limitation, the detailed description of the invention is divided into the following subsections:

(i) GluR2 modulators;

(ii) methods for characterizing GluR2 modulators; and

(iii) uses of GluR2 modulators.

### 5.1 GluR2 Modulators

The present invention provides for GluR2 modulators with binding properties similar to LY451646, but which bind essentially in one orientation to GluR2, for example to GluR2-S1S2J. “Bind essentially in one orientation” means that desirably the modulator binds in only one structural orientation, but in the event that more than one binding orientation is observed (e.g., by X-ray crystallography), there exists one structural orientation in which the modulator is found bound to GluR2 in at least about 75 percent of occurrences.

In particular non-limiting embodiments, the modulator exhibits 2-fold symmetry, which means that it looks the same after being rotated by 180 degrees. Because of this symmetry, even if a modulator at a particular position were labeled and it was found that the labeled molecule occurred sometimes at a first position, and sometimes at a second position in which the modulator is rotated 180 degrees, said modulator would still be said to bind in only one structural orientation, even where several detectable orientations exist.

In other particular non-limiting embodiments, the modulator, which may or may not exhibit 2-fold symmetry, may exhibit, when bound to GluR2, a Fo-Fc electron density which conforms to the Fo-Fc electron density of LY451646 bound to GluR2(S1S2J). “Conforms” means that the Fo-Fe electron density determined for a modulator bound to GluR2(S1S2J) overlaps with the Fo-Fc electron density of LY451646, determined by the same methods (for example, at a resolution of approximately 1.0 Angstroms or less), by at least about 60 percent, at least about 70 percent, at least about 80 percent, at least about 90 percent, or at least about 95 percent. The electron density of LY451646 may be determined using the coordinates set forth in Appendix A/

In particular non-limiting examples of species of any of the above embodiments, a modulator of the invention may be a molecule comprising a linear conformation, in which both ends of the linear conformation show increased electron density relative to the mid-portion of the linear conformation and optionally may exhibit a net negative charge. In further non-limiting examples, the areas of increased electron density may, when bound to GluR2, such as GluR2(S1S2J), form a polar or non-polar attracting interaction with one or more of the following residues of GluR2: Ile 481, Lys 493, Pro 494, Phe 495, Met 496, Ser 497, Leu 498, Ser 729, Lys 730, Gly 731, Leu 751, Ser 754, Leu 759.

In particular non-limiting examples of species of any of the above embodiments, a modulator of the invention may, in GluR2, e.g. GluR2-S1S2J, form an attracting interaction, which may be polar or non-polar, with one or more of the following GluR2 amino acid residues: Ile 481, Lys 493, Pro 494, Phe 495, Met 496, Ser 497, Leu 498, Ser 729, Lys 730, Gly 731, Leu 751, Ser 754, Leu 759.

In particular non-limiting examples of species of any of the above embodiments, a modulator of the invention may, in GluR2, e.g. GluR2-S1S2J, form an attracting interaction, which may be polar or non-polar, with one or more of the following GluR2 amino acid residues, where subscripts A and B denote the different protomers (see Example 1, below, and Appendix A: Ile_{B} 481, Pro_{A} 494, Ser_{B} 497, Ser_{A} 729, Ser_{B} 729, Lys_{A} 730, GIly_{B} 731, Leu_{A} 751, and Ser_{A} 754.

In particular non-limiting embodiments of the invention, the modulator of the invention may be represented as formula 1:
R¹—R²R³R⁴—R⁵

where R¹ and R⁵ are preferably, but not necessarily, the same, and may be alkylaminosulfonyl, alkylaminosulfonylalkyl, alkylsulfonyl, alkylsulfonylalkyl, alkylaminocarbonyl, alkylaminocarbonylalkyl, alkylaminocarboxy, alkylaminocarboxyalkyl;

where R² and R⁴ are preferably, but not necessarily, the same, and are ring structures, optionally substituted, which may be aryl, cycloalkyl, or heterocyclyl; and

where R³ may be a bond between R² and R⁴ which either is shared by or joins the two rings without being shared in the rings themselves.

In non-limiting embodiments, R²R³R⁴ may be a naphthyl group, a piperazine group, or a biaryl group.

In further non-limiting embodiments, R²R³R⁴ may be a naphthyl group, a piperazine group, or a biaryl group, and R¹ and R⁵ may be the same, and may each be an alkylaminosulfonyl or an alkylaminosulfonylalkyl group.

In a particular set of embodiments, the present invention provides for a compound having formula 2:

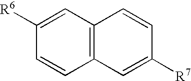

where R⁶ and R⁷ are preferably, but not necessarily, the same, and may be alkylaminosulfonyl, alkylaminosulfonylalkyl, alkylsulfonyl, alkylsulfonylalkyl, alkylaminocarbonyl, alkylaminocarbonylalkyl, alkylaminocarboxy, alkylaminocarboxyalkyl. In one specific non-limiting embodiment, the present invention provides for a compound having formula 3 (also referred to as “SYM1”):

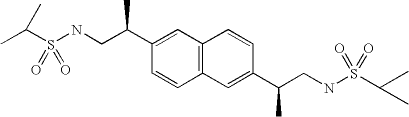

In another particular set of embodiments, the present invention provides for a compound having formula 4:

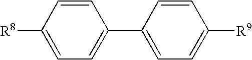

where R⁸ and R⁹ are preferably, but not necessarily, the same, and may be alkylaminosulfonyl, alkylaminosulfonylalkyl, alkylsulfonyl, alkylsulfonylalkyl, alkylaminocarbonyl, alkylaminocarbonylalkyl, alkylaminocarboxy, alkylaminocarboxyalkyl. In one specific non-limiting embodiment, the present invention provides for a compound having formula 5:

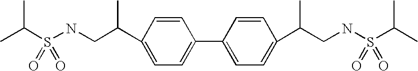

In another set of non-limiting embodiments, the present invention provides for a compound of formula 6: 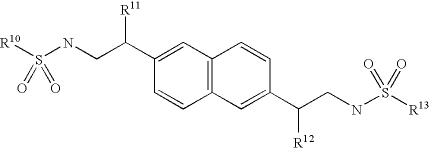
where R¹⁰ and R¹³ are the same and/or R¹¹ and R¹² are the same, and preferably R¹⁰ and R¹³ are the same and R¹¹ and R¹² are the same, and each of R¹⁰, R¹¹, R¹² and R¹³ individually may be H or alkyl, including but not limited to methyl, ethyl, propyl, isopropyl or butyl, and preferably where R¹⁰ and/or R¹³ is propyl where the linkage to the sulfur is via the middle carbon.

In another set of non-limiting embodiments, the present invention provides for a compound of formula 7: 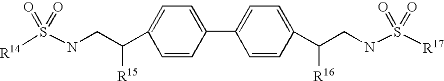
where R¹⁴ and R¹⁷ are the same, or, if they are not, then R¹⁵ and R¹⁶ are the same, and each of R¹⁴, R¹⁵, R¹⁶, and R¹⁷ individually may be H or alkyl, including but not limited to methyl, ethyl, propyl, isopropyl or butyl, and preferably where R¹⁴ and/or R¹⁷ is propyl where the linkage to the sulfur is via the middle carbon.

The foregoing compounds and classes of compounds, in non-limiting embodiments of the invention, (i) may exhibit, when bound to GluR2, a Fo-Fc electron density which conforms to the Fo-Fc electron density of LY451646 bound to GluR2(S1S2J); and/or (ii) may, in GluR2, e.g. GluR2(S1S2J), form an attracting interaction, which may be polar or non-polar, with one or more of the following GluR2 amino acid residues: Ile 481, Lys 493. Pro 494, Phe 495, Met 496, Ser 497, Leu 498, Ser 729, Lys 730, Gly 731, Leu 751, Ser 754, Leu 759; and/or (iii) may exhibit GluR2 agonist activity; and/or (iv) may exhibit GluR2 antagonist activity.

The present invention further provides for methods of identifying GluR2 modulators comprising using the solved crystal structure of GluR2-S1S2 bound to LY451646 to identify additional compounds that bind substantially to the same site as LY451646 but which show advantageous properties, such as increased potency, less side effects, and/or slower deactivation of GluR2 (see below). For example, a virtual model of LY451646 as bound in GluR2 may be used to develop additional compounds which fit, with appropriate electrostatic interactions, into the binding site.

### 5.2 Methods for Characterizing GluR2 Modulators

The present invention encompasses agonists (including partial agonists) as well as antagonists of GluR2.

A modulator of the invention may be identified as an agonist by any method known in the art. For example, upon binding to GluR2(S1S2J), an agonist tends to result in a closure of the cleft between S1 and S2 by approximately 20°, as measured, for example, by determining the crystal structure. A partial agonist, in contrast, would result in closure of the cleft by approximately 10-18°. Binding of an antagonist is not associated with substantial cleft closure. An agonist of the present invention stabilizes the receptor in a closed-cleft state, which resembles the conformation of the receptor when it is bound to an activator, such as glutamate. The modulators of the invention may be used in the presence or absence of an activator.

In particular embodiments, a modulator of the invention may be identified as an agonist (full or partial agonist) by determining that the modulator slows deactivation of GluR2. For example, a slowing of deactivation may be determined using studies as described in Jin et al., 2005, J. Neurosci. 25(39): 9027-9036, in which electrophysiological responses of GluR2(flop), expressed on outside-out patches of transiently transfected HEK 293 cells, were measured. A modulator of the invention may be considered an agonist if it slows deactivation more than 100 μM cyclothiazide, and/or slows deactivation at least as much as 5 mM antiracetam, and/or slows deactivation at least as much as 100 μM CX614. An antagonist, on the other hand, would promote deactivation (whereby the ion-conducting pore of the receptor closes).

### 5.3 Uses of GluR2 Modulators

An agonist of the invention (either a full agonist or a partial agonist) may be used to treat (alleviate) depression, to improve cognition/leaning, and to treat (alleviate) disorders such as Parkinson's disease, Alzheimer's disease, or schizophrenia. An agonist of the invention may be used to promote neurotrophic effects.

An inhibitor of the invention may be used to treat conditions, diseases and disorders associated with glutamate toxicity, including, but not limited to, ischemia, amyotrophic lateral sclerosis, and, notwithstanding the cognitive benefits which may be associated with agonists, Alzheimer's disease, Parkinson's Disease, HIV-associated dementia, and Huntington's chorea.

The present invention provides for the use of any of the compounds set forth herein as formulas 1-7, in effective amounts, for treating a disorder which, in non-limiting embodiments, is selected from the group consisting of anxiety, depression, and a combination thereof. In specific, non-limiting embodiments, an effective amount, which may be administered daily as a single or divided dose, may be between about 0.01 and 100 mg/kg, or between about 0.1 and 10 mg/kg, or between about 0.1 and 5 mg/kg, or between about 0.5 and 5 mg/kg.

Compounds of the invention may be administered by any route known in the art, including, but not limited to, oral, nasal or pulmonary inhalation, subcutaneous, intravenous, transcutaneous, intrathecal, rectal, etc.

Compounds of the invention may be comprised in a formulation together with a suitable pharmaceutical cannier, including, but not limited to, water, saline, etc.

Compounds of the invention may be administered in solid (e.g., tablets, capsule, suppositories) or liquid form.

Compounds of the invention may be administered together with one or more bioactive agent, including other anti-depressant and/or anti-anxiety compounds.

### 6. EXAMPLE 1

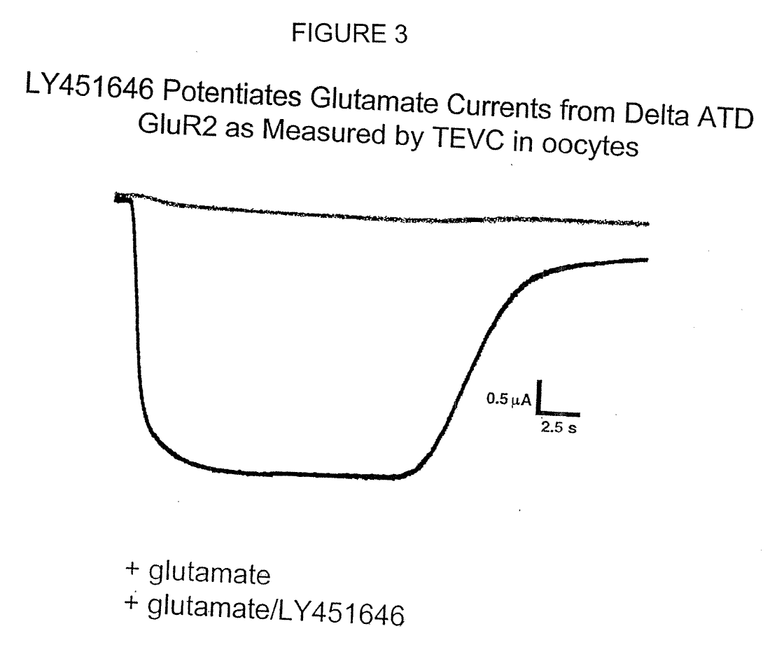

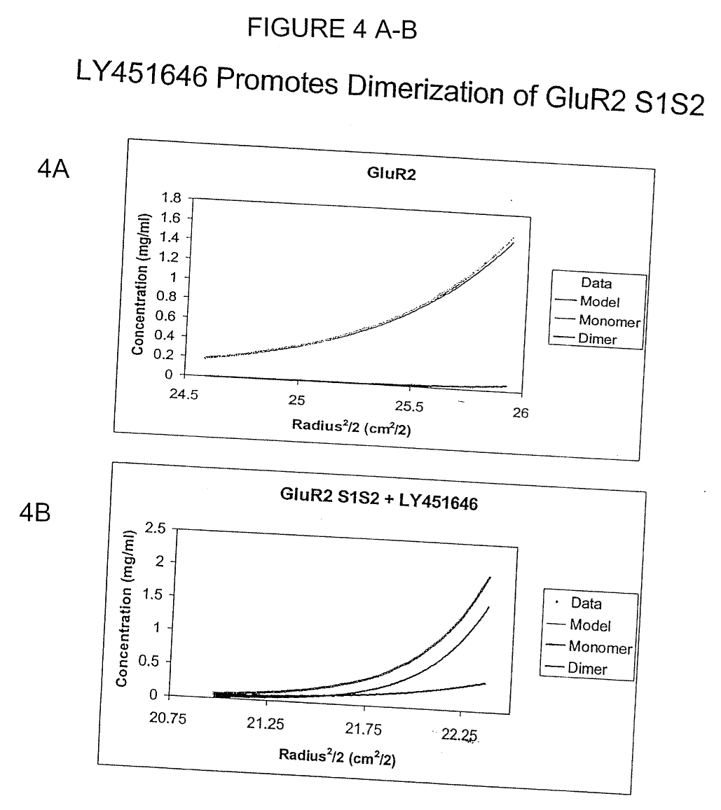

LY451646 is an agonist of GluR2. For example, in experiments using two electrode voltage clamp (“TEVC”) recording, LY451646 has been shown to potentiate glutamate currents from Delta ATD GluR2 in oocytes, as shown in FIG. 3. By showing that the modulator acts on the Delta ATD GluR2 construct, we demonstrate that the binding site of the compound is not within the ATD but rather is within the S1S2 portion of the receptor, as determined by X-ray crystallography. The top tracing represents application of glutamate alone, the bottom tracing glutamate plus LY451646. Further, as shown in FIG. 4A-B. LY451646 promotes dimerization of GluR2 S1S2. In the absence of LY451646, the amount of dimerized GluR2 barely rises above baseline (FIG. 4A). With the addition of LY451646, dimers overtake monomers (FIG. 4B).

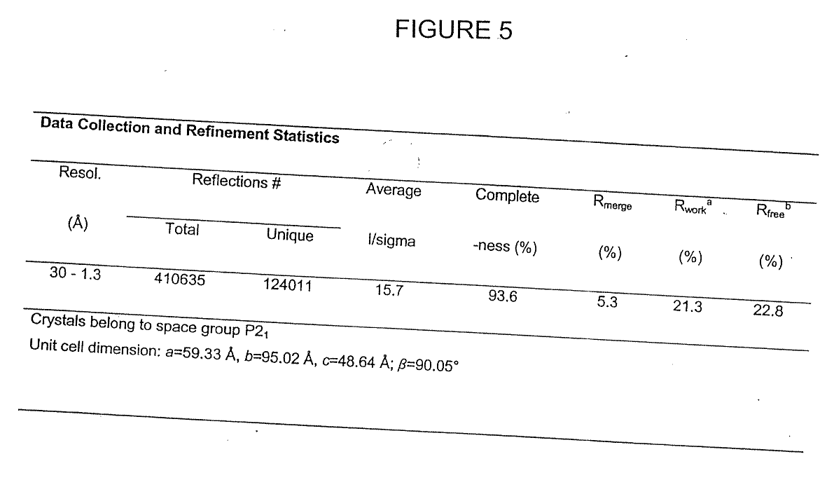

Crystals of LY451646 bound to GluR2-S1S2J were prepared in a manner analogous to the methods used in Jin et al., 2005, J. Neurosci. 25(39):9027-9036), LY451646 was used at a saturating concentration. Briefly, the crystals were grown at 4° C. by vapor diffusion and each drop contained a 1:1 ratio of reservoir solution, which for LY451646, contained 10 mM kainate, 8%-15% PEG1450, and 0.1 M NaOAc at pH 5.0. The resulting crystals were soaked in the crystallization buffer, supplemented with LY451646, and 12-16% glycerol before flash cooling in liquid nitrogen. The data set was collected at 110 K at the NSLS X4A beam line, and was processed with the HKL suite of programs. The obtained coordinates for the LY451646/GluR2-S1S2J structure are attached hereto as Appendix A. The LY451646/GluR2-S1S2J crystals belonged to the P2₁ space group. Data collection and refinement statistics for the crystallographic study are presented in FIG. 5.

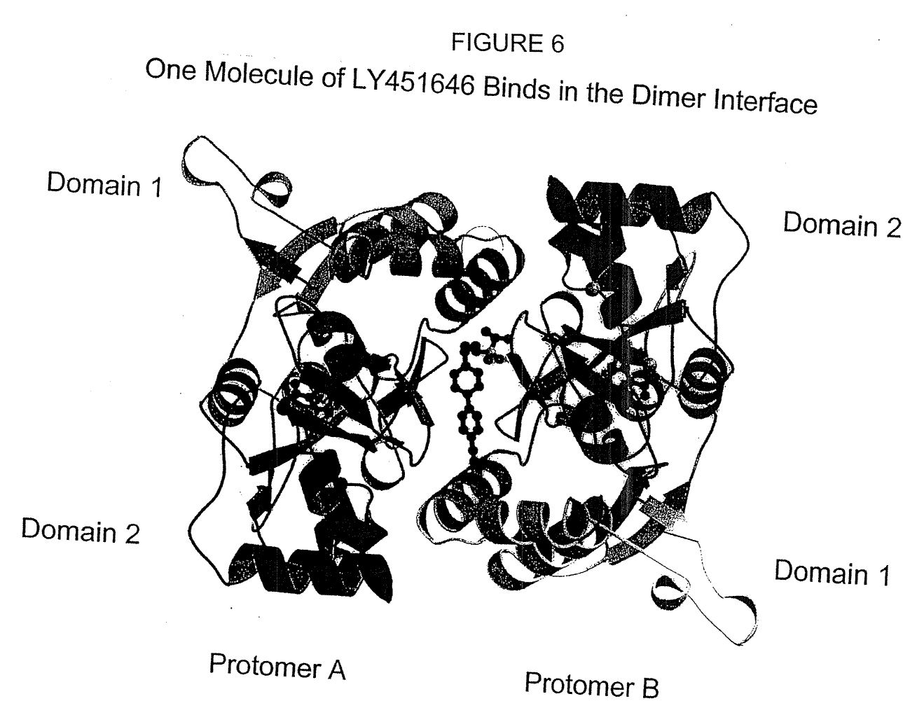

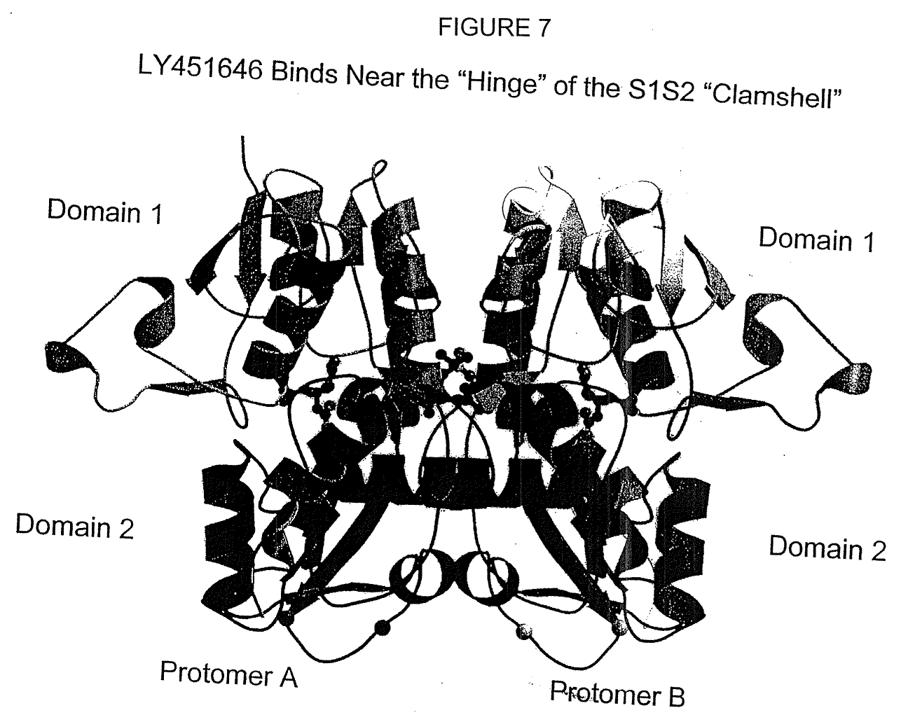

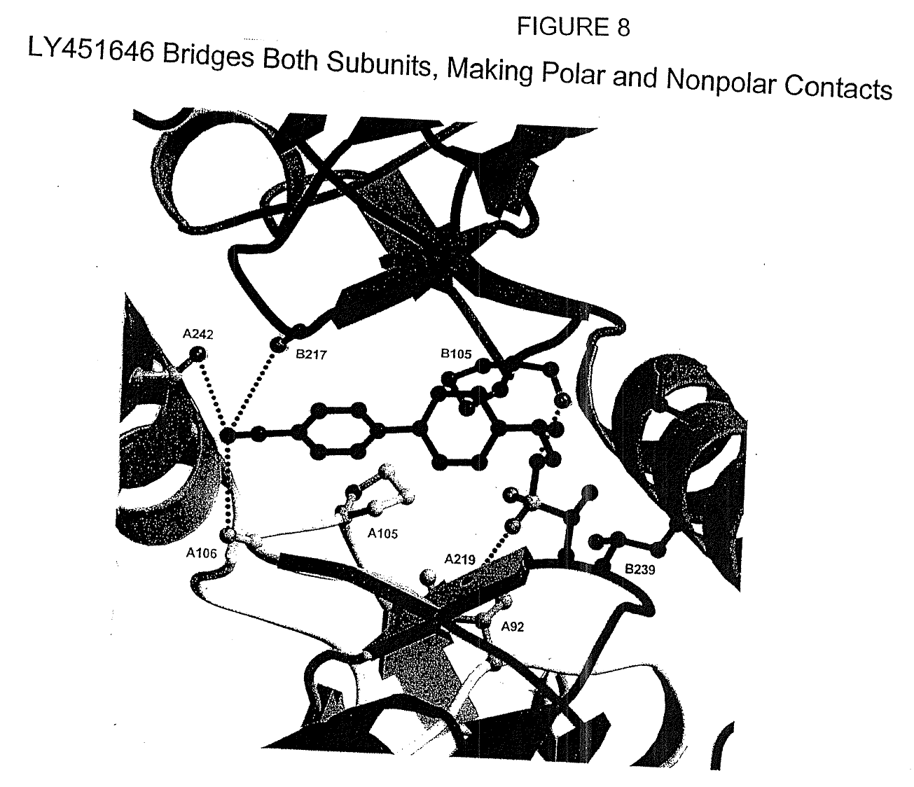

The structural data indicated that LY451646 binds at the interface between domains 1 and 2 (FIG. 6) near the “hinge” of the S1S2 “clamshell” (FIG. 7). The ligand bridges both subunits, binding via polar as well as non-polar interactions (FIG. 8). In particular, The nitrogen of the terminal nitrile group interacts with Phe_{A}106, Ser_{A}242, and Ser_{B}217, wherein the “A” designates protomer A, and “B” designates protomer B, and each residue corresponds to the identifiers and residue numbers provided in the X-ray structure provided in Appendix A. It is noted that the X-ray structure provided in Appendix A is a fragment of the full length receptor, and accordingly the residue numbers do not necessarily correspond to the full length receptor. LY451646 also interacts with Pro_{B}105, Pro_{A}105, Gly_{A}219, Ile_{A}92, and Leu_{B}239.

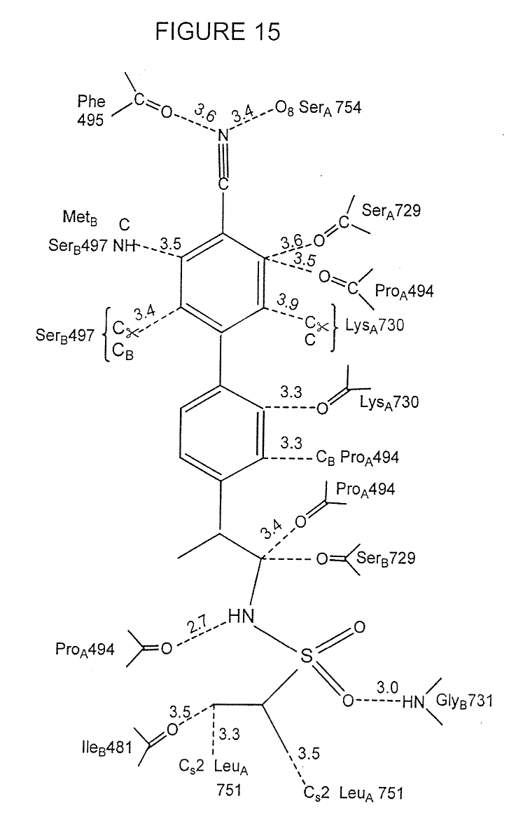

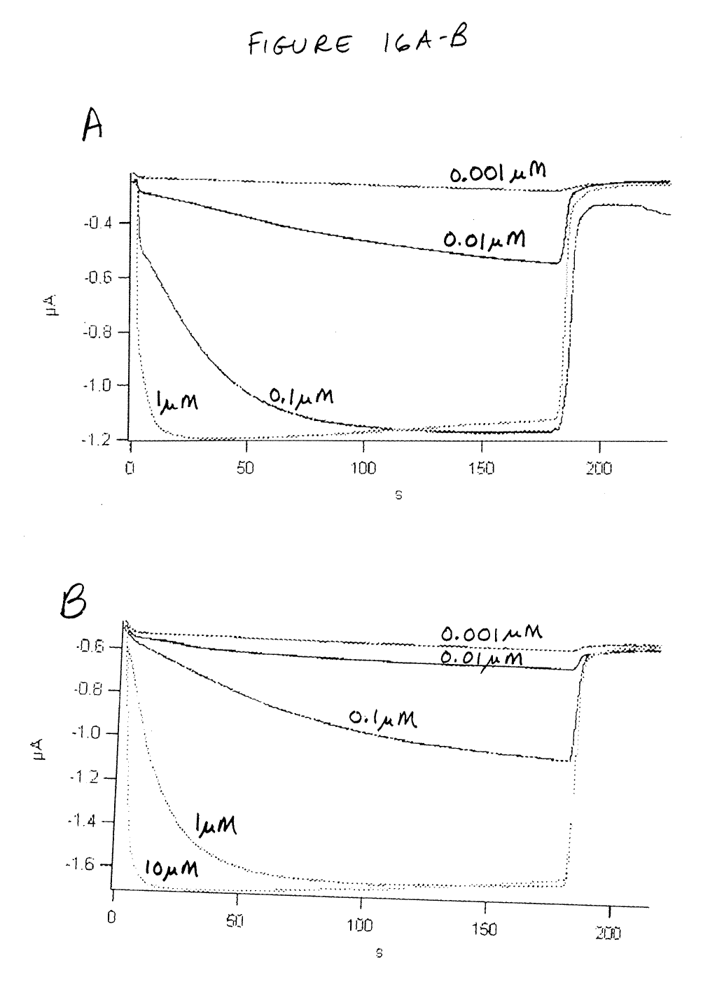

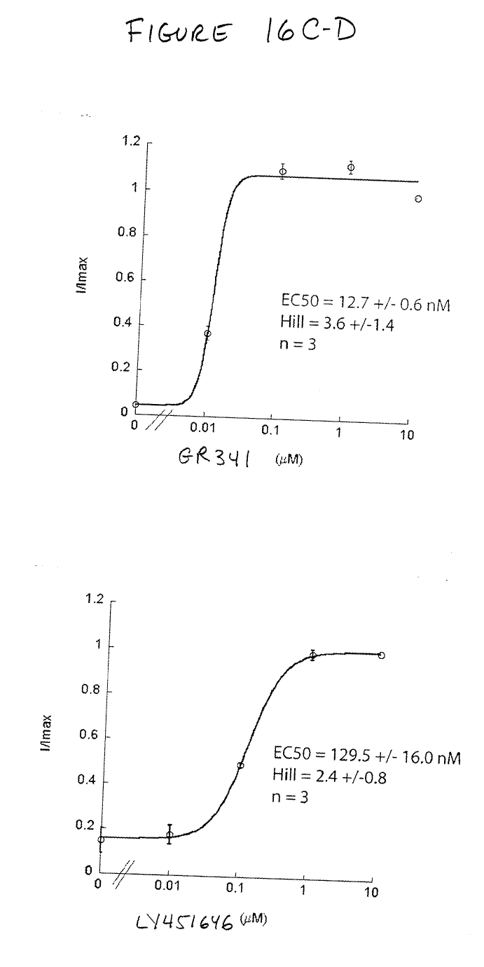

For comparison, LY404187 also interacts at the interface between domains 1 and 2 near the “hinge” of the S1S2 “clamshell” (FIG. 15). In particular, the nitrogen of the terminal nitrile group interacts with the O_{δ} atom of Ser_{A}754 and the carbonyl group of Phe495, the nitrogen in the sulfonamide bond interacts with the carbonyl group of Pro_{A}494, and the oxygen of the sulfonyl group interacts with the nitrogen of Gly_{B}731. The carbons of the isopropyl group adjacent to the sulfonyl group interact with the carbonyl of Ile_{B}481 and/or the C_{δ}2 of Leu_{A}751. The carbon atom adjacent to the nitrogen in the sulfonamide bond interacts with the carbonyl groups of Pro_{A}494 and/or Ser_{B}729. The carbon atoms in the phenyl group adjacent to the nitrile group interact with the carbonyl groups of Ser_{A}729 and Pro_{A}494, the C_{α} and/or C atom of Lys_{A}730, C_{α} and/or C atom of Ser_{B}497, or the nitrogen in Ser_{B}497 and the C atom of the Met_{B}. The carbon atoms of the remaining phenyl group interact with the carbonyl group of Lys_{A}730 or the C_{B} atom of Pro_{A}494. These interactions can be seen in the schematic depicted in FIG. 15.

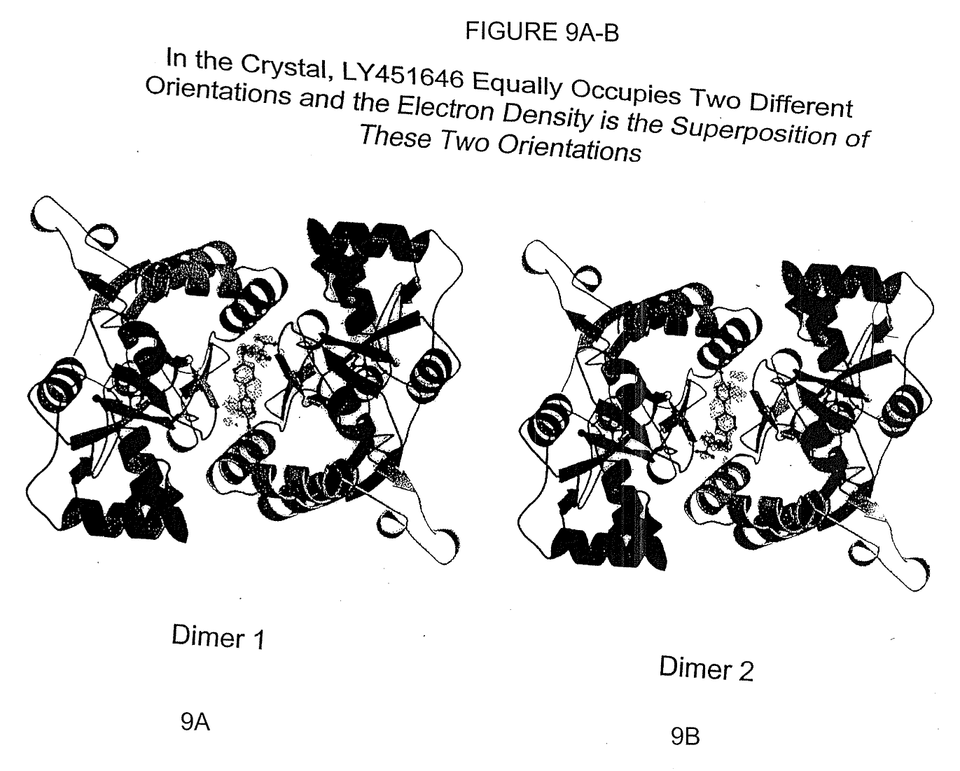

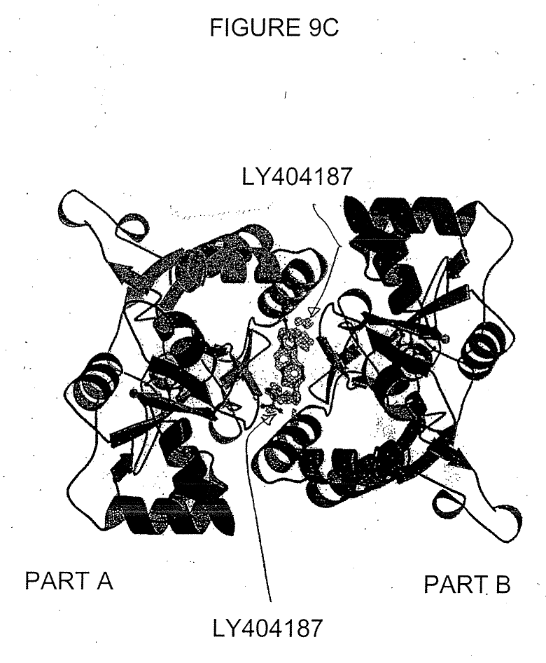

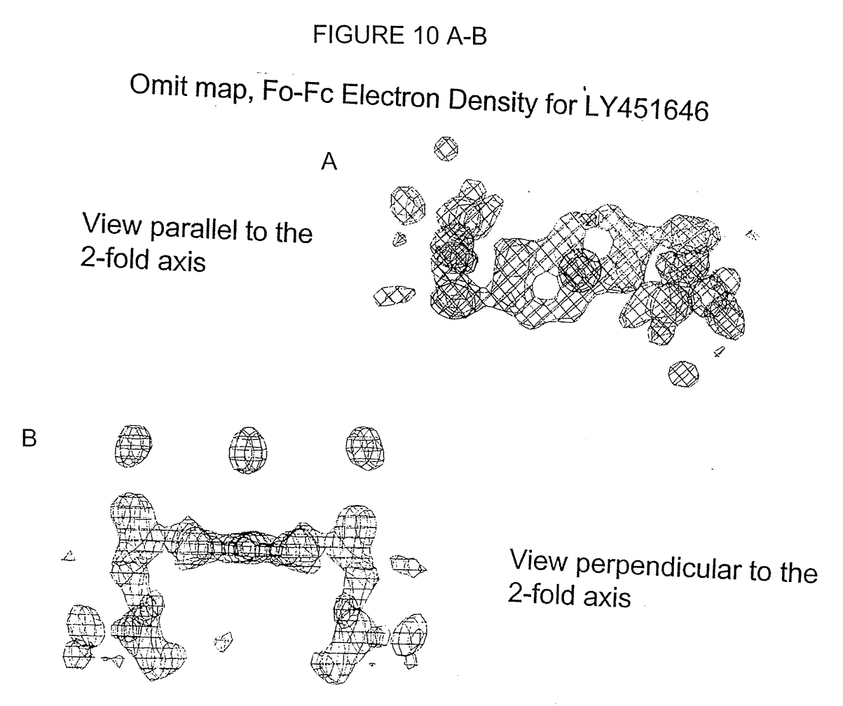

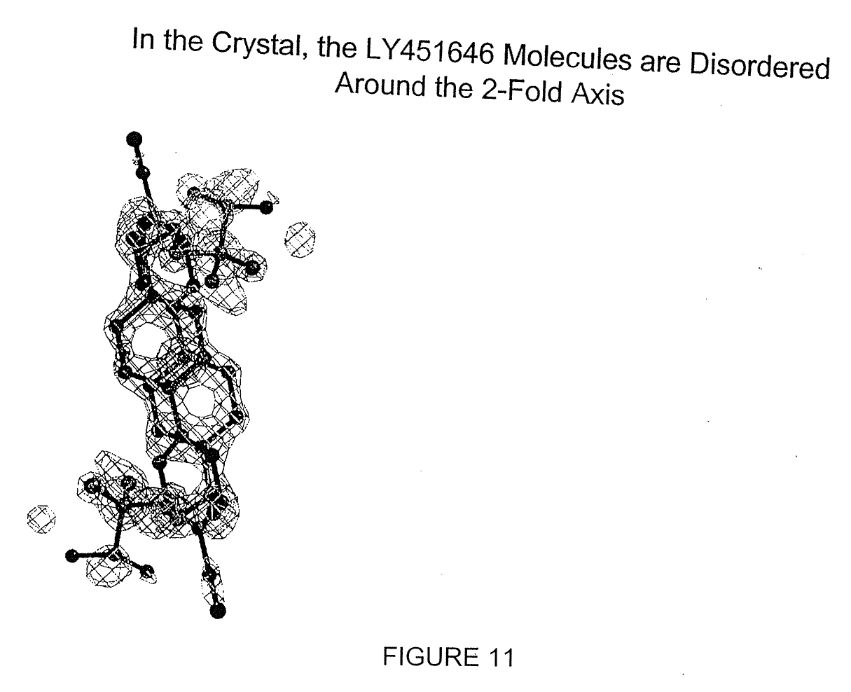

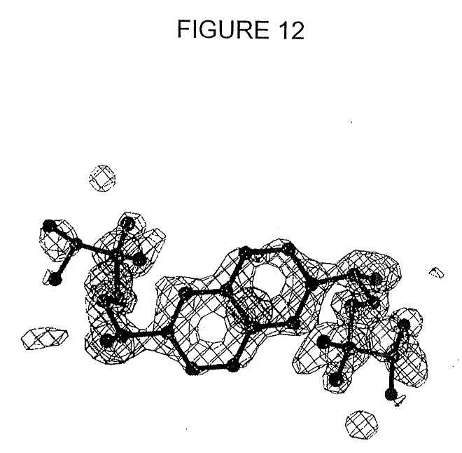

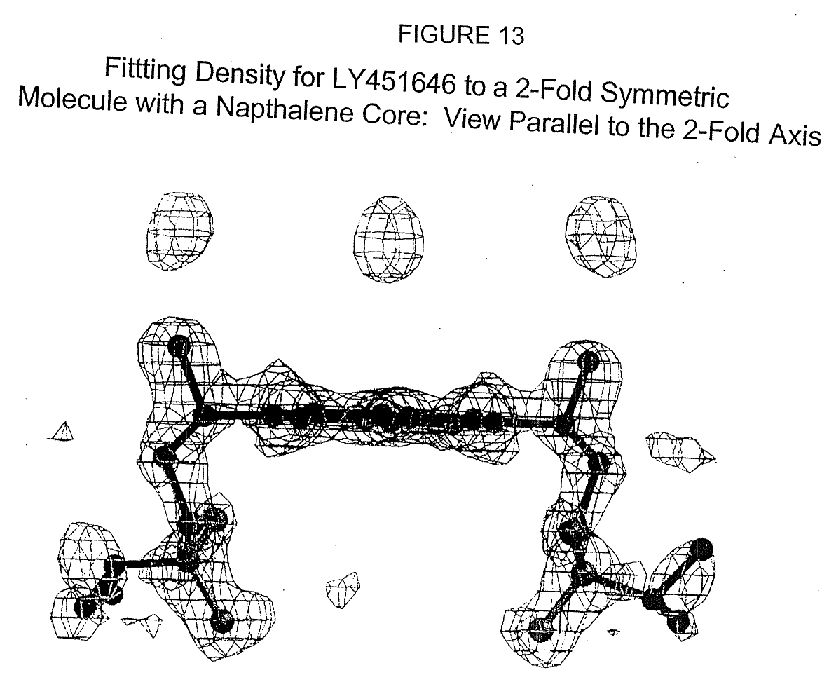

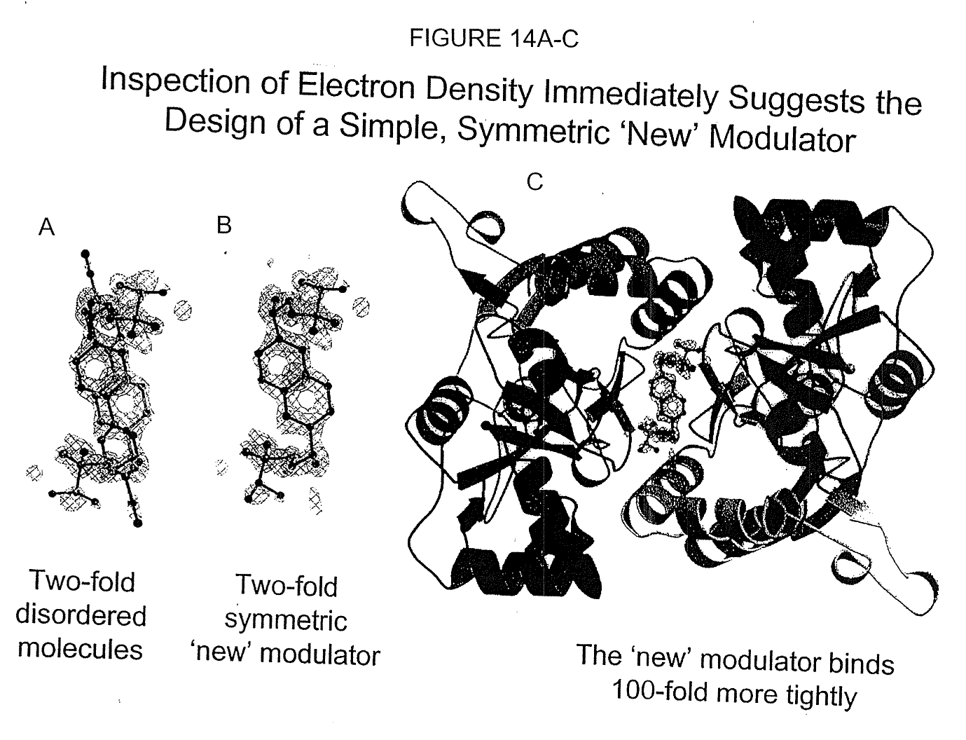

Interestingly, it has been found that the electron density of LY451646, as bound to GluR2(S1S2J), corresponds to a superposition of predicted electron densities for LY451646 bound in two overlapping orientations; the alternative orientations are depicted in FIG. 9A-B. FIG. 10A-B shows the electron densities of the ligand in a view parallel to the 2-fold axis (FIG. 10A) or perpendicular to the 2-fold axis (FIG. 10B). FIG. 11 depicts the electron density observed with structural drawings of LY451646 in the alternative orientations.

### 7. EXAMPLE 2

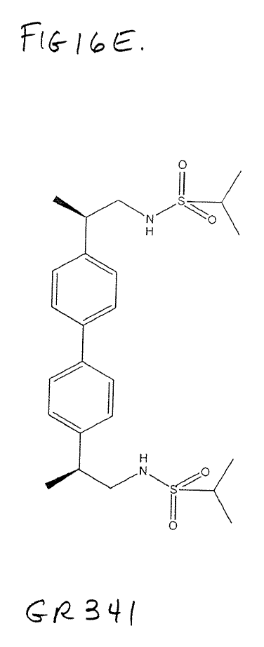

Electrophysiologic studies were performed comparing GR341 (formula 5) with LY451646 in cells expressing the GluR2 receptor, with results shown in FIG. 16A-D. GR341 was observed to have an EC₅₀ of 12.7±0.6 nM, and therefore was more potent than LY451646, which was observed to have an EC₅₀ of 129.5±16.0 nM.

### 8. EXAMPLE 3

Mice (male, 8 weeks of age; 129S6/SvEv from Taconic) were housed in groups of five for one week. Animals were injected (i.p.) with either vehicle (diluent:saline at a ratio of 3:50 v/v), or one of two doses of GR341 (1 mg/kg and 3 mg/kg). After 15 minutes, animals were injected (i.p.) either with saline or Amphetamine at 3 mg/kg. Mice were held individually without food or water for an additional 15 minutes and then placed in Plexiglas activity chambers (model ENV-520; Med Associates, St. Albans, Vt.) (43.2 cm long×43.2 cm wide×30.5 cm high) to assess locomotor activity. Mice were placed into the center of the open field, and activity was recorded for 30 minutes with subtotals for 5-minute increments. Testing was performed in darkness to minimize anxiety.

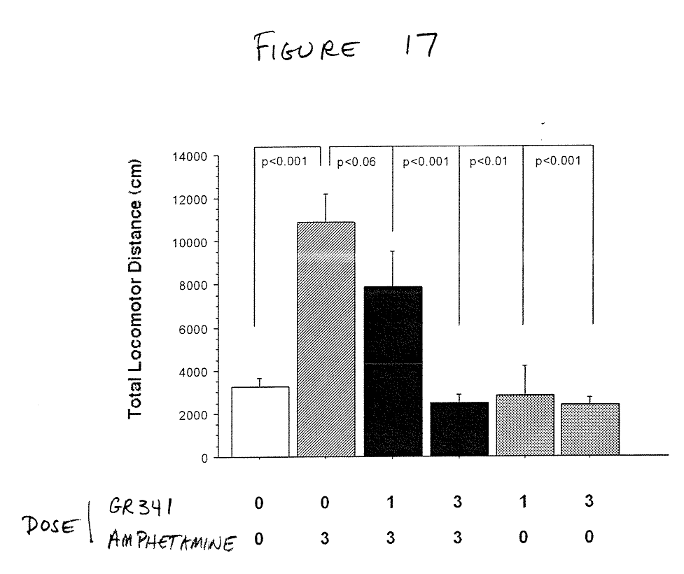

As shown in FIG. 17, each dose of GR341 inhibited amphetamine-induced locomotor activity. There was no significant effect of either dose of GR341 alone on locomotor activity.

Various patents, patent applications, and other publications are cited herein, the contents of which are hereby incorporated by reference in their entireties.

APPENDIX A

| REMARK | coordinates from minimization refinement | |
|---|---|---|
| REMARK | refinemnet resolution: 10.0-1.3 A | |
| REMARK | starting | r= 0.2169 free_r = 0.2298 |
| REMARK | final | r= 0.2141 free_r = 0.2277 |

| REMARK | rmsd bonds= 0.005543 rmsd angles= 1.38837 | |
|---|---|---|
| REMARK | wa = 0.261266 | |
| REMARK | target= mlf cycles= 1 steps = 100 | |
| REMARK | sg = P2(1) a= 59.330 b= 95.018 c= 48.635 alpha= 90.0 beta= 90.004 | |
| gamma= | 90.0 | |
| REMARK | parameter file 1 : CNS_TOPPAR: protein_rep.param | |
| REMARK | parameter file 2 : kai.par | |
| REMARK | parameter file 3 : ly_1.par | |
| REMARK | parameter file 4 : CNS_TOPPAR: water_rep.param | |
| REMARK | molecular structure file: nska1_ly12.mtf | |
| REMARK | input coordinates: nska1_ly12.pdb | |
| REMARK | reflection file = nska1_p21freeR.hkl | |
| REMARK | ncs = none | |
| REMARK | B-correction resolution: 6.0-1.3 | |
| REMARK | initial B-factor correction applied to fobs: | |
| REMARK | B11= 2.006 B22= −1.594 B33= −0.413 | |
| REMARK | B12= 0.000 B13= 0.000 B23= 0.000 | |
| REMARK | B-factor correction applied to coordinate array B: 0.032 | |
| REMARK | bulk solvent: density level = 0.497227 e/A\hat{ }3, B-factor = 61.6121 A\hat{ }2 | |
| REMARK | reflections with \|Fobs\|/sigma_F <0.0 rejected | |
| REMARK | reflections with \|Fobs\| >10000 * rms(Fobs) rejected | |
| REMARK | theoretical total number of refl. in resol. range: 131701(100.0%) | |
| REMARK | number of unobserved reflections (no entry or \|F\| = 0): | 12652 ( |
| 9.6% ) | | |
| REMARK | number of reflections rejected: | 0 ( |
| 0.0% ) | | |
| REMARK | total number of reflections used: | 119049 ( |
| 90.4% ) | | |
| REMARK | number of reflections in working set: | 107100 ( |
| 81.3% ) | | |
| REMARK | number of reflections in test set: | 11949 ( |
| 9.1% ) | | |
| CRYST1 | 59.330 95.018 48.635 90.00 90.00 90.00 P 21 | |
| REMARK | FILENAME = “min_ly12.pdb” | |
| REMARK | DATE: 01-Aug-02 13:52:05 created by user: lsong | |
| REMARK | VERSION: 1.0 | |

| | | | | | | | | | | | |
|---|---|---|---|---|---|---|---|---|---|---|---|
| ATOM | 1 | C | GLY | A | 4 | 22.575 | 72.784 | 45.568 | 1.00 | 26.51 | A |
| ATOM | 2 | O | GLY | A | 4 | 23.484 | 72.192 | 44.987 | 1.00 | 27.75 | A |
| ATOM | 3 | N | GLY | A | 4 | 23.280 | 71.627 | 47.677 | 1.00 | 29.13 | A |
| ATOM | 4 | CA | GLY | A | 4 | 22.412 | 72.676 | 47.072 | 1.00 | 27.67 | A |
| ATOM | 5 | N | THR | A | 5 | 21.688 | 73.543 | 44.934 | 1.00 | 23.94 | A |
| ATOM | 6 | CA | THR | A | 5 | 21.739 | 73.732 | 43.492 | 1.00 | 20.81 | A |
| ATOM | 7 | CB | THR | A | 5 | 20.488 | 74.481 | 42.991 | 1.00 | 20.59 | A |
| ATOM | 8 | OG1 | THR | A | 5 | 19.325 | 73.683 | 43.246 | 1.00 | 21.84 | A |
| ATOM | 9 | CG2 | THR | A | 5 | 20.587 | 74.760 | 41.497 | 1.00 | 20.68 | A |
| ATOM | 10 | C | THR | A | 5 | 22.989 | 74.515 | 43.098 | 1.00 | 18.01 | A |
| ATOM | 11 | O | THR | A | 5 | 23.277 | 75.571 | 43.659 | 1.00 | 19.75 | A |
| ATOM | 12 | N | VAL | A | 6 | 23.730 | 73.978 | 42.136 | 1.00 | 15.06 | A |
| ATOM | 13 | CA | VAL | A | 6 | 24.950 | 74.610 | 41.655 | 1.00 | 14.03 | A |
| ATOM | 14 | CB | VAL | A | 6 | 25.810 | 73.591 | 40.876 | 1.00 | 12.06 | A |
| ATOM | 15 | CG1 | VAL | A | 6 | 26.974 | 74.292 | 40.183 | 1.00 | 13.80 | A |
| ATOM | 16 | CG2 | VAL | A | 6 | 26.324 | 72.522 | 41.831 | 1.00 | 13.53 | A |
| ATOM | 17 | C | VAL | A | 6 | 24.621 | 75.794 | 40.757 | 1.00 | 12.40 | A |
| ATOM | 18 | O | VAL | A | 6 | 23.814 | 75.683 | 39.836 | 1.00 | 13.80 | A |
| ATOM | 19 | N | VAL | A | 7 | 25.239 | 76.935 | 41.044 | 1.00 | 10.97 | A |
| ATOM | 20 | CA | VAL | A | 7 | 25.020 | 78.131 | 40.248 | 1.00 | 11.86 | A |
| ATOM | 21 | CB | VAL | A | 7 | 25.117 | 79.406 | 41.104 | 1.00 | 12.22 | A |
| ATOM | 22 | CG1 | VAL | A | 7 | 24.985 | 80.637 | 40.215 | 1.00 | 13.54 | A |
| ATOM | 23 | CG2 | VAL | A | 7 | 24.022 | 79.395 | 42.163 | 1.00 | 14.32 | A |
| ATOM | 24 | C | VAL | A | 7 | 26.053 | 78.201 | 39.132 | 1.00 | 9.54 | A |
| ATOM | 25 | O | VAL | A | 7 | 27.246 | 78.376 | 39.379 | 1.00 | 10.52 | A |
| ATOM | 26 | N | VAL | A | 8 | 25.580 | 78.054 | 37.902 | 1.00 | 10.17 | A |
| ATOM | 27 | CA | VAL | A | 8 | 26.455 | 78.107 | 36.746 | 1.00 | 10.75 | A |
| ATOM | 28 | CB | VAL | A | 8 | 26.038 | 77.067 | 35.678 | 1.00 | 11.39 | A |
| ATOM | 29 | CG1 | VAL | A | 8 | 26.953 | 77.164 | 34.452 | 1.00 | 12.01 | A |
| ATOM | 30 | CG2 | VAL | A | 8 | 26.080 | 75.670 | 36.274 | 1.00 | 10.32 | A |
| ATOM | 31 | C | VAL | A | 8 | 26.357 | 79.482 | 36.120 | 1.00 | 10.46 | A |
| ATOM | 32 | O | VAL | A | 8 | 25.255 | 79.992 | 35.911 | 1.00 | 10.77 | A |
| ATOM | 33 | N | THR | A | 9 | 27.500 | 80.105 | 35.860 | 1.00 | 9.23 | A |
| ATOM | 34 | CA | THR | A | 9 | 27.478 | 81.390 | 35.189 | 1.00 | 8.97 | A |
| ATOM | 35 | CB | THR | A | 9 | 28.420 | 82.436 | 35.838 | 1.00 | 10.14 | A |
| ATOM | 36 | OG1 | THR | A | 9 | 28.332 | 83.665 | 35.099 | 1.00 | 10.93 | A |
| ATOM | 37 | CG2 | THR | A | 9 | 29.867 | 81.960 | 35.831 | 1.00 | 11.82 | A |
| ATOM | 38 | C | THR | A | 9 | 27.926 | 81.117 | 33.760 | 1.00 | 8.83 | A |
| ATOM | 39 | O | THR | A | 9 | 28.849 | 80.326 | 33.511 | 1.00 | 9.36 | A |
| ATOM | 40 | N | THR | A | 10 | 27.229 | 81.731 | 32.816 | 1.00 | 8.91 | A |
| ATOM | 41 | CA | THR | A | 10 | 27.562 | 81.572 | 31.417 | 1.00 | 8.91 | A |
| ATOM | 42 | CB | THR | A | 10 | 26.830 | 80.351 | 30.792 | 1.00 | 9.80 | A |
| ATOM | 43 | OG1 | THR | A | 10 | 27.387 | 80.067 | 29.502 | 1.00 | 9.14 | A |
| ATOM | 44 | CG2 | THR | A | 10 | 25.342 | 80.602 | 30.668 | 1.00 | 10.14 | A |
| ATOM | 45 | C | THR | A | 10 | 27.218 | 82.883 | 30.724 | 1.00 | 9.78 | A |
| ATOM | 46 | O | THR | A | 10 | 26.866 | 83.864 | 31.384 | 1.00 | 8.65 | A |
| ATOM | 47 | N | ILE | A | 11 | 27.309 | 82.912 | 29.404 | 1.00 | 9.26 | A |
| ATOM | 48 | CA | ILE | A | 11 | 27.068 | 84.147 | 28.675 | 1.00 | 9.83 | A |
| ATOM | 49 | CB | ILE | A | 11 | 28.415 | 84.918 | 28.539 | 1.00 | 9.13 | A |
| ATOM | 50 | CG2 | ILE | A | 11 | 29.381 | 84.119 | 27.675 | 1.00 | 10.83 | A |
| ATOM | 51 | CG1 | ILE | A | 11 | 28.196 | 86.325 | 27.985 | 1.00 | 10.63 | A |
| ATOM | 52 | CD1 | ILE | A | 11 | 29.425 | 87.221 | 28.126 | 1.00 | 12.27 | A |
| ATOM | 53 | C | ILE | A | 11 | 26.476 | 83.819 | 27.312 | 1.00 | 9.66 | A |
| ATOM | 54 | O | ILE | A | 11 | 26.716 | 82.737 | 26.771 | 1.00 | 9.86 | A |
| ATOM | 55 | N | LEU | A | 12 | 25.690 | 84.741 | 26.762 | 1.00 | 10.35 | A |
| ATOM | 56 | CA | LEU | A | 12 | 25.083 | 84.521 | 25.460 | 1.00 | 12.08 | A |
| ATOM | 57 | CB | LEU | A | 12 | 23.825 | 85.391 | 25.334 | 1.00 | 11.83 | A |
| ATOM | 58 | CG | LEU | A | 12 | 22.744 | 85.141 | 26.394 | 1.00 | 11.57 | A |
| ATOM | 59 | CD1 | LEU | A | 12 | 21.572 | 86.085 | 26.158 | 1.00 | 14.26 | A |
| ATOM | 60 | CD2 | LEU | A | 12 | 22.279 | 83.691 | 26.351 | 1.00 | 13.48 | A |
| ATOM | 61 | C | LEU | A | 12 | 26.116 | 84.775 | 24.367 | 1.00 | 12.55 | A |
| ATOM | 62 | O | LEU | A | 12 | 26.608 | 85.894 | 24.206 | 1.00 | 15.65 | A |
| ATOM | 63 | N | GLU | A | 13 | 26.453 | 83.719 | 23.634 | 1.00 | 11.29 | A |
| ATOM | 64 | CA | GLU | A | 13 | 27.416 | 83.777 | 22.534 | 1.00 | 12.00 | A |
| ATOM | 65 | CB | GLU | A | 13 | 28.849 | 83.782 | 23.093 | 1.00 | 11.61 | A |
| ATOM | 66 | CG | GLU | A | 13 | 29.952 | 83.756 | 22.018 | 1.00 | 12.91 | A |
| ATOM | 67 | CD | GLU | A | 13 | 30.165 | 85.099 | 21.328 | 1.00 | 14.45 | A |
| ATOM | 68 | OE1 | GLU | A | 13 | 30.740 | 85.111 | 20.218 | 1.00 | 14.76 | A |
| ATOM | 69 | OE2 | GLU | A | 13 | 29.776 | 86.143 | 21.892 | 1.00 | 14.04 | A |
| ATOM | 70 | C | GLU | A | 13 | 27.114 | 82.557 | 21.668 | 1.00 | 11.99 | A |
| ATOM | 71 | O | GLU | A | 13 | 27.210 | 81.426 | 22.137 | 1.00 | 12.31 | A |
| ATOM | 72 | N | SER | A | 14 | 26.743 | 82.784 | 20.412 | 1.00 | 11.74 | A |
| ATOM | 73 | CA | SER | A | 14 | 26.419 | 81.690 | 19.499 | 1.00 | 10.92 | A |
| ATOM | 74 | CB | SER | A | 14 | 25.658 | 82.255 | 18.300 | 1.00 | 12.79 | A |
| ATOM | 75 | OG | SER | A | 14 | 24.496 | 82.939 | 18.710 | 1.00 | 15.59 | A |
| ATOM | 76 | C | SER | A | 14 | 27.682 | 80.950 | 19.082 | 1.00 | 11.02 | A |
| ATOM | 77 | O | SER | A | 14 | 28.713 | 81.570 | 18.832 | 1.00 | 13.24 | A |
| ATOM | 78 | N | PRO | A | 15 | 27.618 | 79.615 | 18.969 | 1.00 | 10.96 | A |
| ATOM | 79 | CD | PRO | A | 15 | 28.696 | 78.928 | 18.235 | 1.00 | 10.29 | A |
| ATOM | 80 | CA | PRO | A | 15 | 26.493 | 78.705 | 19.193 | 1.00 | 11.07 | A |
| ATOM | 81 | CB | PRO | A | 15 | 26.640 | 77.742 | 18.022 | 1.00 | 10.06 | A |
| ATOM | 82 | CG | PRO | A | 15 | 28.133 | 77.463 | 18.043 | 1.00 | 11.18 | A |
| ATOM | 83 | C | PRO | A | 15 | 26.597 | 78.055 | 20.571 | 1.00 | 10.83 | A |
| ATOM | 84 | O | PRO | A | 15 | 26.046 | 76.981 | 20.794 | 1.00 | 10.59 | A |
| ATOM | 85 | N | TYR | A | 16 | 27.305 | 78.709 | 21.489 | 1.00 | 10.45 | A |
| ATOM | 86 | CA | TYR | A | 16 | 27.517 | 78.184 | 22.840 | 1.00 | 9.96 | A |
| ATOM | 87 | CB | TYR | A | 16 | 28.710 | 78.909 | 23.464 | 1.00 | 11.23 | A |
| ATOM | 88 | CG | TYR | A | 16 | 29.952 | 78.896 | 22.606 | 1.00 | 9.43 | A |
| ATOM | 89 | CD1 | TYR | A | 16 | 30.820 | 77.801 | 22.598 | 1.00 | 9.41 | A |
| ATOM | 90 | CE1 | TYR | A | 16 | 31.967 | 77.799 | 21.787 | 1.00 | 8.96 | A |
| ATOM | 91 | CD2 | TYR | A | 16 | 30.253 | 79.980 | 21.789 | 1.00 | 10.09 | A |
| ATOM | 92 | CE2 | TYR | A | 16 | 31.387 | 79.986 | 20.980 | 1.00 | 10.47 | A |
| ATOM | 93 | CZ | TYR | A | 16 | 32.237 | 78.899 | 20.984 | 1.00 | 10.17 | A |
| ATOM | 94 | OH | TYR | A | 16 | 33.363 | 78.920 | 20.190 | 1.00 | 11.52 | A |
| ATOM | 95 | C | TYR | A | 16 | 26.275 | 78.266 | 23.712 | 1.00 | 10.43 | A |
| ATOM | 96 | O | TYR | A | 16 | 25.892 | 77.283 | 24.342 | 1.00 | 10.31 | A |
| ATOM | 97 | N | VAL | A | 17 | 25.674 | 79.450 | 23.773 | 1.00 | 10.32 | A |
| ATOM | 98 | CA | VAL | A | 17 | 24.465 | 79.671 | 24.557 | 1.00 | 10.55 | A |
| ATOM | 99 | CB | VAL | A | 17 | 24.780 | 80.166 | 25.990 | 1.00 | 9.76 | A |
| ATOM | 100 | CG1 | VAL | A | 17 | 23.489 | 80.287 | 26.792 | 1.00 | 11.30 | A |
| ATOM | 101 | CG2 | VAL | A | 17 | 25.739 | 79.210 | 26.680 | 1.00 | 11.77 | A |
| ATOM | 102 | C | VAL | A | 17 | 23.634 | 80.728 | 23.850 | 1.00 | 10.95 | A |
| ATOM | 103 | O | VAL | A | 17 | 24.127 | 81.814 | 23.543 | 1.00 | 11.05 | A |
| ATOM | 104 | N | MET | A | 18 | 22.377 | 80.390 | 23.586 | 1.00 | 10.77 | A |
| ATOM | 105 | CA | MET | A | 18 | 21.441 | 81.279 | 22.913 | 1.00 | 12.48 | A |
| ATOM | 106 | CB | MET | A | 18 | 21.409 | 80.927 | 21.417 | 1.00 | 15.54 | A |
| ATOM | 107 | CG | MET | A | 18 | 22.783 | 80.824 | 20.761 | 1.00 | 18.73 | A |
| ATOM | 108 | SD | MET | A | 18 | 22.773 | 79.981 | 19.154 | 1.00 | 22.12 | A |
| ATOM | 109 | CE | MET | A | 18 | 22.727 | 78.246 | 19.684 | 1.00 | 15.92 | A |
| ATOM | 110 | C | MET | A | 18 | 20.082 | 81.091 | 23.579 | 1.00 | 12.27 | A |
| ATOM | 111 | O | MET | A | 18 | 19.805 | 80.030 | 24.140 | 1.00 | 12.26 | A |
| ATOM | 112 | N | MET | A | 19 | 19.239 | 82.117 | 23.541 | 1.00 | 12.48 | A |
| ATOM | 113 | CA | MET | A | 19 | 17.904 | 81.974 | 24.101 | 1.00 | 12.14 | A |
| ATOM | 114 | CB | MET | A | 19 | 17.347 | 83.338 | 24.535 | 1.00 | 15.09 | A |
| ATOM | 115 | CG | MET | A | 19 | 17.994 | 83.934 | 25.770 | 1.00 | 13.78 | A |
| ATOM | 116 | SD | MET | A | 19 | 16.874 | 85.087 | 26.613 | 1.00 | 15.38 | A |
| ATOM | 117 | CE | MET | A | 19 | 16.000 | 83.961 | 27.704 | 1.00 | 16.72 | A |
| ATOM | 118 | C | MET | A | 19 | 17.069 | 81.322 | 23.006 | 1.00 | 13.23 | A |
| ATOM | 119 | O | MET | A | 19 | 17.135 | 81.735 | 21.848 | 1.00 | 14.65 | A |
| ATOM | 120 | N | LYS | A | 20 | 16.296 | 80.301 | 23.360 | 1.00 | 13.52 | A |
| ATOM | 121 | CA | LYS | A | 20 | 15.447 | 79.636 | 22.379 | 1.00 | 14.94 | A |
| ATOM | 122 | CB | LYS | A | 20 | 14.725 | 78.453 | 23.029 | 1.00 | 13.74 | A |
| ATOM | 123 | CG | LYS | A | 20 | 15.604 | 77.280 | 23.438 | 1.00 | 15.44 | A |
| ATOM | 124 | CD | LYS | A | 20 | 14.750 | 76.205 | 24.096 | 1.00 | 16.23 | A |
| ATOM | 125 | CE | LYS | A | 20 | 15.567 | 75.003 | 24.533 | 1.00 | 14.95 | A |
| ATOM | 126 | NZ | LYS | A | 20 | 14.725 | 74.058 | 25.322 | 1.00 | 16.86 | A |
| ATOM | 127 | C | LYS | A | 20 | 14.474 | 80.678 | 21.842 | 1.00 | 16.34 | A |
| ATOM | 128 | O | LYS | A | 20 | 14.138 | 81.639 | 22.535 | 1.00 | 15.24 | A |
| ATOM | 129 | N | ALA | A | 21 | 14.012 | 80.483 | 20.613 | 1.00 | 17.51 | A |
| ATOM | 130 | CA | ALA | A | 21 | 13.072 | 81.412 | 20.004 | 1.00 | 18.87 | A |
| ATOM | 131 | CB | ALA | A | 21 | 12.758 | 80.972 | 18.578 | 1.00 | 20.19 | A |
| ATOM | 132 | C | ALA | A | 21 | 11.790 | 81.484 | 20.832 | 1.00 | 18.60 | A |
| ATOM | 133 | O | ALA | A | 21 | 11.182 | 82.548 | 20.963 | 1.00 | 19.20 | A |
| ATOM | 134 | N | ASN | A | 22 | 11.401 | 80.350 | 21.405 | 1.00 | 17.71 | A |
| ATOM | 135 | CA | ASN | A | 22 | 10.187 | 80.263 | 22.213 | 1.00 | 18.07 | A |
| ATOM | 136 | CB | ASN | A | 22 | 9.431 | 78.992 | 21.812 | 1.00 | 18.69 | A |
| ATOM | 137 | CG | ASN | A | 22 | 10.150 | 77.726 | 22.242 | 1.00 | 20.55 | A |
| ATOM | 138 | OD1 | ASN | A | 22 | 11.365 | 77.601 | 22.079 | 1.00 | 20.26 | A |
| ATOM | 139 | ND2 | ASN | A | 22 | 9.397 | 76.773 | 22.786 | 1.00 | 22.20 | A |
| ATOM | 140 | C | ASN | A | 22 | 10.500 | 80.310 | 23.707 | 1.00 | 17.10 | A |
| ATOM | 141 | O | ASN | A | 22 | 9.757 | 79.753 | 24.518 | 1.00 | 19.00 | A |
| ATOM | 142 | N | HIS | A | 23 | 11.588 | 80.986 | 24.072 | 1.00 | 15.85 | A |
| ATOM | 143 | CA | HIS | A | 23 | 11.997 | 81.068 | 25.470 | 1.00 | 15.45 | A |
| ATOM | 144 | CB | HIS | A | 23 | 13.315 | 81.857 | 25.562 | 1.00 | 15.21 | A |
| ATOM | 145 | CG | HIS | A | 23 | 13.186 | 83.314 | 25.234 | 1.00 | 15.86 | A |
| ATOM | 146 | CD2 | HIS | A | 23 | 13.486 | 84.002 | 24.107 | 1.00 | 16.43 | A |
| ATOM | 147 | ND1 | HIS | A | 23 | 12.720 | 84.244 | 26.138 | 1.00 | 15.55 | A |
| ATOM | 148 | CE1 | HIS | A | 23 | 12.742 | 85.443 | 25.584 | 1.00 | 16.33 | A |
| ATOM | 149 | NE2 | HIS | A | 23 | 13.204 | 85.324 | 24.351 | 1.00 | 16.65 | A |
| ATOM | 150 | C | HIS | A | 23 | 10.897 | 81.598 | 26.391 | 1.00 | 15.46 | A |
| ATOM | 151 | O | HIS | A | 23 | 10.841 | 81.232 | 27.561 | 1.00 | 15.24 | A |
| ATOM | 152 | N | ALA | A | 24 | 10.014 | 82.441 | 25.863 | 1.00 | 16.95 | A |
| ATOM | 153 | CA | ALA | A | 24 | 8.932 | 83.001 | 26.670 | 1.00 | 17.83 | A |
| ATOM | 154 | CB | ALA | A | 24 | 8.183 | 84.064 | 25.872 | 1.00 | 19.08 | A |
| ATOM | 155 | C | ALA | A | 24 | 7.952 | 81.943 | 27.178 | 1.00 | 18.59 | A |
| ATOM | 156 | O | ALA | A | 24 | 7.300 | 82.136 | 28.202 | 1.00 | 18.94 | A |
| ATOM | 157 | N | MET | A | 25 | 7.848 | 80.826 | 26.465 | 1.00 | 18.33 | A |
| ATOM | 158 | CA | MET | A | 25 | 6.936 | 79.753 | 26.859 | 1.00 | 18.03 | A |
| ATOM | 159 | CB | MET | A | 25 | 6.331 | 79.113 | 25.608 | 1.00 | 20.26 | A |
| ATOM | 160 | CG | MET | A | 25 | 5.647 | 80.067 | 24.662 | 1.00 | 20.74 | A |
| ATOM | 161 | SD | MET | A | 25 | 4.819 | 79.150 | 23.355 | 1.00 | 24.57 | A |
| ATOM | 162 | CE | MET | A | 25 | 6.205 | 78.477 | 22.466 | 1.00 | 23.59 | A |
| ATOM | 163 | C | MET | A | 25 | 7.653 | 78.723 | 27.718 | 1.00 | 18.31 | A |
| ATOM | 164 | O | MET | A | 25 | 7.079 | 77.686 | 28.068 | 1.00 | 17.96 | A |
| ATOM | 165 | N | LEU | A | 26 | 8.906 | 79.009 | 28.053 | 1.00 | 17.37 | A |
| ATOM | 166 | CA | LEU | A | 26 | 9.717 | 78.091 | 28.834 | 1.00 | 16.23 | A |
| ATOM | 167 | CB | LEU | A | 26 | 10.892 | 77.641 | 27.966 | 1.00 | 15.94 | A |
| ATOM | 168 | CG | LEU | A | 26 | 10.507 | 77.102 | 26.585 | 1.00 | 18.54 | A |
| ATOM | 169 | CD1 | LEU | A | 26 | 11.757 | 76.868 | 25.756 | 1.00 | 18.19 | A |
| ATOM | 170 | CD2 | LEU | A | 26 | 9.719 | 75.812 | 26.738 | 1.00 | 17.91 | A |
| ATOM | 171 | C | LEU | A | 26 | 10.158 | 78.703 | 30.155 | 1.00 | 14.96 | A |
| ATOM | 172 | O | LEU | A | 26 | 9.945 | 79.889 | 30.414 | 1.00 | 15.22 | A |
| ATOM | 173 | N | ALA | A | 27 | 10.786 | 77.883 | 30.987 | 1.00 | 14.45 | A |
| ATOM | 174 | CA | ALA | A | 27 | 11.251 | 78.335 | 32.289 | 1.00 | 14.51 | A |
| ATOM | 175 | CB | ALA | A | 27 | 10.211 | 77.988 | 33.356 | 1.00 | 17.17 | A |
| ATOM | 176 | C | ALA | A | 27 | 12.588 | 77.705 | 32.644 | 1.00 | 14.03 | A |
| ATOM | 177 | O | ALA | A | 27 | 12.923 | 76.619 | 32.176 | 1.00 | 14.44 | A |
| ATOM | 178 | N | GLY | A | 28 | 13.357 | 78.404 | 33.469 | 1.00 | 12.71 | A |
| ATOM | 179 | CA | GLY | A | 28 | 14.641 | 77.885 | 33.907 | 1.00 | 12.56 | A |
| ATOM | 180 | C | GLY | A | 28 | 15.641 | 77.561 | 32.817 | 1.00 | 10.82 | A |
| ATOM | 181 | O | GLY | A | 28 | 15.749 | 78.276 | 31.818 | 1.00 | 10.69 | A |
| ATOM | 182 | N | ASN | A | 29 | 16.373 | 76.468 | 33.009 | 1.00 | 10.83 | A |
| ATOM | 183 | CA | ASN | A | 29 | 17.394 | 76.046 | 32.056 | 1.00 | 10.02 | A |
| ATOM | 184 | CB | ASN | A | 29 | 18.108 | 74.797 | 32.591 | 1.00 | 12.03 | A |
| ATOM | 185 | CG | ASN | A | 29 | 18.886 | 75.050 | 33.875 | 1.00 | 12.74 | A |
| ATOM | 186 | OD1 | ASN | A | 29 | 19.350 | 74.104 | 34.519 | 1.00 | 14.94 | A |
| ATOM | 187 | ND2 | ASN | A | 29 | 19.048 | 76.314 | 34.245 | 1.00 | 10.03 | A |
| ATOM | 188 | C | ASN | A | 29 | 16.823 | 75.817 | 30.659 | 1.00 | 10.58 | A |
| ATOM | 189 | O | ASN | A | 29 | 17.539 | 75.941 | 29.670 | 1.00 | 11.93 | A |
| ATOM | 190 | N | GLU | A | 30 | 15.540 | 75.468 | 30.585 | 1.00 | 10.30 | A |
| ATOM | 191 | CA | GLU | A | 30 | 14.872 | 75.199 | 29.310 | 1.00 | 11.21 | A |
| ATOM | 192 | CB | GLU | A | 30 | 13.458 | 74.677 | 29.598 | 1.00 | 13.90 | A |
| ATOM | 193 | CG | GLU | A | 30 | 13.410 | 73.267 | 30.163 | 1.00 | 18.50 | A |
| ATOM | 194 | CD | GLU | A | 30 | 13.463 | 72.200 | 29.086 | 1.00 | 21.23 | A |
| ATOM | 195 | OE1 | GLU | A | 30 | 13.680 | 72.550 | 27.907 | 1.00 | 23.76 | A |
| ATOM | 196 | OE2 | GLU | A | 30 | 13.290 | 71.007 | 29.420 | 1.00 | 22.98 | A |
| ATOM | 197 | C | GLU | A | 30 | 14.860 | 76.380 | 28.345 | 1.00 | 11.64 | A |
| ATOM | 198 | O | GLU | A | 30 | 14.645 | 76.200 | 27.148 | 1.00 | 12.13 | A |
| ATOM | 199 | N | ARG | A | 31 | 15.082 | 77.585 | 28.860 | 1.00 | 11.14 | A |
| ATOM | 200 | CA | ARG | A | 31 | 15.078 | 78.782 | 28.023 | 1.00 | 11.20 | A |
| ATOM | 201 | CB | ARG | A | 31 | 15.056 | 80.026 | 28.910 | 1.00 | 11.11 | A |
| ATOM | 202 | CG | ARG | A | 31 | 13.806 | 80.249 | 29.730 | 1.00 | 12.33 | A |
| ATOM | 203 | CD | ARG | A | 31 | 14.066 | 81.376 | 30.713 | 1.00 | 11.17 | A |
| ATOM | 204 | NE | ARG | A | 31 | 15.069 | 80.984 | 31.699 | 1.00 | 11.12 | A |
| ATOM | 205 | CZ | ARG | A | 31 | 15.636 | 81.817 | 32.564 | 1.00 | 9.72 | A |
| ATOM | 206 | NH1 | ARG | A | 31 | 15.305 | 83.100 | 32.564 | 1.00 | 11.45 | A |
| ATOM | 207 | NH2 | ARG | A | 31 | 16.520 | 81.367 | 33.447 | 1.00 | 9.95 | A |
| ATOM | 208 | C | ARG | A | 31 | 16.255 | 78.855 | 27.064 | 1.00 | 10.81 | A |
| ATOM | 209 | O | ARG | A | 31 | 16.183 | 79.557 | 26.060 | 1.00 | 11.62 | A |
| ATOM | 210 | N | TYR | A | 32 | 17.327 | 78.133 | 27.380 | 1.00 | 10.66 | A |
| ATOM | 211 | CA | TYR | A | 32 | 18.569 | 78.178 | 26.609 | 1.00 | 9.14 | A |
| ATOM | 212 | CB | TYR | A | 32 | 19.703 | 78.518 | 27.581 | 1.00 | 10.45 | A |
| ATOM | 213 | CG | TYR | A | 32 | 19.446 | 79.736 | 28.438 | 1.00 | 9.84 | A |
| ATOM | 214 | CD1 | TYR | A | 32 | 19.592 | 81.020 | 27.918 | 1.00 | 10.05 | A |
| ATOM | 215 | CE1 | TYR | A | 32 | 19.334 | 82.148 | 28.697 | 1.00 | 11.55 | A |
| ATOM | 216 | CD2 | TYR | A | 32 | 19.032 | 79.604 | 29.764 | 1.00 | 11.10 | A |
| ATOM | 217 | CE2 | TYR | A | 32 | 18.768 | 80.726 | 30.554 | 1.00 | 10.48 | A |
| ATOM | 218 | CZ | TYR | A | 32 | 18.922 | 81.994 | 30.012 | 1.00 | 10.66 | A |
| ATOM | 219 | OH | TYR | A | 32 | 18.653 | 83.107 | 30.779 | 1.00 | 12.60 | A |
| ATOM | 220 | C | TYR | A | 32 | 18.898 | 76.927 | 25.806 | 1.00 | 10.03 | A |
| ATOM | 221 | O | TYR | A | 32 | 18.448 | 75.831 | 26.123 | 1.00 | 10.26 | A |
| ATOM | 222 | N | GLU | A | 33 | 19.714 | 77.111 | 24.771 | 1.00 | 10.24 | A |
| ATOM | 223 | CA | GLU | A | 33 | 20.175 | 76.011 | 23.930 | 1.00 | 9.97 | A |
| ATOM | 224 | CB | GLU | A | 33 | 19.195 | 75.773 | 22.777 | 1.00 | 12.37 | A |
| ATOM | 225 | CG | GLU | A | 33 | 19.079 | 76.948 | 21.824 | 1.00 | 15.72 | A |
| ATOM | 226 | CD | GLU | A | 33 | 18.127 | 76.690 | 20.669 | 1.00 | 18.27 | A |
| ATOM | 227 | OE1 | GLU | A | 33 | 17.767 | 77.664 | 19.973 | 1.00 | 20.76 | A |
| ATOM | 228 | OE2 | GLU | A | 33 | 17.743 | 75.520 | 20.455 | 1.00 | 20.90 | A |
| ATOM | 229 | C | GLU | A | 33 | 21.572 | 76.360 | 23.444 | 1.00 | 10.37 | A |
| ATOM | 230 | O | GLU | A | 33 | 21.979 | 77.527 | 23.468 | 1.00 | 10.72 | A |
| ATOM | 231 | N | GLY | A | 34 | 22.311 | 75.349 | 23.002 | 1.00 | 9.68 | A |
| ATOM | 232 | CA | GLY | A | 34 | 23.652 | 75.600 | 22.508 | 1.00 | 9.45 | A |
| ATOM | 233 | C | GLY | A | 34 | 24.671 | 74.603 | 23.019 | 1.00 | 9.63 | A |
| ATOM | 234 | O | GLY | A | 34 | 24.384 | 73.791 | 23.902 | 1.00 | 10.51 | A |
| ATOM | 235 | N | TYR | A | 35 | 25.875 | 74.682 | 22.465 | 1.00 | 8.89 | A |
| ATOM | 236 | CA | TYR | A | 35 | 26.957 | 73.785 | 22.837 | 1.00 | 9.99 | A |
| ATOM | 237 | CB | TYR | A | 35 | 28.211 | 74.162 | 22.034 | 1.00 | 10.00 | A |
| ATOM | 238 | CG | TYR | A | 35 | 29.438 | 73.363 | 22.414 | 1.00 | 9.08 | A |
| ATOM | 239 | CD1 | TYR | A | 35 | 30.141 | 73.643 | 23.587 | 1.00 | 8.42 | A |
| ATOM | 240 | CE1 | TYR | A | 35 | 31.260 | 72.904 | 23.950 | 1.00 | 9.42 | A |
| ATOM | 241 | CD2 | TYR | A | 35 | 29.889 | 72.315 | 21.611 | 1.00 | 10.27 | A |
| ATOM | 242 | CE2 | TYR | A | 35 | 31.009 | 71.567 | 21.965 | 1.00 | 10.07 | A |
| ATOM | 243 | CZ | TYR | A | 35 | 31.688 | 71.870 | 23.137 | 1.00 | 9.20 | A |
| ATOM | 244 | OH | TYR | A | 35 | 32.794 | 71.145 | 23.506 | 1.00 | 13.71 | A |
| ATOM | 245 | C | TYR | A | 35 | 27.214 | 73.745 | 24.337 | 1.00 | 9.46 | A |
| ATOM | 246 | O | TYR | A | 35 | 27.307 | 72.665 | 24.922 | 1.00 | 9.83 | A |
| ATOM | 247 | N | CYS | A | 36 | 27.338 | 74.912 | 24.963 | 1.00 | 8.94 | A |
| ATOM | 248 | CA | CYS | A | 36 | 27.617 | 74.954 | 26.393 | 1.00 | 9.59 | A |
| ATOM | 249 | CB | CYS | A | 36 | 28.170 | 76.336 | 26.763 | 1.00 | 10.02 | A |
| ATOM | 250 | SG | CYS | A | 36 | 29.984 | 76.573 | 26.253 | 1.00 | 10.91 | A |
| ATOM | 251 | C | CYS | A | 36 | 26.428 | 74.527 | 27.236 | 1.00 | 9.87 | A |
| ATOM | 252 | O | CYS | A | 36 | 26.602 | 74.108 | 28.377 | 1.00 | 9.72 | A |
| ATOM | 253 | N | VAL | A | 37 | 25.224 | 74.649 | 26.688 | 1.00 | 9.91 | A |
| ATOM | 254 | CA | VAL | A | 37 | 24.034 | 74.223 | 27.413 | 1.00 | 10.54 | A |
| ATOM | 255 | CB | VAL | A | 37 | 22.749 | 74.708 | 26.710 | 1.00 | 10.90 | A |
| ATOM | 256 | CG1 | VAL | A | 37 | 21.521 | 74.155 | 27.424 | 1.00 | 12.65 | A |
| ATOM | 257 | CG2 | VAL | A | 37 | 22.709 | 76.232 | 26.715 | 1.00 | 12.51 | A |
| ATOM | 258 | C | VAL | A | 37 | 24.075 | 72.695 | 27.458 | 1.00 | 9.89 | A |
| ATOM | 259 | O | VAL | A | 37 | 23.850 | 72.086 | 28.502 | 1.00 | 11.28 | A |
| ATOM | 260 | N | ASP | A | 38 | 24.393 | 72.082 | 26.320 | 1.00 | 10.26 | A |
| ATOM | 261 | CA | ASP | A | 38 | 24.497 | 70.630 | 26.231 | 1.00 | 10.64 | A |
| ATOM | 262 | CB | ASP | A | 38 | 24.680 | 70.215 | 24.765 | 1.00 | 11.34 | A |
| ATOM | 263 | CG | ASP | A | 38 | 23.425 | 70.391 | 23.931 | 1.00 | 11.41 | A |
| ATOM | 264 | OD1 | ASP | A | 38 | 23.522 | 70.223 | 22.699 | 1.00 | 13.04 | A |
| ATOM | 265 | OD2 | ASP | A | 38 | 23.347 | 70.865 | 24.492 | 1.00 | 14.15 | A |
| ATOM | 266 | C | ASP | A | 38 | 25.652 | 70.157 | 27.108 | 1.00 | 10.10 | A |
| ATOM | 267 | O | ASP | A | 38 | 25.539 | 69.145 | 27.802 | 1.00 | 10.88 | A |
| ATOM | 268 | N | LEU | A | 39 | 26.770 | 70.880 | 27.065 | 1.00 | 10.24 | A |
| ATOM | 269 | CA | LEU | A | 39 | 27.931 | 70.512 | 27.860 | 1.00 | 10.57 | A |
| ATOM | 270 | CB | LEU | A | 39 | 29.109 | 71.423 | 27.496 | 1.00 | 10.29 | A |
| ATOM | 271 | CG | LEU | A | 39 | 30.386 | 71.177 | 28.307 | 1.00 | 10.48 | A |
| ATOM | 272 | CD1 | LEU | A | 39 | 30.832 | 69.725 | 28.174 | 1.00 | 11.52 | A |
| ATOM | 273 | CD2 | LEU | A | 39 | 31.474 | 72.133 | 27.830 | 1.00 | 11.16 | A |
| ATOM | 274 | C | LEU | A | 39 | 27.591 | 70.558 | 29.339 | 1.00 | 9.75 | A |
| ATOM | 275 | O | LEU | A | 39 | 27.937 | 69.646 | 30.094 | 1.00 | 10.20 | A |
| ATOM | 276 | N | ALA | A | 40 | 26.921 | 71.626 | 29.756 | 1.00 | 10.00 | A |
| ATOM | 277 | CA | ALA | A | 40 | 26.543 | 71.776 | 31.157 | 1.00 | 10.04 | A |
| ATOM | 278 | CB | ALA | A | 40 | 25.773 | 73.082 | 31.359 | 1.00 | 11.46 | A |
| ATOM | 279 | C | ALA | A | 40 | 25.707 | 70.584 | 31.625 | 1.00 | 11.75 | A |
| ATOM | 280 | O | ALA | A | 40 | 25.927 | 70.049 | 32.714 | 1.00 | 11.90 | A |
| ATOM | 281 | N | ALA | A | 41 | 24.759 | 70.160 | 30.795 | 1.00 | 11.48 | A |
| ATOM | 282 | CA | ALA | A | 41 | 23.904 | 69.030 | 31.139 | 1.00 | 11.88 | A |
| ATOM | 283 | CB | ALA | A | 41 | 22.835 | 68.837 | 30.068 | 1.00 | 13.22 | A |
| ATOM | 284 | C | ALA | A | 41 | 24.725 | 67.754 | 31.303 | 1.00 | 12.71 | A |
| ATOM | 285 | O | ALA | A | 41 | 24.481 | 66.969 | 32.219 | 1.00 | 13.49 | A |
| ATOM | 286 | N | GLU | A | 42 | 25.696 | 67.545 | 30.417 | 1.00 | 12.51 | A |
| ATOM | 287 | CA | GLU | A | 42 | 26.545 | 66.360 | 30.493 | 1.00 | 13.27 | A |
| ATOM | 288 | CB | GLU | A | 42 | 27.338 | 66.219 | 29.188 | 1.00 | 15.77 | A |
| ATOM | 289 | CG | GLU | A | 42 | 26.575 | 65.510 | 28.082 | 1.00 | 18.63 | A |
| ATOM | 290 | CD | GLU | A | 42 | 26.451 | 64.013 | 28.327 | 1.00 | 19.37 | A |
| ATOM | 291 | OE1 | GLU | A | 42 | 26.787 | 63.556 | 29.440 | 1.00 | 18.40 | A |
| ATOM | 292 | OE2 | GLU | A | 42 | 26.015 | 63.292 | 27.407 | 1.00 | 20.92 | A |
| ATOM | 293 | C | GLU | A | 42 | 27.435 | 66.389 | 31.728 | 1.00 | 13.94 | A |
| ATOM | 294 | O | GLU | A | 42 | 27.607 | 65.375 | 32.399 | 1.00 | 14.45 | A |
| ATOM | 295 | N | ILE | A | 43 | 28.018 | 67.545 | 32.023 | 1.00 | 12.51 | A |
| ATOM | 296 | CA | ILE | A | 43 | 28.878 | 67.665 | 33.193 | 1.00 | 13.29 | A |
| ATOM | 297 | CB | ILE | A | 43 | 29.491 | 69.085 | 33.300 | 1.00 | 13.12 | A |
| ATOM | 298 | CG2 | ILE | A | 43 | 30.204 | 69.245 | 34.634 | 1.00 | 12.80 | A |
| ATOM | 299 | CG1 | ILE | A | 43 | 30.456 | 69.333 | 32.137 | 1.00 | 14.92 | A |
| ATOM | 300 | CD1 | ILE | A | 43 | 31.665 | 68.428 | 32.127 | 1.00 | 14.91 | A |
| ATOM | 301 | C | ILE | A | 43 | 28.059 | 67.376 | 34.452 | 1.00 | 13.51 | A |
| ATOM | 302 | O | ILE | A | 43 | 28.484 | 66.616 | 35.325 | 1.00 | 14.79 | A |
| ATOM | 303 | N | ALA | A | 41 | 26.875 | 67.977 | 34.532 | 1.00 | 12.91 | A |
| ATOM | 304 | CA | ALA | A | 44 | 25.996 | 67.792 | 35.681 | 1.00 | 13.94 | A |
| ATOM | 305 | CB | ALA | A | 44 | 24.763 | 68.661 | 35.534 | 1.00 | 13.58 | A |
| ATOM | 306 | C | ALA | A | 44 | 25.592 | 66.335 | 35.867 | 1.00 | 14.33 | A |
| ATOM | 307 | O | ALA | A | 44 | 25.549 | 65.840 | 69.993 | 1.00 | 15.33 | A |
| ATOM | 308 | N | LYS | A | 45 | 25.296 | 65.651 | 34.766 | 1.00 | 15.00 | A |
| ATOM | 309 | CA | LYS | A | 45 | 24.908 | 64.246 | 34.828 | 1.00 | 17.52 | A |
| ATOM | 310 | CB | LYS | A | 45 | 24.467 | 63.776 | 33.440 | 1.00 | 19.45 | A |
| ATOM | 311 | CG | LYS | A | 45 | 23.146 | 64.350 | 32.965 | 1.00 | 23.23 | A |
| ATOM | 312 | CD | LYS | A | 45 | 22.709 | 63.696 | 31.662 | 1.00 | 26.44 | A |
| ATOM | 313 | CE | LYS | A | 45 | 22.539 | 62.192 | 31.836 | 1.00 | 28.68 | A |
| ATOM | 314 | NZ | LYS | A | 45 | 22.151 | 61.510 | 30.575 | 1.00 | 29.68 | A |
| ATOM | 315 | C | LYS | A | 45 | 26.041 | 63.402 | 35.395 | 1.00 | 17.69 | A |
| ATOM | 316 | O | LYS | A | 45 | 25.828 | 62.585 | 36.289 | 1.00 | 18.84 | A |
| ATOM | 317 | N | HIS | A | 46 | 27.248 | 63.597 | 34.876 | 1.00 | 16.11 | A |
| ATOM | 318 | CA | HIS | A | 46 | 28.396 | 62.828 | 35.340 | 1.00 | 16.88 | A |
| ATOM | 319 | CB | HIS | A | 46 | 29.540 | 62.966 | 34.328 | 1.00 | 15.26 | A |
| ATOM | 320 | CG | HIS | A | 46 | 29.312 | 62.206 | 33.060 | 1.00 | 16.43 | A |
| ATOM | 321 | CD2 | HIS | A | 46 | 29.739 | 60.982 | 32.668 | 1.00 | 18.25 | A |
| ATOM | 322 | ND1 | HIS | A | 46 | 28.511 | 62.675 | 32.042 | 1.00 | 18.48 | A |
| ATOM | 323 | CE1 | HIS | A | 46 | 28.454 | 61.774 | 31.078 | 1.00 | 18.78 | A |
| ATOM | 324 | NE2 | HIS | A | 46 | 29.190 | 60.737 | 31.433 | 1.00 | 21.02 | A |
| ATOM | 325 | C | HIS | A | 46 | 28.823 | 63.157 | 36.766 | 1.00 | 17.40 | A |
| ATOM | 326 | O | HIS | A | 46 | 29.336 | 62.291 | 37.474 | 1.00 | 19.71 | A |
| ATOM | 327 | N | CYS | A | 47 | 28.609 | 64.397 | 37.193 | 1.00 | 17.64 | A |
| ATOM | 328 | CA | CYS | A | 47 | 28.990 | 64.817 | 38.538 | 1.00 | 17.02 | A |
| ATOM | 329 | CB | CYS | A | 47 | 29.478 | 66.265 | 38.489 | 1.00 | 16.75 | A |
| ATOM | 330 | SG | CYS | A | 47 | 31.003 | 66.476 | 37.551 | 1.00 | 16.17 | A |
| ATOM | 331 | C | CYS | A | 47 | 27.862 | 64.610 | 39.534 | 1.00 | 17.55 | A |
| ATOM | 332 | O | CYS | A | 47 | 28.084 | 64.628 | 40.743 | 1.00 | 19.42 | A |
| ATOM | 333 | N | GLY | A | 48 | 26.652 | 64.426 | 39.022 | 1.00 | 16.92 | A |
| ATOM | 334 | CA | GLY | A | 48 | 25.513 | 64.207 | 39.890 | 1.00 | 18.16 | A |
| ATOM | 335 | C | GLY | A | 48 | 25.034 | 65.421 | 40.664 | 1.00 | 17.73 | A |
| ATOM | 336 | O | GLY | A | 48 | 24.782 | 65.329 | 41.865 | 1.00 | 20.39 | A |
| ATOM | 337 | N | PHE | A | 49 | 24.916 | 66.565 | 39.996 | 1.00 | 16.58 | A |
| ATOM | 338 | CA | PHE | A | 49 | 24.429 | 67.758 | 40.673 | 1.00 | 14.80 | A |
| ATOM | 339 | CB | PHE | A | 49 | 25.606 | 68.696 | 41.026 | 1.00 | 15.66 | A |
| ATOM | 340 | CG | PHE | A | 49 | 26.337 | 69.285 | 39.832 | 1.00 | 14.59 | A |
| ATOM | 341 | CD1 | PHE | A | 49 | 25.757 | 70.155 | 39.064 | 1.00 | 13.66 | A |
| ATOM | 342 | CD2 | PHE | A | 49 | 27.572 | 68.955 | 39.615 | 1.00 | 13.77 | A |
| ATOM | 343 | CE1 | PHE | A | 49 | 26.426 | 70.770 | 37.992 | 1.00 | 13.65 | A |
| ATOM | 344 | CE2 | PHE | A | 49 | 28.327 | 69.513 | 38.571 | 1.00 | 14.71 | A |
| ATOM | 345 | CZ | PHE | A | 49 | 27.739 | 70.438 | 37.748 | 1.00 | 14.34 | A |
| ATOM | 346 | C | PHE | A | 49 | 23.333 | 68.436 | 39.868 | 1.00 | 14.31 | A |
| ATOM | 347 | O | PHE | A | 49 | 23.230 | 68.252 | 38.654 | 1.00 | 14.47 | A |
| ATOM | 348 | N | ALA | A | 50 | 22.486 | 69.184 | 40.569 | 1.00 | 13.89 | A |
| ATOM | 349 | CA | ALA | A | 50 | 21.418 | 69.937 | 39.928 | 1.00 | 12.51 | A |
| ATOM | 350 | CB | ALA | A | 50 | 20.184 | 69.989 | 40.824 | 1.00 | 15.91 | A |
| ATOM | 351 | C | ALA | A | 50 | 22.019 | 71.327 | 39.762 | 1.00 | 22.02 | A |
| ATOM | 352 | O | ALA | A | 50 | 22.841 | 71.750 | 40.578 | 1.00 | 12.80 | A |
| ATOM | 353 | N | TYR | A | 51 | 21.621 | 72.040 | 38.717 | 1.00 | 12.82 | A |
| ATOM | 354 | CA | TYR | A | 51 | 22.193 | 73.360 | 38.481 | 1.00 | 11.50 | A |
| ATOM | 355 | CB | TYR | A | 51 | 23.423 | 73.214 | 37.582 | 1.00 | 10.79 | A |
| ATOM | 356 | CG | TYR | A | 51 | 23.074 | 72.687 | 36.211 | 1.00 | 10.07 | A |
| ATOM | 357 | CD1 | TYR | A | 51 | 22.770 | 71.340 | 36.023 | 1.00 | 11.63 | A |
| ATOM | 358 | CE1 | TYR | A | 51 | 22.394 | 70.858 | 34.775 | 1.00 | 11.65 | A |
| ATOM | 359 | CD2 | TYR | A | 51 | 22.996 | 73.544 | 35.111 | 1.00 | 11.90 | A |
| ATOM | 360 | CE2 | TYR | A | 51 | 22.621 | 73.071 | 33.862 | 1.00 | 11.04 | A |
| ATOM | 361 | CZ | TYR | A | 51 | 22.321 | 71.728 | 33.701 | 1.00 | 11.39 | A |
| ATOM | 362 | OH | TYR | A | 51 | 21.941 | 71.251 | 32.475 | 1.00 | 13.99 | A |
| ATOM | 363 | C | TYR | A | 51 | 21.209 | 74.344 | 37.891 | 1.00 | 11.97 | A |
| ATOM | 364 | O | TYR | A | 51 | 20.161 | 73.973 | 37.366 | 1.00 | 11.86 | A |
| ATOM | 365 | N | ALA | A | 52 | 21.594 | 75.612 | 37.961 | 1.00 | 11.76 | A |
| ATOM | 366 | CA | ALA | A | 52 | 20.802 | 76.702 | 37.423 | 1.00 | 11.93 | A |
| ATOM | 367 | CB | ALA | A | 52 | 20.249 | 77.552 | 38.555 | 1.00 | 15.06 | A |
| ATOM | 368 | C | ALA | A | 52 | 21.706 | 77.543 | 36.532 | 1.00 | 11.65 | A |
| ATOM | 369 | O | ALA | A | 52 | 22.748 | 78.021 | 36.975 | 1.00 | 11.80 | A |
| ATOM | 370 | N | LEU | A | 53 | 21.314 | 77.702 | 35.273 | 1.00 | 10.72 | A |
| ATOM | 371 | CA | LEU | A | 53 | 22.081 | 78.517 | 34.336 | 1.00 | 10.99 | A |
| ATOM | 372 | CB | LEU | A | 53 | 21.746 | 78.108 | 32.898 | 1.00 | 11.40 | A |
| ATOM | 373 | CG | LEU | A | 53 | 22.095 | 76.694 | 32.431 | 1.00 | 11.91 | A |
| ATOM | 374 | CD1 | LEU | A | 53 | 21.540 | 76.471 | 31.032 | 1.00 | 13.32 | A |
| ATOM | 375 | CD2 | LEU | A | 53 | 23.601 | 76.503 | 32.442 | 1.00 | 14.04 | A |
| ATOM | 376 | C | LEU | A | 53 | 21.755 | 79.980 | 34.597 | 1.00 | 11.07 | A |
| ATOM | 377 | O | LEU | A | 53 | 20.587 | 80.366 | 34.589 | 1.00 | 11.97 | A |
| ATOM | 378 | N | THR | A | 54 | 22.784 | 80.786 | 34.839 | 1.00 | 10.70 | A |
| ATOM | 379 | CA | THR | A | 54 | 22.601 | 82.213 | 35.073 | 1.00 | 9.61 | A |
| ATOM | 380 | CB | THR | A | 54 | 22.956 | 82.625 | 36.522 | 1.00 | 11.92 | A |
| ATOM | 381 | OG1 | THR | A | 54 | 24.356 | 82.428 | 36.757 | 1.00 | 13.30 | A |
| ATOM | 382 | CG2 | THR | A | 54 | 22.150 | 81.806 | 37.519 | 1.00 | 12.21 | A |
| ATOM | 383 | C | THR | A | 54 | 23.518 | 82.940 | 34.097 | 1.00 | 10.63 | A |
| ATOM | 384 | O | THR | A | 54 | 24.583 | 82.433 | 33.742 | 1.00 | 10.64 | A |
| ATOM | 385 | N | ILE | A | 55 | 23.098 | 84.119 | 33.658 | 1.00 | 10.43 | A |
| ATOM | 386 | CA | ILE | A | 55 | 23.869 | 84.893 | 32.699 | 1.00 | 9.86 | A |
| ATOM | 387 | CB | ILE | A | 55 | 22.929 | 85.569 | 31.682 | 1.00 | 9.45 | A |
| ATOM | 388 | CG2 | ILE | A | 55 | 23.743 | 86.278 | 30.612 | 1.00 | 10.05 | A |
| ATOM | 389 | CG1 | ILE | A | 55 | 21.994 | 84.523 | 31.061 | 1.00 | 12.39 | A |
| ATOM | 390 | CD1 | ILE | A | 55 | 22.700 | 83.370 | 30.354 | 1.00 | 14.56 | A |
| ATOM | 391 | C | ILE | A | 55 | 24.705 | 85.939 | 33.418 | 1.00 | 11.14 | A |
| ATOM | 392 | O | ILE | A | 55 | 21.183 | 86.741 | 34.191 | 1.00 | 11.26 | A |
| ATOM | 393 | N | VAL | A | 56 | 26.009 | 85.926 | 33.160 | 1.00 | 10.84 | A |
| ATOM | 394 | CA | VAL | A | 56 | 26.931 | 86.853 | 33.805 | 1.00 | 10.67 | A |
| ATOM | 395 | CB | VAL | A | 56 | 28.374 | 86.689 | 33.232 | 1.00 | 9.37 | A |
| ATOM | 396 | CG1 | VAL | A | 56 | 28.408 | 86.996 | 31.748 | 1.00 | 8.67 | A |
| ATOM | 397 | CG2 | VAL | A | 56 | 29.335 | 87.577 | 33.996 | 1.00 | 11.63 | A |
| ATOM | 398 | C | VAL | A | 56 | 26.454 | 88.304 | 33.705 | 1.00 | 11.20 | A |
| ATOM | 399 | O | VAL | A | 56 | 26.191 | 88.824 | 32.617 | 1.00 | 12.28 | A |
| ATOM | 400 | N | GLY | A | 57 | 26.354 | 88.944 | 34.866 | 1.00 | 12.47 | A |
| ATOM | 401 | CA | GLY | A | 57 | 25.864 | 90.309 | 34.948 | 1.00 | 13.12 | A |
| ATOM | 402 | C | GLY | A | 57 | 26.506 | 91.389 | 34.102 | 1.00 | 14.20 | A |
| ATOM | 403 | O | GLY | A | 57 | 25.799 | 92.153 | 33.442 | 1.00 | 15.02 | A |
| ATOM | 404 | N | ASP | A | 58 | 27.833 | 91.466 | 34.111 | 1.00 | 14.42 | A |
| ATOM | 405 | CA | ASP | A | 58 | 28.524 | 92.506 | 33.356 | 1.00 | 14.36 | A |
| ATOM | 406 | CB | ASP | A | 58 | 29.749 | 92.969 | 34.156 | 1.00 | 14.26 | A |
| ATOM | 407 | CG | ASP | A | 58 | 30.813 | 91.898 | 34.273 | 1.00 | 13.53 | A |
| ATOM | 408 | OD1 | ASP | A | 58 | 31.796 | 92.125 | 35.008 | 1.00 | 15.20 | A |
| ATOM | 409 | OD2 | ASP | A | 58 | 30.672 | 90.834 | 33.629 | 1.00 | 13.56 | A |
| ATOM | 410 | C | ASP | A | 58 | 28.858 | 92.131 | 31.918 | 1.00 | 14.32 | A |
| ATOM | 411 | O | ASP | A | 58 | 29.520 | 92.898 | 31.218 | 1.00 | 15.28 | A |
| ATOM | 412 | N | GLY | A | 59 | 28.414 | 90.956 | 31.481 | 1.00 | 14.37 | A |
| ATOM | 413 | CA | GLY | A | 59 | 28.666 | 90.525 | 30.116 | 1.00 | 12.51 | A |
| ATOM | 414 | C | GLY | A | 59 | 30.126 | 90.394 | 29.717 | 1.00 | 12.13 | A |
| ATOM | 415 | O | GLY | A | 59 | 30.459 | 90.527 | 28.539 | 1.00 | 13.27 | A |
| ATOM | 416 | N | LYS | A | 60 | 30.994 | 90.123 | 30.688 | 1.00 | 10.36 | A |
| ATOM | 417 | CA | LYS | A | 60 | 32.429 | 89.986 | 30.435 | 1.00 | 12.62 | A |
| ATOM | 418 | CB | LYS | A | 60 | 33.190 | 91.006 | 31.291 | 1.00 | 12.17 | A |
| ATOM | 419 | CG | LYS | A | 60 | 32.771 | 92.459 | 31.118 | 1.00 | 15.61 | A |
| ATOM | 420 | CD | LYS | A | 60 | 33.202 | 93.020 | 29.780 | 1.00 | 19.19 | A |
| ATOM | 421 | CE | LYS | A | 60 | 32.861 | 94.502 | 29.692 | 1.00 | 19.63 | A |
| ATOM | 422 | NZ | LYS | A | 60 | 33.267 | 95.095 | 28.390 | 1.00 | 22.12 | A |
| ATOM | 423 | C | LYS | A | 60 | 32.891 | 88.562 | 30.710 | 1.00 | 10.40 | A |
| ATOM | 424 | O | LYS | A | 60 | 32.187 | 87.781 | 31.354 | 1.00 | 10.69 | A |
| ATOM | 425 | N | TYR | A | 61 | 34.086 | 88.231 | 30.227 | 1.00 | 9.97 | A |
| ATOM | 426 | CA | TYR | A | 61 | 34.647 | 86.904 | 30.440 | 1.00 | 9.43 | A |
| ATOM | 427 | CB | TYR | A | 61 | 35.378 | 86.445 | 29.173 | 1.00 | 9.37 | A |
| ATOM | 428 | CG | TYR | A | 61 | 34.427 | 85.969 | 28.109 | 1.00 | 9.04 | A |
| ATOM | 429 | CD1 | TYR | A | 61 | 33.629 | 86.872 | 27.405 | 1.00 | 9.32 | A |
| ATOM | 430 | CE1 | TYR | A | 61 | 32.698 | 86.432 | 26.471 | 1.00 | 10.34 | A |
| ATOM | 431 | CD2 | TYR | A | 61 | 34.272 | 84.609 | 27.846 | 1.00 | 10.11 | A |
| ATOM | 432 | CE2 | TYR | A | 61 | 33.343 | 84.158 | 26.911 | 1.00 | 9.38 | A |
| ATOM | 433 | CZ | TYR | A | 61 | 32.560 | 85.075 | 26.230 | 1.00 | 11.16 | A |
| ATOM | 434 | OH | TYR | A | 61 | 31.646 | 84.628 | 25.306 | 1.00 | 11.81 | A |
| ATOM | 435 | C | TYR | A | 61 | 35.512 | 86.878 | 31.689 | 1.00 | 9.35 | A |
| ATOM | 436 | O | TYR | A | 61 | 35.276 | 86.075 | 32.588 | 1.00 | 10.72 | A |
| ATOM | 437 | N | GLY | A | 62 | 36.514 | 87.750 | 31.753 | 1.00 | 9.91 | A |
| ATOM | 438 | CA | GLY | A | 62 | 37.359 | 87.782 | 32.931 | 1.00 | 10.68 | A |
| ATOM | 439 | C | GLY | A | 62 | 38.661 | 88.539 | 32.767 | 1.00 | 10.33 | A |
| ATOM | 440 | O | GLY | A | 62 | 39.585 | 88.061 | 32.106 | 1.00 | 10.43 | A |
| ATOM | 441 | N | ALA | A | 63 | 38.735 | 89.718 | 33.380 | 1.00 | 11.66 | A |
| ATOM | 442 | CA | ALA | A | 63 | 39.930 | 90.554 | 33.334 | 1.00 | 11.95 | A |
| ATOM | 443 | CB | ALA | A | 63 | 39.865 | 91.513 | 32.152 | 1.00 | 13.77 | A |
| ATOM | 444 | C | ALA | A | 63 | 39.992 | 91.336 | 34.639 | 1.00 | 13.84 | A |
| ATOM | 445 | O | ALA | A | 63 | 38.968 | 91.556 | 35.286 | 1.00 | 13.50 | A |
| ATOM | 446 | N | ARG | A | 64 | 41.192 | 91.754 | 35.025 | 1.00 | 14.65 | A |
| ATOM | 447 | CA | ARG | A | 64 | 41.366 | 92.509 | 36.257 | 1.00 | 17.59 | A |
| ATOM | 448 | CB | ARG | A | 64 | 42.431 | 91.832 | 37.122 | 1.00 | 19.09 | A |
| ATOM | 449 | CG | ARG | A | 64 | 42.557 | 92.447 | 38.509 | 1.00 | 22.61 | A |
| ATOM | 450 | CD | ARG | A | 64 | 43.792 | 91.954 | 39.238 | 1.00 | 25.53 | A |
| ATOM | 451 | NE | ARG | A | 64 | 45.020 | 92.426 | 38.605 | 1.00 | 28.82 | A |
| ATOM | 452 | CZ | ARG | A | 64 | 46.236 | 92.250 | 39.111 | 1.00 | 31.37 | A |
| ATOM | 453 | NH1 | ARG | A | 64 | 46.390 | 91.608 | 40.262 | 1.00 | 33.09 | A |
| ATOM | 454 | NH2 | ARG | A | 64 | 47.297 | 92.719 | 38.469 | 1.00 | 32.81 | A |
| ATOM | 455 | C | ARG | A | 64 | 47.710 | 93.958 | 35.946 | 1.00 | 18.49 | A |
| ATOM | 456 | O | ARG | A | 64 | 42.621 | 94.233 | 35.163 | 1.00 | 18.63 | A |
| ATOM | 457 | N | ASP | A | 65 | 40.980 | 94.883 | 36.558 | 1.00 | 20.04 | A |
| ATOM | 458 | CA | ASP | A | 65 | 41.235 | 96.303 | 36.351 | 1.00 | 22.22 | A |
| ATOM | 459 | CB | ASP | A | 65 | 40.117 | 97.120 | 37.003 | 1.00 | 22.81 | A |
| ATOM | 460 | CG | ASP | A | 65 | 40.205 | 98.598 | 36.667 | 1.00 | 24.22 | A |
| ATOM | 461 | OD1 | ASP | A | 65 | 39.885 | 98.967 | 35.518 | 1.00 | 26.48 | A |
| ATOM | 462 | OD2 | ASP | A | 65 | 40.603 | 99.387 | 37.548 | 1.00 | 26.15 | A |
| ATOM | 463 | C | ASP | A | 65 | 42.611 | 96.581 | 36.947 | 1.00 | 23.72 | A |
| ATOM | 464 | O | ASP | A | 65 | 42.884 | 96.210 | 38.087 | 1.00 | 23.99 | A |
| ATOM | 465 | N | ALA | A | 66 | 43.477 | 97.229 | 36.176 | 1.00 | 26.04 | A |
| ATOM | 466 | CA | ALA | A | 66 | 44.828 | 97.531 | 36.636 | 1.00 | 28.03 | A |
| ATOM | 467 | CB | ALA | A | 66 | 45.652 | 98.090 | 35.483 | 1.00 | 28.75 | A |
| ATOM | 468 | C | ALA | A | 66 | 44.857 | 98.499 | 37.814 | 1.00 | 28.79 | A |
| ATOM | 469 | O | ALA | A | 66 | 45.809 | 98.504 | 38.595 | 1.00 | 29.26 | A |
| ATOM | 470 | N | ASN | A | 67 | 43.812 | 99.310 | 37.946 | 1.00 | 29.06 | A |
| ATOM | 471 | CA | ASN | A | 67 | 43.740 | 100.288 | 39.025 | 1.00 | 29.85 | A |
| ATOM | 472 | CB | ASN | A | 67 | 43.019 | 101.537 | 38.509 | 1.00 | 30.58 | A |
| ATOM | 473 | CG | ASN | A | 67 | 43.700 | 102.145 | 37.298 | 1.00 | 32.46 | A |
| ATOM | 474 | OD1 | ASN | A | 67 | 44.886 | 102.473 | 37.339 | 1.00 | 32.86 | A |
| ATOM | 475 | ND2 | ASN | A | 67 | 42.952 | 102.300 | 36.212 | 1.00 | 33.03 | A |
| ATOM | 476 | C | ASN | A | 67 | 43.105 | 99.717 | 40.288 | 1.00 | 29.38 | A |
| ATOM | 477 | O | ASN | A | 67 | 43.744 | 99.655 | 41.339 | 1.00 | 29.66 | A |
| ATOM | 478 | N | THR | A | 68 | 41.847 | 99.301 | 40.179 | 1.00 | 27.82 | A |
| ATOM | 479 | CA | THR | A | 68 | 41.109 | 98.755 | 41.314 | 1.00 | 26.63 | A |
| ATOM | 480 | CB | THR | A | 68 | 39.595 | 98.807 | 41.060 | 1.00 | 25.72 | A |
| ATOM | 481 | OG1 | THR | A | 68 | 39.265 | 97.930 | 39.975 | 1.00 | 24.07 | A |
| ATOM | 482 | CG2 | THR | A | 68 | 39.166 | 100.222 | 40.710 | 1.00 | 25.34 | A |
| ATOM | 483 | C | THR | A | 68 | 41.479 | 97.313 | 41.648 | 1.00 | 26.22 | A |
| ATOM | 484 | O | THR | A | 68 | 41.233 | 96.848 | 42.761 | 1.00 | 26.49 | A |
| ATOM | 485 | N | LYS | A | 69 | 42.059 | 96.611 | 40.681 | 1.00 | 25.60 | A |
| ATOM | 486 | CA | LYS | A | 69 | 42.461 | 95.221 | 40.867 | 1.00 | 25.08 | A |
| ATOM | 487 | CB | LYS | A | 69 | 43.465 | 95.142 | 42.022 | 1.00 | 28.90 | A |
| ATOM | 488 | CG | LYS | A | 69 | 44.710 | 95.990 | 41.814 | 1.00 | 33.71 | A |
| ATOM | 489 | CD | LYS | A | 69 | 45.662 | 95.872 | 42.991 | 1.00 | 38.30 | A |
| ATOM | 490 | CE | LYS | A | 69 | 46.885 | 96.758 | 42.808 | 1.00 | 40.62 | A |
| ATOM | 491 | NZ | LYS | A | 69 | 47.821 | 96.646 | 43.961 | 1.00 | 42.64 | A |
| ATOM | 492 | C | LYS | A | 69 | 41.249 | 94.314 | 41.063 | 1.00 | 23.09 | A |
| ATOM | 493 | O | LYS | A | 69 | 41.370 | 93.209 | 41.591 | 1.00 | 22.36 | A |
| ATOM | 494 | N | ILE | A | 70 | 40.082 | 94.785 | 40.636 | 1.00 | 18.73 | A |
| ATOM | 495 | CA | ILE | A | 70 | 38.851 | 94.007 | 40.752 | 1.00 | 17.41 | A |
| ATOM | 496 | CB | ILE | A | 70 | 37.619 | 94.926 | 40.921 | 1.00 | 18.61 | A |
| ATOM | 497 | CG2 | ILE | A | 70 | 36.343 | 94.096 | 40.924 | 1.00 | 19.24 | A |
| ATOM | 498 | CG1 | ILE | A | 70 | 37.743 | 95.733 | 42.215 | 1.00 | 19.23 | A |
| ATOM | 499 | CD1 | ILE | A | 70 | 36.620 | 96.736 | 42.423 | 1.00 | 20.47 | A |
| ATOM | 500 | C | ILE | A | 70 | 38.656 | 93.173 | 39.490 | 1.00 | 15.83 | A |
| ATOM | 501 | O | ILE | A | 70 | 38.887 | 93.656 | 38.383 | 1.00 | 15.20 | A |
| ATOM | 502 | N | TRP | A | 71 | 38.240 | 91.921 | 39.663 | 1.00 | 13.08 | A |
| ATOM | 503 | CA | TRP | A | 71 | 38.003 | 91.026 | 38.535 | 1.00 | 12.55 | A |
| ATOM | 504 | CB | TRP | A | 71 | 38.318 | 89.583 | 38.942 | 1.00 | 12.03 | A |
| ATOM | 505 | CG | TRP | A | 71 | 39.772 | 89.255 | 38.964 | 1.00 | 13.66 | A |
| ATOM | 506 | CD2 | TRP | A | 71 | 40.549 | 88.766 | 37.868 | 1.00 | 13.45 | A |
| ATOM | 507 | CE2 | TRP | A | 71 | 41.874 | 88.607 | 38.330 | 1.00 | 15.39 | A |
| ATOM | 508 | CE3 | TRP | A | 71 | 40.254 | 88.450 | 36.536 | 1.00 | 13.75 | A |
| ATOM | 509 | CD1 | TRP | A | 71 | 40.630 | 89.371 | 40.022 | 1.00 | 13.77 | A |
| ATOM | 510 | NE1 | TRP | A | 71 | 41.896 | 88.981 | 39.649 | 1.00 | 15.77 | A |
| ATOM | 511 | CZ2 | TRP | A | 71 | 42.905 | 88.142 | 37.505 | 1.00 | 15.44 | A |
| ATOM | 512 | CZ3 | TRP | A | 71 | 41.280 | 87.987 | 35.715 | 1.00 | 14.18 | A |
| ATOM | 513 | CH2 | TRP | A | 71 | 42.588 | 87.839 | 36.206 | 1.00 | 15.37 | A |
| ATOM | 514 | C | TRP | A | 71 | 36.576 | 91.164 | 38.036 | 1.00 | 12.49 | A |
| ATOM | 515 | O | TRP | A | 71 | 35.641 | 91.188 | 38.831 | 1.00 | 12.74 | A |
| ATOM | 516 | N | ASN | A | 72 | 36.407 | 91.244 | 36.718 | 1.00 | 11.07 | A |
| ATOM | 517 | CA | ASN | A | 72 | 35.075 | 91.350 | 36.133 | 1.00 | 11.34 | A |
| ATOM | 518 | CB | ASN | A | 72 | 35.022 | 92.534 | 35.155 | 1.00 | 10.91 | A |
| ATOM | 519 | CG | ASN | A | 72 | 35.893 | 92.322 | 33.928 | 1.00 | 12.43 | A |
| ATOM | 520 | OD1 | ASN | A | 72 | 36.262 | 91.195 | 33.599 | 1.00 | 12.97 | A |
| ATOM | 521 | ND2 | ASN | A | 72 | 36.209 | 93.410 | 33.233 | 1.00 | 14.86 | A |
| ATOM | 522 | C | ASN | A | 72 | 34.692 | 90.034 | 35.463 | 1.00 | 10.96 | A |
| ATOM | 523 | O | ASN | A | 72 | 35.432 | 89.049 | 35.524 | 1.00 | 11.26 | A |
| ATOM | 524 | N | GLY | A | 73 | 33.521 | 90.034 | 34.836 | 1.00 | 11.00 | A |
| ATOM | 525 | CA | GLY | A | 73 | 33.038 | 88.871 | 34.112 | 1.00 | 10.39 | A |
| ATOM | 526 | C | GLY | A | 73 | 32.798 | 87.602 | 34.904 | 1.00 | 9.77 | A |
| ATOM | 527 | O | GLY | A | 73 | 32.636 | 87.625 | 36.126 | 1.00 | 9.76 | A |
| ATOM | 528 | N | MET | A | 74 | 32.783 | 86.480 | 34.189 | 1.00 | 8.85 | A |
| ATOM | 529 | CA | MET | A | 74 | 32.551 | 85.179 | 34.798 | 1.00 | 9.58 | A |
| ATOM | 530 | CB | MET | A | 74 | 32.388 | 84.130 | 33.696 | 1.00 | 9.64 | A |
| ATOM | 531 | CG | MET | A | 74 | 31.126 | 84.314 | 32.874 | 1.00 | 10.68 | A |
| ATOM | 532 | SD | MET | A | 74 | 30.972 | 83.136 | 31.533 | 1.00 | 9.47 | A |
| ATOM | 533 | CE | MET | A | 74 | 32.107 | 83.851 | 30.331 | 1.00 | 10.20 | A |
| ATOM | 534 | C | MET | A | 74 | 33.644 | 84.841 | 35.792 | 1.00 | 9.16 | A |
| ATOM | 535 | O | MET | A | 74 | 33.374 | 84.250 | 36.835 | 1.00 | 9.49 | A |
| ATOM | 536 | N | VAL | A | 75 | 34.882 | 85.203 | 35.469 | 1.00 | 9.50 | A |
| ATOM | 537 | CA | VAL | A | 75 | 35.979 | 84.938 | 36.383 | 1.00 | 10.09 | A |
| ATOM | 538 | CB | VAL | A | 75 | 37.334 | 85.382 | 35.784 | 1.00 | 9.77 | A |
| ATOM | 539 | CG1 | VAL | A | 75 | 38.440 | 85.254 | 36.831 | 1.00 | 11.29 | A |
| ATOM | 540 | CG2 | VAL | A | 75 | 37.663 | 84.512 | 34.568 | 1.00 | 11.69 | A |
| ATOM | 541 | C | VAL | A | 75 | 35.690 | 85.687 | 37.683 | 1.00 | 8.71 | A |
| ATOM | 542 | O | VAL | A | 75 | 35.847 | 85.131 | 38.771 | 1.00 | 9.77 | A |
| ATOM | 543 | N | GLY | A | 76 | 35.243 | 86.936 | 37.566 | 1.00 | 9.90 | A |
| ATOM | 544 | CA | GLY | A | 76 | 34.919 | 87.713 | 38.753 | 1.00 | 10.44 | A |
| ATOM | 545 | C | GLY | A | 76 | 33.828 | 87.064 | 39.590 | 1.00 | 9.56 | A |
| ATOM | 546 | O | GLY | A | 76 | 33.922 | 87.026 | 40.815 | 1.00 | 9.93 | A |
| ATOM | 547 | N | GLU | A | 77 | 32.789 | 86.547 | 38.943 | 1.00 | 9.57 | A |
| ATOM | 548 | CA | GLU | A | 77 | 31.707 | 85.905 | 39.680 | 1.00 | 10.67 | A |
| ATOM | 549 | CB | GLU | A | 77 | 30.583 | 85.522 | 38.715 | 1.00 | 12.21 | A |
| ATOM | 550 | CG | GLU | A | 77 | 29.957 | 96.719 | 38.025 | 1.00 | 14.32 | A |
| ATOM | 551 | CD | GLU | A | 77 | 28.570 | 86.433 | 37.489 | 1.00 | 16.51 | A |
| ATOM | 552 | OE1 | GLU | A | 77 | 28.226 | 85.246 | 37.321 | 1.00 | 18.52 | A |
| ATOM | 553 | OE2 | GLU | A | 77 | 27.825 | 87.397 | 37.228 | 1.00 | 16.53 | A |
| ATOM | 554 | C | GLU | A | 77 | 32.241 | 84.732 | 40.490 | 1.00 | 16.91 | A |
| ATOM | 555 | O | GLU | A | 77 | 31.785 | 84.483 | 41.604 | 1.00 | 11.18 | A |
| ATOM | 556 | N | LEU | A | 78 | 33.209 | 84.014 | 39.932 | 1.00 | 9.49 | A |
| ATOM | 557 | CA | LEU | A | 78 | 33.815 | 82.880 | 40.619 | 1.00 | 10.07 | A |
| ATOM | 558 | CB | LEU | A | 78 | 34.630 | 82.074 | 39.607 | 1.00 | 10.23 | A |
| ATOM | 559 | CG | LEU | A | 78 | 34.212 | 80.693 | 39.093 | 1.00 | 16.68 | A |
| ATOM | 560 | CD1 | LEU | A | 78 | 32.707 | 80.498 | 39.069 | 1.00 | 11.70 | A |
| ATOM | 561 | CD2 | LEU | A | 78 | 34.814 | 80.533 | 37.707 | 1.00 | 12.46 | A |
| ATOM | 562 | C | LEU | A | 78 | 34.669 | 83.375 | 41.782 | 1.00 | 10.32 | A |
| ATOM | 563 | O | LEU | A | 78 | 34.561 | 82.883 | 42.902 | 1.00 | 10.65 | A |
| ATOM | 564 | N | VAL | A | 79 | 35.530 | 84.344 | 41.495 | 1.00 | 9.96 | A |
| ATOM | 565 | CA | VAL | A | 79 | 36.433 | 84.906 | 42.490 | 1.00 | 10.88 | A |
| ATOM | 566 | CB | VAL | A | 79 | 37.312 | 86.008 | 41.842 | 1.00 | 10.55 | A |
| ATOM | 567 | CG1 | VAL | A | 79 | 38.072 | 86.795 | 42.909 | 1.00 | 12.96 | A |
| ATOM | 568 | CG2 | VAL | A | 79 | 38.292 | 85.361 | 40.867 | 1.00 | 12.38 | A |
| ATOM | 569 | C | VAL | A | 79 | 35.702 | 85.474 | 43.703 | 1.00 | 11.38 | A |
| ATOM | 570 | O | VAL | A | 79 | 36.130 | 85.276 | 44.843 | 1.00 | 11.63 | A |
| ATOM | 571 | N | TYR | A | 80 | 34.594 | 86.165 | 43.459 | 1.00 | 11.08 | A |
| ATOM | 572 | CA | TYR | A | 80 | 33.828 | 86.777 | 44.542 | 1.00 | 11.80 | A |
| ATOM | 573 | CB | TYR | A | 80 | 33.337 | 88.151 | 44.075 | 1.00 | 11.15 | A |
| ATOM | 574 | CG | TYR | A | 80 | 34.484 | 89.044 | 43.675 | 1.00 | 11.18 | A |
| ATOM | 575 | CD1 | TYR | A | 80 | 35.504 | 89.342 | 44.580 | 1.00 | 12.28 | A |
| ATOM | 576 | CE1 | TYR | A | 80 | 36.612 | 90.094 | 44.197 | 1.00 | 13.38 | A |
| ATOM | 577 | CD2 | TYR | A | 80 | 34.593 | 89.531 | 42.374 | 1.00 | 11.57 | A |
| ATOM | 578 | CE2 | TYR | A | 80 | 35.696 | 90.284 | 41.981 | 1.00 | 12.40 | A |
| ATOM | 579 | CZ | TYR | A | 80 | 36.703 | 90.557 | 42.894 | 1.00 | 11.80 | A |
| ATOM | 580 | OH | TYR | A | 80 | 37.815 | 91.267 | 42.500 | 1.00 | 14.10 | A |
| ATOM | 581 | C | TYR | A | 80 | 32.724 | 85.889 | 45.105 | 1.00 | 12.27 | A |
| ATOM | 582 | O | TYR | A | 80 | 31.949 | 86.330 | 45.959 | 1.00 | 12.74 | A |
| ATOM | 583 | N | GLY | A | 81 | 32.650 | 84.650 | 44.625 | 1.00 | 12.12 | A |
| ATOM | 584 | CA | GLY | A | 81 | 31.662 | 83.703 | 45.121 | 1.00 | 13.73 | A |
| ATOM | 585 | C | GLY | A | 81 | 30.223 | 83.897 | 44.685 | 1.00 | 13.22 | A |
| ATOM | 586 | O | GLY | A | 81 | 29.306 | 83.441 | 45.373 | 1.00 | 15.91 | A |
| ATOM | 587 | N | LYS | A | 82 | 30.016 | 84.551 | 43.548 | 1.00 | 12.33 | A |
| ATOM | 588 | CA | LYS | A | 82 | 28.670 | 84.796 | 43.038 | 1.00 | 13.83 | A |
| ATOM | 589 | CB | LYS | A | 82 | 28.671 | 86.099 | 42.232 | 1.00 | 14.46 | A |
| ATOM | 590 | CG | LYS | A | 82 | 29.157 | 87.319 | 43.004 | 1.00 | 18.53 | A |
| ATOM | 591 | CD | LYS | A | 82 | 28.213 | 87.684 | 44.134 | 1.00 | 21.87 | A |
| ATOM | 592 | CE | LYS | A | 82 | 28.698 | 88.929 | 44.864 | 1.00 | 24.28 | A |
| ATOM | 593 | NZ | LYS | A | 82 | 27.763 | 89.348 | 45.947 | 1.00 | 25.39 | A |
| ATOM | 594 | C | LYS | A | 82 | 28.168 | 83.602 | 42.232 | 1.00 | 12.74 | A |
| ATOM | 595 | O | LYS | A | 82 | 26.965 | 83.452 | 42.013 | 1.00 | 14.81 | A |
| ATOM | 596 | N | ALA | A | 83 | 29.096 | 82.763 | 41.780 | 1.00 | 12.25 | A |
| ATOM | 597 | CA | ALA | A | 83 | 28.753 | 81.571 | 41.011 | 1.00 | 11.32 | A |
| ATOM | 598 | CB | ALA | A | 83 | 28.886 | 81.847 | 39.516 | 1.00 | 12.14 | A |
| ATOM | 599 | C | ALA | A | 83 | 29.681 | 80.434 | 41.424 | 1.00 | 10.84 | A |
| ATOM | 600 | O | ALA | A | 83 | 30.773 | 80.679 | 41.941 | 1.00 | 10.45 | A |
| ATOM | 601 | N | ASP | A | 84 | 29.244 | 79.197 | 41.195 | 1.00 | 10.99 | A |
| ATOM | 602 | CA | ASP | A | 84 | 30.026 | 78.021 | 41.562 | 1.00 | 10.42 | A |
| ATOM | 603 | CB | ASP | A | 84 | 29.091 | 76.929 | 42.093 | 1.00 | 12.81 | A |
| ATOM | 604 | CG | ASP | A | 84 | 28.252 | 77.389 | 43.264 | 1.00 | 14.10 | A |
| ATOM | 605 | OD1 | ASP | A | 84 | 28.829 | 77.871 | 44.261 | 1.00 | 18.48 | A |
| ATOM | 606 | OD2 | ASP | A | 84 | 27.014 | 77.258 | 43.186 | 1.00 | 15.34 | A |
| ATOM | 607 | C | ASP | A | 84 | 30.873 | 77.493 | 40.420 | 1.00 | 10.51 | A |
| ATOM | 608 | O | ILE | A | 85 | 31.906 | 76.864 | 40.648 | 1.00 | 10.54 | A |
| ATOM | 609 | N | ILE | A | 85 | 30.433 | 77.735 | 39.192 | 1.00 | 9.58 | A |
| ATOM | 610 | CA | ILE | A | 85 | 31.148 | 77.224 | 38.031 | 1.00 | 10.06 | A |
| ATOM | 611 | CB | ILE | A | 85 | 30.740 | 75.740 | 37.802 | 1.00 | 10.69 | A |
| ATOM | 612 | CG2 | ILE | A | 85 | 29.253 | 75.664 | 37.530 | 1.00 | 12.85 | A |
| ATOM | 613 | CG1 | ILE | A | 85 | 31.559 | 75.099 | 36.679 | 1.00 | 10.70 | A |
| ATOM | 614 | CD1 | ILE | A | 85 | 31.458 | 73.580 | 36.685 | 1.00 | 14.57 | A |
| ATOM | 615 | C | ILE | A | 85 | 30.812 | 78.062 | 36.802 | 1.00 | 9.88 | A |
| ATOM | 616 | O | ILE | A | 85 | 29.707 | 78.594 | 36.694 | 1.00 | 10.58 | A |
| ATOM | 617 | N | ALA | A | 86 | 31.776 | 78.201 | 35.895 | 1.00 | 8.99 | A |
| ATOM | 618 | CA | ALA | A | 86 | 31.569 | 78.949 | 34.659 | 1.00 | 8.68 | A |
| ATOM | 619 | CB | ALA | A | 86 | 32.602 | 80.066 | 34.521 | 1.00 | 8.41 | A |
| ATOM | 620 | C | ALA | A | 86 | 31.713 | 77.961 | 33.512 | 1.00 | 8.34 | A |
| ATOM | 621 | O | ALA | A | 86 | 32.723 | 77.261 | 33.414 | 1.00 | 8.88 | A |
| ATOM | 622 | N | ILE | A | 87 | 30.691 | 77.885 | 32.667 | 1.00 | 8.85 | A |
| ATOM | 623 | CA | ILE | A | 87 | 30.692 | 76.986 | 31.518 | 1.00 | 8.53 | A |
| ATOM | 624 | CB | ILE | A | 87 | 29.648 | 75.857 | 31.693 | 1.00 | 10.55 | A |
| ATOM | 625 | CG2 | ILE | A | 87 | 29.684 | 74.918 | 30.490 | 1.00 | 10.47 | A |
| ATOM | 626 | CG1 | ILE | A | 87 | 29.954 | 75.079 | 32.978 | 1.00 | 9.16 | A |
| ATOM | 627 | CD1 | ILE | A | 87 | 28.994 | 73.948 | 33.269 | 1.00 | 11.00 | A |
| ATOM | 628 | C | ILE | A | 87 | 30.371 | 77.856 | 30.311 | 1.00 | 9.03 | A |
| ATOM | 629 | O | ILE | A | 87 | 29.214 | 78.208 | 30.060 | 1.00 | 9.01 | A |
| ATOM | 630 | N | ALA | A | 88 | 31.421 | 78.206 | 29.576 | 1.00 | 8.95 | A |
| ATOM | 631 | CA | ALA | A | 88 | 31.309 | 79.098 | 28.433 | 1.00 | 8.86 | A |
| ATOM | 632 | CB | ALA | A | 88 | 31.149 | 80.523 | 28.948 | 1.00 | 11.38 | A |
| ATOM | 633 | C | ALA | A | 88 | 32.589 | 78.994 | 27.609 | 1.00 | 8.27 | A |
| ATOM | 634 | O | ALA | A | 88 | 33.492 | 78.240 | 27.960 | 1.00 | 9.26 | A |
| ATOM | 635 | N | PRO | A | 89 | 32.668 | 79.730 | 26.485 | 1.00 | 9.01 | A |
| ATOM | 636 | CD | PRO | A | 89 | 31.561 | 80.392 | 25.770 | 1.00 | 9.13 | A |
| ATOM | 637 | CA | PRO | A | 89 | 33.883 | 79.688 | 25.659 | 1.00 | 8.61 | A |
| ATOM | 638 | CB | PRO | A | 89 | 33.378 | 80.165 | 24.294 | 1.00 | 8.98 | A |
| ATOM | 639 | CG | PRO | A | 89 | 32.312 | 81.179 | 24.635 | 1.00 | 9.79 | A |
| ATOM | 640 | C | PRO | A | 89 | 34.921 | 80.580 | 26.345 | 1.00 | 7.75 | A |
| ATOM | 641 | O | PRO | A | 89 | 35.333 | 81.614 | 25.812 | 1.00 | 9.37 | A |
| ATOM | 642 | N | LEU | A | 90 | 35.324 | 80.169 | 27.544 | 1.00 | 8.01 | A |
| ATOM | 643 | CA | LEU | A | 90 | 36.275 | 80.911 | 28.362 | 1.00 | 8.41 | A |
| ATOM | 644 | CB | LEU | A | 90 | 35.867 | 80.769 | 29.833 | 1.00 | 9.56 | A |
| ATOM | 645 | CG | LEU | A | 90 | 36.698 | 81.544 | 30.860 | 1.00 | 7.48 | A |
| ATOM | 646 | CD1 | LEU | A | 90 | 36.513 | 83.048 | 30.672 | 1.00 | 8.55 | A |
| ATOM | 647 | CD2 | LEU | A | 90 | 36.265 | 81.138 | 32.258 | 1.00 | 9.99 | A |
| ATOM | 648 | C | LEU | A | 90 | 37.679 | 80.408 | 28.084 | 1.00 | 8.10 | A |
| ATOM | 649 | O | LEU | A | 90 | 38.040 | 79.293 | 28.460 | 1.00 | 8.62 | A |
| ATOM | 650 | N | THR | A | 91 | 38.483 | 81.250 | 27.452 | 1.00 | 7.27 | A |
| ATOM | 651 | CA | THR | A | 91 | 39.836 | 80.876 | 27.092 | 1.00 | 8.12 | A |
| ATOM | 652 | CB | THR | A | 91 | 40.400 | 81.907 | 26.114 | 1.00 | 9.08 | A |
| ATOM | 653 | OG1 | THR | A | 91 | 39.507 | 82.005 | 24.999 | 1.00 | 9.56 | A |
| ATOM | 654 | CG2 | THR | A | 91 | 41.785 | 81.497 | 25.622 | 1.00 | 10.14 | A |
| ATOM | 655 | C | THR | A | 91 | 40.745 | 80.732 | 28.300 | 1.00 | 7.70 | A |
| ATOM | 656 | O | THR | A | 91 | 40.746 | 81.572 | 29.199 | 1.00 | 8.49 | A |
| ATOM | 657 | N | ILE | A | 92 | 41.505 | 79.645 | 28.324 | 1.00 | 8.89 | A |
| ATOM | 658 | CA | ILE | A | 92 | 42.436 | 79.391 | 29.409 | 1.00 | 9.54 | A |
| ATOM | 659 | CB | ILE | A | 92 | 42.883 | 77.904 | 29.418 | 1.00 | 8.47 | A |
| ATOM | 660 | CG2 | ILE | A | 92 | 43.970 | 77.690 | 30.460 | 1.00 | 9.72 | A |
| ATOM | 661 | CG1 | ILE | A | 92 | 41.681 | 77.000 | 29.699 | 1.00 | 8.90 | A |
| ATOM | 662 | CD1 | ILE | A | 92 | 41.977 | 75.524 | 29.544 | 1.00 | 11.06 | A |
| ATOM | 663 | C | ILE | A | 92 | 43.652 | 80.290 | 29.200 | 1.00 | 10.70 | A |
| ATOM | 664 | O | ILE | A | 92 | 44.369 | 80.160 | 28.203 | 1.00 | 11.28 | A |
| ATOM | 665 | N | THR | A | 93 | 43.860 | 81.221 | 30.126 | 1.00 | 9.30 | A |
| ATOM | 666 | CA | THR | A | 93 | 44.995 | 82.135 | 30.053 | 1.00 | 10.20 | A |
| ATOM | 667 | CB | THR | A | 93 | 44.561 | 83.577 | 29.717 | 1.00 | 10.42 | A |
| ATOM | 668 | OG1 | THR | A | 93 | 43.804 | 84.120 | 30.807 | 1.00 | 11.85 | A |
| ATOM | 669 | CG2 | THR | A | 93 | 43.720 | 83.602 | 28.448 | 1.00 | 10.24 | A |
| ATOM | 670 | C | THR | A | 93 | 45.709 | 82.156 | 31.394 | 1.00 | 10.29 | A |
| ATOM | 671 | O | THR | A | 93 | 45.131 | 81.803 | 32.423 | 1.00 | 11.44 | A |
| ATOM | 672 | N | LEU | A | 94 | 46.965 | 82.586 | 31.376 | 1.00 | 10.68 | A |
| ATOM | 673 | CA | LEU | A | 94 | 47.771 | 82.644 | 32.583 | 1.00 | 10.99 | A |
| ATOM | 674 | CB | LEU | A | 94 | 49.185 | 83.111 | 32.223 | 1.00 | 11.64 | A |
| ATOM | 675 | CG | LEU | A | 94 | 50.083 | 83.424 | 33.423 | 1.00 | 13.09 | A |
| ATOM | 676 | CD1 | LEU | A | 94 | 50.364 | 82.151 | 34.203 | 1.00 | 14.45 | A |
| ATOM | 677 | CD2 | LEU | A | 94 | 51.380 | 84.051 | 32.935 | 1.00 | 16.24 | A |
| ATOM | 678 | C | LEU | A | 94 | 47.148 | 83.501 | 33.677 | 1.00 | 10.53 | A |
| ATOM | 679 | O | LEU | A | 94 | 47.025 | 83.055 | 34.812 | 1.00 | 10.79 | A |
| ATOM | 680 | N | VAL | A | 95 | 46.768 | 84.730 | 33.345 | 1.00 | 11.04 | A |
| ATOM | 681 | CA | VAL | A | 95 | 46.202 | 85.610 | 34.360 | 1.00 | 11.98 | A |
| ATOM | 682 | CB | VAL | A | 95 | 45.950 | 87.038 | 33.807 | 1.00 | 12.84 | A |
| ATOM | 683 | CG1 | VAL | A | 95 | 47.273 | 87.682 | 33.419 | 1.00 | 17.48 | A |
| ATOM | 684 | CG2 | VAL | A | 95 | 45.006 | 86.993 | 32.625 | 1.00 | 13.38 | A |
| ATOM | 685 | C | VAL | A | 95 | 44.927 | 85.059 | 34.991 | 1.00 | 10.75 | A |
| ATOM | 686 | O | VAL | A | 95 | 44.688 | 85.262 | 36.181 | 1.00 | 12.45 | A |
| ATOM | 687 | N | ARG | A | 96 | 44.119 | 84.350 | 34.206 | 1.00 | 10.25 | A |
| ATOM | 688 | CA | ARG | A | 96 | 42.890 | 83.764 | 34.734 | 1.00 | 10.47 | A |
| ATOM | 689 | CB | ARG | A | 96 | 41.944 | 83.429 | 33.575 | 1.00 | 9.65 | A |
| ATOM | 690 | CG | ARG | A | 96 | 41.356 | 84.636 | 32.883 | 1.00 | 10.28 | A |
| ATOM | 691 | CD | ARG | A | 96 | 40.559 | 84.214 | 31.668 | 1.00 | 9.89 | A |
| ATOM | 692 | NE | ARG | A | 96 | 39.948 | 85.350 | 30.993 | 1.00 | 8.57 | A |
| ATOM | 693 | CZ | ARG | A | 96 | 39.434 | 85.296 | 29.772 | 1.00 | 8.23 | A |
| ATOM | 694 | NH1 | ARG | A | 96 | 39.458 | 84.160 | 29.087 | 1.00 | 9.93 | A |
| ATOM | 695 | NH2 | ARG | A | 96 | 38.897 | 86.380 | 29.234 | 1.00 | 8.91 | A |
| ATOM | 696 | C | ARG | A | 96 | 43.231 | 82.551 | 35.596 | 1.00 | 9.78 | A |
| ATOM | 697 | O | ARG | A | 96 | 42.658 | 82.357 | 36.668 | 1.00 | 11.31 | A |
| ATOM | 698 | N | GLU | A | 97 | 44.172 | 81.740 | 35.122 | 1.00 | 10.06 | A |
| ATOM | 699 | CA | GLU | A | 97 | 44.589 | 80.539 | 35.841 | 1.00 | 9.63 | A |
| ATOM | 700 | CB | GLU | A | 97 | 45.602 | 79.771 | 34.984 | 1.00 | 10.82 | A |
| ATOM | 701 | CG | GLU | A | 97 | 46.124 | 78.475 | 35.602 | 1.00 | 12.19 | A |
| ATOM | 702 | CD | GLU | A | 97 | 45.075 | 77.380 | 35.668 | 1.00 | 13.87 | A |
| ATOM | 703 | OE1 | GLU | A | 97 | 44.169 | 77.468 | 36.522 | 1.00 | 13.02 | A |
| ATOM | 704 | OE2 | GLU | A | 97 | 45.156 | 76.429 | 34.859 | 1.00 | 13.68 | A |
| ATOM | 705 | C | GLU | A | 97 | 45.140 | 80.882 | 37.217 | 1.00 | 10.77 | A |
| ATOM | 706 | O | GLU | A | 97 | 45.077 | 80.073 | 38.137 | 1.00 | 11.69 | A |
| ATOM | 707 | N | GLU | A | 98 | 45.691 | 82.084 | 37.349 | 1.00 | 11.36 | A |
| ATOM | 708 | CA | GLU | A | 98 | 46.266 | 82.522 | 38.612 | 1.00 | 11.91 | A |
| ATOM | 709 | CB | GLU | A | 98 | 47.142 | 83.755 | 38.352 | 1.00 | 13.62 | A |
| ATOM | 710 | CG | GLU | A | 98 | 48.450 | 83.437 | 37.640 | 1.00 | 15.61 | A |
| ATOM | 711 | CD | GLU | A | 98 | 49.211 | 84.675 | 37.211 | 1.00 | 17.68 | A |
| ATOM | 712 | OE1 | GLU | A | 98 | 50.372 | 84.531 | 36.771 | 1.00 | 19.08 | A |
| ATOM | 713 | OE2 | GLU | A | 98 | 48.655 | 85.790 | 37.302 | 1.00 | 18.26 | A |
| ATOM | 714 | C | GLU | A | 98 | 45.223 | 82.763 | 39.697 | 1.00 | 12.62 | A |
| ATOM | 715 | O | GLU | A | 98 | 45.542 | 82.687 | 40.887 | 1.00 | 12.91 | A |
| ATOM | 716 | N | VAL | A | 99 | 43.980 | 83.034 | 39.303 | 1.00 | 11.67 | A |
| ATOM | 717 | CA | VAL | A | 99 | 42.941 | 83.321 | 40.287 | 1.00 | 10.90 | A |
| ATOM | 718 | CB | VAL | A | 99 | 42.364 | 84.740 | 40.071 | 1.00 | 11.56 | A |
| ATOM | 719 | CG1 | VAL | A | 99 | 43.480 | 85.766 | 40.189 | 1.00 | 13.71 | A |
| ATOM | 720 | CG2 | VAL | A | 99 | 41.682 | 84.836 | 38.714 | 1.00 | 11.03 | A |
| ATOM | 721 | C | VAL | A | 99 | 41.795 | 82.318 | 40.405 | 1.00 | 11.51 | A |
| ATOM | 722 | O | VAL | A | 99 | 41.082 | 82.320 | 41.405 | 1.00 | 11.13 | A |
| ATOM | 723 | N | ILE | A | 100 | 41.595 | 81.481 | 39.393 | 1.00 | 10.46 | A |
| ATOM | 724 | CA | ILE | A | 100 | 40.552 | 80.457 | 39.464 | 1.00 | 9.90 | A |
| ATOM | 725 | CB | ILE | A | 100 | 39.286 | 80.834 | 38.631 | 1.00 | 9.71 | A |
| ATOM | 726 | CG2 | ILE | A | 100 | 38.515 | 81.951 | 39.338 | 1.00 | 10.50 | A |
| ATOM | 727 | CG1 | ILE | A | 100 | 39.667 | 81.254 | 37.204 | 1.00 | 11.01 | A |
| ATOM | 728 | CD1 | ILE | A | 100 | 40.105 | 80.117 | 36.296 | 1.00 | 12.75 | A |
| ATOM | 729 | C | ILE | A | 100 | 41.116 | 79.136 | 38.959 | 1.00 | 10.00 | A |
| ATOM | 730 | O | ILE | A | 100 | 42.239 | 79.101 | 38.456 | 1.00 | 10.35 | A |
| ATOM | 731 | N | ASP | A | 101 | 40.359 | 78.053 | 39.131 | 1.00 | 10.22 | A |
| ATOM | 732 | CA | ASP | A | 101 | 40.775 | 76.727 | 38.661 | 1.00 | 11.33 | A |
| ATOM | 733 | CB | ASP | A | 101 | 40.368 | 75.639 | 39.672 | 1.00 | 12.26 | A |
| ATOM | 734 | CG | ASP | A | 101 | 41.200 | 75.651 | 40.937 | 1.00 | 12.57 | A |
| ATOM | 735 | OD1 | ASP | A | 101 | 42.299 | 76.244 | 40.944 | 1.00 | 15.62 | A |
| ATOM | 736 | OD2 | ASP | A | 101 | 40.747 | 75.036 | 41.927 | 1.00 | 16.24 | A |
| ATOM | 737 | C | ASP | A | 101 | 40.144 | 76.460 | 37.294 | 1.00 | 11.64 | A |
| ATOM | 738 | O | ASP | A | 101 | 38.968 | 76.761 | 37.083 | 1.00 | 13.13 | A |
| ATOM | 739 | N | PHE | A | 102 | 40.927 | 75.894 | 36.377 | 1.00 | 11.49 | A |
| ATOM | 740 | CA | PHE | A | 102 | 40.457 | 75.544 | 35.035 | 1.00 | 11.37 | A |
| ATOM | 741 | CB | PHE | A | 102 | 41.356 | 76.177 | 33.965 | 1.00 | 10.36 | A |
| ATOM | 742 | CG | PHE | A | 102 | 40.941 | 77.549 | 33.515 | 1.00 | 11.81 | A |
| ATOM | 743 | CD1 | PHE | A | 102 | 39.781 | 77.742 | 32.964 | 1.00 | 9.78 | A |
| ATOM | 744 | CD2 | PHE | A | 102 | 41.785 | 78.523 | 33.604 | 1.00 | 11.62 | A |
| ATOM | 745 | CE1 | PHE | A | 102 | 39.408 | 78.988 | 32.442 | 1.00 | 9.57 | A |
| ATOM | 746 | CE2 | PHE | A | 102 | 41.505 | 79.800 | 33.114 | 1.00 | 11.74 | A |
| ATOM | 747 | CZ | PHE | A | 102 | 40.292 | 80.033 | 32.520 | 1.00 | 10.93 | A |
| ATOM | 748 | C | PHE | A | 102 | 40.489 | 74.029 | 34.880 | 1.00 | 11.55 | A |
| ATOM | 749 | O | PHE | A | 102 | 41.405 | 73.366 | 35.369 | 1.00 | 12.64 | A |
| ATOM | 750 | N | SER | A | 103 | 39.497 | 73.487 | 34.188 | 1.00 | 10.67 | A |
| ATOM | 751 | CA | SER | A | 103 | 39.470 | 72.064 | 33.895 | 1.00 | 11.23 | A |
| ATOM | 752 | CB | SER | A | 103 | 38.057 | 71.657 | 33.485 | 1.00 | 11.53 | A |
| ATOM | 753 | OG | SER | A | 103 | 37.750 | 72.193 | 32.198 | 1.00 | 11.77 | A |
| ATOM | 754 | C | SER | A | 103 | 40.421 | 71.950 | 32.712 | 1.00 | 11.41 | A |
| ATOM | 755 | O | SER | A | 103 | 40.866 | 72.960 | 32.167 | 1.00 | 12.01 | A |
| ATOM | 756 | N | LYS | A | 104 | 40.737 | 70.723 | 32.314 | 1.00 | 12.11 | A |
| ATOM | 757 | CA | LYS | A | 104 | 41.548 | 70.528 | 31.127 | 1.00 | 13.10 | A |
| ATOM | 758 | CB | LYS | A | 104 | 41.866 | 69.037 | 30.969 | 1.00 | 14.27 | A |
| ATOM | 759 | CG | LYS | A | 104 | 42.804 | 68.479 | 32.033 | 1.00 | 16.73 | A |
| ATOM | 760 | CD | LYS | A | 104 | 44.135 | 69.216 | 32.026 | 1.00 | 16.89 | A |
| ATOM | 761 | CE | LYS | A | 104 | 45.140 | 68.607 | 33.008 | 1.00 | 16.32 | A |
| ATOM | 762 | NZ | LYS | A | 104 | 44.760 | 68.757 | 34.448 | 1.00 | 15.33 | A |
| ATOM | 763 | C | LYS | A | 104 | 40.632 | 71.099 | 30.037 | 1.00 | 11.89 | A |
| ATOM | 764 | O | LYS | A | 104 | 39.415 | 71.156 | 30.212 | 1.00 | 11.44 | A |
| ATOM | 765 | N | PRO | A | 105 | 41.191 | 71.519 | 28.899 | 1.00 | 11.77 | A |
| ATOM | 766 | CD | PRO | A | 105 | 42.611 | 71.548 | 28.522 | 1.00 | 13.43 | A |
| ATOM | 767 | CA | PRO | A | 105 | 40.350 | 72.085 | 27.839 | 1.00 | 12.57 | A |
| ATOM | 768 | CB | PRO | A | 105 | 41.374 | 72.559 | 26.806 | 1.00 | 14.53 | A |
| ATOM | 769 | CG | PRO | A | 105 | 42.655 | 72.794 | 27.577 | 1.00 | 16.88 | A |
| ATOM | 770 | C | PRO | A | 105 | 39.288 | 71.146 | 27.281 | 1.00 | 9.98 | A |
| ATOM | 771 | O | PRO | A | 105 | 39.530 | 69.954 | 27.108 | 1.00 | 10.01 | A |
| ATOM | 772 | N | PHE | A | 106 | 38.111 | 71.696 | 26.993 | 1.00 | 9.32 | A |
| ATOM | 773 | CA | PHE | A | 106 | 37.035 | 70.899 | 26.423 | 1.00 | 9.75 | A |
| ATOM | 774 | CB | PHE | A | 106 | 35.718 | 71.162 | 27.177 | 1.00 | 8.88 | A |
| ATOM | 775 | CG | PHE | A | 106 | 35.210 | 72.583 | 27.087 | 1.00 | 8.44 | A |
| ATOM | 776 | CD1 | PHE | A | 106 | 34.605 | 73.001 | 26.013 | 1.00 | 8.87 | A |
| ATOM | 777 | CD2 | PHE | A | 106 | 35.328 | 73.370 | 28.111 | 1.00 | 9.33 | A |
| ATOM | 778 | CE1 | PHE | A | 106 | 34.055 | 74.295 | 25.921 | 1.00 | 8.35 | A |
| ATOM | 779 | CE2 | PHE | A | 106 | 34.813 | 74.677 | 28.115 | 1.00 | 9.88 | A |
| ATOM | 780 | CZ | PHE | A | 106 | 34.165 | 75.142 | 26.999 | 1.00 | 8.63 | A |
| ATOM | 781 | C | PHE | A | 106 | 36.935 | 71.147 | 24.926 | 1.00 | 8.06 | A |
| ATOM | 782 | O | PHE | A | 106 | 36.241 | 70.422 | 24.222 | 1.00 | 9.73 | A |
| ATOM | 783 | N | MET | A | 107 | 37.652 | 72.159 | 24.439 | 1.00 | 8.72 | A |
| ATOM | 784 | CA | MET | A | 107 | 37.636 | 72.506 | 23.021 | 1.00 | 9.16 | A |
| ATOM | 785 | CB | MET | A | 107 | 36.377 | 73.331 | 22.711 | 1.00 | 12.85 | A |
| ATOM | 786 | CG | MET | A | 107 | 36.224 | 73.735 | 21.245 | 1.00 | 12.90 | A |
| ATOM | 787 | SD | MET | A | 107 | 34.755 | 74.745 | 20.904 | 1.00 | 15.47 | A |
| ATOM | 788 | CE | MET | A | 107 | 33.499 | 73.506 | 20.828 | 1.00 | 15.97 | A |
| ATOM | 789 | C | MET | A | 107 | 38.916 | 73.255 | 22.683 | 1.00 | 9.96 | A |
| ATOM | 790 | O | MET | A | 107 | 39.394 | 74.071 | 23.474 | 1.00 | 8.85 | A |
| ATOM | 791 | N | SER | A | 108 | 39.463 | 72.978 | 21.506 | 1.00 | 8.50 | A |
| ATOM | 792 | CA | SER | A | 108 | 40.679 | 73.639 | 21.056 | 1.00 | 9.44 | A |
| ATOM | 793 | CB | SER | A | 108 | 41.524 | 72.639 | 20.273 | 1.00 | 11.24 | A |
| ATOM | 794 | OG | SER | A | 108 | 40.862 | 72.290 | 19.072 | 1.00 | 14.99 | A |
| ATOM | 795 | C | SER | A | 108 | 40.332 | 74.858 | 20.212 | 1.00 | 10.05 | A |
| ATOM | 796 | O | SER | A | 108 | 39.270 | 74.929 | 19.593 | 1.00 | 10.23 | A |
| ATOM | 797 | N | LEU | A | 109 | 41.244 | 75.819 | 20.189 | 1.00 | 11.63 | A |
| ATOM | 798 | CA | LEU | A | 109 | 41.057 | 77.008 | 19.376 | 1.00 | 9.32 | A |
| ATOM | 799 | CB | LEU | A | 109 | 39.908 | 77.859 | 19.924 | 1.00 | 11.66 | A |
| ATOM | 800 | CG | LEU | A | 109 | 40.249 | 78.706 | 21.148 | 1.00 | 13.88 | A |
| ATOM | 801 | CD1 | LEU | A | 109 | 39.330 | 79.886 | 21.191 | 1.00 | 19.27 | A |
| ATOM | 802 | CD2 | LEU | A | 109 | 40.156 | 77.885 | 22.415 | 1.00 | 14.66 | A |
| ATOM | 803 | C | LEU | A | 109 | 42.353 | 77.786 | 19.313 | 1.00 | 9.33 | A |
| ATOM | 804 | O | LEU | A | 109 | 43.278 | 77.553 | 20.087 | 1.00 | 9.96 | A |
| ATOM | 805 | N | GLY | A | 110 | 42.396 | 78.715 | 18.370 | 1.00 | 8.83 | A |
| ATOM | 806 | CA | GLY | A | 110 | 43.541 | 79.582 | 18.207 | 1.00 | 8.86 | A |
| ATOM | 807 | C | GLY | A | 110 | 43.043 | 80.860 | 17.570 | 1.00 | 9.39 | A |
| ATOM | 808 | O | GLY | A | 110 | 42.017 | 80.852 | 16.890 | 1.00 | 9.15 | A |
| ATOM | 809 | N | ILE | A | 111 | 43.740 | 81.969 | 17.802 | 1.00 | 9.81 | A |
| ATOM | 810 | CA | ILE | A | 111 | 43.344 | 83.226 | 17.186 | 1.00 | 10.07 | A |
| ATOM | 811 | CB | ILE | A | 111 | 44.243 | 84.383 | 17.669 | 1.00 | 9.38 | A |
| ATOM | 812 | CG2 | ILE | A | 111 | 43.991 | 85.640 | 16.840 | 1.00 | 11.77 | A |
| ATOM | 813 | CG1 | ILE | A | 111 | 43.964 | 84.639 | 19.151 | 1.00 | 10.08 | A |
| ATOM | 814 | CD1 | ILE | A | 111 | 44.944 | 85.579 | 19.802 | 1.00 | 11.40 | A |
| ATOM | 815 | C | ILE | A | 111 | 43.483 | 82.993 | 15.684 | 1.00 | 8.75 | A |
| ATOM | 816 | O | ILE | A | 111 | 44.429 | 82.340 | 15.231 | 1.00 | 9.13 | A |
| ATOM | 817 | N | SER | A | 112 | 42.531 | 83.512 | 14.918 | 1.00 | 8.66 | A |
| ATOM | 818 | CA | SER | A | 112 | 42.510 | 83.305 | 13.479 | 1.00 | 9.16 | A |
| ATOM | 819 | CB | SER | A | 112 | 41.622 | 82.096 | 13.175 | 1.00 | 8.16 | A |
| ATOM | 820 | OG | SER | A | 112 | 40.264 | 82.396 | 13.473 | 1.00 | 10.60 | A |
| ATOM | 821 | C | SER | A | 112 | 42.036 | 84.546 | 12.750 | 1.00 | 9.12 | A |
| ATOM | 822 | O | SER | A | 112 | 41.534 | 85.492 | 13.356 | 1.00 | 9.91 | A |
| ATOM | 823 | N | ILE | A | 113 | 42.165 | 84.510 | 11.430 | 1.00 | 10.69 | A |
| ATOM | 824 | CA | ILE | A | 113 | 41.763 | 85.622 | 10.583 | 1.00 | 11.17 | A |
| ATOM | 825 | CB | ILE | A | 113 | 42.876 | 85.965 | 9.568 | 1.00 | 11.13 | A |
| ATOM | 826 | CG2 | ILE | A | 113 | 42.430 | 87.120 | 8.676 | 1.00 | 12.54 | A |
| ATOM | 827 | CG1 | ILE | A | 113 | 44.168 | 86.320 | 10.304 | 1.00 | 10.82 | A |
| ATOM | 828 | CD1 | ILE | A | 113 | 45.405 | 86.307 | 9.420 | 1.00 | 10.07 | A |
| ATOM | 829 | C | ILE | A | 113 | 40.495 | 85.307 | 9.791 | 1.00 | 10.13 | A |
| ATOM | 830 | O | ILE | A | 113 | 40.408 | 84.267 | 9.136 | 1.00 | 10.81 | A |
| ATOM | 831 | N | MET | A | 114 | 39.512 | 86.200 | 9.872 | 1.00 | 10.04 | A |
| ATOM | 832 | CA | MET | A | 114 | 38.286 | 86.053 | 9.099 | 1.00 | 10.61 | A |
| ATOM | 833 | CB | MET | A | 114 | 37.045 | 86.178 | 9.988 | 1.00 | 10.16 | A |
| ATOM | 834 | CG | MET | A | 114 | 35.760 | 86.220 | 9.157 | 1.00 | 10.60 | A |
| ATOM | 835 | SD | MET | A | 114 | 34.283 | 86.713 | 10.057 | 1.00 | 12.18 | A |
| ATOM | 836 | CE | MET | A | 114 | 34.047 | 85.278 | 11.121 | 1.00 | 13.11 | A |
| ATOM | 837 | C | MET | A | 114 | 38.310 | 87.142 | 8.038 | 1.00 | 11.63 | A |
| ATOM | 838 | O | MET | A | 114 | 38.488 | 88.320 | 8.356 | 1.00 | 10.82 | A |
| ATOM | 839 | N | ILE | A | 115 | 38.133 | 86.749 | 6.782 | 1.00 | 11.48 | A |
| ATOM | 840 | CA | ILE | A | 115 | 38.105 | 87.705 | 5.687 | 1.00 | 11.96 | A |
| ATOM | 841 | CB | ILE | A | 115 | 39.272 | 87.499 | 4.685 | 1.00 | 11.71 | A |
| ATOM | 842 | CG2 | ILE | A | 115 | 40.595 | 87.827 | 5.340 | 1.00 | 13.51 | A |
| ATOM | 843 | CG1 | ILE | A | 115 | 39.241 | 86.067 | 4.140 | 1.00 | 12.50 | A |
| ATOM | 844 | CD1 | ILE | A | 115 | 40.170 | 85.830 | 2.954 | 1.00 | 16.93 | A |
| ATOM | 845 | C | ILE | A | 115 | 36.825 | 87.521 | 4.894 | 1.00 | 11.78 | A |
| ATOM | 846 | O | ILE | A | 115 | 36.151 | 86.495 | 5.001 | 1.00 | 11.27 | A |
| ATOM | 847 | N | LYS | A | 116 | 36.494 | 88.541 | 4.112 | 1.00 | 13.14 | A |
| ATOM | 848 | CA | LYS | A | 116 | 35.351 | 88.500 | 3.220 | 1.00 | 12.56 | A |
| ATOM | 849 | CB | LYS | A | 116 | 34.941 | 89.936 | 2.880 | 1.00 | 13.86 | A |
| ATOM | 850 | CG | LYS | A | 116 | 33.922 | 90.095 | 1.758 | 1.00 | 18.08 | A |
| ATOM | 851 | CD | LYS | A | 116 | 32.539 | 89.615 | 2.142 | 1.00 | 20.52 | A |
| ATOM | 852 | CE | LYS | A | 116 | 31.476 | 90.375 | 1.351 | 1.00 | 21.38 | A |
| ATOM | 853 | NZ | LYS | A | 116 | 31.798 | 90.486 | −0.103 | 1.00 | 22.41 | A |
| ATOM | 854 | C | LYS | A | 116 | 35.954 | 87.735 | 2.046 | 1.00 | 12.06 | A |
| ATOM | 855 | O | LYS | A | 116 | 37.093 | 88.000 | 1.662 | 1.00 | 12.42 | A |
| ATOM | 856 | N | LYS | A | 117 | 35.221 | 86.780 | 1.488 | 1.00 | 12.31 | A |
| ATOM | 857 | CA | LYS | A | 117 | 35.751 | 86.009 | 0.366 | 1.00 | 12.17 | A |
| ATOM | 858 | CB | LYS | A | 117 | 34.659 | 85.090 | −0.188 | 1.00 | 13.23 | A |
| ATOM | 859 | CG | LYS | A | 117 | 34.338 | 83.905 | 0.701 | 1.00 | 13.86 | A |
| ATOM | 860 | CD | LYS | A | 117 | 33.367 | 82.955 | 0.020 | 1.00 | 13.48 | A |
| ATOM | 861 | CE | LYS | A | 117 | 33.108 | 81.727 | 0.879 | 1.00 | 15.63 | A |
| ATOM | 862 | NZ | LYS | A | 117 | 32.169 | 80.780 | 0.215 | 1.00 | 16.49 | A |
| ATOM | 863 | C | LYS | A | 117 | 36.341 | 86.924 | −0.697 | 1.00 | 11.97 | A |
| ATOM | 864 | O | LYS | A | 117 | 35.742 | 87.938 | −1.063 | 1.00 | 12.59 | A |
| ATOM | 865 | N | GLY | A | 118 | 37.525 | 86.565 | −1.183 | 1.00 | 13.45 | A |
| ATOM | 866 | CA | GLY | A | 118 | 38.167 | 87.359 | −2.216 | 1.00 | 13.64 | A |
| ATOM | 867 | C | GLY | A | 118 | 39.234 | 88.317 | −1.721 | 1.00 | 14.42 | A |
| ATOM | 868 | O | GLY | A | 118 | 40.003 | 88.860 | −2.515 | 1.00 | 14.72 | A |
| ATOM | 869 | N | THR | A | 119 | 39.287 | 88.532 | −0.410 | 1.00 | 13.34 | A |
| ATOM | 870 | CA | THR | A | 119 | 40.283 | 89.429 | 0.171 | 1.00 | 13.21 | A |
| ATOM | 871 | CB | THR | A | 119 | 40.025 | 89.633 | 1.676 | 1.00 | 13.24 | A |
| ATOM | 872 | OG1 | THR | A | 119 | 38.732 | 90.220 | 1.858 | 1.00 | 14.61 | A |
| ATOM | 873 | CG2 | THR | A | 119 | 41.075 | 90.551 | 2.279 | 1.00 | 14.14 | A |
| ATOM | 874 | C | THR | A | 119 | 41.684 | 88.845 | −0.032 | 1.00 | 13.53 | A |
| ATOM | 875 | O | THR | A | 119 | 41.934 | 87.685 | 0.292 | 1.00 | 13.93 | A |
| ATOM | 876 | N | PRO | A | 120 | 42.619 | 89.649 | −0.569 | 1.00 | 14.13 | A |
| ATOM | 877 | CD | PRO | A | 120 | 42.403 | 90.986 | −1.148 | 1.00 | 14.29 | A |
| ATOM | 878 | CA | PRO | A | 120 | 43.991 | 89.195 | −0.813 | 1.00 | 15.21 | A |
| ATOM | 879 | CB | PRO | A | 120 | 44.466 | 90.166 | −1.893 | 1.00 | 16.18 | A |
| ATOM | 880 | CG | PRO | A | 120 | 43.871 | 91.469 | −1.451 | 1.00 | 15.91 | A |
| ATOM | 881 | C | PRO | A | 120 | 44.856 | 89.133 | 0.442 | 1.00 | 14.83 | A |
| ATOM | 882 | O | PRO | A | 120 | 45.859 | 89.842 | 0.557 | 1.00 | 16.28 | A |
| ATOM | 883 | N | ILE | A | 121 | 44.461 | 88.469 | 1.373 | 1.00 | 15.05 | A |
| ATOM | 884 | CA | ILE | A | 121 | 45.173 | 88.072 | 2.634 | 1.00 | 14.62 | A |
| ATOM | 885 | CB | ILE | A | 121 | 44.468 | 88.816 | 3.801 | 1.00 | 14.17 | A |
| ATOM | 886 | CG2 | ILE | A | 121 | 45.026 | 88.343 | 5.135 | 1.00 | 13.82 | A |
| ATOM | 887 | CG1 | ILE | A | 121 | 44.628 | 90.330 | 3.643 | 1.00 | 14.82 | A |
| ATOM | 888 | CD1 | ILE | A | 121 | 46.058 | 90.828 | 3.780 | 1.00 | 14.64 | A |
| ATOM | 889 | C | ILE | A | 121 | 45.193 | 86.576 | 2.945 | 1.00 | 15.30 | A |
| ATOM | 890 | O | ILE | A | 121 | 44.154 | 85.917 | 2.902 | 1.00 | 15.51 | A |
| ATOM | 891 | N | GLU | A | 122 | 46.370 | 86.044 | 3.263 | 1.00 | 18.03 | A |
| ATOM | 892 | CA | GLU | A | 122 | 46.507 | 84.622 | 3.563 | 1.00 | 19.65 | A |
| ATOM | 893 | CB | GLU | A | 122 | 47.231 | 83.936 | 2.398 | 1.00 | 23.87 | A |
| ATOM | 894 | CG | GLU | A | 122 | 46.538 | 84.052 | 1.051 | 1.00 | 29.93 | A |
| ATOM | 895 | CD | GLU | A | 122 | 47.314 | 83.362 | −0.055 | 1.00 | 34.09 | A |
| ATOM | 896 | OE1 | GLU | A | 122 | 48.492 | 83.720 | −0.268 | 1.00 | 36.67 | A |
| ATOM | 897 | OE2 | GLU | A | 122 | 46.749 | 82.462 | −0.712 | 1.00 | 36.93 | A |
| ATOM | 898 | C | GLU | A | 122 | 47.200 | 84.348 | 4.894 | 1.00 | 18.90 | A |
| ATOM | 899 | O | GLU | A | 122 | 47.339 | 83.189 | 5.290 | 1.00 | 20.61 | A |
| ATOM | 900 | N | SER | A | 123 | 47.633 | 85.396 | 5.590 | 1.00 | 16.61 | A |
| ATOM | 901 | CA | SER | A | 123 | 48.331 | 85.205 | 6.858 | 1.00 | 15.22 | A |
| ATOM | 902 | CB | SER | A | 123 | 49.$$37 | 84.683 | 6.569 | 1.00 | 15.40 | A |
| ATOM | 903 | OG | SER | A | 123 | 50.528 | 85.721 | 6.012 | 1.00 | 17.88 | A |
| ATOM | 904 | C | SER | A | 123 | 48.380 | 86.481 | 7.681 | 1.00 | 13.61 | A |
| ATOM | 905 | O | SER | A | 123 | 48.069 | 87.567 | 7.190 | 1.00 | 13.29 | A |
| ATOM | 906 | N | ALA | A | 124 | 48.798 | 86.335 | 8.936 | 1.00 | 14.71 | A |
| ATOM | 907 | CA | ALA | A | 124 | 48.931 | 87.475 | 9.833 | 1.00 | 15.10 | A |
| ATOM | 908 | CB | ALA | A | 124 | 49.289 | 87.000 | 11.235 | 1.00 | 15.67 | A |
| ATOM | 909 | C | ALA | A | 124 | 50.037 | 88.371 | 9.281 | 1.00 | 14.20 | A |
| ATOM | 910 | O | ALA | A | 124 | 49.943 | 89.595 | 9.337 | 1.00 | 14.14 | A |
| ATOM | 911 | N | ALA | A | 125 | 51.089 | 87.751 | 8.752 | 1.00 | 14.72 | A |
| ATOM | 912 | CA | ALA | A | 125 | 52.196 | 88.505 | 8.172 | 1.00 | 14.98 | A |
| ATOM | 913 | CB | ALA | A | 125 | 53.245 | 87.552 | 7.613 | 1.00 | 17.46 | A |
| ATOM | 914 | C | ALA | A | 125 | 51.654 | 89.412 | 7.068 | 1.00 | 14.92 | A |
| ATOM | 915 | O | ALA | A | 125 | 52.038 | 90.578 | 6.970 | 1.00 | 15.73 | A |
| ATOM | 916 | N | ASP | A | 126 | 50.758 | 88.876 | 6.240 | 1.00 | 15.00 | A |
| ATOM | 917 | CA | ASP | A | 126 | 50.151 | 89.654 | 5.160 | 1.00 | 13.86 | A |
| ATOM | 918 | CB | ASP | A | 126 | 49.142 | 88.792 | 4.389 | 1.00 | 16.41 | A |
| ATOM | 919 | CG | ASP | A | 126 | 49.799 | 87.817 | 3.430 | 1.00 | 16.11 | A |
| ATOM | 920 | OD1 | ASP | A | 126 | 51.019 | 87.929 | 3.176 | 1.00 | 17.69 | A |
| ATOM | 921 | OD2 | ASP | A | 126 | 49.074 | 86.940 | 2.915 | 1.00 | 18.28 | A |
| ATOM | 922 | C | ASP | A | 126 | 49.494 | 90.914 | 5.721 | 1.00 | 14.64 | A |
| ATOM | 923 | O | ASP | A | 126 | 49.682 | 92.011 | 5.198 | 1.00 | 16.49 | A |
| ATOM | 924 | N | LEU | A | 127 | 48.702 | 90.749 | 6.776 | 1.00 | 14.07 | A |
| ATOM | 925 | CA | LEU | A | 127 | 48.017 | 91.878 | 7.397 | 1.00 | 13.65 | A |
| ATOM | 926 | CB | LEU | A | 127 | 47.061 | 91.367 | 8.480 | 1.00 | 14.34 | A |
| ATOM | 927 | CG | LEU | A | 127 | 45.763 | 90.664 | 8.087 | 1.00 | 13.53 | A |
| ATOM | 928 | CD1 | LEU | A | 127 | 45.078 | 90.165 | 9.349 | 1.00 | 13.86 | A |
| ATOM | 929 | CD2 | LEU | A | 127 | 44.857 | 91.617 | 7.323 | 1.00 | 12.53 | A |
| ATOM | 930 | C | LEU | A | 127 | 48.997 | 92.901 | 7.955 | 1.00 | 14.30 | A |
| ATOM | 931 | O | LEU | A | 127 | 48.853 | 94.098 | 7.724 | 1.00 | 14.36 | A |
| ATOM | 932 | N | SER | A | 128 | 49.986 | 92.416 | 8.696 | 1.00 | 15.05 | A |
| ATOM | 933 | CA | SER | A | 128 | 50.973 | 93.282 | 9.331 | 1.00 | 15.56 | A |
| ATOM | 934 | CB | SER | A | 128 | 51.931 | 92.419 | 10.159 | 1.00 | 15.97 | A |
| ATOM | 935 | OG | SER | A | 128 | 52.837 | 91.719 | 9.321 | 1.00 | 17.93 | A |
| ATOM | 936 | C | SER | A | 128 | 51.722 | 94.188 | 8.362 | 1.00 | 17.20 | A |
| ATOM | 937 | O | SER | A | 128 | 52.102 | 95.301 | 8.724 | 1.00 | 17.49 | A |
| ATOM | 938 | N | LYS | A | 129 | 51.928 | 93.712 | 7.138 | 1.00 | 17.72 | A |
| ATOM | 939 | CA | LYS | A | 129 | 52.672 | 94.464 | 6.129 | 1.00 | 20.04 | A |
| ATOM | 940 | CB | LYS | A | 129 | 53.389 | 93.469 | 5.210 | 1.00 | 20.87 | A |
| ATOM | 941 | CG | LYS | A | 129 | 54.392 | 92.568 | 5.902 | 1.00 | 23.87 | A |
| ATOM | 942 | CD | LYS | A | 129 | 55.000 | 91.592 | 4.907 | 1.00 | 27.53 | A |
| ATOM | 943 | CE | LYS | A | 129 | 56.036 | 90.695 | 5.561 | 1.00 | 29.36 | A |
| ATOM | 944 | NZ | LYS | A | 129 | 56.599 | 89.713 | 4.592 | 1.00 | 30.61 | A |
| ATOM | 945 | C | LYS | A | 129 | 51.860 | 95.474 | 5.329 | 1.00 | 21.23 | A |
| ATOM | 946 | O | LYS | A | 129 | 52.348 | 95.992 | 4.324 | 1.00 | 23.30 | A |
| ATOM | 947 | N | GLN | A | 130 | 50.636 | 95.771 | 5.753 | 1.00 | 19.95 | A |
| ATOM | 948 | CA | GLN | A | 130 | 49.824 | 96.721 | 5.003 | 1.00 | 18.30 | A |
| ATOM | 949 | CB | GLN | A | 130 | 49.225 | 95.991 | 3.795 | 1.00 | 18.41 | A |
| ATOM | 950 | CG | GLN | A | 130 | 48.329 | 94.814 | 4.160 | 1.00 | 16.67 | A |
| ATOM | 951 | CD | GLN | A | 130 | 46.934 | 95.248 | 4.557 | 1.00 | 16.07 | A |
| ATOM | 952 | OE1 | GLN | A | 130 | 46.181 | 95.769 | 3.736 | 1.00 | 16.30 | A |
| ATOM | 953 | NE2 | GLN | A | 130 | 46.583 | 95.039 | 5.822 | 1.00 | 14.90 | A |
| ATOM | 954 | C | GLN | A | 130 | 48.781 | 97.420 | 5.865 | 1.00 | 17.82 | A |
| ATOM | 955 | O | GLN | A | 130 | 48.595 | 97.083 | 7.037 | 1.00 | 18.78 | A |
| ATOM | 956 | N | THR | A | 131 | 48.108 | 98.404 | 5.274 | 1.00 | 18.29 | A |
| ATOM | 957 | CA | THR | A | 131 | 47.083 | 99.166 | 5.975 | 1.00 | 18.33 | A |
| ATOM | 958 | CB | THR | A | 131 | 47.611 | 100.552 | 6.404 | 1.00 | 19.51 | A |
| ATOM | 959 | OG1 | THR | A | 131 | 48.105 | 101.250 | 5.255 | 1.00 | 19.09 | A |
| ATOM | 960 | CG2 | THR | A | 131 | 48.731 | 100.404 | 7.426 | 1.00 | 19.39 | A |
| ATOM | 961 | C | THR | A | 131 | 45.824 | 99.367 | 5.133 | 1.00 | 18.32 | A |
| ATOM | 962 | O | THR | A | 131 | 44.800 | 99.819 | 5.645 | 1.00 | 18.96 | A |
| ATOM | 963 | N | GLU | A | 132 | 45.904 | 99.029 | 3.848 | 1.00 | 18.96 | A |
| ATOM | 964 | CA | GLU | A | 132 | 44.767 | 99.165 | 2.936 | 1.00 | 18.71 | A |
| ATOM | 965 | CB | GLU | A | 132 | 45.192 | 98.712 | 1.535 | 1.00 | 21.48 | A |
| ATOM | 966 | CG | GLU | A | 132 | 44.076 | 98.733 | 0.498 | 1.00 | 27.82 | A |
| ATOM | 967 | CD | GLU | A | 132 | 43.467 | 100.110 | 0.317 | 1.00 | 31.71 | A |
| ATOM | 968 | OE1 | GLU | A | 132 | 44.223 | 101.062 | 0.031 | 1.00 | 34.52 | A |
| ATOM | 969 | OE2 | GLU | A | 132 | 42.232 | 100.240 | 0.458 | 1.00 | 34.23 | A |
| ATOM | 970 | C | GLU | A | 132 | 43.583 | 98.375 | 3.483 | 1.00 | 16.80 | A |
| ATOM | 971 | O | GLU | A | 132 | 42.447 | 98.854 | 3.501 | 1.00 | 16.77 | A |
| ATOM | 972 | N | ILE | A | 133 | 43.858 | 97.150 | 3.912 | 1.00 | 14.67 | A |
| ATOM | 973 | CA | ILE | A | 133 | 42.834 | 96.297 | 4.488 | 1.00 | 13.77 | A |
| ATOM | 974 | CB | ILE | A | 133 | 43.047 | 94.823 | 4.089 | 1.00 | 13.06 | A |
| ATOM | 975 | CG2 | ILE | A | 133 | 41.967 | 93.948 | 4.716 | 1.00 | 13.29 | A |
| ATOM | 976 | CG1 | ILE | A | 133 | 43.007 | 94.691 | 2.564 | 1.00 | 15.61 | A |
| ATOM | 977 | CD1 | ILE | A | 133 | 43.390 | 93.322 | 2.050 | 1.00 | 16.33 | A |
| ATOM | 978 | C | ILE | A | 133 | 42.974 | 96.453 | 5.997 | 1.00 | 12.06 | A |
| ATOM | 979 | O | ILE | A | 133 | 43.949 | 95.998 | 6.595 | 1.00 | 13.27 | A |
| ATOM | 980 | N | ALA | A | 134 | 42.010 | 97.131 | 6.606 | 1.00 | 12.33 | A |
| ATOM | 981 | CA | ALA | A | 134 | 42.040 | 97.350 | 8.044 | 1.00 | 12.13 | A |
| ATOM | 982 | CB | ALA | A | 134 | 41.011 | 98.398 | 8.429 | 1.00 | 12.49 | A |
| ATOM | 983 | C | ALA | A | 134 | 41.749 | 96.035 | 8.754 | 1.00 | 11.32 | A |
| ATOM | 984 | O | ALA | A | 134 | 41.132 | 95.136 | 8.179 | 1.00 | 13.18 | A |
| ATOM | 985 | N | TYR | A | 135 | 42.200 | 95.916 | 9.998 | 1.00 | 10.47 | A |
| ATOM | 986 | CA | TYR | A | 135 | 41.964 | 94.694 | 10.754 | 1.00 | 9.42 | A |
| ATOM | 987 | CB | TYR | A | 135 | 43.006 | 93.637 | 10.351 | 1.00 | 9.29 | A |
| ATOM | 988 | CG | TYR | A | 135 | 44.446 | 94.040 | 10.576 | 1.00 | 9.02 | A |
| ATOM | 989 | CD1 | TYR | A | 135 | 45.090 | 93.755 | 11.779 | 1.00 | 10.76 | A |
| ATOM | 990 | CE1 | TYR | A | 135 | 46.421 | 94.111 | 11.987 | 1.00 | 11.52 | A |
| ATOM | 991 | CD2 | TYR | A | 135 | 45.171 | 94.699 | 9.582 | 1.00 | 11.38 | A |
| ATOM | 992 | CE2 | TYR | A | 135 | 46.504 | 95.061 | 9.781 | 1.00 | 10.76 | A |
| ATOM | 993 | CZ | TYR | A | 135 | 47.122 | 94.763 | 10.982 | 1.00 | 11.29 | A |
| ATOM | 994 | OH | TYR | A | 135 | 48.437 | 95.127 | 11.183 | 1.00 | 11.63 | A |
| ATOM | 995 | C | TYR | A | 135 | 41.924 | 94.991 | 12.244 | 1.00 | 10.12 | A |
| ATOM | 996 | O | TYR | A | 135 | 42.615 | 95.882 | 12.730 | 1.00 | 10.23 | A |
| ATOM | 997 | N | GLY | A | 136 | 41.104 | 94.238 | 12.969 | 1.00 | 10.76 | A |
| ATOM | 998 | CA | GLY | A | 136 | 40.988 | 94.464 | 14.394 | 1.00 | 9.01 | A |
| ATOM | 999 | C | GLY | A | 136 | 40.458 | 93.275 | 15.167 | 1.00 | 10.02 | A |
| ATOM | 1000 | O | GLY | A | 136 | 40.264 | 92.186 | 14.617 | 1.00 | 10.81 | A |
| ATOM | 1001 | N | THR | A | 137 | 40.212 | 93.508 | 16.453 | 1.00 | 10.56 | A |
| ATOM | 1002 | CA | THR | A | 137 | 39.721 | 92.486 | 17.372 | 1.00 | 11.45 | A |
| ATOM | 1003 | CB | THR | A | 137 | 40.820 | 92.075 | 18.379 | 1.00 | 13.34 | A |
| ATOM | 1004 | OG1 | THR | A | 137 | 41.070 | 93.168 | 19.276 | 1.00 | 15.72 | A |
| ATOM | 1005 | CG2 | THR | A | 137 | 42.115 | 91.724 | 17.663 | 1.00 | 13.60 | A |
| ATOM | 1006 | C | THR | A | 137 | 38.570 | 93.052 | 18.200 | 1.00 | 10.89 | A |
| ATOM | 1007 | O | THR | A | 137 | 38.135 | 94.192 | 18.000 | 1.00 | 11.43 | A |
| ATOM | 1008 | N | LEU | A | 138 | 38.077 | 92.241 | 19.130 | 1.00 | 11.39 | A |
| ATOM | 1009 | CA | LEU | A | 138 | 37.026 | 92.684 | 20.032 | 1.00 | 11.58 | A |
| ATOM | 1010 | CB | LEU | A | 138 | 36.451 | 91.476 | 20.774 | 1.00 | 12.41 | A |
| ATOM | 1011 | CG | LEU | A | 138 | 35.496 | 90.568 | 20.003 | 1.00 | 14.07 | A |
| ATOM | 1012 | CD1 | LEU | A | 138 | 35.299 | 89.276 | 20.784 | 1.00 | 14.31 | A |
| ATOM | 1013 | CD2 | LEU | A | 138 | 34.174 | 91.283 | 19.771 | 1.00 | 15.53 | A |
| ATOM | 1014 | C | LEU | A | 138 | 37.703 | 93.691 | 20.955 | 1.00 | 14.74 | A |
| ATOM | 1015 | O | LEU | A | 138 | 38.923 | 93.641 | 21.151 | 1.00 | 15.25 | A |
| ATOM | 1016 | N | ASP | A | 139 | 36.922 | 94.603 | 21.525 | 1.00 | 15.55 | A |
| ATOM | 1017 | CA | ASP | A | 139 | 37.483 | 95.618 | 22.411 | 1.00 | 19.69 | A |
| ATOM | 1018 | CB | ASP | A | 139 | 36.539 | 96.823 | 22.455 | 1.00 | 24.00 | A |
| ATOM | 1019 | CG | ASP | A | 139 | 35.192 | 96.487 | 23.050 | 1.00 | 29.20 | A |
| ATOM | 1020 | OD1 | ASP | A | 139 | 34.547 | 95.536 | 22.560 | 1.00 | 32.68 | A |
| ATOM | 1021 | OD2 | ASP | A | 139 | 34.776 | 97.178 | 24.005 | 1.00 | 32.36 | A |
| ATOM | 1022 | C | ASP | A | 139 | 37.815 | 95.061 | 23.792 | 1.00 | 19.51 | A |
| ATOM | 1023 | O | ASP | A | 139 | 38.333 | 95.777 | 24.648 | 1.00 | 20.23 | A |
| ATOM | 1024 | N | SER | A | 140 | 37.512 | 93.787 | 24.009 | 1.00 | 17.95 | A |
| ATOM | 1025 | CA | SER | A | 140 | 37.802 | 93.135 | 25.280 | 1.00 | 17.27 | A |
| ATOM | 1026 | CB | SER | A | 140 | 36.722 | 93.499 | 26.305 | 1.00 | 19.12 | A |
| ATOM | 1027 | OG | SER | A | 140 | 35.477 | 92.929 | 25.958 | 1.00 | 24.29 | A |
| ATOM | 1028 | C | SER | A | 140 | 37.908 | 91.636 | 25.025 | 1.00 | 15.40 | A |
| ATOM | 1029 | O | SER | A | 140 | 37.276 | 91.110 | 24.109 | 1.00 | 16.77 | A |
| ATOM | 1030 | N | GLY | A | 141 | 38.707 | 90.951 | 25.835 | 1.00 | 14.00 | A |
| ATOM | 1031 | CA | GLY | A | 141 | 38.863 | 89.520 | 25.658 | 1.00 | 11.82 | A |
| ATOM | 1032 | C | GLY | A | 141 | 40.307 | 89.091 | 25.482 | 1.00 | 11.66 | A |
| ATOM | 1033 | O | GLY | A | 141 | 41.208 | 89.922 | 25.354 | 1.00 | 12.35 | A |
| ATOM | 1034 | N | SER | A | 142 | 40.521 | 87.780 | 25.445 | 1.00 | 10.38 | A |
| ATOM | 1035 | CA | SER | A | 142 | 41.860 | 87.215 | 25.323 | 1.00 | 10.71 | A |
| ATOM | 1036 | CB | SER | A | 142 | 41.788 | 85.707 | 25.573 | 1.00 | 12.33 | A |
| ATOM | 1037 | OG | SER | A | 142 | 41.281 | 85.427 | 26.861 | 1.00 | 20.39 | A |
| ATOM | 1038 | C | SER | A | 142 | 42.582 | 87.540 | 24.025 | 1.00 | 10.09 | A |
| ATOM | 1039 | O | SER | A | 142 | 43.809 | 87.576 | 23.995 | 1.00 | 11.57 | A |
| ATOM | 1040 | N | THR | A | 143 | 41.840 | 87.758 | 22.947 | 1.00 | 8.66 | A |
| ATOM | 1041 | CA | THR | A | 143 | 42.491 | 88.067 | 21.683 | 1.00 | 10.00 | A |
| ATOM | 1042 | CB | THR | A | 143 | 41.506 | 87.996 | 20.517 | 1.00 | 9.34 | A |
| ATOM | 1043 | OG1 | THR | A | 143 | 40.996 | 86.660 | 20.424 | 1.00 | 10.10 | A |
| ATOM | 1044 | CG2 | THR | A | 143 | 42.199 | 88.361 | 19.216 | 1.00 | 11.12 | A |
| ATOM | 1045 | C | THR | A | 143 | 43.128 | 89.447 | 21.738 | 1.00 | 9.95 | A |
| ATOM | 1046 | O | THR | A | 143 | 44.276 | 89.617 | 21.333 | 1.00 | 11.74 | A |
| ATOM | 1047 | N | LYS | A | 144 | 42.394 | 90.432 | 22.248 | 1.00 | 10.61 | A |
| ATOM | 1048 | CA | LYS | A | 144 | 42.940 | 91.779 | 22.361 | 1.00 | 12.70 | A |
| ATOM | 1049 | CB | LYS | A | 144 | 41.858 | 92.730 | 22.881 | 1.00 | 14.41 | A |
| ATOM | 1050 | CG | LYS | A | 144 | 42.307 | 94.186 | 22.949 | 1.00 | 16.73 | A |
| ATOM | 1051 | CD | LYS | A | 144 | 41.216 | 95.069 | 23.544 | 1.00 | 21.27 | A |
| ATOM | 1052 | CE | LYS | A | 144 | 41.672 | 96.512 | 23.677 | 1.00 | 23.82 | A |
| ATOM | 1053 | NZ | LYS | A | 144 | 40.633 | 97.363 | 24.322 | 1.00 | 25.58 | A |
| ATOM | 1054 | C | LYS | A | 144 | 44.162 | 91.715 | 23.271 | 1.00 | 12.70 | A |
| ATOM | 1055 | O | LYS | A | 144 | 45.197 | 92.314 | 22.980 | 1.00 | 13.06 | A |
| ATOM | 1056 | N | GLU | A | 145 | 44.040 | 90.976 | 24.369 | 1.00 | 13.84 | A |
| ATOM | 1057 | CA | GLU | A | 145 | 45.141 | 90.840 | 25.317 | 1.00 | 14.37 | A |
| ATOM | 1058 | CB | GLU | A | 145 | 44.646 | 90.108 | 26.567 | 1.00 | 18.35 | A |
| ATOM | 1059 | CG | GLU | A | 145 | 43.661 | 90.914 | 27.394 | 1.00 | 23.95 | A |
| ATOM | 1060 | CD | GLU | A | 145 | 44.154 | 92.323 | 27.666 | 1.00 | 27.85 | A |
| ATOM | 1061 | OE1 | GLU | A | 145 | 45.300 | 92.474 | 28.140 | 1.00 | 29.32 | A |
| ATOM | 1062 | OE2 | GLU | A | 145 | 43.393 | 93.279 | 27.408 | 1.00 | 31.79 | A |
| ATOM | 1063 | C | GLU | A | 145 | 46.350 | 90.172 | 24.677 | 1.00 | 13.58 | A |
| ATOM | 1064 | O | GLU | A | 145 | 47.489 | 90.542 | 24.963 | 1.00 | 13.47 | A |
| ATOM | 1065 | N | PHE | A | 146 | 46.105 | 89.189 | 23.814 | 1.00 | 11.98 | A |
| ATOM | 1066 | CA | PHE | A | 146 | 47.188 | 88.488 | 23.135 | 1.00 | 12.54 | A |
| ATOM | 1067 | CB | PHE | A | 146 | 46.609 | 87.440 | 22.178 | 1.00 | 11.57 | A |
| ATOM | 1068 | CG | PHE | A | 146 | 47.635 | 86.830 | 21.255 | 1.00 | 12.50 | A |
| ATOM | 1069 | CD1 | PHE | A | 146 | 48.313 | 85.795 | 21.634 | 1.00 | 12.88 | A |
| ATOM | 1070 | CD2 | PHE | A | 146 | 47.850 | 87.354 | 20.089 | 1.00 | 12.84 | A |
| ATOM | 1071 | CE1 | PHE | A | 146 | 49.290 | 85.202 | 20.820 | 1.00 | 13.90 | A |
| ATOM | 1072 | CE2 | PHE | A | 146 | 48.807 | 86.837 | 19.201 | 1.00 | 13.55 | A |
| ATOM | 1073 | CZ | PHE | A | 146 | 49.539 | 85.737 | 19.581 | 1.00 | 14.05 | A |
| ATOM | 1074 | C | PHE | A | 146 | 48.049 | 89.509 | 22.410 | 1.00 | 11.89 | A |
| ATOM | 1075 | O | PHE | A | 146 | 49.269 | 89.521 | 22.553 | 1.00 | 12.44 | A |
| ATOM | 1076 | N | PHE | A | 147 | 47.401 | 90.363 | 21.626 | 1.00 | 11.00 | A |
| ATOM | 1077 | CA | PHE | A | 147 | 48.110 | 91.377 | 20.861 | 1.00 | 12.11 | A |
| ATOM | 1078 | CB | PHE | A | 147 | 47.148 | 91.380 | 19.831 | 1.00 | 10.46 | A |
| ATOM | 1079 | CG | PHE | A | 147 | 46.901 | 91.088 | 18.640 | 1.00 | 11.19 | A |
| ATOM | 1080 | CD1 | PHE | A | 147 | 47.825 | 90.940 | 17.745 | 1.00 | 10.73 | A |
| ATOM | 1081 | CD2 | PHE | A | 147 | 45.774 | 90.457 | 18.522 | 1.00 | 10.42 | A |
| ATOM | 1082 | CE1 | PHE | A | 147 | 47.658 | 90.111 | 16.634 | 1.00 | 10.56 | A |
| ATOM | 1083 | CE2 | PHE | A | 147 | 45.509 | 89.603 | 17.446 | 1.00 | 11.65 | A |
| ATOM | 1084 | CZ | PHE | A | 147 | 46.472 | 89.428 | 16.486 | 1.00 | 10.21 | A |
| ATOM | 1085 | C | PHE | A | 147 | 48.776 | 92.416 | 21.752 | 1.00 | 13.23 | A |
| ATOM | 1086 | O | PHE | A | 147 | 49.932 | 92.769 | 21.531 | 1.00 | 12.92 | A |
| ATOM | 1087 | N | ARG | A | 148 | 48.054 | 92.901 | 22.755 | 1.00 | 12.51 | A |
| ATOM | 1088 | CA | ARG | A | 148 | 48.594 | 93.909 | 23.660 | 1.00 | 13.44 | A |
| ATOM | 1089 | CB | ARG | A | 148 | 47.506 | 94.320 | 24.660 | 1.00 | 15.03 | A |
| ATOM | 1090 | CG | ARG | A | 148 | 47.954 | 95.387 | 25.653 | 1.00 | 17.55 | A |
| ATOM | 1091 | CD | ARG | A | 148 | 47.032 | 95.439 | 26.863 | 1.00 | 23.81 | A |
| ATOM | 1092 | NE | ARG | A | 148 | 45.666 | 95.820 | 26.517 | 1.00 | 27.85 | A |
| ATOM | 1093 | CZ | ARG | A | 148 | 45.297 | 97.046 | 26.162 | 1.00 | 29.59 | A |
| ATOM | 1094 | NH1 | ARG | A | 148 | 46.195 | 98.020 | 26.105 | 1.00 | 31.38 | A |
| ATOM | 1095 | NH2 | ARG | A | 148 | 44.028 | 97.302 | 25.870 | 1.00 | 30.78 | A |
| ATOM | 1096 | C | ARG | A | 148 | 49.868 | 93.442 | 24.355 | 1.00 | 14.63 | A |
| ATOM | 1097 | O | ARG | A | 148 | 50.803 | 94.224 | 24.529 | 1.00 | 15.99 | A |
| ATOM | 1098 | N | ARG | A | 149 | 49.912 | 92.169 | 24.739 | 1.00 | 14.85 | A |
| ATOM | 1099 | CA | ARG | A | 149 | 51.060 | 91.622 | 25.461 | 1.00 | 16.63 | A |
| ATOM | 1100 | CB | ARG | A | 149 | 50.530 | 90.711 | 26.575 | 1.00 | 18.90 | A |
| ATOM | 1101 | CG | ARG | A | 149 | 49.718 | 91.426 | 27.642 | 1.00 | 23.96 | A |
| ATOM | 1102 | CD | ARG | A | 149 | 49.027 | 90.436 | 28.571 | 1.00 | 28.31 | A |
| ATOM | 1103 | NE | ARG | A | 149 | 49.958 | 89.477 | 29.163 | 1.00 | 32.87 | A |
| ATOM | 1104 | CZ | ARG | A | 149 | 49.601 | 88.510 | 30.004 | 1.00 | 34.71 | A |
| ATOM | 1105 | NH1 | ARG | A | 149 | 48.330 | 88.370 | 30.356 | 1.00 | 35.27 | A |
| ATOM | 1106 | NH2 | ARG | A | 149 | 50.512 | 87.678 | 30.491 | 1.00 | 35.30 | A |
| ATOM | 1107 | C | ARG | A | 149 | 52.119 | 90.918 | 24.617 | 1.00 | 14.67 | A |
| ATOM | 1108 | O | ARG | A | 149 | 53.164 | 90.531 | 25.171 | 1.00 | 16.69 | A |
| ATOM | 1109 | N | SER | A | 150 | 51.877 | 90.748 | 23.323 | 1.00 | 14.80 | A |
| ATOM | 1110 | CA | SER | A | 150 | 52.843 | 90.038 | 22.494 | 1.00 | 15.33 | A |
| ATOM | 1111 | CB | SER | A | 150 | 52.209 | 89.706 | 21.142 | 1.00 | 16.25 | A |
| ATOM | 1112 | OG | SER | A | 150 | 53.102 | 88.924 | 20.362 | 1.00 | 16.71 | A |
| ATOM | 1113 | C | SER | A | 150 | 54.187 | 90.738 | 22.335 | 1.00 | 16.91 | A |
| ATOM | 1114 | O | SER | A | 150 | 54.266 | 91.967 | 22.254 | 1.00 | 18.19 | A |
| ATOM | 1115 | N | ALA | A | 151 | 55.240 | 89.926 | 22.288 | 1.00 | 17.17 | A |
| ATOM | 1116 | CA | ALA | A | 151 | 56.605 | 90.401 | 22.100 | 1.00 | 17.74 | A |
| ATOM | 1117 | CB | ALA | A | 151 | 57.504 | 89.905 | 23.233 | 1.00 | 17.92 | A |
| ATOM | 1118 | C | ALA | A | 151 | 57.085 | 89.849 | 20.757 | 1.00 | 18.11 | A |
| ATOM | 1119 | O | ALA | A | 151 | 58.243 | 90.018 | 20.376 | 1.00 | 19.71 | A |
| ATOM | 1120 | N | ILE | A | 152 | 56.179 | 89.171 | 20.053 | 1.00 | 16.75 | A |
| ATOM | 1121 | CA | ILE | A | 152 | 56.469 | 88.599 | 18.739 | 1.00 | 16.86 | A |
| ATOM | 1122 | CB | ILE | A | 152 | 55.448 | 87.494 | 18.380 | 1.00 | 16.79 | A |
| ATOM | 1123 | CG2 | ILE | A | 152 | 55.760 | 86.920 | 17.008 | 1.00 | 15.71 | A |
| ATOM | 1124 | CG1 | ILE | A | 152 | 55.458 | 86.396 | 19.451 | 1.00 | 19.51 | A |
| ATOM | 1125 | CD1 | ILE | A | 152 | 56.784 | 85.681 | 19.602 | 1.00 | 22.08 | A |
| ATOM | 1126 | C | ILE | A | 152 | 56.367 | 89.727 | 17.709 | 1.00 | 15.48 | A |
| ATOM | 1127 | O | ILE | A | 152 | 55.329 | 90.373 | 17.596 | 1.00 | 15.67 | A |
| ATOM | 1128 | N | ALA | A | 153 | 57.438 | 89.950 | 16.954 | 1.00 | 15.61 | A |
| ATOM | 1129 | CA | ALA | A | 153 | 57.485 | 91.028 | 15.962 | 1.00 | 14.18 | A |
| ATOM | 1130 | CB | ALA | A | 153 | 58.690 | 90.836 | 15.052 | 1.00 | 14.47 | A |
| ATOM | 1131 | C | ALA | A | 153 | 56.231 | 91.241 | 15.114 | 1.00 | 12.53 | A |
| ATOM | 1132 | O | ALA | A | 153 | 55.697 | 92.353 | 15.051 | 1.00 | 13.88 | A |
| ATOM | 1133 | N | VAL | A | 154 | 55.762 | 90.193 | 14.448 | 1.00 | 13.33 | A |
| ATOM | 1134 | CA | VAL | A | 154 | 54.576 | 90.330 | 13.613 | 1.00 | 12.94 | A |
| ATOM | 1135 | CB | VAL | A | 154 | 54.255 | 89.008 | 12.870 | 1.00 | 14.20 | A |
| ATOM | 1136 | CG1 | VAL | A | 154 | 52.905 | 89.108 | 12.179 | 1.00 | 14.95 | A |
| ATOM | 1137 | CG2 | VAL | A | 154 | 55.342 | 88.715 | 11.849 | 1.00 | 16.93 | A |
| ATOM | 1138 | C | VAL | A | 154 | 53.352 | 90.758 | 14.420 | 1.00 | 11.60 | A |
| ATOM | 1139 | O | VAL | A | 154 | 52.606 | 91.649 | 14.006 | 1.00 | 12.26 | A |
| ATOM | 1140 | N | PHE | A | 155 | 53.161 | 90.141 | 15.582 | 1.00 | 11.98 | A |
| ATOM | 1141 | CA | PHE | A | 155 | 52.004 | 90.448 | 16.413 | 1.00 | 12.64 | A |
| ATOM | 1142 | CB | PHE | A | 155 | 51.753 | 89.287 | 17.387 | 1.00 | 12.89 | A |
| ATOM | 1143 | CG | PHE | A | 155 | 51.641 | 87.936 | 16.718 | 1.00 | 12.14 | A |
| ATOM | 1144 | CD1 | PHE | A | 155 | 51.079 | 87.824 | 15.556 | 1.00 | 13.16 | A |
| ATOM | 1145 | CD2 | PHE | A | 155 | 52.103 | 86.888 | 17.317 | 1.00 | 13.67 | A |
| ATOM | 1146 | CE1 | PHE | A | 155 | 50.950 | 86.587 | 14.903 | 1.00 | 13.25 | A |
| ATOM | 1147 | CE2 | PHE | A | 155 | 52.024 | 85.609 | 16.756 | 1.00 | 14.31 | A |
| ATOM | 1148 | CZ | PHE | A | 155 | 51.436 | 85.459 | 15.523 | 1.00 | 13.23 | A |
| ATOM | 1149 | C | PHE | A | 155 | 52.088 | 91.801 | 17.110 | 1.00 | 12.95 | A |
| ATOM | 1150 | O | PHE | A | 155 | 51.076 | 92.498 | 17.223 | 1.00 | 12.80 | A |
| ATOM | 1151 | N | ASP | A | 156 | 53.268 | 92.182 | 17.597 | 1.00 | 14.04 | A |
| ATOM | 1152 | CA | ASP | A | 156 | 53.364 | 93.473 | 18.262 | 1.00 | 14.31 | A |
| ATOM | 1153 | CB | ASP | A | 156 | 54.637 | 93.553 | 19.130 | 1.00 | 17.42 | A |
| ATOM | 1154 | CG | ASP | A | 156 | 55.910 | 93.340 | 18.347 | 1.00 | 20.36 | A |
| ATOM | 1155 | OD1 | ASP | A | 156 | 56.114 | 94.029 | 17.330 | 1.00 | 21.14 | A |
| ATOM | 1156 | OD2 | ASP | A | 156 | 56.721 | 92.488 | 18.770 | 1.00 | 23.78 | A |
| ATOM | 1157 | C | ASP | A | 156 | 53.208 | 94.598 | 17.243 | 1.00 | 13.14 | A |
| ATOM | 1158 | O | ASP | A | 156 | 52.723 | 95.677 | 17.587 | 1.00 | 13.21 | A |
| ATOM | 1159 | N | LYS | A | 157 | 53.588 | 94.349 | 15.991 | 1.00 | 12.71 | A |
| ATOM | 1160 | CA | LYS | A | 157 | 53.422 | 95.363 | 14.955 | 1.00 | 13.09 | A |
| ATOM | 1161 | CB | LYS | A | 157 | 54.214 | 94.978 | 13.703 | 1.00 | 14.27 | A |
| ATOM | 1162 | CG | LYS | A | 157 | 54.016 | 95.968 | 12.566 | 1.00 | 15.61 | A |
| ATOM | 1163 | CD | LYS | A | 157 | 54.948 | 95.701 | 11.398 | 1.00 | 20.68 | A |
| ATOM | 1164 | CE | LYS | A | 157 | 54.727 | 96.719 | 10.290 | 1.00 | 22.88 | A |
| ATOM | 1165 | NZ | LYS | A | 157 | 55.582 | 96.451 | 9.104 | 1.00 | 25.73 | A |
| ATOM | 1166 | C | LYS | A | 157 | 51.924 | 95.507 | 14.688 | 1.00 | 12.13 | A |
| ATOM | 1167 | O | LYS | A | 157 | 51.418 | 96.610 | 14.479 | 1.00 | 12.98 | A |
| ATOM | 1168 | N | MET | A | 158 | 51.217 | 94.380 | 14.691 | 1.00 | 11.25 | A |
| ATOM | 1169 | CA | MET | A | 158 | 49.777 | 94.383 | 14.465 | 1.00 | 11.17 | A |
| ATOM | 1170 | CB | MET | A | 158 | 49.279 | 92.936 | 14.346 | 1.00 | 10.94 | A |
| ATOM | 1171 | CG | MET | A | 158 | 49.636 | 92.265 | 13.024 | 1.00 | 10.67 | A |
| ATOM | 1172 | SD | MET | A | 158 | 49.209 | 90.507 | 12.972 | 1.00 | 12.05 | A |
| ATOM | 1173 | CE | MET | A | 158 | 47.417 | 90.562 | 12.875 | 1.00 | 12.53 | A |
| ATOM | 1174 | C | MET | A | 158 | 49.084 | 95.170 | 15.570 | 1.00 | 9.74 | A |
| ATOM | 1175 | O | MET | A | 158 | 48.154 | 95.931 | 15.301 | 1.00 | 10.03 | A |
| ATOM | 1176 | N | TRP | A | 159 | 49.537 | 94.995 | 16.809 | 1.00 | 10.33 | A |
| ATOM | 1177 | CA | TRP | A | 159 | 48.938 | 95.708 | 17.933 | 1.00 | 11.27 | A |
| ATOM | 1178 | CB | TRP | A | 159 | 49.509 | 95.184 | 19.252 | 1.00 | 10.95 | A |
| ATOM | 1179 | CG | TRP | A | 159 | 49.008 | 95.972 | 20.431 | 1.00 | 12.47 | A |
| ATOM | 1180 | CD2 | TRP | A | 159 | 47.647 | 96.090 | 20.867 | 1.00 | 12.97 | A |
| ATOM | 1181 | CE2 | TRP | A | 159 | 47.638 | 96.966 | 21.976 | 1.00 | 14.72 | A |
| ATOM | 1182 | CE3 | TRP | A | 159 | 46.433 | 95.541 | 20.428 | 1.00 | 13.48 | A |
| ATOM | 1183 | CD1 | TRP | A | 159 | 49.749 | 96.757 | 21.271 | 1.00 | 15.07 | A |
| ATOM | 1184 | NE1 | TRP | A | 159 | 48.932 | 97.358 | 22.201 | 1.00 | 15.53 | A |
| ATOM | 1185 | CZ2 | TRP | A | 159 | 46.460 | 97.307 | 22.653 | 1.00 | 17.62 | A |
| ATOM | 1186 | CZ3 | TRP | A | 159 | 45.259 | 95.882 | 21.104 | 1.00 | 16.05 | A |
| ATOM | 1187 | CH2 | TRP | A | 159 | 45.285 | 96.757 | 22.203 | 1.00 | 17.88 | A |
| ATOM | 1188 | C | TRP | A | 159 | 49.138 | 97.208 | 17.785 | 1.00 | 11.68 | A |
| ATOM | 1189 | O | TRP | A | 159 | 48.205 | 97.991 | 17.958 | 1.00 | 11.37 | A |
| ATOM | 1190 | N | THR | A | 160 | 50.366 | 97.609 | 17.479 | 1.00 | 11.80 | A |
| ATOM | 1191 | CA | THR | A | 160 | 50.672 | 99.023 | 17.318 | 1.00 | 11.05 | A |
| ATOM | 1192 | CB | THR | A | 160 | 52.154 | 99.221 | 16.973 | 1.00 | 14.07 | A |
| ATOM | 1193 | OG1 | THR | A | 160 | 52.951 | 98.816 | 18.094 | 1.00 | 16.78 | A |
| ATOM | 1194 | CG2 | THR | A | 160 | 52.441 | 100.682 | 16.647 | 1.00 | 15.23 | A |
| ATOM | 1195 | C | THR | A | 160 | 49.790 | 99.621 | 16.228 | 1.00 | 11.18 | A |
| ATOM | 1196 | O | THR | A | 160 | 49.295 | 100.740 | 16.367 | 1.00 | 11.33 | A |
| ATOM | 1197 | N | TYR | A | 161 | 49.591 | 98.872 | 15.149 | 1.00 | 10.84 | A |
| ATOM | 1198 | CA | TYR | A | 161 | 48.736 | 99.325 | 14.062 | 1.00 | 11.33 | A |
| ATOM | 1199 | CB | TYR | A | 161 | 48.813 | 98.346 | 12.881 | 1.00 | 11.26 | A |
| ATOM | 1200 | CG | TYR | A | 161 | 47.562 | 98.409 | 12.034 | 1.00 | 12.15 | A |
| ATOM | 1201 | CD1 | TYR | A | 161 | 47.415 | 99.374 | 11.042 | 1.00 | 11.53 | A |
| ATOM | 1202 | CE2 | TYR | A | 161 | 46.217 | 99.517 | 10.351 | 1.00 | 12.22 | A |
| ATOM | 1203 | CD2 | TYR | A | 161 | 46.475 | 97.578 | 12.307 | 1.00 | 10.65 | A |
| ATOM | 1204 | CE2 | TYR | A | 161 | 45.275 | 97.713 | 11.625 | 1.00 | 12.74 | A |
| ATOM | 1205 | CZ | TYR | A | 161 | 45.150 | 98.686 | 10.651 | 1.00 | 12.21 | A |
| ATOM | 1206 | OH | TYR | A | 161 | 43.948 | 98.846 | 10.006 | 1.00 | 14.64 | A |
| ATOM | 1207 | C | TYR | A | 161 | 47.298 | 99.465 | 14.563 | 1.00 | 11.26 | A |
| ATOM | 1208 | O | TYR | A | 161 | 46.643 | 100.477 | 14.323 | 1.00 | 12.43 | A |
| ATOM | 1209 | N | MET | A | 162 | 46.805 | 98.433 | 15.243 | 1.00 | 11.09 | A |
| ATOM | 1210 | CA | MET | A | 162 | 45.431 | 98.433 | 15.740 | 1.00 | 12.07 | A |
| ATOM | 1211 | CB | MET | A | 162 | 45.127 | 97.092 | 16.411 | 1.00 | 13.59 | A |
| ATOM | 1212 | CG | MET | A | 162 | 44.945 | 95.934 | 15.464 | 1.00 | 12.93 | A |
| ATOM | 1213 | SD | MET | A | 162 | 44.421 | 94.492 | 16.402 | 1.00 | 18.46 | A |
| ATOM | 1214 | CE | MET | A | 162 | 45.964 | 93.644 | 16.600 | 1.00 | 21.20 | A |
| ATOM | 1215 | C | MET | A | 162 | 45.129 | 99.592 | 16.661 | 1.00 | 12.55 | A |
| ATOM | 1216 | O | MET | A | 162 | 44.095 | 100.246 | 16.530 | 1.00 | 12.30 | A |
| ATOM | 1217 | N | ARG | A | 163 | 46.031 | 99.846 | 17.598 | 1.00 | 11.90 | A |
| ATOM | 1218 | CA | ARG | A | 163 | 45.811 | 100.913 | 18.556 | 1.00 | 12.87 | A |
| ATOM | 1219 | CB | ARG | A | 163 | 46.821 | 100.766 | 19.696 | 1.00 | 16.20 | A |
| ATOM | 1220 | CG | ARG | A | 163 | 46.382 | 101.452 | 20.975 | 1.00 | 19.85 | A |
| ATOM | 1221 | CD | ARG | A | 163 | 47.426 | 101.347 | 22.070 | 1.00 | 23.43 | A |
| ATOM | 1222 | NE | ARG | A | 163 | 46.933 | 101.936 | 23.310 | 1.00 | 26.48 | A |
| ATOM | 1223 | CZ | ARG | A | 163 | 47.693 | 102.223 | 24.361 | 1.00 | 27.67 | A |
| ATOM | 1224 | NH1 | ARG | A | 163 | 48.996 | 101.978 | 24.330 | 1.00 | 27.83 | A |
| ATOM | 1225 | NH2 | ARG | A | 163 | 47.145 | 102.758 | 25.443 | 1.00 | 29.17 | A |
| ATOM | 1226 | C | ARG | A | 163 | 45.826 | 102.299 | 17.920 | 1.00 | 13.03 | A |
| ATOM | 1227 | O | ARG | A | 163 | 45.173 | 103.216 | 18.419 | 1.00 | 13.70 | A |
| ATOM | 1228 | N | SER | A | 164 | 46.550 | 102.453 | 16.816 | 1.00 | 11.77 | A |
| ATOM | 1229 | CA | SER | A | 164 | 46.645 | 103.754 | 16.157 | 1.00 | 11.84 | A |
| ATOM | 1230 | CB | SER | A | 164 | 48.124 | 104.065 | 15.902 | 1.00 | 12.55 | A |
| ATOM | 1231 | OG | SER | A | 164 | 48.682 | 103.162 | 14.961 | 1.00 | 14.03 | A |
| ATOM | 1232 | C | SER | A | 164 | 45.800 | 103.911 | 14.894 | 1.00 | 12.15 | A |
| ATOM | 1233 | O | SER | A | 164 | 45.750 | 104.995 | 14.316 | 1.00 | 13.15 | A |
| ATOM | 1234 | N | ALA | A | 165 | 45.127 | 102.847 | 14.468 | 1.00 | 12.42 | A |
| ATOM | 1235 | CA | ALA | A | 165 | 44.315 | 102.906 | 13.256 | 1.00 | 12.32 | A |
| ATOM | 1236 | CB | ALA | A | 165 | 43.963 | 101.495 | 12.803 | 1.00 | 13.52 | A |
| ATOM | 1237 | C | ALA | A | 165 | 43.040 | 103.742 | 13.380 | 1.00 | 12.82 | A |
| ATOM | 1238 | O | ALA | A | 165 | 42.374 | 103.731 | 14.413 | 1.00 | 13.31 | A |
| ATOM | 1239 | N | GLU | A | 166 | 42.710 | 104.459 | 12.307 | 1.00 | 13.69 | A |
| ATOM | 1240 | CA | GLU | A | 166 | 41.518 | 105.300 | 12.257 | 1.00 | 14.95 | A |
| ATOM | 1241 | CB | GLU | A | 166 | 41.923 | 106.767 | 12.392 | 1.00 | 14.11 | A |
| ATOM | 1242 | CG | GLU | A | 166 | 42.629 | 107.113 | 13.680 | 1.00 | 14.49 | A |
| ATOM | 1243 | CD | GLU | A | 166 | 42.760 | 108.609 | 13.864 | 1.00 | 13.93 | A |
| ATOM | 1244 | OE1 | GLU | A | 166 | 43.460 | 109.257 | 13.055 | 1.00 | 14.76 | A |
| ATOM | 1245 | OE2 | GLU | A | 166 | 42.149 | 109.138 | 14.813 | 1.00 | 14.89 | A |
| ATOM | 1246 | C | GLU | A | 166 | 40.776 | 105.033 | 10.952 | 1.00 | 15.15 | A |
| ATOM | 1247 | O | GLU | A | 166 | 41.384 | 105.028 | 9.881 | 1.00 | 17.39 | A |
| ATOM | 1248 | N | PRO | A | 167 | 39.453 | 104.808 | 11.020 | 1.00 | 15.65 | A |
| ATOM | 1249 | CD | PRO | A | 167 | 38.614 | 104.781 | 9.813 | 1.00 | 15.88 | A |
| ATOM | 1250 | CA | PRO | A | 167 | 38.617 | 104.783 | 12.226 | 1.00 | 14.51 | A |
| ATOM | 1251 | CB | PRO | A | 167 | 37.191 | 104.766 | 11.655 | 1.00 | 15.59 | A |
| ATOM | 1252 | CG | PRO | A | 167 | 37.307 | 105.477 | 10.328 | 1.00 | 19.02 | A |
| ATOM | 1253 | C | PRO | A | 167 | 38.980 | 103.575 | 13.082 | 1.00 | 13.89 | A |
| ATOM | 1254 | O | PRO | A | 167 | 39.716 | 102.697 | 12.635 | 1.00 | 13.09 | A |
| ATOM | 1255 | N | SER | A | 168 | 38.462 | 103.530 | 14.306 | 1.00 | 14.02 | A |
| ATOM | 1256 | CA | SER | A | 168 | 38.744 | 102.419 | 15.210 | 1.00 | 13.10 | A |
| ATOM | 1257 | CB | SER | A | 168 | 37.928 | 102.582 | 16.490 | 1.00 | 14.12 | A |
| ATOM | 1258 | OG | SER | A | 168 | 38.111 | 101.448 | 17.317 | 1.00 | 15.45 | A |
| ATOM | 1259 | C | SER | A | 168 | 38.482 | 101.080 | 14.538 | 1.00 | 11.80 | A |
| ATOM | 1260 | O | SER | A | 168 | 37.459 | 100.895 | 13.875 | 1.00 | 13.09 | A |
| ATOM | 1261 | N | VAL | A | 169 | 39.409 | 100.146 | 14.727 | 1.00 | 11.45 | A |
| ATOM | 1262 | CA | VAL | A | 169 | 39.291 | 98.813 | 14.147 | 1.00 | 11.66 | A |
| ATOM | 1263 | CB | VAL | A | 169 | 40.660 | 98.291 | 13.646 | 1.00 | 11.62 | A |
| ATOM | 1264 | CG1 | VAL | A | 169 | 41.163 | 99.175 | 12.515 | 1.00 | 12.91 | A |
| ATOM | 1265 | CG2 | VAL | A | 169 | 41.668 | 98.251 | 14.795 | 1.00 | 11.90 | A |
| ATOM | 1266 | C | VAL | A | 169 | 38.730 | 97.814 | 15.150 | 1.00 | 11.83 | A |
| ATOM | 1267 | O | VAL | A | 169 | 38.575 | 96.633 | 14.840 | 1.00 | 11.71 | A |
| ATOM | 1268 | N | PHE | A | 170 | 38.424 | 98.287 | 16.353 | 1.00 | 11.38 | A |
| ATOM | 1269 | CA | PHE | A | 170 | 37.881 | 97.413 | 17.384 | 1.00 | 12.11 | A |
| ATOM | 1270 | CB | PHE | A | 170 | 38.343 | 97.905 | 18.762 | 1.00 | 11.45 | A |
| ATOM | 1271 | CG | PHE | A | 170 | 39.834 | 97.882 | 18.943 | 1.00 | 12.69 | A |
| ATOM | 1272 | CD1 | PHE | A | 170 | 40.454 | 96.752 | 19.066 | 1.00 | 13.27 | A |
| ATOM | 1273 | CD2 | PHE | A | 170 | 40.499 | 98.993 | 18.954 | 1.00 | 13.73 | A |
| ATOM | 1274 | CE1 | PHE | A | 170 | 41.845 | 96.679 | 19.213 | 1.00 | 13.67 | A |
| ATOM | 1275 | CE2 | PHE | A | 170 | 41.890 | 99.030 | 19.095 | 1.00 | 14.80 | A |
| ATOM | 1276 | CZ | PHE | A | 170 | 42.574 | 97.845 | 19.227 | 1.00 | 14.22 | A |
| ATOM | 1277 | C | PHE | A | 170 | 36.368 | 97.324 | 17.266 | 1.00 | 12.92 | A |
| ATOM | 1278 | O | PHE | A | 170 | 35.707 | 98.276 | 16.843 | 1.00 | 13.95 | A |
| ATOM | 1279 | N | VAL | A | 171 | 35.828 | 96.164 | 17.627 | 1.00 | 13.05 | A |
| ATOM | 1280 | CA | VAL | A | 171 | 34.388 | 95.933 | 17.599 | 1.00 | 13.17 | A |
| ATOM | 1281 | CB | VAL | A | 171 | 34.006 | 94.876 | 16.535 | 1.00 | 13.33 | A |
| ATOM | 1282 | CG1 | VAL | A | 171 | 34.385 | 95.385 | 15.147 | 1.00 | 13.29 | A |
| ATOM | 1283 | CG2 | VAL | A | 171 | 34.696 | 93.554 | 16.830 | 1.00 | 13.56 | A |
| ATOM | 1284 | C | VAL | A | 171 | 33.941 | 95.486 | 18.990 | 1.00 | 13.90 | A |
| ATOM | 1285 | O | VAL | A | 171 | 34.747 | 94.994 | 19.778 | 1.00 | 13.56 | A |
| ATOM | 1286 | N | ALA | A | 172 | 32.658 | 95.668 | 19.291 | 1.00 | 15.24 | A |
| ATOM | 1287 | CA | ALA | A | 172 | 32.123 | 95.320 | 20.604 | 1.00 | 15.32 | A |
| ATOM | 1288 | CB | ALA | A | 172 | 30.989 | 96.277 | 20.961 | 1.00 | 16.56 | A |
| ATOM | 1289 | C | ALA | A | 172 | 31.643 | 93.883 | 20.754 | 1.00 | 14.93 | A |
| ATOM | 1290 | O | ALA | A | 172 | 31.708 | 93.318 | 21.846 | 1.00 | 15.25 | A |
| ATOM | 1291 | N | THR | A | 173 | 31.162 | 93.293 | 19.666 | 1.00 | 14.48 | A |
| ATOM | 1292 | CA | THR | A | 173 | 30.650 | 91.928 | 19.713 | 1.00 | 14.40 | A |
| ATOM | 1293 | CB | THR | A | 173 | 29.120 | 91.917 | 19.736 | 1.00 | 14.21 | A |
| ATOM | 1294 | OG1 | THR | A | 173 | 28.639 | 92.402 | 18.477 | 1.00 | 15.42 | A |
| ATOM | 1295 | CG2 | THR | A | 173 | 28.586 | 92.804 | 20.853 | 1.00 | 15.19 | A |
| ATOM | 1296 | C | THR | A | 173 | 31.077 | 91.135 | 18.489 | 1.00 | 13.03 | A |
| ATOM | 1297 | O | THR | A | 173 | 31.485 | 91.707 | 17.477 | 1.00 | 13.31 | A |
| ATOM | 1298 | N | THR | A | 174 | 30.959 | 89.815 | 18.578 | 1.00 | 12.27 | A |
| ATOM | 1299 | CA | THR | A | 174 | 31.320 | 88.952 | 17.465 | 1.00 | 13.54 | A |
| ATOM | 1300 | CB | THR | A | 174 | 31.082 | 87.475 | 17.820 | 1.00 | 12.72 | A |
| ATOM | 1301 | OG1 | THR | A | 174 | 31.902 | 87.124 | 18.941 | 1.00 | 13.63 | A |
| ATOM | 1302 | CG2 | THR | A | 174 | 31.437 | 86.572 | 16.643 | 1.00 | 12.54 | A |
| ATOM | 1303 | C | THR | A | 174 | 30.478 | 89.326 | 16.249 | 1.00 | 13.64 | A |
| ATOM | 1304 | O | THR | A | 174 | 30.994 | 89.446 | 15.136 | 1.00 | 12.34 | A |
| ATOM | 1305 | N | ALA | A | 175 | 29.182 | 89.528 | 16.470 | 1.00 | 13.64 | A |
| ATOM | 1306 | CA | ALA | A | 175 | 28.267 | 89.889 | 15.392 | 1.00 | 14.24 | A |
| ATOM | 1307 | CB | ALA | A | 175 | 26.849 | 90.026 | 15.939 | 1.00 | 15.47 | A |
| ATOM | 1308 | C | ALA | A | 175 | 28.689 | 91.183 | 14.697 | 1.00 | 14.01 | A |
| ATOM | 1309 | O | ALA | A | 175 | 28.568 | 91.308 | 13.478 | 1.00 | 14.70 | A |
| ATOM | 1310 | N | GLU | A | 176 | 29.181 | 92.143 | 15.476 | 1.00 | 13.28 | A |
| ATOM | 1311 | CA | GLU | A | 176 | 29.624 | 93.423 | 14.930 | 1.00 | 14.06 | A |
| ATOM | 1312 | CB | GLU | A | 176 | 29.903 | 94.387 | 16.085 | 1.00 | 15.36 | A |
| ATOM | 1313 | CG | GLU | A | 176 | 30.259 | 95.796 | 15.652 | 1.00 | 21.05 | A |
| ATOM | 1314 | CD | GLU | A | 176 | 30.652 | 96.674 | 16.823 | 1.00 | 23.31 | A |
| ATOM | 1315 | OE1 | GLU | A | 176 | 29.946 | 96.641 | 17.852 | 1.00 | 25.59 | A |
| ATOM | 1316 | OE2 | GLU | A | 176 | 31.660 | 97.402 | 16.713 | 1.00 | 25.39 | A |
| ATOM | 1317 | C | GLU | A | 176 | 30.841 | 93.202 | 14.036 | 1.00 | 13.19 | A |
| ATOM | 1318 | O | GLU | A | 176 | 30.954 | 93.799 | 12.965 | 1.00 | 12.97 | A |
| ATOM | 1319 | N | GLY | A | 177 | 31.757 | 92.351 | 14.491 | 1.00 | 11.87 | A |
| ATOM | 1320 | CA | GLY | A | 177 | 32.948 | 92.062 | 13.711 | 1.00 | 12.60 | A |
| ATOM | 1321 | C | GLY | A | 177 | 32.588 | 91.401 | 12.394 | 1.00 | 12.97 | A |
| ATOM | 1322 | O | GLY | A | 177 | 33.098 | 91.779 | 11.334 | 1.00 | 11.59 | A |
| ATOM | 1323 | N | VAL | A | 178 | 31.699 | 90.416 | 12.460 | 1.00 | 12.50 | A |
| ATOM | 1324 | CA | VAL | A | 178 | 31.257 | 89.697 | 11.272 | 1.00 | 12.49 | A |
| ATOM | 1325 | CB | VAL | A | 178 | 30.294 | 88.547 | 11.645 | 1.00 | 13.51 | A |
| ATOM | 1326 | CG1 | VAL | A | 178 | 29.691 | 87.941 | 10.384 | 1.00 | 12.61 | A |
| ATOM | 1327 | CG2 | VAL | A | 178 | 31.035 | 87.484 | 12.434 | 1.00 | 14.57 | A |
| ATOM | 1328 | C | VAL | A | 178 | 30.557 | 90.637 | 10.293 | 1.00 | 12.98 | A |
| ATOM | 1329 | O | VAL | A | 178 | 30.807 | 90.590 | 9.089 | 1.00 | 13.29 | A |
| ATOM | 1330 | N | ALA | A | 179 | 29.677 | 91.489 | 10.810 | 1.00 | 12.81 | A |
| ATOM | 1331 | CA | ALA | A | 179 | 28.962 | 92.436 | 9.961 | 1.00 | 13.11 | A |
| ATOM | 1332 | CB | ALA | A | 179 | 27.945 | 93.223 | 10.788 | 1.00 | 13.80 | A |
| ATOM | 1333 | C | ALA | A | 179 | 29.938 | 93.394 | 9.288 | 1.00 | 14.25 | A |
| ATOM | 1334 | O | ALA | A | 179 | 29.770 | 93.745 | 8.122 | 1.00 | 14.57 | A |
| ATOM | 1335 | N | ARG | A | 180 | 30.962 | 93.816 | 10.025 | 1.00 | 12.82 | A |
| ATOM | 1336 | CA | ARG | A | 180 | 31.946 | 94.737 | 9.472 | 1.00 | 12.12 | A |
| ATOM | 1337 | CB | ARG | A | 180 | 32.859 | 95.239 | 10.589 | 1.00 | 12.50 | A |
| ATOM | 1338 | CG | ARG | A | 180 | 33.793 | 96.343 | 10.145 | 1.00 | 13.41 | A |
| ATOM | 1339 | CD | ARG | A | 180 | 34.401 | 97.029 | 11.346 | 1.00 | 13.48 | A |
| ATOM | 1340 | NE | ARG | A | 180 | 35.338 | 98.071 | 10.948 | 1.00 | 14.00 | A |
| ATOM | 1341 | CZ | ARG | A | 180 | 36.000 | 98.838 | 11.805 | 1.00 | 14.74 | A |
| ATOM | 1342 | NH1 | ARG | A | 180 | 35.821 | 98.682 | 13.110 | 1.00 | 15.16 | A |
| ATOM | 1343 | NH2 | ARG | A | 180 | 36.855 | 99.746 | 11.355 | 1.00 | 16.18 | A |
| ATOM | 1344 | C | ARG | A | 180 | 32.692 | 94.070 | 8.326 | 1.00 | 12.59 | A |
| ATOM | 1345 | O | ARG | A | 180 | 32.947 | 94.694 | 7.298 | 1.00 | 13.27 | A |
| ATOM | 1346 | N | VAL | A | 181 | 33.048 | 92.802 | 8.501 | 1.00 | 11.72 | A |
| ATOM | 1347 | CA | VAL | A | 181 | 33.738 | 92.076 | 7.441 | 1.00 | 11.47 | A |
| ATOM | 1348 | CB | VAL | A | 181 | 34.089 | 90.636 | 7.883 | 1.00 | 11.29 | A |
| ATOM | 1349 | CG1 | VAL | A | 181 | 34.543 | 89.810 | 6.685 | 1.00 | 12.77 | A |
| ATOM | 1350 | CG2 | VAL | A | 181 | 35.180 | 90.669 | 8.944 | 1.00 | 11.42 | A |
| ATOM | 1351 | C | VAL | A | 181 | 32.832 | 92.000 | 6.209 | 1.00 | 12.09 | A |
| ATOM | 1352 | O | VAL | A | 181 | 33.257 | 92.286 | 5.088 | 1.00 | 12.61 | A |
| ATOM | 1353 | N | ARG | A | 182 | 31.574 | 91.640 | 6.433 | 1.00 | 11.83 | A |
| ATOM | 1354 | CA | ARG | A | 182 | 30.607 | 91.492 | 5.350 | 1.00 | 12.81 | A |
| ATOM | 1355 | CB | ARG | A | 182 | 29.352 | 90.805 | 5.899 | 1.00 | 13.78 | A |
| ATOM | 1356 | CG | ARG | A | 182 | 29.567 | 89.354 | 6.291 | 1.00 | 14.34 | A |
| ATOM | 1357 | CD | ARG | A | 182 | 28.296 | 88.715 | 6.820 | 1.00 | 13.02 | A |
| ATOM | 1358 | NE | ARG | A | 182 | 28.426 | 87.264 | 6.889 | 1.00 | 13.79 | A |
| ATOM | 1359 | CZ | ARG | A | 182 | 27.743 | 86.491 | 7.726 | 1.00 | 15.56 | A |
| ATOM | 1360 | NH1 | ARG | A | 182 | 26.878 | 87.028 | 8.576 | 1.00 | 15.17 | A |
| ATOM | 1361 | NH2 | ARG | A | 182 | 27.924 | 85.178 | 7.711 | 1.00 | 16.94 | A |
| ATOM | 1362 | C | ARG | A | 182 | 30.272 | 92.769 | 4.596 | 1.00 | 14.15 | A |
| ATOM | 1363 | O | ARG | A | 182 | 30.014 | 92.724 | 3.392 | 1.00 | 15.65 | A |
| ATOM | 1364 | N | ALA | A | 183 | 30.275 | 93.903 | 5.289 | 1.00 | 14.36 | A |
| ATOM | 1365 | CA | ALA | A | 183 | 29.922 | 95.168 | 4.655 | 1.00 | 15.55 | A |
| ATOM | 1366 | CB | ALA | A | 183 | 28.981 | 95.942 | 5.570 | 1.00 | 16.27 | A |
| ATOM | 1367 | C | ALA | A | 183 | 31.081 | 96.067 | 4.242 | 1.00 | 16.15 | A |
| ATOM | 1368 | O | ALA | A | 183 | 30.852 | 97.151 | 3.701 | 1.00 | 18.41 | A |
| ATOM | 1369 | N | SER | A | 184 | 32.316 | 95.628 | 4.465 | 1.00 | 15.26 | A |
| ATOM | 1370 | CA | SER | A | 184 | 33.470 | 96.462 | 4.133 | 1.00 | 15.42 | A |
| ATOM | 1371 | CB | SER | A | 184 | 34.459 | 96.412 | 5.303 | 1.00 | 15.55 | A |
| ATOM | 1372 | OG | SER | A | 184 | 33.877 | 96.983 | 6.466 | 1.00 | 16.22 | A |
| ATOM | 1373 | C | SER | A | 184 | 34.125 | 96.194 | 2.780 | 1.00 | 14.72 | A |
| ATOM | 1374 | O | SER | A | 184 | 35.223 | 96.681 | 2.517 | 1.00 | 15.02 | A |
| ATOM | 1375 | N | ALA | A | 185 | 33.451 | 95.430 | 1.925 | 1.00 | 15.34 | A |
| ATOM | 1376 | CA | ALA | A | 185 | 33.961 | 95.129 | 0.588 | 1.00 | 16.07 | A |
| ATOM | 1377 | CB | ALA | A | 185 | 33.834 | 96.364 | −0.299 | 1.00 | 16.92 | A |
| ATOM | 1378 | C | ALA | A | 185 | 35.403 | 94.628 | 0.569 | 1.00 | 16.48 | A |
| ATOM | 1379 | O | ALA | A | 185 | 36.176 | 94.967 | −0.334 | 1.00 | 17.01 | A |
| ATOM | 1380 | N | GLY | A | 186 | 35.761 | 93.824 | 1.565 | 1.00 | 15.70 | A |
| ATOM | 1381 | CA | GLY | A | 186 | 37.104 | 93.282 | 1.640 | 1.00 | 15.00 | A |
| ATOM | 1382 | C | GLY | A | 186 | 38.130 | 94.205 | 2.272 | 1.00 | 12.92 | A |
| ATOM | 1383 | O | GLY | A | 186 | 39.321 | 93.894 | 2.260 | 1.00 | 15.00 | A |
| ATOM | 1384 | N | LYS | A | 187 | 37.678 | 95.326 | 2.831 | 1.00 | 12.80 | A |
| ATOM | 1385 | CA | LYS | A | 187 | 38.588 | 96.285 | 3.452 | 1.00 | 13.16 | A |
| ATOM | 1386 | CB | LYS | A | 187 | 38.183 | 97.706 | 3.044 | 1.00 | 14.68 | A |
| ATOM | 1387 | CG | LYS | A | 187 | 38.165 | 97.942 | 1.541 | 1.00 | 16.39 | A |
| ATOM | 1388 | CD | LYS | A | 187 | 39.530 | 97.720 | 0.916 | 1.00 | 19.92 | A |
| ATOM | 1389 | CE | LYS | A | 187 | 39.476 | 97.944 | −0.590 | 1.00 | 22.39 | A |
| ATOM | 1390 | NZ | LYS | A | 187 | 40.797 | 97.715 | −1.237 | 1.00 | 24.55 | A |
| ATOM | 1391 | C | LYS | A | 187 | 38.710 | 96.122 | 4.962 | 1.00 | 13.20 | A |
| ATOM | 1392 | O | LYS | A | 187 | 39.306 | 96.960 | 5.630 | 1.00 | 12.61 | A |
| ATOM | 1393 | N | TYR | A | 188 | 38.131 | 95.059 | 5.506 | 1.00 | 11.47 | A |
| ATOM | 1394 | CA | TYR | A | 188 | 38.238 | 94.798 | 6.936 | 1.00 | 10.48 | A |
| ATOM | 1395 | CB | TYR | A | 188 | 37.025 | 95.349 | 7.697 | 1.00 | 9.76 | A |
| ATOM | 1396 | CG | TYR | A | 188 | 37.156 | 95.179 | 9.205 | 1.00 | 9.93 | A |
| ATOM | 1397 | CD1 | TYR | A | 188 | 37.983 | 96.021 | 9.956 | 1.00 | 11.85 | A |
| ATOM | 1398 | CE1 | TYR | A | 188 | 38.152 | 95.837 | 11.332 | 1.00 | 11.50 | A |
| ATOM | 1399 | CD2 | TYR | A | 188 | 36.498 | 94.144 | 9.875 | 1.00 | 9.54 | A |
| ATOM | 1400 | CE2 | TYR | A | 188 | 36.664 | 93.950 | 11.252 | 1.00 | 10.41 | A |
| ATOM | 1401 | CZ | TYR | A | 188 | 37.491 | 94.801 | 11.973 | 1.00 | 9.98 | A |
| ATOM | 1402 | OH | TYR | A | 188 | 37.657 | 94.612 | 13.324 | 1.00 | 10.94 | A |
| ATOM | 1403 | C | TYR | A | 188 | 38.390 | 93.309 | 7.155 | 1.00 | 9.90 | A |
| ATOM | 1404 | O | TYR | A | 188 | 37.642 | 92.514 | 6.587 | 1.00 | 11.93 | A |
| ATOM | 1405 | N | ALA | A | 189 | 39.365 | 92.943 | 7.977 | 1.00 | 9.93 | A |
| ATOM | 1406 | CA | ALA | A | 189 | 39.605 | 91.549 | 8.334 | 1.00 | 10.69 | A |
| ATOM | 1407 | CB | ALA | A | 189 | 41.013 | 91.124 | 7.947 | 1.00 | 11.59 | A |
| ATOM | 1408 | C | ALA | A | 189 | 39.426 | 91.504 | 9.849 | 1.00 | 9.86 | A |
| ATOM | 1409 | O | ALA | A | 189 | 39.845 | 92.421 | 10.556 | 1.00 | 11.37 | A |
| ATOM | 1410 | N | TYR | A | 190 | 38.807 | 90.441 | 10.348 | 1.00 | 9.99 | A |
| ATOM | 1411 | CA | TYR | A | 190 | 38.535 | 90.327 | 11.772 | 1.00 | 9.79 | A |
| ATOM | 1412 | CB | TYR | A | 190 | 37.011 | 90.171 | 11.928 | 1.00 | 9.92 | A |
| ATOM | 1413 | CG | TYR | A | 190 | 36.500 | 89.957 | 13.337 | 1.00 | 10.09 | A |
| ATOM | 1414 | CD1 | TYR | A | 190 | 37.043 | 90.648 | 14.415 | 1.00 | 11.70 | A |
| ATOM | 1415 | CE1 | TYR | A | 190 | 36.576 | 90.437 | 15.714 | 1.00 | 12.47 | A |
| ATOM | 1416 | CD2 | TYR | A | 190 | 35.468 | 89.052 | 13.587 | 1.00 | 11.76 | A |
| ATOM | 1417 | CE2 | TYR | A | 190 | 34.995 | 88.833 | 14.876 | 1.00 | 12.05 | A |
| ATOM | 1418 | CZ | TYR | A | 190 | 35.555 | 89.523 | 15.934 | 1.00 | 12.04 | A |
| ATOM | 1419 | OH | TYR | A | 190 | 35.124 | 89.258 | 17.215 | 1.00 | 14.89 | A |
| ATOM | 1420 | C | TYR | A | 190 | 39.351 | 89.225 | 12.439 | 1.00 | 8.97 | A |
| ATOM | 1421 | O | TYR | A | 190 | 39.390 | 88.095 | 11.957 | 1.00 | 10.33 | A |
| ATOM | 1422 | N | LEU | A | 191 | 40.020 | 89.568 | 13.537 | 1.00 | 9.20 | A |
| ATOM | 1423 | CA | LEU | A | 191 | 40.817 | 88.606 | 14.295 | 1.00 | 9.62 | A |
| ATOM | 1424 | CB | LEU | A | 191 | 42.075 | 89.294 | 14.825 | 1.00 | 10.20 | A |
| ATOM | 1425 | CG | LEU | A | 191 | 43.170 | 89.563 | 13.791 | 1.00 | 11.97 | A |
| ATOM | 1426 | CD1 | LEU | A | 191 | 44.047 | 90.708 | 14.253 | 1.00 | 15.67 | A |
| ATOM | 1427 | CD2 | LEU | A | 191 | 43.984 | 88.297 | 13.565 | 1.00 | 12.16 | A |
| ATOM | 1428 | C | LEU | A | 191 | 39.928 | 88.062 | 15.400 | 1.00 | 10.23 | A |
| ATOM | 1429 | O | LEU | A | 191 | 39.533 | 88.803 | 16.301 | 1.00 | 10.12 | A |
| ATOM | 1430 | N | LEU | A | 192 | 39.624 | 86.769 | 15.329 | 1.00 | 10.06 | A |
| ATOM | 1431 | CA | LEU | A | 192 | 38.761 | 86.128 | 16.315 | 1.00 | 9.01 | A |
| ATOM | 1432 | CB | LEU | A | 192 | 37.301 | 86.240 | 15.861 | 1.00 | 10.17 | A |
| ATOM | 1433 | CG | LEU | A | 192 | 36.844 | 85.219 | 14.813 | 1.00 | 9.95 | A |
| ATOM | 1434 | CD1 | LEU | A | 192 | 35.344 | 85.357 | 14.609 | 1.00 | 10.24 | A |
| ATOM | 1435 | CD2 | LEU | A | 192 | 37.582 | 85.425 | 13.495 | 1.00 | 10.31 | A |
| ATOM | 1436 | C | LEU | A | 192 | 39.183 | 84.682 | 16.532 | 1.00 | 8.95 | A |
| ATOM | 1437 | O | LEU | A | 192 | 40.000 | 84.136 | 14.788 | 1.00 | 8.84 | A |
| ATOM | 1438 | N | GLU | A | 193 | 38.616 | 84.042 | 17.546 | 1.00 | 9.42 | A |
| ATOM | 1439 | CA | GLU | A | 193 | 38.969 | 82.658 | 17.815 | 1.00 | 9.32 | A |
| ATOM | 1440 | CB | GLU | A | 193 | 38.484 | 82.303 | 19.226 | 1.00 | 9.15 | A |
| ATOM | 1441 | CG | GLU | A | 193 | 39.210 | 83.161 | 20.264 | 1.00 | 9.00 | A |
| ATOM | 1442 | CD | GLU | A | 193 | 38.783 | 82.924 | 21.698 | 1.00 | 7.63 | A |
| ATOM | 1443 | OE1 | GLU | A | 193 | 37.618 | 82.541 | 21.929 | 1.00 | 8.76 | A |
| ATOM | 1444 | OE2 | GLU | A | 193 | 39.619 | 83.143 | 22.596 | 1.00 | 8.73 | A |
| ATOM | 1445 | C | GLU | A | 193 | 38.477 | 81.746 | 16.686 | 1.00 | 9.08 | A |
| ATOM | 1446 | O | GLU | A | 193 | 37.391 | 81.937 | 16.132 | 1.00 | 8.87 | A |
| ATOM | 1447 | N | SER | A | 194 | 39.312 | 80.769 | 16.338 | 1.00 | 9.00 | A |
| ATOM | 1448 | CA | SER | A | 194 | 39.031 | 79.836 | 15.247 | 1.00 | 9.40 | A |
| ATOM | 1449 | CB | SER | A | 194 | 40.172 | 78.819 | 15.167 | 1.00 | 9.63 | A |
| ATOM | 1450 | OG | SER | A | 194 | 40.297 | 78.090 | 16.375 | 1.00 | 9.18 | A |
| ATOM | 1451 | C | SER | A | 194 | 37.659 | 79.183 | 15.344 | 1.00 | 9.07 | A |
| ATOM | 1452 | O | SER | A | 194 | 37.016 | 78.919 | 14.327 | 1.00 | 8.31 | A |
| ATOM | 1453 | N | THR | A | 195 | 37.226 | 78.913 | 16.569 | 1.00 | 8.83 | A |
| ATOM | 1454 | CA | THR | A | 195 | 35.928 | 78.314 | 16.827 | 1.00 | 9.21 | A |
| ATOM | 1455 | CB | THR | A | 195 | 35.771 | 78.100 | 18.328 | 1.00 | 11.82 | A |
| ATOM | 1456 | OG1 | THR | A | 195 | 36.318 | 79.236 | 19.005 | 1.00 | 10.54 | A |
| ATOM | 1457 | CG2 | THR | A | 195 | 36.518 | 76.845 | 18.773 | 1.00 | 11.99 | A |
| ATOM | 1458 | C | THR | A | 195 | 34.797 | 79.203 | 16.287 | 1.00 | 9.86 | A |
| ATOM | 1459 | O | THR | A | 195 | 33.918 | 78.723 | 15.580 | 1.00 | 9.91 | A |
| ATOM | 1460 | N | MET | A | 196 | 34.814 | 80.496 | 16.607 | 1.00 | 10.11 | A |
| ATOM | 1461 | CA | MET | A | 196 | 33.776 | 81.396 | 16.100 | 1.00 | 11.13 | A |
| ATOM | 1462 | CB | MET | A | 196 | 33.866 | 82.767 | 16.781 | 1.00 | 10.81 | A |
| ATOM | 1463 | CG | MET | A | 196 | 33.280 | 82.832 | 18.176 | 1.00 | 12.64 | A |
| ATOM | 1464 | SD | MET | A | 196 | 34.294 | 81.964 | 19.362 | 1.00 | 12.02 | A |
| ATOM | 1465 | CE | MET | A | 196 | 33.945 | 82.894 | 20.858 | 1.00 | 13.21 | A |
| ATOM | 1466 | C | MET | A | 196 | 33.895 | 81.525 | 14.589 | 1.00 | 10.05 | A |
| ATOM | 1467 | O | MET | A | 196 | 32.892 | 81.583 | 13.878 | 1.00 | 11.28 | A |
| ATOM | 1468 | N | ASN | A | 197 | 35.133 | 81.585 | 14.111 | 1.00 | 10.27 | A |
| ATOM | 1469 | CA | ASN | A | 197 | 35.415 | 81.724 | 12.685 | 1.00 | 10.27 | A |
| ATOM | 1470 | CB | ASN | A | 197 | 36.936 | 81.798 | 12.506 | 1.00 | 10.10 | A |
| ATOM | 1471 | CG | ASN | A | 197 | 37.349 | 82.493 | 11.223 | 1.00 | 11.64 | A |
| ATOM | 1472 | OD1 | ASN | A | 197 | 36.534 | 82.715 | 10.330 | 1.00 | 10.77 | A |
| ATOM | 1473 | ND2 | ASN | A | 197 | 38.628 | 82.833 | 11.125 | 1.00 | 9.60 | A |
| ATOM | 1474 | C | ASN | A | 197 | 34.777 | 80.572 | 11.902 | 1.00 | 10.52 | A |
| ATOM | 1475 | O | ASN | A | 197 | 34.086 | 80.786 | 10.901 | 1.00 | 10.37 | A |
| ATOM | 1476 | N | GLU | A | 198 | 35.022 | 79.349 | 12.367 | 1.00 | 11.10 | A |
| ATOM | 1477 | CA | GLU | A | 198 | 34.493 | 78.140 | 11.732 | 1.00 | 11.31 | A |
| ATOM | 1478 | CB | GLU | A | 198 | 35.101 | 76.914 | 12.420 | 1.00 | 13.92 | A |
| ATOM | 1479 | CG | GLU | A | 198 | 36.607 | 76.801 | 12.283 | 1.00 | 15.71 | A |
| ATOM | 1480 | CD | GLU | A | 198 | 37.231 | 75.942 | 13.370 | 1.00 | 19.06 | A |
| ATOM | 1481 | OE1 | GLU | A | 198 | 38.462 | 75.737 | 13.329 | 1.00 | 20.07 | A |
| ATOM | 1482 | OE2 | GLU | A | 198 | 36.497 | 75.477 | 14.269 | 1.00 | 19.02 | A |
| ATOM | 1483 | C | GLU | A | 198 | 32.970 | 78.125 | 11.762 | 1.00 | 10.82 | A |
| ATOM | 1484 | O | GLU | A | 198 | 32.322 | 77.698 | 10.803 | 1.00 | 12.14 | A |
| ATOM | 1485 | N | TYR | A | 199 | 32.402 | 78.583 | 12.872 | 1.00 | 11.22 | A |
| ATOM | 1486 | CA | TYR | A | 199 | 30.956 | 78.622 | 13.024 | 1.00 | 11.52 | A |
| ATOM | 1487 | CB | TYR | A | 199 | 30.615 | 79.051 | 14.449 | 1.00 | 12.35 | A |
| ATOM | 1488 | CG | TYR | A | 199 | 29.172 | 79.466 | 14.616 | 1.00 | 11.56 | A |
| ATOM | 1489 | CD1 | TYR | A | 199 | 28.144 | 78.523 | 14.590 | 1.00 | 13.00 | A |
| ATOM | 1490 | CE1 | TYR | A | 199 | 26.807 | 78.911 | 14.722 | 1.00 | 12.91 | A |
| ATOM | 1491 | CD2 | TYR | A | 199 | 28.832 | 80.809 | 14.778 | 1.00 | 12.75 | A |
| ATOM | 1492 | CE2 | TYR | A | 199 | 27.504 | 81.207 | 14.910 | 1.00 | 14.56 | A |
| ATOM | 1493 | CZ | TYR | A | 199 | 26.499 | 80.254 | 14.882 | 1.00 | 13.63 | A |
| ATOM | 1494 | OH | TYR | A | 199 | 25.189 | 80.651 | 15.018 | 1.00 | 17.35 | A |
| ATOM | 1495 | C | TYR | A | 199 | 30.343 | 79.544 | 11.979 | 1.00 | 11.53 | A |
| ATOM | 1496 | O | TYR | A | 199 | 29.429 | 79.155 | 11.254 | 1.00 | 13.28 | A |
| ATOM | 1497 | N | ILE | A | 200 | 30.854 | 80.770 | 11.912 | 1.00 | 12.00 | A |
| ATOM | 1498 | CA | ILE | A | 200 | 30.359 | 81.771 | 10.972 | 1.00 | 12.68 | A |
| ATOM | 1499 | CB | ILE | A | 200 | 31.058 | 83.135 | 11.214 | 1.00 | 13.01 | A |
| ATOM | 1500 | CG2 | ILE | A | 200 | 30.630 | 84.144 | 10.155 | 1.00 | 12.44 | A |
| ATOM | 1501 | CG1 | ILE | A | 200 | 30.715 | 83.664 | 12.614 | 1.00 | 14.61 | A |
| ATOM | 1502 | CD1 | ILE | A | 200 | 29.244 | 84.000 | 12.829 | 1.00 | 14.43 | A |
| ATOM | 1503 | C | ILE | A | 200 | 30.563 | 81.350 | 9.515 | 1.00 | 12.69 | A |
| ATOM | 1504 | O | ILE | A | 200 | 29.748 | 81.668 | 8.646 | 1.00 | 12.52 | A |
| ATOM | 1505 | N | GLU | A | 201 | 31.651 | 80.634 | 9.255 | 1.00 | 12.45 | A |
| ATOM | 1506 | CA | GLU | A | 201 | 31.964 | 80.170 | 7.910 | 1.00 | 15.01 | A |
| ATOM | 1507 | CB | GLU | A | 201 | 33.330 | 79.483 | 7.942 | 1.00 | 15.01 | A |
| ATOM | 1508 | CG | GLU | A | 201 | 33.910 | 79.134 | 6.586 | 1.00 | 20.19 | A |
| ATOM | 1509 | CD | GLU | A | 201 | 35.322 | 78.593 | 6.698 | 1.00 | 21.08 | A |
| ATOM | 1510 | OE1 | GLU | A | 201 | 35.507 | 77.530 | 7.328 | 1.00 | 24.42 | A |
| ATOM | 1511 | OE2 | GLU | A | 201 | 36.249 | 79.234 | 6.164 | 1.00 | 25.13 | A |
| ATOM | 1512 | C | GLU | A | 201 | 30.857 | 79.272 | 7.366 | 1.00 | 15.85 | A |
| ATOM | 1513 | O | GLU | A | 201 | 30.718 | 79.120 | 6.151 | 1.00 | 15.98 | A |
| ATOM | 1514 | N | GLN | A | 202 | 30.070 | 78.682 | 8.263 | 1.00 | 15.03 | A |
| ATOM | 1515 | CA | GLN | A | 202 | 28.982 | 77.792 | 7.864 | 1.00 | 17.87 | A |
| ATOM | 1516 | CB | GLN | A | 202 | 29.070 | 76.499 | 8.687 | 1.00 | 18.85 | A |
| ATOM | 1517 | CG | GLN | A | 202 | 30.394 | 75.758 | 8.569 | 1.00 | 22.01 | A |
| ATOM | 1518 | CD | GLN | A | 202 | 30.753 | 75.418 | 7.135 | 1.00 | 25.59 | A |
| ATOM | 1519 | OE1 | GLN | A | 202 | 29.948 | 74.848 | 6.399 | 1.00 | 28.46 | A |
| ATOM | 1520 | NE2 | GLN | A | 202 | 31.973 | 75.758 | 6.734 | 1.00 | 26.17 | A |
| ATOM | 1521 | C | GLN | A | 202 | 27.622 | 78.479 | 7.987 | 1.00 | 18.30 | A |
| ATOM | 1522 | O | GLN | A | 202 | 26.586 | 77.813 | 8.026 | 1.00 | 21.15 | A |
| ATOM | 1523 | N | ARG | A | 203 | 27.624 | 79.806 | 8.044 | 1.00 | 17.72 | A |
| ATOM | 1524 | CA | ARG | A | 203 | 26.384 | 80.569 | 8.167 | 1.00 | 17.74 | A |
| ATOM | 1525 | CB | ARG | A | 203 | 26.448 | 81.435 | 9.432 | 1.00 | 19.84 | A |
| ATOM | 1526 | CG | ARG | A | 203 | 26.722 | 80.670 | 10.716 | 1.00 | 20.83 | A |
| ATOM | 1527 | CD | ARG | A | 203 | 25.507 | 79.882 | 11.176 | 1.00 | 24.68 | A |
| ATOM | 1528 | NE | ARG | A | 203 | 24.412 | 80.761 | 11.576 | 1.00 | 27.02 | A |
| ATOM | 1529 | CZ | ARG | A | 203 | 23.261 | 80.336 | 12.087 | 1.00 | 28.82 | A |
| ATOM | 1530 | NH1 | ARG | A | 203 | 23.050 | 79.039 | 12.262 | 1.00 | 28.91 | A |
| ATOM | 1531 | NH2 | ARG | A | 203 | 22.322 | 81.210 | 12.426 | 1.00 | 28.42 | A |
| ATOM | 1532 | C | ARG | A | 203 | 26.159 | 81.401 | 6.912 | 1.00 | 17.54 | A |
| ATOM | 1533 | O | ARG | A | 203 | 27.105 | 81.941 | 6.337 | 1.00 | 16.78 | A |
| ATOM | 1534 | N | LYS | A | 204 | 24.904 | 81.502 | 6.487 | 1.00 | 19.44 | A |
| ATOM | 1535 | CA | LYS | A | 204 | 24.568 | 82.301 | 5.316 | 1.00 | 20.89 | A |
| ATOM | 1536 | CB | LYS | A | 204 | 23.086 | 82.103 | 4.981 | 1.00 | 22.65 | A |
| ATOM | 1537 | CG | LYS | A | 204 | 22.733 | 80.694 | 4.520 | 1.00 | 26.49 | A |
| ATOM | 1538 | CD | LYS | A | 204 | 21.223 | 80.488 | 4.438 | 1.00 | 29.93 | A |
| ATOM | 1539 | CE | LYS | A | 204 | 20.572 | 81.455 | 3.461 | 1.00 | 32.13 | A |
| ATOM | 1540 | NZ | LYS | A | 204 | 19.090 | 81.293 | 3.421 | 1.00 | 34.80 | A |
| ATOM | 1541 | C | LYS | A | 204 | 24.926 | 83.737 | 5.682 | 1.00 | 20.57 | A |
| ATOM | 1542 | O | LYS | A | 204 | 24.876 | 84.113 | 6.855 | 1.00 | 21.17 | A |
| ATOM | 1543 | N | PRO | A | 205 | 25.259 | 84.573 | 4.687 | 1.00 | 20.27 | A |
| ATOM | 1544 | CD | PRO | A | 205 | 25.404 | 86.015 | 4.951 | 1.00 | 20.25 | A |
| ATOM | 1545 | CA | PRO | A | 205 | 25.332 | 84.310 | 3.247 | 1.00 | 19.98 | A |
| ATOM | 1546 | CB | PRO | A | 205 | 25.111 | 85.700 | 2.659 | 1.00 | 20.23 | A |
| ATOM | 1547 | CG | PRO | A | 205 | 25.918 | 86.568 | 3.569 | 1.00 | 22.10 | A |
| ATOM | 1548 | C | PRO | A | 205 | 26.578 | 83.585 | 2.727 | 1.00 | 19.52 | A |
| ATOM | 1549 | O | PRO | A | 205 | 26.893 | 83.678 | 1.541 | 1.00 | 20.02 | A |
| ATOM | 1550 | N | CYS | A | 206 | 27.292 | 82.877 | 3.597 | 1.00 | 17.94 | A |
| ATOM | 1551 | CA | CYS | A | 206 | 28.474 | 82.127 | 3.171 | 1.00 | 17.10 | A |
| ATOM | 1552 | C | CYS | A | 206 | 29.475 | 83.007 | 2.435 | 1.00 | 16.19 | A |
| ATOM | 1553 | O | CYS | A | 206 | 30.006 | 82.610 | 1.396 | 1.00 | 16.94 | A |
| ATOM | 1554 | CB | CYS | A | 206 | 28.018 | 80.945 | 2.310 | 1.00 | 18.59 | A |
| ATOM | 1555 | SG | CYS | A | 206 | 26.707 | 79.952 | 3.079 | 1.00 | 20.55 | A |
| ATOM | 1556 | N | ASP | A | 207 | 29.744 | 84.189 | 2.976 | 1.00 | 15.45 | A |
| ATOM | 1557 | CA | ASP | A | 207 | 30.666 | 85.116 | 2.334 | 1.00 | 14.32 | A |
| ATOM | 1558 | CB | ASP | A | 207 | 29.907 | 86.408 | 2.009 | 1.00 | 16.26 | A |
| ATOM | 1559 | CG | ASP | A | 207 | 29.412 | 87.120 | 3.252 | 1.00 | 15.80 | A |
| ATOM | 1560 | OD1 | ASP | A | 207 | 29.376 | 86.488 | 4.330 | 1.00 | 16.40 | A |
| ATOM | 1561 | OD2 | ASP | A | 207 | 29.048 | 88.311 | 3.150 | 1.00 | 18.95 | A |
| ATOM | 1562 | C | ASP | A | 207 | 31.937 | 85.361 | 3.139 | 1.00 | 14.39 | A |
| ATOM | 1563 | O | ASP | A | 207 | 32.711 | 86.261 | 2.817 | 1.00 | 13.94 | A |
| ATOM | 1564 | N | THR | A | 208 | 32.155 | 84.573 | 4.187 | 1.00 | 14.10 | A |
| ATOM | 1565 | CA | THR | A | 208 | 33.358 | 84.734 | 4.997 | 1.00 | 13.35 | A |
| ATOM | 1566 | CB | THR | A | 208 | 33.027 | 84.977 | 6.483 | 1.00 | 13.59 | A |
| ATOM | 1567 | OG1 | THR | A | 208 | 32.375 | 83.823 | 7.026 | 1.00 | 15.09 | A |
| ATOM | 1568 | CG2 | THR | A | 208 | 32.123 | 86.193 | 6.634 | 1.00 | 14.70 | A |
| ATOM | 1569 | C | THR | A | 208 | 24.226 | 83.487 | 4.891 | 1.00 | 13.65 | A |
| ATOM | 1570 | O | THR | A | 208 | 33.753 | 82.414 | 4.506 | 1.00 | 14.29 | A |
| ATOM | 1571 | N | MET | A | 209 | 35.497 | 83.627 | 5.241 | 1.00 | 13.49 | A |
| ATOM | 1572 | CA | MET | A | 209 | 36.419 | 82.509 | 5.166 | 1.00 | 14.22 | A |
| ATOM | 1573 | CB | MET | A | 209 | 36.919 | 82.399 | 3.723 | 1.00 | 17.35 | A |
| ATOM | 1574 | CG | MET | A | 209 | 38.022 | 81.385 | 3.508 | 1.00 | 20.95 | A |
| ATOM | 1575 | SD | MET | A | 209 | 38.507 | 81.287 | 1.773 | 1.00 | 25.45 | A |
| ATOM | 1576 | CE | MET | A | 209 | 39.516 | 82.733 | 1.609 | 1.00 | 25.90 | A |
| ATOM | 1577 | C | MET | A | 209 | 37.550 | 82.654 | 6.168 | 1.00 | 13.82 | A |
| ATOM | 1578 | O | MET | A | 209 | 38.030 | 83.762 | 6.417 | 1.00 | 13.46 | A |
| ATOM | 1579 | N | LYS | A | 210 | 37.963 | 81.531 | 6.749 | 1.00 | 15.10 | A |
| ATOM | 1580 | CA | LYS | A | 210 | 39.076 | 81.515 | 7.690 | 1.00 | 15.37 | A |
| ATOM | 1581 | CB | LYS | A | 210 | 38.870 | 80.410 | 8.734 | 1.00 | 16.35 | A |
| ATOM | 1582 | CG | LYS | A | 210 | 40.045 | 80.256 | 9.702 | 1.00 | 19.49 | A |
| ATOM | 1583 | CD | LYS | A | 210 | 39.752 | 79.249 | 10.813 | 1.00 | 21.65 | A |
| ATOM | 1584 | CE | LYS | A | 210 | 39.665 | 77.824 | 10.288 | 1.00 | 23.45 | A |
| ATOM | 1585 | NZ | LYS | A | 210 | 40.975 | 77.345 | 9.768 | 1.00 | 24.20 | A |
| ATOM | 1586 | C | LYS | A | 210 | 40.316 | 81.294 | 6.834 | 1.00 | 15.15 | A |
| ATOM | 1587 | O | LYS | A | 210 | 40.356 | 80.361 | 6.032 | 1.00 | 16.64 | A |
| ATOM | 1588 | N | VAL | A | 211 | 41.323 | 82.148 | 6.990 | 1.00 | 14.55 | A |
| ATOM | 1589 | CA | VAL | A | 211 | 42.544 | 82.007 | 6.202 | 1.00 | 15.65 | A |
| ATOM | 1590 | CB | VAL | A | 211 | 42.695 | 83.150 | 5.167 | 1.00 | 15.98 | A |
| ATOM | 1591 | CG1 | VAL | A | 211 | 41.593 | 83.061 | 4.133 | 1.00 | 17.55 | A |
| ATOM | 1592 | CG2 | VAL | A | 211 | 42.666 | 84.500 | 5.867 | 1.00 | 15.82 | A |
| ATOM | 1593 | C | VAL | A | 211 | 43.806 | 81.967 | 7.047 | 1.00 | 16.70 | A |
| ATOM | 1594 | O | VAL | A | 211 | 43.872 | 82.574 | 8.117 | 1.00 | 15.53 | A |
| ATOM | 1595 | N | GLY | A | 212 | 44.809 | 81.250 | 6.547 | 1.00 | 18.12 | A |
| ATOM | 1596 | CA | GLY | A | 212 | 46.072 | 81.134 | 7.249 | 1.00 | 19.13 | A |
| ATOM | 1597 | C | GLY | A | 212 | 45.986 | 80.237 | 8.465 | 1.00 | 18.29 | A |
| ATOM | 1598 | O | GLY | A | 212 | 44.898 | 79.830 | 8.876 | 1.00 | 20.17 | A |
| ATOM | 1599 | N | GLY | A | 213 | 47.139 | 79.912 | 9.036 | 1.00 | 17.49 | A |
| ATOM | 1600 | CA | GLY | A | 213 | 47.147 | 79.076 | 10.218 | 1.00 | 16.45 | A |
| ATOM | 1601 | C | GLY | A | 213 | 46.769 | 79.928 | 11.411 | 1.00 | 14.83 | A |
| ATOM | 1602 | O | GLY | A | 213 | 46.769 | 81.161 | 11.328 | 1.00 | 17.42 | A |
| ATOM | 1603 | N | ASN | A | 214 | 46.433 | 79.288 | 12.522 | 1.00 | 13.24 | A |
| ATOM | 1604 | CA | ASN | A | 214 | 46.073 | 80.039 | 13.713 | 1.00 | 11.96 | A |
| ATOM | 1605 | CB | ASN | A | 214 | 45.423 | 79.103 | 14.735 | 1.00 | 13.42 | A |
| ATOM | 1606 | CG | ASN | A | 214 | 44.093 | 78.554 | 14.262 | 1.00 | 14.31 | A |
| ATOM | 1607 | OD1 | ASN | A | 214 | 43.445 | 79.128 | 13.386 | 1.00 | 14.64 | A |
| ATOM | 1608 | ND2 | ASN | A | 214 | 43.671 | 77.444 | 14.854 | 1.00 | 16.40 | A |
| ATOM | 1609 | C | ASN | A | 214 | 47.329 | 80.722 | 14.238 | 1.00 | 11.82 | A |
| ATOM | 1610 | O | ASN | A | 214 | 48.442 | 80.237 | 14.027 | 1.00 | 13.30 | A |
| ATOM | 1611 | N | LEU | A | 215 | 47.145 | 81.842 | 14.927 | 1.00 | 10.88 | A |
| ATOM | 1612 | CA | LEU | A | 215 | 48.267 | 82.592 | 15.478 | 1.00 | 11.07 | A |
| ATOM | 1613 | CB | LEU | A | 215 | 47.867 | 84.061 | 15.644 | 1.00 | 11.85 | A |
| ATOM | 1614 | CG | LEU | A | 215 | 47.760 | 84.915 | 14.375 | 1.00 | 12.48 | A |
| ATOM | 1615 | CD1 | LEU | A | 215 | 46.698 | 84.359 | 13.439 | 1.00 | 15.01 | A |
| ATOM | 1616 | CD2 | LEU | A | 215 | 47.430 | 86.344 | 14.768 | 1.00 | 13.40 | A |
| ATOM | 1617 | C | LEU | A | 215 | 48.784 | 81.968 | 16.768 | 1.00 | 11.18 | A |
| ATOM | 1618 | O | LEU | A | 215 | 49.931 | 82.197 | 17.151 | 1.00 | 13.06 | A |
| ATOM | 1619 | N | ASP | A | 216 | 47.935 | 81.203 | 17.451 | 1.00 | 10.77 | A |
| ATOM | 1620 | CA | ASP | A | 216 | 48.328 | 80.531 | 18.686 | 1.00 | 10.05 | A |
| ATOM | 1621 | CB | ASP | A | 216 | 48.230 | 81.498 | 19.887 | 1.00 | 10.46 | A |
| ATOM | 1622 | CG | ASP | A | 216 | 46.805 | 81.909 | 20.231 | 1.00 | 9.55 | A |
| ATOM | 1623 | OD1 | ASP | A | 216 | 45.849 | 81.513 | 19.528 | 1.00 | 9.84 | A |
| ATOM | 1624 | OD2 | ASP | A | 216 | 46.658 | 82.649 | 21.231 | 1.00 | 10.37 | A |
| ATOM | 1625 | C | ASP | A | 216 | 47.519 | 79.250 | 18.841 | 1.00 | 9.18 | A |
| ATOM | 1626 | O | ASP | A | 216 | 46.689 | 78.937 | 17.990 | 1.00 | 10.30 | A |
| ATOM | 1627 | N | SER | A | 217 | 47.782 | 78.511 | 19.916 | 1.00 | 10.24 | A |
| ATOM | 1628 | CA | SER | A | 217 | 47.103 | 77.244 | 20.179 | 1.00 | 10.49 | A |
| ATOM | 1629 | CB | SER | A | 217 | 48.017 | 76.095 | 19.747 | 1.00 | 13.97 | A |
| ATOM | 1630 | OG | SER | A | 217 | 48.283 | 76.142 | 18.362 | 1.00 | 20.86 | A |
| ATOM | 1631 | C | SER | A | 217 | 46.729 | 77.169 | 21.649 | 1.00 | 10.89 | A |
| ATOM | 1632 | O | SER | A | 217 | 47.595 | 77.133 | 22.519 | 1.00 | 12.10 | A |
| ATOM | 1633 | N | LYS | A | 218 | 45.431 | 77.124 | 21.926 | 1.00 | 11.01 | A |
| ATOM | 1634 | CA | LYS | A | 218 | 44.961 | 77.075 | 23.300 | 1.00 | 10.66 | A |
| ATOM | 1635 | CB | LYS | A | 218 | 44.917 | 78.507 | 23.845 | 1.00 | 10.42 | A |
| ATOM | 1636 | CG | LYS | A | 218 | 44.102 | 79.476 | 22.999 | 1.00 | 10.17 | A |
| ATOM | 1637 | CD | LYS | A | 218 | 44.537 | 80.910 | 23.264 | 1.00 | 9.85 | A |
| ATOM | 1638 | CE | LYS | A | 218 | 43.734 | 81.900 | 22.438 | 1.00 | 8.64 | A |
| ATOM | 1639 | NZ | LYS | A | 218 | 44.316 | 83.269 | 22.501 | 1.00 | 8.85 | A |
| ATOM | 1640 | C | LYS | A | 218 | 43.628 | 76.348 | 23.369 | 1.00 | 9.98 | A |
| ATOM | 1641 | O | LYS | A | 218 | 43.281 | 75.584 | 23.469 | 1.00 | 9.15 | A |
| ATOM | 1642 | N | GLY | A | 219 | 42.877 | 76.587 | 24.438 | 1.00 | 10.04 | A |
| ATOM | 1643 | CA | GLY | A | 219 | 41.597 | 75.924 | 24.582 | 1.00 | 9.98 | A |
| ATOM | 1644 | C | GLY | A | 219 | 40.682 | 76.612 | 25.570 | 1.00 | 9.33 | A |
| ATOM | 1645 | O | GLY | A | 219 | 41.098 | 77.525 | 26.286 | 1.00 | 9.61 | A |
| ATOM | 1646 | N | TYR | A | 220 | 39.424 | 76.182 | 25.575 | 1.00 | 8.52 | A |
| ATOM | 1647 | CA | TYR | A | 220 | 38.418 | 76.700 | 26.502 | 1.00 | 9.25 | A |
| ATOM | 1648 | CB | TYR | A | 220 | 37.034 | 76.765 | 25.852 | 1.00 | 8.84 | A |
| ATOM | 1649 | CG | TYR | A | 220 | 36.885 | 77.684 | 24.677 | 1.00 | 8.82 | A |
| ATOM | 1650 | CD1 | TYR | A | 220 | 37.475 | 78.946 | 24.664 | 1.00 | 7.89 | A |
| ATOM | 1651 | CE1 | TYR | A | 220 | 37.277 | 79.813 | 23.606 | 1.00 | 8.92 | A |
| ATOM | 1652 | CD2 | TYR | A | 220 | 36.094 | 77.312 | 23.596 | 1.00 | 9.12 | A |
| ATOM | 1653 | CE2 | TYR | A | 220 | 35.887 | 78.172 | 22.531 | 1.00 | 9.33 | A |
| ATOM | 1654 | CZ | TYR | A | 220 | 36.483 | 79.421 | 22.543 | 1.00 | 8.81 | A |
| ATOM | 1655 | OH | TYR | A | 220 | 36.288 | 80.272 | 21.483 | 1.00 | 9.44 | A |
| ATOM | 1656 | C | TYR | A | 220 | 38.386 | 78.740 | 27.670 | 1.00 | 8.73 | A |
| ATOM | 1657 | O | TYR | A | 220 | 38.493 | 74.524 | 27.484 | 1.00 | 8.20 | A |
| ATOM | 1658 | N | GLY | A | 221 | 38.221 | 76.277 | 28.870 | 1.00 | 8.26 | A |
| ATOM | 1659 | CA | GLY | A | 221 | 38.146 | 75.423 | 30.035 | 1.00 | 8.92 | A |
| ATOM | 1660 | C | GLY | A | 221 | 36.988 | 75.801 | 30.934 | 1.00 | 7.99 | A |
| ATOM | 1661 | O | GLY | A | 221 | 36.505 | 76.936 | 30.900 | 1.00 | 8.94 | A |
| ATOM | 1662 | N | ILE | A | 222 | 36.516 | 74.838 | 31.716 | 1.00 | 9.12 | A |
| ATOM | 1663 | CA | ILE | A | 222 | 35.442 | 75.092 | 32.663 | 1.00 | 9.00 | A |
| ATOM | 1664 | CB | ILE | A | 222 | 34.692 | 73.787 | 33.019 | 1.00 | 9.58 | A |
| ATOM | 1665 | CG2 | ILE | A | 222 | 33.793 | 74.015 | 34.229 | 1.00 | 9.65 | A |
| ATOM | 1666 | CG1 | ILE | A | 222 | 33.871 | 73.324 | 31.809 | 1.00 | 12.96 | A |
| ATOM | 1667 | CD1 | ILE | A | 222 | 33.042 | 72.072 | 32.047 | 1.00 | 14.76 | A |
| ATOM | 1668 | C | ILE | A | 222 | 36.147 | 75.659 | 33.893 | 1.00 | 9.66 | A |
| ATOM | 1669 | O | ILE | A | 222 | 37.166 | 75.120 | 34.334 | 1.00 | 11.79 | A |
| ATOM | 1670 | N | ALA | A | 223 | 35.622 | 76.746 | 34.445 | 1.00 | 9.36 | A |
| ATOM | 1671 | CA | ALA | A | 223 | 36.260 | 77.373 | 35.590 | 1.00 | 9.69 | A |
| ATOM | 1672 | CB | ALA | A | 223 | 36.540 | 78.843 | 35.280 | 1.00 | 9.35 | A |
| ATOM | 1673 | C | ALA | A | 223 | 35.460 | 77.258 | 36.879 | 1.00 | 9.65 | A |
| ATOM | 1674 | O | ALA | A | 223 | 34.230 | 77.289 | 36.877 | 1.00 | 9.55 | A |
| ATOM | 1675 | N | THR | A | 224 | 36.188 | 77.124 | 37.982 | 1.00 | 10.12 | A |
| ATOM | 1676 | CA | THR | A | 224 | 35.601 | 77.035 | 39.314 | 1.00 | 10.80 | A |
| ATOM | 1677 | CB | THR | A | 224 | 35.577 | 75.578 | 39.829 | 1.00 | 11.59 | A |
| ATOM | 1678 | OG1 | THR | A | 224 | 36.849 | 74.967 | 39.589 | 1.00 | 12.02 | A |
| ATOM | 1679 | CG2 | THR | A | 224 | 34.489 | 74.778 | 39.141 | 1.00 | 11.59 | A |
| ATOM | 1680 | C | THR | A | 224 | 36.473 | 77.867 | 40.251 | 1.00 | 11.60 | A |
| ATOM | 1681 | O | THR | A | 224 | 37.629 | 78.153 | 39.940 | 1.00 | 11.33 | A |
| ATOM | 1682 | N | PRO | A | 225 | 35.928 | 78.290 | 41.403 | 1.00 | 11.75 | A |
| ATOM | 1683 | CD | PRO | A | 225 | 34.573 | 78.100 | 41.944 | 1.00 | 12.51 | A |
| ATOM | 1684 | CA | PRO | A | 225 | 36.755 | 79.082 | 42.317 | 1.00 | 12.26 | A |
| ATOM | 1685 | CB | PRO | A | 225 | 35.744 | 79.535 | 43.372 | 1.00 | 14.55 | A |
| ATOM | 1686 | CG | PRO | A | 225 | 34.792 | 78.384 | 43.476 | 1.00 | 17.48 | A |
| ATOM | 1687 | C | PRO | A | 225 | 37.904 | 78.220 | 42.829 | 1.00 | 14.13 | A |
| ATOM | 1688 | O | PRO | A | 225 | 37.762 | 77.004 | 42.948 | 1.00 | 13.19 | A |
| ATOM | 1689 | N | LYS | A | 226 | 39.043 | 78.838 | 43.118 | 1.00 | 15.99 | A |
| ATOM | 1690 | CA | LYS | A | 226 | 40.187 | 78.087 | 43.617 | 1.00 | 19.79 | A |
| ATOM | 1691 | CB | LYS | A | 226 | 41.352 | 79.040 | 43.890 | 1.00 | 20.79 | A |
| ATOM | 1692 | CG | LYS | A | 226 | 42.257 | 79.278 | 42.705 | 1.00 | 22.75 | A |
| ATOM | 1693 | CD | LYS | A | 226 | 43.553 | 79.930 | 43.156 | 1.00 | 24.00 | A |
| ATOM | 1694 | CE | LYS | A | 226 | 44.558 | 80.005 | 42.025 | 1.00 | 25.83 | A |
| ATOM | 1695 | NZ | LYS | A | 226 | 45.870 | 80.514 | 42.503 | 1.00 | 28.01 | A |
| ATOM | 1696 | C | LYS | A | 226 | 39.807 | 77.277 | 44.845 | 1.00 | 21.58 | A |
| ATOM | 1697 | O | LYS | A | 226 | 39.138 | 77.778 | 45.750 | 1.00 | 21.79 | A |
| ATOM | 1698 | N | GLY | A | 227 | 40.236 | 76.019 | 44.869 | 1.00 | 22.89 | A |
| ATOM | 1699 | CA | GLY | A | 227 | 39.950 | 75.159 | 46.001 | 1.00 | 23.86 | A |
| ATOM | 1700 | C | GLY | A | 227 | 38.567 | 74.540 | 46.021 | 1.00 | 24.69 | A |
| ATOM | 1701 | O | GLY | A | 227 | 38.251 | 73.767 | 46.924 | 1.00 | 25.45 | A |
| ATOM | 1702 | N | SER | A | 228 | 37.738 | 74.871 | 45.034 | 1.00 | 23.96 | A |
| ATOM | 1703 | CA | SER | A | 228 | 36.386 | 74.326 | 44.964 | 1.00 | 24.91 | A |
| ATOM | 1704 | CB | SER | A | 228 | 35.671 | 74.896 | 43.738 | 1.00 | 24.44 | A |
| ATOM | 1705 | OG | SER | A | 228 | 34.442 | 74.224 | 43.526 | 1.00 | 27.04 | A |
| ATOM | 1706 | C | SER | A | 228 | 36.406 | 72.804 | 44.966 | 1.00 | 25.46 | A |
| ATOM | 1707 | O | SER | A | 228 | 37.220 | 72.183 | 44.284 | 1.00 | 24.95 | A |
| ATOM | 1708 | N | SER | A | 229 | 35.493 | 72.210 | 45.725 | 1.00 | 26.15 | A |
| ATOM | 1709 | CA | SER | A | 229 | 35.400 | 70.759 | 45.821 | 1.00 | 26.28 | A |
| ATOM | 1710 | CB | SER | A | 229 | 34.715 | 70.393 | 47.141 | 1.00 | 27.58 | A |
| ATOM | 1711 | OG | SER | A | 229 | 33.401 | 70.921 | 47.192 | 1.00 | 29.90 | A |
| ATOM | 1712 | C | SER | A | 229 | 34.684 | 70.183 | 44.604 | 1.00 | 25.17 | A |
| ATOM | 1713 | O | SER | A | 229 | 34.470 | 68.975 | 44.518 | 1.00 | 25.95 | A |
| ATOM | 1714 | N | LEU | A | 230 | 34.312 | 71.050 | 43.669 | 1.00 | 22.39 | A |
| ATOM | 1715 | CA | LEU | A | 230 | 33.619 | 70.621 | 42.461 | 1.00 | 21.14 | A |
| ATOM | 1716 | CB | LEU | A | 230 | 32.500 | 71.625 | 42.148 | 1.00 | 23.01 | A |
| ATOM | 1717 | CG | LEU | A | 230 | 31.110 | 71.083 | 41.797 | 1.00 | 25.72 | A |
| ATOM | 1718 | CD1 | LEU | A | 230 | 31.209 | 70.108 | 40.638 | 1.00 | 26.03 | A |
| ATOM | 1719 | CD2 | LEU | A | 230 | 30.507 | 70.401 | 43.012 | 1.00 | 26.48 | A |
| ATOM | 1720 | C | LEU | A | 230 | 34.636 | 70.494 | 41.333 | 1.00 | 19.19 | A |
| ATOM | 1721 | O | LEU | A | 230 | 34.412 | 69.776 | 40.357 | 1.00 | 18.25 | A |
| ATOM | 1722 | N | GLY | A | 231 | 35.760 | 71.189 | 41.489 | 1.00 | 17.43 | A |
| ATOM | 1723 | CA | GLY | A | 231 | 36.808 | 71.184 | 40.483 | 1.00 | 18.17 | A |
| ATOM | 1724 | C | GLY | A | 231 | 37.296 | 69.838 | 39.983 | 1.00 | 17.92 | A |
| ATOM | 1725 | O | GLY | A | 231 | 37.350 | 69.611 | 38.774 | 1.00 | 15.95 | A |
| ATOM | 1726 | N | ASN | A | 232 | 37.655 | 68.944 | 40.898 | 1.00 | 17.85 | A |
| ATOM | 1727 | CA | ASN | A | 232 | 38.158 | 67.632 | 40.508 | 1.00 | 18.27 | A |
| ATOM | 1728 | CB | ASN | A | 232 | 38.634 | 66.880 | 41.754 | 1.00 | 21.28 | A |
| ATOM | 1729 | CG | ASN | A | 232 | 39.138 | 65.484 | 41.432 | 1.00 | 23.57 | A |
| ATOM | 1730 | OD1 | ASN | A | 232 | 40.094 | 65.315 | 40.673 | 1.00 | 24.62 | A |
| ATOM | 1731 | ND2 | ASN | A | 232 | 38.491 | 64.474 | 42.005 | 1.00 | 24.98 | A |
| ATOM | 1732 | C | ASN | A | 232 | 37.137 | 66.841 | 39.699 | 1.00 | 16.30 | A |
| ATOM | 1733 | O | ASN | A | 232 | 37.456 | 66.323 | 38.628 | 1.00 | 15.12 | A |
| ATOM | 1734 | N | ALA | A | 233 | 35.914 | 66.742 | 40.214 | 1.00 | 15.63 | A |
| ATOM | 1735 | CA | ALA | A | 233 | 34.861 | 66.000 | 39.532 | 1.00 | 14.98 | A |
| ATOM | 1736 | CB | ALA | A | 233 | 33.581 | 66.015 | 40.364 | 1.00 | 16.59 | A |
| ATOM | 1737 | C | ALA | A | 233 | 34.586 | 66.559 | 38.140 | 1.00 | 13.06 | A |
| ATOM | 1738 | O | ALA | A | 233 | 34.446 | 65.810 | 37.176 | 1.00 | 13.37 | A |
| ATOM | 1739 | N | VAL | A | 234 | 34.513 | 67.881 | 38.039 | 1.00 | 12.94 | A |
| ATOM | 1740 | CA | VAL | A | 234 | 34.250 | 68.521 | 36.758 | 1.00 | 13.25 | A |
| ATOM | 1741 | CB | VAL | A | 234 | 34.108 | 70.048 | 36.928 | 1.00 | 14.43 | A |
| ATOM | 1742 | CG1 | VAL | A | 234 | 34.200 | 70.742 | 35.577 | 1.00 | 16.13 | A |
| ATOM | 1743 | CG2 | VAL | A | 234 | 32.780 | 70.362 | 37.598 | 1.00 | 16.27 | A |
| ATOM | 1744 | C | VAL | A | 234 | 35.347 | 68.210 | 35.751 | 1.00 | 11.85 | A |
| ATOM | 1745 | O | VAL | A | 234 | 35.064 | 67.942 | 34.582 | 1.00 | 11.28 | A |
| ATOM | 1746 | N | ASN | A | 235 | 36.597 | 68.240 | 36.202 | 1.00 | 12.84 | A |
| ATOM | 1747 | CA | ASN | A | 235 | 37.712 | 67.947 | 35.311 | 1.00 | 12.21 | A |
| ATOM | 1748 | CB | ASN | A | 235 | 39.034 | 68.259 | 36.013 | 1.00 | 13.94 | A |
| ATOM | 1749 | CG | ASN | A | 235 | 40.228 | 68.035 | 35.112 | 1.00 | 14.29 | A |
| ATOM | 1750 | OD1 | ASN | A | 235 | 40.335 | 68.647 | 34.050 | 1.00 | 12.99 | A |
| ATOM | 1751 | ND2 | ASN | A | 235 | 41.129 | 67.146 | 35.521 | 1.00 | 15.45 | A |
| ATOM | 1752 | C | ASN | A | 235 | 37.638 | 66.502 | 34.822 | 1.00 | 12.06 | A |
| ATOM | 1753 | O | ASN | A | 235 | 37.816 | 66.231 | 33.636 | 1.00 | 12.54 | A |
| ATOM | 1754 | N | LEU | A | 236 | 37.383 | 65.572 | 35.737 | 1.00 | 12.33 | A |
| ATOM | 1755 | CA | LEU | A | 236 | 37.289 | 64.170 | 35.356 | 1.00 | 12.19 | A |
| ATOM | 1756 | CB | LEU | A | 236 | 37.142 | 63.304 | 36.610 | 1.00 | 12.94 | A |
| ATOM | 1757 | CG | LEU | A | 236 | 38.330 | 63.274 | 37.571 | 1.00 | 14.02 | A |
| ATOM | 1758 | CD1 | LEU | A | 236 | 38.016 | 62.336 | 38.727 | 1.00 | 16.16 | A |
| ATOM | 1759 | CD2 | LEU | A | 236 | 39.582 | 62.816 | 36.838 | 1.00 | 15.03 | A |
| ATOM | 1760 | C | LEU | A | 236 | 36.144 | 63.990 | 34.369 | 1.00 | 11.11 | A |
| ATOM | 1761 | O | LEU | A | 236 | 36.234 | 63.181 | 33.450 | 1.00 | 11.50 | A |
| ATOM | 1762 | N | ALA | A | 237 | 35.064 | 64.744 | 34.555 | 1.00 | 10.73 | A |
| ATOM | 1763 | CA | ALA | A | 237 | 33.922 | 64.641 | 33.653 | 1.00 | 11.06 | A |
| ATOM | 1764 | CB | ALA | A | 237 | 32.761 | 65.476 | 34.174 | 1.00 | 11.63 | A |
| ATOM | 1765 | C | ALA | A | 237 | 34.314 | 65.092 | 32.246 | 1.00 | 8.82 | A |
| ATOM | 1766 | O | ALA | A | 237 | 33.938 | 64.466 | 31.254 | 1.00 | 10.10 | A |
| ATOM | 1767 | N | VAL | A | 238 | 35.069 | 66.181 | 32.155 | 1.00 | 9.53 | A |
| ATOM | 1768 | CA | VAL | A | 238 | 35.514 | 66.669 | 30.855 | 1.00 | 9.58 | A |
| ATOM | 1769 | CB | VAL | A | 238 | 36.331 | 67.979 | 30.992 | 1.00 | 9.67 | A |
| ATOM | 1770 | CG1 | VAL | A | 238 | 37.014 | 68.306 | 29.673 | 1.00 | 11.56 | A |
| ATOM | 1771 | CG2 | VAL | A | 238 | 35.412 | 69.124 | 31.410 | 1.00 | 10.33 | A |
| ATOM | 1772 | C | VAL | A | 238 | 36.375 | 65.602 | 30.166 | 1.00 | 9.59 | A |
| ATOM | 1773 | O | VAL | A | 238 | 36.224 | 65.344 | 28.974 | 1.00 | 10.14 | A |
| ATOM | 1774 | N | LEU | A | 239 | 37.268 | 64.975 | 30.924 | 1.00 | 10.44 | A |
| ATOM | 1775 | CA | LEU | A | 239 | 38.131 | 63.946 | 30.363 | 1.00 | 10.94 | A |
| ATOM | 1776 | CB | LEU | A | 239 | 39.161 | 63.531 | 31.417 | 1.00 | 12.73 | A |
| ATOM | 1777 | CG | LEU | A | 239 | 40.180 | 64.613 | 31.790 | 1.00 | 16.19 | A |
| ATOM | 1778 | CD1 | LEU | A | 239 | 41.084 | 64.109 | 32.887 | 1.00 | 18.37 | A |
| ATOM | 1779 | CD2 | LEU | A | 239 | 40.998 | 64.978 | 30.587 | 1.00 | 19.19 | A |
| ATOM | 1780 | C | LEU | A | 239 | 37.300 | 62.778 | 29.842 | 1.00 | 10.02 | A |
| ATOM | 1781 | O | LEU | A | 239 | 37.566 | 62.255 | 28.760 | 1.00 | 10.81 | A |
| ATOM | 1782 | N | LYS | A | 240 | 36.292 | 62.370 | 30.609 | 1.00 | 10.83 | A |
| ATOM | 1783 | CA | LYS | A | 240 | 35.445 | 61.261 | 30.196 | 1.00 | 11.07 | A |
| ATOM | 1784 | CB | LYS | A | 240 | 34.516 | 60.869 | 31.349 | 1.00 | 13.99 | A |
| ATOM | 1785 | CG | LYS | A | 240 | 33.663 | 59.642 | 31.052 | 1.00 | 19.14 | A |
| ATOM | 1786 | CD | LYS | A | 240 | 32.876 | 59.203 | 32.278 | 1.00 | 25.66 | A |
| ATOM | 1787 | CE | LYS | A | 240 | 32.113 | 57.914 | 32.011 | 1.00 | 29.89 | A |
| ATOM | 1788 | NZ | LYS | A | 240 | 31.381 | 57.433 | 33.218 | 1.00 | 33.32 | A |
| ATOM | 1789 | C | LYS | A | 240 | 34.699 | 61.620 | 28.919 | 1.00 | 10.37 | A |
| ATOM | 1790 | O | LYS | A | 240 | 34.679 | 60.841 | 27.972 | 1.00 | 11.74 | A |
| ATOM | 1791 | N | LEU | A | 241 | 34.082 | 62.800 | 28.889 | 1.00 | 10.03 | A |
| ATOM | 1792 | CA | LEU | A | 241 | 33.344 | 63.226 | 27.702 | 1.00 | 9.53 | A |
| ATOM | 1793 | CB | LEU | A | 241 | 32.672 | 64.576 | 27.979 | 1.00 | 10.84 | A |
| ATOM | 1794 | CG | LEU | A | 241 | 31.605 | 64.579 | 29.081 | 1.00 | 11.02 | A |
| ATOM | 1795 | CD1 | LEU | A | 241 | 31.140 | 66.000 | 29.344 | 1.00 | 12.25 | A |
| ATOM | 1796 | CD2 | LEU | A | 241 | 30.437 | 63.698 | 28.669 | 1.00 | 12.82 | A |
| ATOM | 1797 | C | LEU | A | 241 | 34.275 | 63.255 | 26.495 | 1.00 | 9.51 | A |
| ATOM | 1798 | O | LEU | A | 241 | 33.883 | 62.890 | 25.389 | 1.00 | 10.36 | A |
| ATOM | 1799 | N | SER | A | 242 | 35.507 | 63.705 | 26.714 | 1.00 | 9.60 | A |
| ATOM | 1800 | CA | SER | A | 242 | 36.500 | 63.760 | 25.653 | 1.00 | 10.00 | A |
| ATOM | 1801 | CB | SER | A | 242 | 37.769 | 64.418 | 26.191 | 1.00 | 10.04 | A |
| ATOM | 1802 | OG | SER | A | 242 | 38.843 | 64.252 | 25.274 | 1.00 | 11.08 | A |
| ATOM | 1803 | C | SER | A | 242 | 36.768 | 62.363 | 25.111 | 1.00 | 9.77 | A |
| ATOM | 1804 | O | SER | A | 242 | 36.660 | 62.120 | 23.911 | 1.00 | 10.50 | A |
| ATOM | 1805 | N | GLU | A | 243 | 37.122 | 61.455 | 26.017 | 1.00 | 10.78 | A |
| ATOM | 1806 | CA | GLU | A | 243 | 37.441 | 60.082 | 25.654 | 1.00 | 12.16 | A |
| ATOM | 1807 | CB | GLU | A | 243 | 38.086 | 59.396 | 26.865 | 1.00 | 12.47 | A |
| ATOM | 1808 | CG | GLU | A | 243 | 39.500 | 59.917 | 27.109 | 1.00 | 13.04 | A |
| ATOM | 1809 | CD | GLU | A | 243 | 40.207 | 59.282 | 28.288 | 1.00 | 13.31 | A |
| ATOM | 1810 | OE1 | GLU | A | 243 | 39.712 | 58.267 | 28.825 | 1.00 | 14.76 | A |
| ATOM | 1811 | OE2 | GLU | A | 243 | 41.278 | 59.801 | 28.671 | 1.00 | 13.41 | A |
| ATOM | 1812 | C | GLU | A | 243 | 36.284 | 59.307 | 25.043 | 1.00 | 11.98 | A |
| ATOM | 1813 | O | GLU | A | 243 | 36.511 | 58.373 | 24.275 | 1.00 | 13.80 | A |
| ATOM | 1814 | N | GLN | A | 244 | 35.053 | 59.692 | 25.368 | 1.00 | 12.01 | A |
| ATOM | 1815 | CA | GLN | A | 244 | 33.880 | 59.015 | 24.816 | 1.00 | 12.65 | A |
| ATOM | 1816 | CB | GLN | A | 244 | 32.699 | 59.154 | 25.782 | 1.00 | 15.70 | A |
| ATOM | 1817 | CG | GLN | A | 244 | 32.901 | 58.503 | 27.137 | 1.00 | 21.85 | A |
| ATOM | 1818 | CD | GLN | A | 244 | 31.694 | 58.672 | 28.041 | 1.00 | 26.51 | A |
| ATOM | 1819 | OE1 | GLN | A | 244 | 31.224 | 59.788 | 28.270 | 1.00 | 30.47 | A |
| ATOM | 1820 | NE2 | GLN | A | 244 | 31.188 | 57.563 | 28.565 | 1.00 | 29.00 | A |
| ATOM | 1821 | C | GLN | A | 244 | 33.542 | 59.545 | 23.431 | 1.00 | 12.25 | A |
| ATOM | 1822 | O | GLN | A | 244 | 32.700 | 58.978 | 22.740 | 1.00 | 14.55 | A |
| ATOM | 1823 | N | GLY | A | 245 | 34.193 | 60.634 | 23.035 | 1.00 | 12.61 | A |
| ATOM | 1824 | CA | GLY | A | 245 | 33.934 | 61.229 | 21.734 | 1.00 | 12.75 | A |
| ATOM | 1825 | C | GLY | A | 245 | 32.721 | 62.146 | 21.742 | 1.00 | 11.07 | A |
| ATOM | 1826 | O | GLY | A | 245 | 32.313 | 62.667 | 20.702 | 1.00 | 10.93 | A |
| ATOM | 1827 | N | LEU | A | 246 | 32.155 | 62.359 | 22.925 | 1.00 | 12.32 | A |
| ATOM | 1828 | CA | LEU | A | 246 | 30.971 | 63.196 | 23.075 | 1.00 | 12.25 | A |
| ATOM | 1829 | CB | LEU | A | 246 | 30.386 | 62.969 | 24.473 | 1.00 | 13.71 | A |
| ATOM | 1830 | CG | LEU | A | 246 | 29.713 | 61.612 | 24.706 | 1.00 | 16.08 | A |
| ATOM | 1831 | CD1 | LEU | A | 246 | 29.284 | 61.490 | 26.158 | 1.00 | 20.32 | A |
| ATOM | 1832 | CD2 | LEU | A | 246 | 28.511 | 61.472 | 23.782 | 1.00 | 18.93 | A |
| ATOM | 1833 | C | LEU | A | 246 | 31.229 | 64.665 | 22.754 | 1.00 | 11.72 | A |
| ATOM | 1834 | O | LEU | A | 246 | 30.354 | 65.346 | 22.224 | 1.00 | 11.90 | A |
| ATOM | 1835 | N | LEU | A | 247 | 32.420 | 65.158 | 23.076 | 1.00 | 10.24 | A |
| ATOM | 1836 | CA | LEU | A | 247 | 32.741 | 66.548 | 22.784 | 1.00 | 10.90 | A |
| ATOM | 1837 | CB | LEU | A | 247 | 34.016 | 66.951 | 23.532 | 1.00 | 12.45 | A |
| ATOM | 1838 | CG | LEU | A | 247 | 33.937 | 66.865 | 25.059 | 1.00 | 10.89 | A |
| ATOM | 1839 | CD1 | LEU | A | 247 | 35.259 | 67.333 | 25.661 | 1.00 | 12.02 | A |
| ATOM | 1840 | CD2 | LEU | A | 247 | 32.785 | 67.714 | 25.578 | 1.00 | 13.73 | A |
| ATOM | 1841 | C | LEU | A | 247 | 32.830 | 66.729 | 21.273 | 1.00 | 10.25 | A |
| ATOM | 1842 | O | LEU | A | 247 | 32.450 | 67.775 | 20.747 | 1.00 | 11.03 | A |
| ATOM | 1843 | N | ASP | A | 248 | 33.334 | 65.716 | 20.568 | 1.00 | 10.83 | A |
| ATOM | 1844 | CA | ASP | A | 248 | 33.431 | 65.796 | 19.113 | 1.00 | 12.99 | A |
| ATOM | 1845 | CB | ASP | A | 248 | 34.254 | 64.622 | 18.565 | 1.00 | 12.21 | A |
| ATOM | 1846 | CG | ASP | A | 248 | 35.707 | 64.672 | 18.983 | 1.00 | 13.84 | A |
| ATOM | 1847 | OD1 | ASP | A | 248 | 36.291 | 65.777 | 18.999 | 1.00 | 15.46 | A |
| ATOM | 1848 | OD2 | ASP | A | 248 | 36.271 | 63.597 | 19.278 | 1.00 | 17.21 | A |
| ATOM | 1849 | C | ASP | A | 248 | 32.024 | 65.841 | 18.526 | 1.00 | 12.51 | A |
| ATOM | 1850 | O | ASP | A | 248 | 31.750 | 66.614 | 17.610 | 1.00 | 13.15 | A |
| ATOM | 1851 | N | LYS | A | 249 | 31.136 | 65.005 | 19.058 | 1.00 | 12.00 | A |
| ATOM | 1852 | CA | LYS | A | 249 | 29.757 | 64.957 | 18.589 | 1.00 | 13.30 | A |
| ATOM | 1853 | CB | LYS | A | 249 | 29.030 | 63.802 | 19.287 | 1.00 | 15.41 | A |
| ATOM | 1854 | CG | LYS | A | 249 | 27.582 | 63.635 | 18.860 | 1.00 | 22.53 | A |
| ATOM | 1855 | CD | LYS | A | 249 | 26.910 | 62.498 | 19.611 | 1.00 | 25.97 | A |
| ATOM | 1856 | CE | LYS | A | 249 | 25.468 | 62.317 | 19.163 | 1.00 | 28.10 | A |
| ATOM | 1857 | NZ | LYS | A | 249 | 25.374 | 62.030 | 17.702 | 1.00 | 29.71 | A |
| ATOM | 1858 | C | LYS | A | 249 | 29.073 | 66.304 | 18.812 | 1.00 | 12.10 | A |
| ATOM | 1859 | O | LYS | A | 249 | 28.326 | 66.779 | 17.956 | 1.00 | 12.22 | A |
| ATOM | 1860 | N | LEU | A | 250 | 29.339 | 66.912 | 19.963 | 1.00 | 11.38 | A |
| ATOM | 1861 | CA | LEU | A | 250 | 28.747 | 68.192 | 20.320 | 1.00 | 13.81 | A |
| ATOM | 1862 | CB | LEU | A | 250 | 29.052 | 68.476 | 21.791 | 1.00 | 15.34 | A |
| ATOM | 1863 | CG | LEU | A | 250 | 28.126 | 69.460 | 22.502 | 1.00 | 19.52 | A |
| ATOM | 1864 | CD1 | LEU | A | 250 | 26.693 | 68.947 | 22.444 | 1.00 | 19.78 | A |
| ATOM | 1865 | CD2 | LEU | A | 250 | 28.575 | 69.624 | 23.642 | 1.00 | 18.91 | A |
| ATOM | 1866 | C | LEU | A | 250 | 29.207 | 69.299 | 19.380 | 1.00 | 11.31 | A |
| ATOM | 1867 | O | LEU | A | 250 | 28.409 | 70.147 | 18.983 | 1.00 | 11.72 | A |
| ATOM | 1868 | N | LYS | A | 251 | 30.489 | 69.303 | 19.015 | 1.00 | 11.59 | A |
| ATOM | 1869 | CA | LYS | A | 251 | 30.966 | 70.338 | 18.104 | 1.00 | 12.31 | A |
| ATOM | 1870 | CB | LYS | A | 251 | 32.499 | 70.357 | 18.036 | 1.00 | 13.49 | A |
| ATOM | 1871 | CG | LYS | A | 251 | 32.995 | 71.489 | 17.133 | 1.00 | 16.13 | A |
| ATOM | 1872 | CD | LYS | A | 251 | 34.501 | 71.665 | 17.107 | 1.00 | 16.95 | A |
| ATOM | 1873 | CE | LYS | A | 251 | 34.851 | 72.951 | 16.360 | 1.00 | 18.24 | A |
| ATOM | 1874 | NZ | LYS | A | 251 | 36.310 | 73.139 | 16.173 | 1.00 | 21.00 | A |
| ATOM | 1875 | C | LYS | A | 251 | 30.332 | 70.150 | 16.730 | 1.00 | 11.48 | A |
| ATOM | 1876 | O | LYS | A | 251 | 29.900 | 71.116 | 16.105 | 1.00 | 11.25 | A |
| ATOM | 1877 | N | ASN | A | 252 | 30.284 | 68.909 | 16.252 | 1.00 | 11.86 | A |
| ATOM | 1878 | CA | ASN | A | 252 | 29.687 | 68.649 | 14.947 | 1.00 | 13.33 | A |
| ATOM | 1879 | CB | ASN | A | 252 | 29.837 | 67.168 | 14.586 | 1.00 | 16.75 | A |
| ATOM | 1880 | CG | ASN | A | 252 | 31.266 | 66.787 | 14.287 | 1.00 | 20.29 | A |
| ATOM | 1881 | OD1 | ASN | A | 252 | 32.017 | 67.571 | 13.709 | 1.00 | 23.39 | A |
| ATOM | 1882 | ND2 | ASN | A | 252 | 31.648 | 65.572 | 14.663 | 1.00 | 22.44 | A |
| ATOM | 1883 | C | ASN | A | 252 | 28.240 | 69.106 | 14.939 | 1.00 | 13.81 | A |
| ATOM | 1884 | O | ASN | A | 252 | 27.777 | 69.704 | 13.975 | 1.00 | 13.29 | A |
| ATOM | 1885 | N | LYS | A | 253 | 27.529 | 68.819 | 16.023 | 1.00 | 12.19 | A |
| ATOM | 1886 | CA | LYS | A | 253 | 26.127 | 69.199 | 16.134 | 1.00 | 13.41 | A |
| ATOM | 1887 | CB | LYS | A | 253 | 25.583 | 68.695 | 17.475 | 1.00 | 14.37 | A |
| ATOM | 1888 | CG | LYS | A | 253 | 24.183 | 69.195 | 17.808 | 1.00 | 13.86 | A |
| ATOM | 1889 | CD | LYS | A | 253 | 23.730 | 68.716 | 19.185 | 1.00 | 14.28 | A |
| ATOM | 1890 | CE | LYS | A | 253 | 22.346 | 69.242 | 19.527 | 1.00 | 16.70 | A |
| ATOM | 1891 | NZ | LYS | A | 253 | 21.928 | 68.869 | 20.909 | 1.00 | 20.13 | A |
| ATOM | 1892 | C | LYS | A | 253 | 25.905 | 70.696 | 15.953 | 1.00 | 13.48 | A |
| ATOM | 1893 | O | LYS | A | 253 | 25.082 | 71.113 | 15.139 | 1.00 | 14.39 | A |
| ATOM | 1894 | N | TRP | A | 254 | 26.657 | 71.500 | 16.699 | 1.00 | 11.82 | A |
| ATOM | 1895 | CA | TRP | A | 254 | 26.484 | 72.945 | 16.660 | 1.00 | 12.08 | A |
| ATOM | 1896 | CB | TRP | A | 254 | 26.754 | 73.493 | 18.068 | 1.00 | 11.54 | A |
| ATOM | 1897 | CG | TRP | A | 254 | 25.733 | 73.030 | 19.059 | 1.00 | 10.58 | A |
| ATOM | 1898 | CD2 | TRP | A | 254 | 24.393 | 73.514 | 19.185 | 1.00 | 9.75 | A |
| ATOM | 1899 | CE2 | TRP | A | 254 | 23.766 | 72.760 | 20.201 | 1.00 | 10.91 | A |
| ATOM | 1900 | CE3 | TRP | A | 254 | 23.658 | 74.512 | 18.531 | 1.00 | 10.72 | A |
| ATOM | 1901 | CD1 | TRP | A | 254 | 25.867 | 72.028 | 19.981 | 1.00 | 10.86 | A |
| ATOM | 1902 | NE1 | TRP | A | 254 | 24.686 | 71.859 | 20.672 | 1.00 | 10.73 | A |
| ATOM | 1903 | CZ2 | TRP | A | 254 | 22.438 | 72.974 | 20.580 | 1.00 | 11.83 | A |
| ATOM | 1904 | CZ3 | TRP | A | 254 | 22.340 | 74.725 | 18.907 | 1.00 | 12.00 | A |
| ATOM | 1905 | CH2 | TRP | A | 254 | 21.745 | 73.959 | 19.923 | 1.00 | 11.75 | A |
| ATOM | 1906 | C | TRP | A | 254 | 27.238 | 73.701 | 15.572 | 1.00 | 11.48 | A |
| ATOM | 1907 | O | TRP | A | 254 | 26.907 | 74.853 | 15.287 | 1.00 | 12.01 | A |
| ATOM | 1908 | N | TRP | A | 255 | 28.226 | 73.062 | 14.957 | 1.00 | 11.38 | A |
| ATOM | 1909 | CA | TRP | A | 255 | 29.018 | 73.707 | 13.912 | 1.00 | 13.24 | A |
| ATOM | 1910 | CB | TRP | A | 255 | 30.506 | 73.489 | 14.201 | 1.00 | 13.18 | A |
| ATOM | 1911 | CG | TRP | A | 255 | 31.156 | 74.556 | 14.996 | 1.00 | 11.40 | A |
| ATOM | 1912 | CD2 | TRP | A | 255 | 31.010 | 74.782 | 16.398 | 1.00 | 10.32 | A |
| ATOM | 1913 | CE2 | TRP | A | 255 | 31.820 | 75.890 | 16.727 | 1.00 | 10.52 | A |
| ATOM | 1914 | CE3 | TRP | A | 255 | 30.271 | 74.154 | 17.409 | 1.00 | 11.07 | A |
| ATOM | 1915 | CD1 | TRP | A | 255 | 32.022 | 75.507 | 14.539 | 1.00 | 11.83 | A |
| ATOM | 1916 | NE1 | TRP | A | 255 | 32.426 | 76.312 | 15.574 | 1.00 | 10.82 | A |
| ATOM | 1917 | CZ2 | TRP | A | 255 | 31.914 | 76.387 | 18.029 | 1.00 | 9.89 | A |
| ATOM | 1918 | CZ3 | TRP | A | 255 | 30.363 | 74.647 | 18.706 | 1.00 | 10.99 | A |
| ATOM | 1919 | CH2 | TRP | A | 255 | 31.181 | 75.756 | 19.005 | 1.00 | 10.54 | A |
| ATOM | 1920 | C | TRP | A | 255 | 28.693 | 73.270 | 12.494 | 1.00 | 14.87 | A |
| ATOM | 1921 | O | TRP | A | 255 | 28.460 | 74.104 | 11.622 | 1.00 | 15.77 | A |
| ATOM | 1922 | N | TYR | A | 256 | 28.679 | 71.960 | 12.269 | 1.00 | 17.01 | A |
| ATOM | 1923 | CA | TYR | A | 256 | 28.487 | 71.426 | 10.927 | 1.00 | 18.48 | A |
| ATOM | 1924 | CB | TYR | A | 256 | 29.692 | 70.530 | 10.639 | 1.00 | 17.86 | A |
| ATOM | 1925 | CG | TYR | A | 256 | 31.002 | 71.189 | 11.015 | 1.00 | 17.74 | A |
| ATOM | 1926 | CD1 | TYR | A | 256 | 31.445 | 72.332 | 10.350 | 1.00 | 19.92 | A |
| ATOM | 1927 | CE1 | TYR | A | 256 | 32.626 | 72.971 | 10.725 | 1.00 | 19.08 | A |
| ATOM | 1928 | CD2 | TYR | A | 256 | 31.776 | 70.697 | 12.065 | 1.00 | 18.11 | A |
| ATOM | 1929 | CE2 | TYR | A | 256 | 32.959 | 71.329 | 12.447 | 1.00 | 20.70 | A |
| ATOM | 1930 | CZ | TYR | A | 256 | 33.375 | 72.466 | 11.773 | 1.00 | 19.95 | A |
| ATOM | 1931 | OH | TYR | A | 256 | 34.536 | 73.104 | 12.151 | 1.00 | 23.67 | A |
| ATOM | 1932 | C | TYR | A | 256 | 27.158 | 70.754 | 10.605 | 1.00 | 21.44 | A |
| ATOM | 1933 | O | TYR | A | 256 | 26.645 | 70.903 | 9.495 | 1.00 | 22.92 | A |
| ATOM | 1934 | N | ASP | A | 257 | 26.608 | 70.006 | 11.554 | 1.00 | 23.80 | A |
| ATOM | 1935 | CA | ASP | A | 257 | 25.342 | 69.312 | 11.337 | 1.00 | 27.48 | A |
| ATOM | 1936 | CB | ASP | A | 257 | 24.902 | 68.668 | 12.655 | 1.00 | 27.47 | A |
| ATOM | 1937 | CG | ASP | A | 257 | 25.709 | 67.427 | 12.997 | 1.00 | 28.50 | A |
| ATOM | 1938 | OD1 | ASP | A | 257 | 26.875 | 67.328 | 12.561 | 1.00 | 29.59 | A |
| ATOM | 1939 | OD2 | ASP | A | 257 | 25.179 | 66.552 | 13.713 | 1.00 | 28.83 | A |
| ATOM | 1940 | C | ASP | A | 257 | 24.292 | 70.241 | 10.733 | 1.00 | 29.42 | A |
| ATOM | 1941 | O | ASP | A | 257 | 23.423 | 69.802 | 9.978 | 1.00 | 31.16 | A |
| ATOM | 1942 | N | GLY | A | 258 | 24.380 | 71.525 | 11.062 | 1.00 | 31.16 | A |
| ATOM | 1943 | CA | GLY | A | 258 | 23.429 | 72.487 | 10.536 | 1.00 | 32.33 | A |
| ATOM | 1944 | C | GLY | A | 258 | 24.059 | 73.441 | 9.539 | 1.00 | 33.05 | A |
| ATOM | 1945 | O | GLY | A | 258 | 23.484 | 74.482 | 9.220 | 1.00 | 34.08 | A |
| ATOM | 1946 | N | GLY | A | 259 | 25.242 | 73.082 | 9.048 | 1.00 | 33.37 | A |
| ATOM | 1947 | CA | GLY | A | 259 | 25.941 | 73.919 | 8.087 | 1.00 | 32.81 | A |
| ATOM | 1948 | C | GLY | A | 259 | 25.036 | 74.455 | 6.995 | 1.00 | 33.01 | A |
| ATOM | 1949 | O | GLY | A | 259 | 24.345 | 73.692 | 6.317 | 1.00 | 32.94 | A |
| ATOM | 1950 | N | GLU | A | 260 | 25.044 | 75.773 | 6.823 | 1.00 | 32.45 | A |
| ATOM | 1951 | CA | GLU | A | 260 | 24.217 | 76.427 | 5.817 | 1.00 | 31.91 | A |
| ATOM | 1952 | CB | GLU | A | 260 | 23.638 | 77.713 | 6.414 | 1.00 | 32.09 | A |
| ATOM | 1953 | CG | GLU | A | 260 | 22.761 | 77.502 | 7.635 | 1.00 | 32.14 | A |
| ATOM | 1954 | CD | GLU | A | 260 | 22.348 | 78.810 | 8.285 | 1.00 | 32.27 | A |
| ATOM | 1955 | OE1 | GLU | A | 260 | 21.521 | 78.777 | 9.223 | 1.00 | 32.26 | A |
| ATOM | 1956 | OE2 | GLU | A | 260 | 22.853 | 79.871 | 7.862 | 1.00 | 33.19 | A |
| ATOM | 1957 | C | GLU | A | 260 | 25.002 | 76.681 | 4.537 | 1.00 | 31.22 | A |
| ATOM | 1958 | O | GLU | A | 260 | 24.459 | 77.212 | 3.569 | 1.00 | 32.04 | A |
| ATOM | 1959 | N | CYS | A | 261 | 26.275 | 76.303 | 4.532 | 1.00 | 30.64 | A |
| ATOM | 1960 | CA | CYS | A | 261 | 27.120 | 76.519 | 3.365 | 1.00 | 30.66 | A |
| ATOM | 1961 | CB | CYS | A | 261 | 28.266 | 77.457 | 3.761 | 1.00 | 27.88 | A |
| ATOM | 1962 | SG | CYS | A | 261 | 27.700 | 78.992 | 4.563 | 1.00 | 22.41 | A |
| ATOM | 1963 | C | CYS | A | 261 | 27.595 | 75.199 | 2.768 | 1.00 | 32.66 | A |
| ATOM | 1964 | OT1 | CYS | A | 261 | 28.824 | 74.998 | 2.684 | 1.00 | 34.27 | A |
| ATOM | 1965 | OT2 | CYS | A | 261 | 26.729 | 74.384 | 2.386 | 1.00 | 33.85 | A |
| ATOM | 1966 | N | KAI | D | 999 | 52.259 | 58.965 | 24.382 | 1.00 | 8.63 | D |
| ATOM | 1967 | CD | KAI | D | 999 | 53.638 | 58.501 | 23.988 | 1.00 | 10.80 | D |
| ATOM | 1968 | CD1 | KAI | D | 999 | 55.552 | 56.535 | 23.071 | 1.00 | 12.20 | D |
| ATOM | 1969 | CD2 | KAI | D | 999 | 54.558 | 54.578 | 24.118 | 1.00 | 11.04 | D |
| ATOM | 1970 | CA | KAI | D | 999 | 51.419 | 57.830 | 24.582 | 1.00 | 9.63 | D |
| ATOM | 1971 | CB | KAI | D | 999 | 52.093 | 56.616 | 23.960 | 1.00 | 10.19 | D |
| ATOM | 1972 | CB1 | KAI | D | 999 | 51.980 | 56.522 | 22.435 | 1.00 | 10.60 | D |
| ATOM | 1973 | CG | KAI | D | 999 | 53.489 | 56.931 | 24.227 | 1.00 | 10.61 | D |
| ATOM | 1974 | CG1 | KAI | D | 999 | 50.770 | 55.761 | 21.918 | 1.00 | 10.59 | D |
| ATOM | 1975 | CG2 | KAI | D | 999 | 54.632 | 56.001 | 23.768 | 1.00 | 10.26 | D |
| ATOM | 1976 | C | KAI | D | 999 | 51.075 | 57.648 | 26.071 | 1.00 | 9.43 | D |
| ATOM | 1977 | O | KAI | D | 999 | 51.316 | 58.626 | 26.818 | 1.00 | 8.26 | D |
| ATOM | 1978 | OD1 | KAI | D | 999 | 50.585 | 55.802 | 20.683 | 1.00 | 10.86 | D |
| ATOM | 1979 | OD2 | KAI | D | 999 | 50.054 | 55.152 | 22.744 | 1.00 | 12.88 | D |
| ATOM | 1980 | OT | KAI | D | 999 | 50.572 | 56.547 | 26.391 | 1.00 | 10.08 | D |
| ATOM | 1981 | C | GLY | B | 4 | 66.467 | 69.848 | 45.552 | 1.00 | 25.87 | B |
| ATOM | 1982 | O | GLY | B | 4 | 65.518 | 70.397 | 44.992 | 1.00 | 26.65 | B |
| ATOM | 1983 | N | GLY | B | 4 | 65.769 | 70.992 | 47.668 | 1.00 | 28.44 | B |
| ATOM | 1984 | CA | GLY | B | 4 | 66.664 | 69.975 | 47.051 | 1.00 | 26.72 | B |
| ATOM | 1985 | N | THR | B | 5 | 67.368 | 69.121 | 44.902 | 1.00 | 23.55 | B |
| ATOM | 1986 | CA | THR | B | 5 | 67.290 | 68.918 | 43.462 | 1.00 | 20.59 | B |
| ATOM | 1987 | CB | THR | B | 5 | 68.519 | 68.144 | 42.948 | 1.00 | 20.71 | B |
| ATOM | 1988 | OG1 | THR | B | 5 | 69.704 | 68.909 | 43.207 | 1.00 | 22.39 | B |
| ATOM | 1989 | CG2 | THR | B | 5 | 68.403 | 67.883 | 41.451 | 1.00 | 19.82 | B |
| ATOM | 1990 | C | THR | B | 5 | 66.025 | 68.147 | 43.097 | 1.00 | 18.19 | B |
| ATOM | 1991 | O | THR | B | 5 | 65.721 | 67.109 | 43.684 | 1.00 | 18.92 | B |
| ATOM | 1992 | N | VAL | B | 6 | 65.289 | 68.671 | 42.125 | 1.00 | 15.33 | B |
| ATOM | 1993 | CA | VAL | B | 6 | 64.056 | 68.045 | 41.669 | 1.00 | 14.60 | B |
| ATOM | 1994 | CB | VAL | B | 6 | 63.195 | 69.063 | 40.889 | 1.00 | 12.72 | B |
| ATOM | 1995 | CG1 | VAL | B | 6 | 61.990 | 68.376 | 40.267 | 1.00 | 13.98 | B |
| ATOM | 1996 | CG2 | VAL | B | 6 | 62.754 | 70.184 | 41.826 | 1.00 | 13.51 | B |
| ATOM | 1997 | C | VAL | B | 6 | 64.373 | 66.851 | 40.778 | 1.00 | 13.37 | B |
| ATOM | 1998 | O | VAL | B | 6 | 65.198 | 66.948 | 39.872 | 1.00 | 14.05 | B |
| ATOM | 1999 | N | VAL | B | 7 | 63.732 | 65.719 | 41.049 | 1.00 | 11.84 | B |
| ATOM | 2000 | CA | VAL | B | 7 | 63.952 | 64.526 | 40.248 | 1.00 | 11.72 | B |
| ATOM | 2001 | CB | VAL | B | 7 | 63.857 | 63.247 | 41.096 | 1.00 | 12.00 | B |
| ATOM | 2002 | CG1 | VAL | B | 7 | 63.972 | 62.018 | 40.198 | 1.00 | 13.16 | B |
| ATOM | 2003 | CG2 | VAL | B | 7 | 64.962 | 63.244 | 42.138 | 1.00 | 13.65 | B |
| ATOM | 2004 | C | VAL | B | 7 | 62.924 | 64.461 | 39.131 | 1.00 | 10.82 | B |
| ATOM | 2005 | O | VAL | B | 7 | 61.726 | 64.290 | 39.373 | 1.00 | 11.42 | B |
| ATOM | 2006 | N | VAL | B | 8 | 63.404 | 64.612 | 37.904 | 1.00 | 11.28 | B |
| ATOM | 2007 | CA | VAL | B | 8 | 62.545 | 64.571 | 36.737 | 1.00 | 11.25 | B |
| ATOM | 2008 | CB | VAL | B | 8 | 62.989 | 65.613 | 35.679 | 1.00 | 11.15 | B |
| ATOM | 2009 | CG1 | VAL | B | 8 | 62.102 | 65.517 | 34.436 | 1.00 | 12.30 | B |
| ATOM | 2010 | CG2 | VAL | B | 8 | 62.937 | 67.010 | 36.272 | 1.00 | 10.83 | B |
| ATOM | 2011 | C | VAL | B | 8 | 62.634 | 63.198 | 36.106 | 1.00 | 11.06 | B |
| ATOM | 2012 | O | VAL | B | 8 | 63.735 | 62.692 | 35.870 | 1.00 | 11.58 | B |
| ATOM | 2013 | N | THR | B | 9 | 61.489 | 62.570 | 35.871 | 1.00 | 9.47 | B |
| ATOM | 2014 | CA | THR | B | 9 | 61.508 | 61.287 | 35.196 | 1.00 | 9.39 | B |
| ATOM | 2015 | CB | THR | B | 9 | 60.576 | 60.227 | 35.847 | 1.00 | 10.31 | B |
| ATOM | 2016 | OG1 | THR | B | 9 | 60.678 | 59.000 | 35.106 | 1.00 | 11.00 | B |
| ATOM | 2017 | CG2 | THR | B | 9 | 59.121 | 60.683 | 35.852 | 1.00 | 11.65 | B |
| ATOM | 2018 | C | THR | B | 9 | 61.059 | 61.550 | 33.768 | 1.00 | 9.23 | B |
| ATOM | 2019 | O | THR | B | 9 | 60.139 | 62.340 | 33.518 | 1.00 | 9.76 | B |
| ATOM | 2020 | N | THR | B | 10 | 61.747 | 60.921 | 32.826 | 1.00 | 8.57 | B |
| ATOM | 2021 | CA | THR | B | 10 | 61.413 | 61.075 | 31.427 | 1.00 | 8.66 | B |
| ATOM | 2022 | CB | THR | B | 10 | 62.136 | 62.304 | 30.801 | 1.00 | 9.52 | B |
| ATOM | 2023 | OG1 | THR | B | 10 | 61.581 | 62.579 | 29.510 | 1.00 | 9.17 | B |
| ATOM | 2024 | CG2 | THR | B | 10 | 63.632 | 62.070 | 30.681 | 1.00 | 9.62 | B |
| ATOM | 2025 | C | THR | B | 10 | 61.766 | 59.771 | 30.729 | 1.00 | 9.19 | B |
| ATOM | 2026 | O | THR | B | 10 | 62.116 | 58.793 | 31.388 | 1.00 | 8.78 | B |
| ATOM | 2027 | N | ILE | B | 11 | 61.676 | 59.746 | 29.408 | 1.00 | 8.42 | B |
| ATOM | 2028 | CA | ILE | B | 11 | 61.925 | 58.516 | 28.674 | 1.00 | 9.57 | B |
| ATOM | 2029 | CB | ILE | B | 11 | 60.584 | 57.731 | 28.534 | 1.00 | 10.19 | B |
| ATOM | 2030 | CG2 | ILE | B | 11 | 59.605 | 58.521 | 27.674 | 1.00 | 10.71 | B |
| ATOM | 2031 | CG1 | ILE | B | 11 | 60.817 | 56.335 | 27.958 | 1.00 | 9.87 | B |
| ATOM | 2032 | CD1 | ILE | B | 11 | 59.587 | 55.440 | 28.079 | 1.00 | 12.32 | B |
| ATOM | 2033 | C | ILE | B | 11 | 62.507 | 58.851 | 27.310 | 1.00 | 10.25 | B |
| ATOM | 2034 | O | ILE | B | 11 | 62.250 | 59.928 | 26.768 | 1.00 | 10.08 | B |
| ATOM | 2035 | N | LEU | B | 12 | 63.306 | 57.937 | 26.765 | 1.00 | 10.50 | B |
| ATOM | 2036 | CA | LEU | B | 12 | 63.905 | 58.152 | 25.459 | 1.00 | 12.34 | B |
| ATOM | 2037 | CB | LEU | B | 12 | 65.150 | 57.266 | 25.313 | 1.00 | 12.17 | B |
| ATOM | 2038 | CG | LEU | B | 12 | 66.280 | 57.520 | 26.317 | 1.00 | 12.21 | B |
| ATOM | 2039 | CD1 | LEU | B | 12 | 67.424 | 56.543 | 26.062 | 1.00 | 13.28 | B |
| ATOM | 2040 | CD2 | LEU | B | 12 | 66.766 | 58.958 | 26.204 | 1.00 | 15.41 | B |
| ATOM | 2041 | C | LEU | B | 12 | 62.850 | 57.900 | 24.391 | 1.00 | 12.73 | B |
| ATOM | 2042 | O | LEU | B | 12 | 62.301 | 56.802 | 24.287 | 1.00 | 15.53 | B |
| ATOM | 2043 | N | GLU | B | 13 | 62.551 | 58.940 | 23.621 | 1.00 | 11.26 | B |
| ATOM | 2044 | CA | GLU | B | 13 | 61.581 | 58.878 | 22.529 | 1.00 | 11.65 | B |
| ATOM | 2045 | CB | GLU | B | 13 | 60.151 | 58.869 | 23.090 | 1.00 | 11.63 | B |
| ATOM | 2046 | CG | GLU | B | 13 | 59.056 | 58.898 | 22.008 | 1.00 | 12.30 | B |
| ATOM | 2047 | CD | GLU | B | 13 | 58.836 | 57.558 | 21.315 | 1.00 | 13.74 | B |
| ATOM | 2048 | OE1 | GLU | B | 13 | 58.263 | 57.556 | 20.205 | 1.00 | 15.08 | B |
| ATOM | 2049 | OE2 | GLU | B | 13 | 59.215 | 56.506 | 21.873 | 1.00 | 13.46 | B |
| ATOM | 2050 | C | GLU | B | 13 | 61.877 | 60.097 | 21.668 | 1.00 | 10.94 | B |
| ATOM | 2051 | O | GLU | B | 13 | 61.802 | 61.226 | 22.145 | 1.00 | 11.44 | B |
| ATOM | 2052 | N | SER | B | 14 | 62.221 | 59.875 | 20.403 | 1.00 | 11.40 | B |
| ATOM | 2053 | CA | SER | B | 14 | 62.540 | 60.975 | 19.496 | 1.00 | 10.45 | B |
| ATOM | 2054 | CB | SER | B | 14 | 63.303 | 60.417 | 18.292 | 1.00 | 12.41 | B |
| ATOM | 2055 | OG | SER | B | 14 | 64.464 | 59.727 | 18.699 | 1.00 | 14.94 | B |
| ATOM | 2056 | C | SER | B | 14 | 61.277 | 61.718 | 19.085 | 1.00 | 10.94 | B |
| ATOM | 2057 | O | SER | B | 14 | 60.239 | 61.105 | 18.845 | 1.00 | 12.89 | B |
| ATOM | 2058 | N | PRO | B | 15 | 61.348 | 63.054 | 18.976 | 1.00 | 11.14 | B |
| ATOM | 2059 | CD | PRO | B | 15 | 60.292 | 63.756 | 18.224 | 1.00 | 11.21 | B |
| ATOM | 2060 | CA | PRO | B | 15 | 62.488 | 63.943 | 19.200 | 1.00 | 11.12 | B |
| ATOM | 2061 | CB | PRO | B | 15 | 62.361 | 64.903 | 18.029 | 1.00 | 10.05 | B |
| ATOM | 2062 | CG | PRO | B | 15 | 60.879 | 65.212 | 18.052 | 1.00 | 10.43 | B |
| ATOM | 2063 | C | PRO | B | 15 | 62.392 | 64.599 | 20.576 | 1.00 | 11.05 | B |
| ATOM | 2064 | O | PRO | B | 15 | 62.964 | 65.665 | 20.801 | 1.00 | 11.55 | B |
| ATOM | 2065 | N | TYR | B | 16 | 61.667 | 63.958 | 21.488 | 1.00 | 10.36 | B |
| ATOM | 2066 | CA | TYR | B | 16 | 61.458 | 64.483 | 22.837 | 1.00 | 10.25 | B |
| ATOM | 2067 | CB | TYR | B | 16 | 60.265 | 63.755 | 23.461 | 1.00 | 10.31 | B |
| ATOM | 2068 | CG | TYR | B | 16 | 59.030 | 63.764 | 22.591 | 1.00 | 8.58 | B |
| ATOM | 2069 | CD1 | TYR | B | 16 | 58.156 | 64.853 | 22.586 | 1.00 | 9.19 | B |
| ATOM | 2070 | CE1 | TYR | B | 16 | 57.020 | 64.853 | 21.775 | 1.00 | 8.77 | B |
| ATOM | 2071 | CD2 | TYR | B | 16 | 58.741 | 62.682 | 21.764 | 1.00 | 9.76 | B |
| ATOM | 2072 | CE2 | TYR | B | 16 | 57.618 | 62.674 | 20.954 | 1.00 | 10.41 | B |
| ATOM | 2073 | CZ | TYR | B | 16 | 56.761 | 63.758 | 20.965 | 1.00 | 9.82 | B |
| ATOM | 2074 | OH | TYR | B | 16 | 55.641 | 63.734 | 20.171 | 1.00 | 11.39 | B |
| ATOM | 2075 | C | TYR | B | 16 | 62.705 | 64.404 | 23.702 | 1.00 | 10.28 | B |
| ATOM | 2076 | O | TYR | B | 16 | 63.099 | 65.394 | 24.315 | 1.00 | 9.88 | B |
| ATOM | 2077 | N | VAL | B | 17 | 63.303 | 63.218 | 23.773 | 1.00 | 10.38 | B |
| ATOM | 2078 | CA | VAL | B | 17 | 64.518 | 63.004 | 24.553 | 1.00 | 10.31 | B |
| ATOM | 2079 | CB | VAL | B | 17 | 64.216 | 62.510 | 25.987 | 1.00 | 10.46 | B |
| ATOM | 2080 | CG1 | VAL | B | 17 | 65.503 | 62.456 | 26.800 | 1.00 | 11.76 | B |
| ATOM | 2081 | CG2 | VAL | B | 17 | 63.207 | 63.429 | 26.655 | 1.00 | 11.78 | B |
| ATOM | 2082 | C | VAL | B | 17 | 65.357 | 61.948 | 23.850 | 1.00 | 9.86 | B |
| ATOM | 2083 | O | VAL | B | 17 | 64.873 | 60.854 | 23.551 | 1.00 | 10.88 | B |
| ATOM | 2084 | N | MET | B | 18 | 66.612 | 62.294 | 23.581 | 1.00 | 10.26 | B |
| ATOM | 2085 | CA | MET | B | 18 | 67.549 | 61.406 | 22.906 | 1.00 | 12.06 | B |
| ATOM | 2086 | CB | MET | B | 18 | 67.583 | 61.761 | 21.409 | 1.00 | 15.22 | B |
| ATOM | 2087 | CG | MET | B | 18 | 66.216 | 61.838 | 20.728 | 1.00 | 19.02 | B |
| ATOM | 2088 | SD | MET | B | 18 | 66.244 | 62.690 | 19.120 | 1.00 | 21.27 | B |
| ATOM | 2089 | CE | MET | B | 18 | 66.218 | 64.416 | 19.671 | 1.00 | 17.44 | B |
| ATOM | 2090 | C | MET | B | 18 | 68.909 | 61.592 | 23.566 | 1.00 | 11.87 | B |
| ATOM | 2091 | O | MET | B | 18 | 69.196 | 62.662 | 24.102 | 1.00 | 11.59 | B |
| ATOM | 2092 | N | MET | B | 19 | 69.744 | 60.558 | 23.553 | 1.00 | 12.57 | B |
| ATOM | 2093 | CA | MET | B | 19 | 71.082 | 60.700 | 24.109 | 1.00 | 12.32 | B |
| ATOM | 2094 | CB | MET | B | 19 | 71.641 | 59.334 | 24.535 | 1.00 | 15.07 | B |
| ATOM | 2095 | CG | MET | B | 19 | 70.992 | 58.722 | 25.765 | 1.00 | 13.93 | B |
| ATOM | 2096 | SD | MET | B | 19 | 72.099 | 57.536 | 26.585 | 1.00 | 15.99 | B |
| ATOM | 2097 | CE | MET | B | 19 | 72.981 | 58.624 | 27.712 | 1.00 | 16.50 | B |
| ATOM | 2098 | C | MET | B | 19 | 71.911 | 61.354 | 23.008 | 1.00 | 12.90 | B |
| ATOM | 2099 | O | MET | B | 19 | 71.833 | 60.944 | 21.850 | 1.00 | 14.84 | B |
| ATOM | 2100 | N | LYS | B | 20 | 72.690 | 62.372 | 23.356 | 1.00 | 12.52 | B |
| ATOM | 2101 | CA | LYS | B | 20 | 73.533 | 63.038 | 22.367 | 1.00 | 14.47 | B |
| ATOM | 2102 | CB | LYS | B | 20 | 74.250 | 64.229 | 23.005 | 1.00 | 12.95 | B |
| ATOM | 2103 | CG | LYS | B | 20 | 73.374 | 65.417 | 23.367 | 1.00 | 13.71 | B |
| ATOM | 2104 | CD | LYS | B | 20 | 74.223 | 66.480 | 24.046 | 1.00 | 15.05 | B |
| ATOM | 2105 | CE | LYS | B | 20 | 73.396 | 67.668 | 24.509 | 1.00 | 14.15 | B |
| ATOM | 2106 | NZ | LYS | B | 20 | 74.229 | 68.600 | 25.320 | 1.00 | 15.71 | B |
| ATOM | 2107 | C | LYS | B | 20 | 74.511 | 62.008 | 21.827 | 1.00 | 16.21 | B |
| ATOM | 2108 | O | LYS | B | 20 | 74.881 | 61.068 | 22.529 | 1.00 | 14.97 | B |
| ATOM | 2109 | N | ALA | B | 21 | 74.942 | 62.196 | 20.584 | 1.00 | 18.29 | B |
| ATOM | 2110 | CA | ALA | B | 21 | 75.882 | 61.275 | 19.965 | 1.00 | 19.51 | B |
| ATOM | 2111 | CB | ALA | B | 21 | 76.177 | 61.721 | 18.535 | 1.00 | 21.05 | B |
| ATOM | 2112 | C | ALA | B | 21 | 77.176 | 61.201 | 20.773 | 1.00 | 19.76 | B |
| ATOM | 2113 | O | ALA | B | 21 | 77.802 | 60.144 | 20.864 | 1.00 | 21.42 | B |
| ATOM | 2114 | N | ASN | B | 22 | 77.557 | 62.325 | 21.372 | 1.00 | 19.02 | B |
| ATOM | 2115 | CA | ASN | B | 22 | 78.781 | 62.413 | 22.163 | 1.00 | 18.97 | B |
| ATOM | 2116 | CB | ASN | B | 22 | 79.521 | 63.693 | 21.766 | 1.00 | 20.30 | B |
| ATOM | 2117 | CG | ASN | B | 22 | 78.804 | 64.946 | 22.235 | 1.00 | 21.39 | B |
| ATOM | 2118 | OD1 | ASN | B | 22 | 77.584 | 65.061 | 22.104 | 1.00 | 22.04 | B |
| ATOM | 2119 | ND2 | ASN | B | 22 | 79.560 | 65.895 | 22.780 | 1.00 | 22.05 | B |
| ATOM | 2120 | C | ASN | B | 22 | 78.479 | 62.360 | 23.658 | 1.00 | 17.27 | B |
| ATOM | 2121 | O | ASN | B | 22 | 79.224 | 62.925 | 24.462 | 1.00 | 17.70 | B |
| ATOM | 2122 | N | HIS | B | 23 | 77.400 | 61.678 | 24.033 | 1.00 | 16.09 | B |
| ATOM | 2123 | CA | HIS | B | 23 | 77.013 | 61.598 | 25.438 | 1.00 | 16.05 | B |
| ATOM | 2124 | CB | HIS | B | 23 | 75.685 | 60.827 | 25.551 | 1.00 | 15.26 | B |
| ATOM | 2125 | CG | HIS | B | 23 | 75.793 | 59.364 | 25.245 | 1.00 | 14.60 | B |
| ATOM | 2126 | CD2 | HIS | B | 23 | 75.483 | 58.662 | 24.129 | 1.00 | 15.86 | B |
| ATOM | 2127 | ND1 | HIS | B | 23 | 76.238 | 58.440 | 26.165 | 1.00 | 14.48 | B |
| ATOM | 2128 | CE1 | HIS | B | 23 | 76.195 | 57.232 | 25.632 | 1.00 | 17.13 | B |
| ATOM | 2129 | NE2 | HIS | B | 23 | 75.740 | 57.339 | 24.397 | 1.00 | 15.98 | B |
| ATOM | 2130 | C | HIS | B | 23 | 78.118 | 61.062 | 26.351 | 1.00 | 15.69 | B |
| ATOM | 2131 | O | HIS | B | 23 | 78.184 | 61.428 | 27.521 | 1.00 | 15.50 | B |
| ATOM | 2132 | N | ALA | B | 24 | 78.995 | 60.217 | 25.817 | 1.00 | 17.18 | B |
| ATOM | 2133 | CA | ALA | B | 24 | 80.079 | 59.650 | 26.616 | 1.00 | 18.81 | B |
| ATOM | 2134 | CB | ALA | B | 24 | 80.816 | 58.586 | 25.810 | 1.00 | 19.51 | B |
| ATOM | 2135 | C | ALA | B | 24 | 81.070 | 60.702 | 27.117 | 1.00 | 18.71 | B |
| ATOM | 2136 | O | ALA | B | 24 | 81.774 | 60.482 | 28.102 | 1.00 | 18.45 | B |
| ATOM | 2137 | N | MET | B | 25 | 81.126 | 61.844 | 26.439 | 1.00 | 18.56 | B |
| ATOM | 2138 | CA | MET | B | 25 | 82.037 | 62.919 | 26.822 | 1.00 | 18.29 | B |
| ATOM | 2139 | CB | MET | B | 25 | 82.622 | 63.549 | 25.560 | 1.00 | 19.57 | B |
| ATOM | 2140 | CG | MET | B | 25 | 83.465 | 62.621 | 24.725 | 1.00 | 21.10 | B |
| ATOM | 2141 | SD | MET | B | 25 | 84.120 | 63.508 | 23.313 | 1.00 | 23.59 | B |
| ATOM | 2142 | CE | MET | B | 25 | 85.415 | 64.460 | 24.100 | 1.00 | 24.46 | B |
| ATOM | 2143 | C | MET | B | 25 | 81.326 | 63.950 | 27.684 | 1.00 | 18.23 | B |
| ATOM | 2144 | O | MET | B | 25 | 81.903 | 64.985 | 28.033 | 1.00 | 18.60 | B |
| ATOM | 2145 | N | LEU | B | 26 | 80.074 | 63.666 | 28.026 | 1.00 | 18.31 | B |
| ATOM | 2146 | CA | LEU | B | 26 | 79.266 | 64.587 | 28.812 | 1.00 | 16.85 | B |
| ATOM | 2147 | CB | LEU | B | 26 | 78.088 | 65.041 | 27.947 | 1.00 | 16.92 | B |
| ATOM | 2148 | CG | LEU | B | 26 | 78.475 | 65.583 | 26.567 | 1.00 | 17.51 | B |
| ATOM | 2149 | CD1 | LEU | B | 26 | 77.230 | 65.836 | 25.739 | 1.00 | 16.45 | B |
| ATOM | 2150 | CD2 | LEU | B | 26 | 79.279 | 66.862 | 26.726 | 1.00 | 17.51 | B |
| ATOM | 2151 | C | LEU | B | 26 | 78.830 | 63.976 | 30.137 | 1.00 | 16.24 | B |
| ATOM | 2152 | O | LEU | B | 26 | 79.043 | 62.791 | 30.394 | 1.00 | 15.00 | B |
| ATOM | 2153 | N | ALA | B | 27 | 78.207 | 64.800 | 30.971 | 1.00 | 16.05 | B |
| ATOM | 2154 | CA | ALA | B | 27 | 77.743 | 64.357 | 32.278 | 1.00 | 15.70 | B |
| ATOM | 2155 | CB | ALA | B | 27 | 78.774 | 64.724 | 33.340 | 1.00 | 16.75 | B |
| ATOM | 2156 | C | ALA | B | 27 | 76.397 | 64.975 | 32.633 | 1.00 | 14.51 | B |
| ATOM | 2157 | O | ALA | B | 27 | 76.049 | 66.059 | 32.165 | 1.00 | 14.31 | B |
| ATOM | 2158 | N | GLY | B | 28 | 75.634 | 64.272 | 33.461 | 1.00 | 12.99 | B |
| ATOM | 2159 | CA | GLY | B | 28 | 74.345 | 64.784 | 33.893 | 1.00 | 12.86 | B |
| ATOM | 2160 | C | GLY | B | 28 | 73.346 | 65.108 | 32.799 | 1.00 | 10.18 | B |
| ATOM | 2161 | O | GLY | B | 28 | 73.243 | 64.402 | 31.792 | 1.00 | 10.92 | B |
| ATOM | 2162 | N | ASN | B | 29 | 72.606 | 66.194 | 32.997 | 1.00 | 10.97 | B |
| ATOM | 2163 | CA | ASN | B | 29 | 71.588 | 66.617 | 32.042 | 1.00 | 11.28 | B |
| ATOM | 2164 | CB | ASN | B | 29 | 70.871 | 67.866 | 32.575 | 1.00 | 12.52 | B |
| ATOM | 2165 | CG | ASN | B | 29 | 70.099 | 67.613 | 33.862 | 1.00 | 13.16 | B |
| ATOM | 2166 | OD1 | ASN | B | 29 | 69.615 | 68.557 | 34.496 | 1.00 | 16.07 | B |
| ATOM | 2167 | ND2 | ASN | B | 29 | 69.968 | 66.351 | 34.249 | 1.00 | 10.44 | B |
| ATOM | 2168 | C | ASN | B | 29 | 72.159 | 66.854 | 30.647 | 1.00 | 11.11 | B |
| ATOM | 2169 | O | ASN | B | 29 | 71.440 | 66.751 | 29.662 | 1.00 | 12.20 | B |
| ATOM | 2170 | N | GLU | B | 30 | 73.445 | 67.190 | 30.574 | 1.00 | 11.25 | B |
| ATOM | 2171 | CA | GLU | B | 30 | 74.116 | 67.468 | 29.302 | 1.00 | 11.59 | B |
| ATOM | 2172 | CB | GLU | B | 30 | 75.531 | 67.982 | 29.592 | 1.00 | 13.28 | B |
| ATOM | 2173 | CG | GLU | B | 30 | 75.600 | 69.370 | 30.208 | 1.00 | 18.28 | B |
| ATOM | 2174 | CD | GLU | B | 30 | 75.494 | 70.476 | 29.176 | 1.00 | 22.46 | B |
| ATOM | 2175 | OE1 | GLU | B | 30 | 75.433 | 70.163 | 27.969 | 1.00 | 23.11 | B |
| ATOM | 2176 | OE2 | GLU | B | 30 | 75.482 | 71.662 | 29.575 | 1.00 | 24.65 | B |
| ATOM | 2177 | C | GLU | B | 30 | 74.135 | 66.296 | 28.324 | 1.00 | 10.36 | B |
| ATOM | 2178 | O | GLU | B | 30 | 74.352 | 66.487 | 27.128 | 1.00 | 11.64 | B |
| ATOM | 2179 | N | ARG | B | 31 | 73.923 | 65.086 | 28.834 | 1.00 | 9.81 | B |
| ATOM | 2180 | CA | ARG | B | 31 | 73.930 | 63.887 | 28.003 | 1.00 | 10.32 | B |
| ATOM | 2181 | CB | ARG | B | 31 | 73.955 | 62.647 | 28.899 | 1.00 | 10.62 | B |
| ATOM | 2182 | CG | ARG | B | 31 | 75.192 | 62.443 | 29.743 | 1.00 | 10.62 | B |
| ATOM | 2183 | CD | ARG | B | 31 | 74.937 | 61.315 | 30.732 | 1.00 | 9.95 | B |
| ATOM | 2184 | NE | ARG | B | 31 | 73.920 | 61.704 | 31.706 | 1.00 | 10.06 | B |
| ATOM | 2185 | CZ | ARG | B | 31 | 73.348 | 60.876 | 32.574 | 1.00 | 9.21 | B |
| ATOM | 2186 | NH1 | ARG | B | 31 | 73.681 | 59.595 | 32.597 | 1.00 | 11.18 | B |
| ATOM | 2187 | NH2 | ARG | B | 31 | 72.452 | 61.338 | 33.433 | 1.00 | 10.47 | B |
| ATOM | 2188 | C | ARG | B | 31 | 72.744 | 63.814 | 27.057 | 1.00 | 10.20 | B |
| ATOM | 2189 | O | ARG | B | 31 | 72.797 | 63.101 | 26.058 | 1.00 | 11.31 | B |
| ATOM | 2190 | N | TYR | B | 32 | 71.676 | 64.538 | 27.387 | 1.00 | 9.98 | B |
| ATOM | 2191 | CA | TYR | B | 32 | 70.425 | 64.496 | 26.627 | 1.00 | 8.83 | B |
| ATOM | 2192 | CB | TYR | B | 32 | 69.291 | 64.157 | 27.603 | 1.00 | 10.20 | B |
| ATOM | 2193 | CG | TYR | B | 32 | 69.544 | 62.935 | 28.451 | 1.00 | 9.07 | B |
| ATOM | 2194 | CD1 | TYR | B | 32 | 69.397 | 61.655 | 27.923 | 1.00 | 10.68 | B |
| ATOM | 2195 | CE1 | TYR | B | 32 | 69.645 | 60.522 | 28.698 | 1.00 | 10.81 | B |
| ATOM | 2196 | CD2 | TYR | B | 32 | 69.949 | 63.059 | 29.778 | 1.00 | 10.74 | B |
| ATOM | 2197 | CE2 | TYR | B | 32 | 70.203 | 61.935 | 30.562 | 1.00 | 10.68 | B |
| ATOM | 2198 | CZ | TYR | B | 32 | 70.048 | 60.670 | 30.015 | 1.00 | 9.71 | B |
| ATOM | 2199 | OH | TYR | B | 32 | 70.304 | 59.557 | 30.786 | 1.00 | 12.33 | B |
| ATOM | 2200 | C | TYR | B | 32 | 70.086 | 65.741 | 25.825 | 1.00 | 9.96 | B |
| ATOM | 2201 | O | TYR | B | 32 | 70.530 | 66.840 | 26.137 | 1.00 | 9.85 | B |
| ATOM | 2202 | N | GLU | B | 33 | 69.267 | 65.547 | 24.794 | 1.00 | 9.25 | B |
| ATOM | 2203 | CA | GLU | B | 33 | 68.802 | 66.637 | 23.945 | 1.00 | 9.61 | B |
| ATOM | 2204 | CB | GLU | B | 33 | 69.789 | 66.877 | 22.801 | 1.00 | 12.38 | B |
| ATOM | 2205 | CG | GLU | B | 33 | 69.882 | 65.719 | 21.823 | 1.00 | 15.84 | B |
| ATOM | 2206 | CD | GLU | B | 33 | 70.830 | 65.994 | 20.670 | 1.00 | 19.62 | B |
| ATOM | 2207 | OE1 | GLU | B | 33 | 71.160 | 65.039 | 19.936 | 1.00 | 21.62 | B |
| ATOM | 2208 | OE2 | GLU | B | 33 | 71.240 | 67.161 | 20.495 | 1.00 | 22.03 | B |
| ATOM | 2209 | C | GLU | B | 33 | 67.412 | 66.291 | 23.441 | 1.00 | 10.42 | B |
| ATOM | 2210 | O | GLU | B | 33 | 67.012 | 65.118 | 23.431 | 1.00 | 10.16 | B |
| ATOM | 2211 | N | GLY | B | 34 | 66.672 | 67.314 | 23.028 | 1.00 | 9.75 | B |
| ATOM | 2212 | CA | GLY | B | 34 | 65.339 | 67.083 | 22.510 | 1.00 | 9.73 | B |
| ATOM | 2213 | C | GLY | B | 34 | 64.321 | 68.084 | 23.016 | 1.00 | 8.85 | B |
| ATOM | 2214 | O | GLY | B | 34 | 64.616 | 68.904 | 23.887 | 1.00 | 10.30 | B |
| ATOM | 2215 | N | TYR | B | 35 | 63.115 | 68.003 | 22.468 | 1.00 | 8.53 | B |
| ATOM | 2216 | CA | TYR | B | 35 | 62.032 | 68.902 | 22.838 | 1.00 | 9.68 | B |
| ATOM | 2217 | CB | TYR | B | 35 | 60.782 | 68.528 | 22.031 | 1.00 | 10.10 | B |
| ATOM | 2218 | CG | TYR | B | 35 | 59.547 | 69.312 | 22.415 | 1.00 | 9.72 | B |
| ATOM | 2219 | CD1 | TYR | B | 35 | 58.848 | 69.024 | 23.590 | 1.00 | 9.32 | B |
| ATOM | 2220 | CE1 | TYR | B | 35 | 57.721 | 69.755 | 23.954 | 1.00 | 10.16 | B |
| ATOM | 2221 | CD2 | TYR | B | 35 | 59.086 | 70.355 | 21.613 | 1.00 | 10.84 | B |
| ATOM | 2222 | CE2 | TYR | B | 35 | 57.962 | 71.094 | 21.970 | 1.00 | 11.51 | B |
| ATOM | 2223 | CZ | TYR | B | 35 | 57.287 | 70.789 | 23.140 | 1.00 | 9.88 | B |
| ATOM | 2224 | OH | TYR | B | 35 | 56.185 | 71.530 | 23.507 | 1.00 | 12.71 | B |
| ATOM | 2225 | C | TYR | B | 35 | 61.773 | 68.928 | 24.341 | 1.00 | 9.67 | B |
| ATOM | 2226 | O | TYR | B | 35 | 61.676 | 70.000 | 24.943 | 1.00 | 10.24 | B |
| ATOM | 2227 | N | CYS | B | 36 | 61.644 | 67.755 | 24.952 | 1.00 | 9.69 | B |
| ATOM | 2228 | CA | CYS | B | 36 | 61.369 | 67.698 | 26.383 | 1.00 | 9.64 | B |
| ATOM | 2229 | CB | CYS | B | 36 | 60.823 | 66.312 | 26.744 | 1.00 | 11.19 | B |
| ATOM | 2230 | SG | CYS | B | 36 | 59.098 | 66.081 | 26.239 | 1.00 | 11.34 | B |
| ATOM | 2231 | C | CYS | B | 36 | 62.553 | 68.128 | 27.232 | 1.00 | 10.65 | B |
| ATOM | 2232 | O | CYS | B | 36 | 62.377 | 68.564 | 28.366 | 1.00 | 10.55 | B |
| ATOM | 2233 | N | VAL | B | 37 | 63.760 | 67.997 | 26.695 | 1.00 | 9.52 | B |
| ATOM | 2234 | CA | VAL | B | 37 | 64.945 | 68.430 | 27.423 | 1.00 | 10.61 | B |
| ATOM | 2235 | CB | VAL | B | 37 | 66.235 | 67.947 | 26.721 | 1.00 | 9.82 | B |
| ATOM | 2236 | CG1 | VAL | B | 37 | 67.459 | 68.492 | 27.442 | 1.00 | 11.76 | B |
| ATOM | 2237 | CG2 | VAL | B | 37 | 66.273 | 66.424 | 26.714 | 1.00 | 12.23 | B |
| ATOM | 2238 | C | VAL | B | 37 | 64.908 | 69.962 | 27.465 | 1.00 | 10.09 | B |
| ATOM | 2239 | O | VAL | B | 37 | 65.147 | 70.577 | 28.507 | 1.00 | 11.08 | B |
| ATOM | 2240 | N | ASP | B | 38 | 64.592 | 70.572 | 26.326 | 1.00 | 10.59 | B |
| ATOM | 2241 | CA | ASP | B | 38 | 64.495 | 72.024 | 26.232 | 1.00 | 9.97 | B |
| ATOM | 2242 | CB | ASP | B | 38 | 64.314 | 72.438 | 24.768 | 1.00 | 11.07 | B |
| ATOM | 2243 | CG | ASP | B | 38 | 65.571 | 72.264 | 23.939 | 1.00 | 10.71 | B |
| ATOM | 2244 | OD1 | ASP | B | 38 | 65.470 | 72.430 | 22.707 | 1.00 | 12.33 | B |
| ATOM | 2245 | OD2 | ASP | B | 38 | 66.644 | 71.977 | 24.506 | 1.00 | 13.75 | B |
| ATOM | 2246 | C | ASP | B | 38 | 63.336 | 72.504 | 27.100 | 1.00 | 10.54 | B |
| ATOM | 2247 | O | ASP | B | 38 | 63.442 | 73.529 | 27.776 | 1.00 | 10.16 | B |
| ATOM | 2248 | N | LEU | B | 39 | 62.222 | 71.772 | 27.066 | 1.00 | 9.79 | B |
| ATOM | 2249 | CA | LEU | B | 39 | 61.055 | 72.147 | 27.854 | 1.00 | 10.05 | B |
| ATOM | 2250 | CB | LEU | B | 39 | 59.870 | 71.244 | 27.490 | 1.00 | 11.14 | B |
| ATOM | 2251 | CG | LEU | B | 39 | 58.603 | 71.478 | 28.324 | 1.00 | 10.08 | B |
| ATOM | 2252 | CD1 | LEU | B | 39 | 58.137 | 72.921 | 28.178 | 1.00 | 11.75 | B |
| ATOM | 2253 | CD2 | LEU | B | 39 | 57.516 | 70.503 | 27.885 | 1.00 | 11.65 | B |
| ATOM | 2254 | C | LEU | B | 39 | 61.393 | 72.098 | 29.334 | 1.00 | 9.39 | B |
| ATOM | 2255 | O | LEU | B | 39 | 61.048 | 73.010 | 30.086 | 1.00 | 10.29 | B |
| ATOM | 2256 | N | ALA | B | 40 | 62.061 | 71.030 | 29.758 | 1.00 | 9.61 | B |
| ATOM | 2257 | CA | ALA | B | 40 | 62.432 | 70.896 | 31.162 | 1.00 | 10.73 | B |
| ATOM | 2258 | CB | ALA | B | 40 | 63.196 | 69.595 | 31.380 | 1.00 | 11.25 | B |
| ATOM | 2259 | C | ALA | B | 40 | 63.270 | 72.088 | 31.622 | 1.00 | 11.73 | B |
| ATOM | 2260 | O | ALA | B | 40 | 63.046 | 72.638 | 32.701 | 1.00 | 12.59 | B |
| ATOM | 2261 | N | ALA | B | 41 | 64.230 | 72.498 | 30.801 | 1.00 | 11.86 | B |
| ATOM | 2262 | CA | ALA | B | 41 | 65.082 | 73.626 | 31.153 | 1.00 | 12.18 | B |
| ATOM | 2263 | CB | ALA | B | 41 | 66.173 | 73.809 | 30.098 | 1.00 | 13.52 | B |
| ATOM | 2264 | C | ALA | B | 41 | 64.268 | 74.909 | 31.311 | 1.00 | 12.68 | B |
| ATOM | 2265 | O | ALA | B | 41 | 64.528 | 75.704 | 32.214 | 1.00 | 13.44 | B |
| ATOM | 2266 | N | GLU | B | 42 | 63.289 | 75.111 | 30.433 | 1.00 | 12.40 | B |
| ATOM | 2267 | CA | GLU | B | 42 | 62.445 | 76.299 | 30.499 | 1.00 | 13.32 | B |
| ATOM | 2268 | CB | GLU | B | 42 | 61.657 | 76.433 | 29.192 | 1.00 | 14.71 | B |
| ATOM | 2269 | CG | GLU | B | 42 | 62.430 | 77.116 | 28.081 | 1.00 | 18.34 | B |
| ATOM | 2270 | CD | GLU | B | 42 | 62.565 | 78.614 | 28.306 | 1.00 | 20.10 | B |
| ATOM | 2271 | OE1 | GLU | B | 42 | 62.279 | 79.083 | 29.429 | 1.00 | 20.21 | B |
| ATOM | 2272 | OE2 | GLU | B | 42 | 62.962 | 79.326 | 27.361 | 1.00 | 21.49 | B |
| ATOM | 2273 | C | GLU | B | 42 | 61.552 | 76.273 | 31.730 | 1.00 | 13.78 | B |
| ATOM | 2274 | O | GLU | B | 42 | 61.370 | 77.293 | 32.392 | 1.00 | 14.79 | B |
| ATOM | 2275 | N | ILE | B | 43 | 60.978 | 75.113 | 32.030 | 1.00 | 13.34 | B |
| ATOM | 2276 | CA | ILE | B | 43 | 60.115 | 74.987 | 33.199 | 1.00 | 13.46 | B |
| ATOM | 2277 | CB | ILE | B | 43 | 59.490 | 73.572 | 33.289 | 1.00 | 13.33 | B |
| ATOM | 2278 | CG2 | ILE | B | 43 | 58.751 | 73.408 | 34.613 | 1.00 | 12.28 | B |
| ATOM | 2279 | CG1 | ILE | B | 43 | 58.541 | 73.339 | 32.110 | 1.00 | 14.86 | B |
| ATOM | 2280 | CD1 | ILE | B | 43 | 57.323 | 74.242 | 32.101 | 1.00 | 16.43 | B |
| ATOM | 2281 | C | ILE | B | 43 | 60.925 | 75.263 | 34.468 | 1.00 | 13.03 | B |
| ATOM | 2282 | O | ILE | B | 43 | 60.498 | 76.023 | 35.339 | 1.00 | 15.24 | B |
| ATOM | 2283 | N | ALA | B | 44 | 62.102 | 74.653 | 34.563 | 1.00 | 13.22 | B |
| ATOM | 2284 | CA | ALA | B | 44 | 62.966 | 74.840 | 35.725 | 1.00 | 13.12 | B |
| ATOM | 2285 | CB | ALA | B | 44 | 64.197 | 73.962 | 35.599 | 1.00 | 13.62 | B |
| ATOM | 2286 | C | ALA | B | 44 | 63.379 | 76.298 | 35.886 | 1.00 | 14.36 | B |
| ATOM | 2287 | O | ALA | B | 44 | 63.439 | 76.812 | 37.001 | 1.00 | 14.64 | B |
| ATOM | 2288 | N | LYS | B | 45 | 63.666 | 76.959 | 34.769 | 1.00 | 15.50 | B |
| ATOM | 2289 | CA | LYS | B | 45 | 64.073 | 78.358 | 34.784 | 1.00 | 18.35 | B |
| ATOM | 2290 | CB | LYS | B | 45 | 64.464 | 78.775 | 33.366 | 1.00 | 19.84 | B |
| ATOM | 2291 | CG | LYS | B | 45 | 65.067 | 80.167 | 33.270 | 1.00 | 24.18 | B |
| ATOM | 2292 | CD | LYS | B | 45 | 65.213 | 80.612 | 31.819 | 1.00 | 28.35 | B |
| ATOM | 2293 | CE | LYS | B | 45 | 65.838 | 79.523 | 30.960 | 1.00 | 30.41 | B |
| ATOM | 2294 | NZ | LYS | B | 45 | 67.850 | 78.941 | 31.597 | 1.00 | 32.84 | B |
| ATOM | 2295 | C | LYS | B | 45 | 62.962 | 79.225 | 35.360 | 1.00 | 18.22 | B |
| ATOM | 2296 | O | LYS | B | 45 | 63.206 | 80.084 | 36.208 | 1.00 | 18.79 | B |
| ATOM | 2297 | N | HIS | B | 46 | 61.738 | 79.002 | 34.893 | 1.00 | 16.85 | B |
| ATOM | 2298 | CA | HIS | B | 46 | 60.597 | 79.781 | 35.357 | 1.00 | 18.06 | B |
| ATOM | 2299 | CB | HIS | B | 46 | 59.451 | 79.650 | 34.346 | 1.00 | 16.99 | B |
| ATOM | 2300 | CG | HIS | B | 46 | 59.668 | 80.432 | 33.089 | 1.00 | 18.62 | B |
| ATOM | 2301 | CD2 | HIS | B | 46 | 59.222 | 81.655 | 32.716 | 1.00 | 19.23 | B |
| ATOM | 2302 | ND1 | HIS | B | 46 | 60.476 | 79.990 | 32.064 | 1.00 | 20.49 | B |
| ATOM | 2303 | CE1 | HIS | B | 46 | 60.520 | 80.908 | 31.115 | 1.00 | 20.66 | B |
| ATOM | 2304 | NE2 | HIS | B | 46 | 59.768 | 81.928 | 31.486 | 1.00 | 22.31 | B |
| ATOM | 2305 | C | HIS | B | 46 | 60.166 | 79.459 | 36.783 | 1.00 | 18.25 | B |
| ATOM | 2306 | O | HIS | B | 46 | 59.643 | 80.328 | 37.481 | 1.00 | 20.05 | B |
| ATOM | 2307 | N | CYS | B | 47 | 60.381 | 78.224 | 37.224 | 1.00 | 18.44 | B |
| ATOM | 2308 | CA | CYS | B | 47 | 59.993 | 77.827 | 38.575 | 1.00 | 17.80 | B |
| ATOM | 2309 | CB | CYS | B | 47 | 59.473 | 76.389 | 38.549 | 1.00 | 18.10 | B |
| ATOM | 2310 | SG | CYS | B | 47 | 57.992 | 76.181 | 37.546 | 1.00 | 16.20 | B |
| ATOM | 2311 | C | CYS | B | 47 | 61.129 | 78.027 | 39.564 | 1.00 | 18.32 | B |
| ATOM | 2312 | O | CYS | B | 47 | 60.924 | 77.976 | 40.777 | 1.00 | 19.27 | B |
| ATOM | 2313 | N | GLY | B | 48 | 62.329 | 78.237 | 39.039 | 1.00 | 17.47 | B |
| ATOM | 2314 | CA | GLY | B | 48 | 63.483 | 78.460 | 39.887 | 1.00 | 17.87 | B |
| ATOM | 2315 | C | GLY | B | 48 | 63.989 | 77.250 | 40.647 | 1.00 | 17.16 | B |
| ATOM | 2316 | O | GLY | B | 48 | 64.315 | 77.357 | 41.829 | 1.00 | 19.29 | B |
| ATOM | 2317 | N | PHE | B | 49 | 64.056 | 76.096 | 39.991 | 1.00 | 16.57 | B |
| ATOM | 2318 | CA | PHE | B | 49 | 64.563 | 74.908 | 40.662 | 1.00 | 15.09 | B |
| ATOM | 2319 | CB | PHE | B | 49 | 63.397 | 73.957 | 41.021 | 1.00 | 15.47 | B |
| ATOM | 2320 | CG | PHE | B | 49 | 62.679 | 73.346 | 39.832 | 1.00 | 14.74 | B |
| ATOM | 2321 | CD1 | PHE | B | 49 | 63.277 | 72.476 | 39.080 | 1.00 | 13.45 | B |
| ATOM | 2322 | CD2 | PHE | B | 49 | 61.443 | 73.662 | 39.599 | 1.00 | 13.69 | B |
| ATOM | 2323 | CE1 | PHE | B | 49 | 62.630 | 71.844 | 38.006 | 1.00 | 15.09 | B |
| ATOM | 2324 | CE2 | PHE | B | 49 | 60.709 | 73.086 | 38.549 | 1.00 | 15.17 | B |
| ATOM | 2325 | CZ | PHE | B | 49 | 61.319 | 72.159 | 37.741 | 1.00 | 14.08 | B |
| ATOM | 2326 | C | PHE | B | 49 | 65.657 | 74.240 | 39.847 | 1.00 | 14.16 | B |
| ATOM | 2327 | O | PHE | B | 49 | 65.747 | 74.429 | 38.634 | 1.00 | 13.98 | B |
| ATOM | 2328 | N | ALA | B | 50 | 66.512 | 73.493 | 40.540 | 1.00 | 13.90 | B |
| ATOM | 2329 | CA | ALA | B | 50 | 67.580 | 72.740 | 39.896 | 1.00 | 13.70 | B |
| ATOM | 2330 | CB | ALA | B | 50 | 68.814 | 72.695 | 40.785 | 1.00 | 16.32 | B |
| ATOM | 2331 | C | ALA | B | 50 | 66.979 | 71.350 | 39.743 | 1.00 | 13.30 | B |
| ATOM | 2332 | O | ALA | B | 50 | 66.143 | 70.945 | 40.553 | 1.00 | 13.71 | B |
| ATOM | 2333 | N | TYR | B | 51 | 67.391 | 70.614 | 38.719 | 1.00 | 13.03 | B |
| ATOM | 2334 | CA | TYR | B | 51 | 66.812 | 69.296 | 38.500 | 1.00 | 11.76 | B |
| ATOM | 2335 | CB | TYR | B | 51 | 65.573 | 69.443 | 37.617 | 1.00 | 11.44 | B |
| ATOM | 2336 | CG | TYR | B | 51 | 65.911 | 69.967 | 36.243 | 1.00 | 11.49 | B |
| ATOM | 2337 | CD1 | TYR | B | 51 | 66.193 | 71.315 | 36.048 | 1.00 | 12.38 | B |
| ATOM | 2338 | CE1 | TYR | B | 51 | 66.566 | 71.799 | 34.802 | 1.00 | 12.32 | B |
| ATOM | 2339 | CD2 | TYR | B | 51 | 66.010 | 69.106 | 35.148 | 1.00 | 12.07 | B |
| ATOM | 2340 | CE2 | TYR | B | 51 | 66.386 | 69.583 | 33.895 | 1.00 | 11.64 | B |
| ATOM | 2341 | CZ | TYR | B | 51 | 66.662 | 70.930 | 33.732 | 1.00 | 12.48 | B |
| ATOM | 2342 | OH | TYR | B | 51 | 67.034 | 71.413 | 32.504 | 1.00 | 14.13 | B |
| ATOM | 2343 | C | TYR | B | 51 | 67.784 | 68.307 | 37.898 | 1.00 | 11.11 | B |
| ATOM | 2344 | O | TYR | B | 51 | 68.827 | 68.673 | 37.353 | 1.00 | 11.36 | B |
| ATOM | 2345 | N | ALA | B | 52 | 67.402 | 67.040 | 37.983 | 1.00 | 11.04 | B |
| ATOM | 2346 | CA | ALA | B | 52 | 68.190 | 65.953 | 37.435 | 1.00 | 11.50 | B |
| ATOM | 2347 | CB | ALA | B | 52 | 68.743 | 65.097 | 38.556 | 1.00 | 14.07 | B |
| ATOM | 2348 | C | ALA | B | 52 | 67.282 | 65.120 | 36.542 | 1.00 | 12.04 | B |
| ATOM | 2349 | O | ALA | B | 52 | 66.230 | 64.655 | 36.983 | 1.00 | 11.18 | B |
| ATOM | 2350 | N | LEU | B | 53 | 67.677 | 64.952 | 35.283 | 1.00 | 11.19 | B |
| ATOM | 2351 | CA | LEU | B | 53 | 66.907 | 64.145 | 34.342 | 1.00 | 11.11 | B |
| ATOM | 2352 | CB | LEU | B | 53 | 67.237 | 64.557 | 32.902 | 1.00 | 12.06 | B |
| ATOM | 2353 | CG | LEU | B | 53 | 66.890 | 65.967 | 32.423 | 1.00 | 11.30 | B |
| ATOM | 2354 | CD1 | LEU | B | 53 | 67.398 | 66.148 | 30.995 | 1.00 | 13.51 | B |
| ATOM | 2355 | CD2 | LEU | B | 53 | 65.389 | 66.189 | 32.492 | 1.00 | 12.62 | B |
| ATOM | 2356 | C | LEU | B | 53 | 67.235 | 62.683 | 34.591 | 1.00 | 11.07 | B |
| ATOM | 2357 | O | LEU | B | 53 | 68.401 | 62.291 | 34.550 | 1.00 | 12.22 | B |
| ATOM | 2358 | N | THR | B | 54 | 66.210 | 61.881 | 34.858 | 1.00 | 10.41 | B |
| ATOM | 2359 | CA | THR | B | 54 | 66.394 | 60.452 | 35.079 | 1.00 | 10.10 | B |
| ATOM | 2360 | CB | THR | B | 54 | 66.036 | 60.030 | 36.525 | 1.00 | 11.69 | B |
| ATOM | 2361 | OG1 | THR | B | 54 | 64.630 | 60.203 | 36.752 | 1.00 | 12.12 | B |
| ATOM | 2362 | CG2 | THR | B | 54 | 66.816 | 60.862 | 37.527 | 1.00 | 12.54 | B |
| ATOM | 2363 | C | THR | B | 54 | 65.482 | 59.730 | 34.095 | 1.00 | 10.22 | B |
| ATOM | 2364 | O | THR | B | 54 | 64.430 | 60.247 | 33.721 | 1.00 | 10.58 | B |
| ATOM | 2365 | N | ILE | B | 55 | 65.898 | 58.546 | 33.661 | 1.00 | 11.35 | B |
| ATOM | 2366 | CA | ILE | B | 55 | 65.123 | 57.775 | 32.701 | 1.00 | 9.56 | B |
| ATOM | 2367 | CB | ILE | B | 55 | 66.054 | 57.099 | 31.677 | 1.00 | 10.28 | B |
| ATOM | 2368 | CG2 | ILE | B | 55 | 65.230 | 56.422 | 30.596 | 1.00 | 10.52 | B |
| ATOM | 2369 | CG1 | ILE | B | 55 | 66.997 | 58.141 | 31.066 | 1.00 | 12.28 | B |
| ATOM | 2370 | CD1 | ILE | B | 55 | 66.297 | 59.307 | 30.389 | 1.00 | 13.29 | B |
| ATOM | 2371 | C | ILE | B | 55 | 64.293 | 56.721 | 33.415 | 1.00 | 11.06 | B |
| ATOM | 2372 | O | ILE | B | 55 | 64.822 | 55.907 | 34.175 | 1.00 | 10.82 | B |
| ATOM | 2373 | N | VAL | B | 56 | 62.986 | 56.743 | 33.170 | 1.00 | 11.05 | B |
| ATOM | 2374 | CA | VAL | B | 56 | 62.066 | 55.809 | 33.807 | 1.00 | 11.37 | B |
| ATOM | 2375 | CB | VAL | B | 56 | 60.629 | 55.969 | 33.228 | 1.00 | 9.97 | B |
| ATOM | 2376 | CG1 | VAL | B | 56 | 60.615 | 55.684 | 31.744 | 1.00 | 9.92 | B |
| ATOM | 2377 | CG2 | VAL | B | 56 | 59.667 | 55.064 | 33.966 | 1.00 | 11.61 | B |
| ATOM | 2378 | C | VAL | B | 56 | 62.543 | 54.360 | 33.703 | 1.00 | 11.84 | B |
| ATOM | 2379 | O | VAL | B | 56 | 62.792 | 53.835 | 32.616 | 1.00 | 11.48 | B |
| ATOM | 2380 | N | GLY | B | 57 | 62.657 | 53.723 | 34.862 | 1.00 | 12.37 | B |
| ATOM | 2381 | CA | GLY | B | 57 | 63.146 | 52.360 | 34.937 | 1.00 | 13.63 | B |
| ATOM | 2382 | C | GLY | B | 57 | 62.508 | 51.288 | 34.080 | 1.00 | 14.12 | B |
| ATOM | 2383 | O | GLY | B | 57 | 63.221 | 50.521 | 33.430 | 1.00 | 15.65 | B |
| ATOM | 2384 | N | ASP | B | 58 | 61.178 | 51.223 | 34.068 | 1.00 | 14.76 | B |
| ATOM | 2385 | CA | ASP | B | 58 | 60.486 | 50.189 | 33.307 | 1.00 | 14.59 | B |
| ATOM | 2386 | CB | ASP | B | 58 | 59.266 | 49.713 | 34.110 | 1.00 | 14.42 | B |
| ATOM | 2387 | CG | ASP | B | 58 | 58.191 | 50.776 | 34.243 | 1.00 | 13.18 | B |
| ATOM | 2388 | OD1 | ASP | B | 58 | 57.230 | 50.545 | 35.005 | 1.00 | 16.08 | B |
| ATOM | 2389 | OD2 | ASP | B | 58 | 58.296 | 51.835 | 33.586 | 1.00 | 12.74 | B |
| ATOM | 2390 | C | ASP | B | 58 | 60.143 | 50.567 | 31.872 | 1.00 | 14.19 | B |
| ATOM | 2391 | O | ASP | B | 58 | 59.470 | 49.805 | 31.180 | 1.00 | 15.43 | B |
| ATOM | 2392 | N | GLY | B | 59 | 60.594 | 51.737 | 31.428 | 1.00 | 14.19 | B |
| ATOM | 2393 | CA | GLY | B | 59 | 60.331 | 52.162 | 30.062 | 1.00 | 11.83 | B |
| ATOM | 2394 | C | GLY | B | 59 | 58.867 | 52.283 | 29.684 | 1.00 | 12.48 | B |
| ATOM | 2395 | O | GLY | B | 59 | 58.514 | 52.155 | 28.510 | 1.00 | 12.94 | B |
| ATOM | 2396 | N | LYS | B | 60 | 58.017 | 52.551 | 30.672 | 1.00 | 11.56 | B |
| ATOM | 2397 | CA | LYS | B | 60 | 56.577 | 52.677 | 30.450 | 1.00 | 12.01 | B |
| ATOM | 2398 | CB | LYS | B | 60 | 55.846 | 51.652 | 31.326 | 1.00 | 13.09 | B |
| ATOM | 2399 | CG | LYS | B | 60 | 56.245 | 50.200 | 31.110 | 1.00 | 15.47 | B |
| ATOM | 2400 | CD | LYS | B | 60 | 55.738 | 49.675 | 29.786 | 1.00 | 18.86 | B |
| ATOM | 2401 | CE | LYS | B | 60 | 56.022 | 48.185 | 29.656 | 1.00 | 20.26 | B |
| ATOM | 2402 | NZ | LYS | B | 60 | 55.522 | 47.641 | 28.365 | 1.00 | 21.95 | B |
| ATOM | 2403 | C | LYS | B | 60 | 56.104 | 54.098 | 30.731 | 1.00 | 10.84 | B |
| ATOM | 2404 | O | LYS | B | 60 | 56.786 | 54.875 | 31.408 | 1.00 | 11.54 | B |
| ATOM | 2405 | N | TYR | B | 61 | 54.923 | 54.432 | 30.217 | 1.00 | 10.31 | B |
| ATOM | 2406 | CA | TYR | B | 61 | 54.352 | 55.754 | 30.435 | 1.00 | 10.95 | B |
| ATOM | 2407 | CB | TYR | B | 61 | 53.617 | 56.209 | 29.172 | 1.00 | 10.41 | B |
| ATOM | 2408 | CG | TYR | B | 61 | 54.567 | 56.686 | 28.106 | 1.00 | 10.06 | B |
| ATOM | 2409 | CD1 | TYR | B | 61 | 55.372 | 55.783 | 27.409 | 1.00 | 10.42 | B |
| ATOM | 2410 | CE1 | TYR | B | 61 | 56.304 | 56.224 | 26.479 | 1.00 | 10.50 | B |
| ATOM | 2411 | CD2 | TYR | B | 61 | 54.717 | 58.047 | 27.837 | 1.00 | 10.35 | B |
| ATOM | 2412 | CE2 | TYR | B | 61 | 55.648 | 58.499 | 26.906 | 1.00 | 10.24 | B |
| ATOM | 2413 | CZ | TYR | B | 61 | 56.435 | 57.581 | 26.234 | 1.00 | 11.07 | B |
| ATOM | 2414 | OH | TYR | B | 61 | 57.349 | 58.030 | 25.310 | 1.00 | 12.79 | B |
| ATOM | 2415 | C | TYR | B | 61 | 53.487 | 55.771 | 31.685 | 1.00 | 9.95 | B |
| ATOM | 2416 | O | TYR | B | 61 | 53.714 | 56.578 | 32.585 | 1.00 | 11.01 | B |
| ATOM | 2417 | N | GLY | B | 62 | 52.499 | 54.884 | 31.750 | 1.00 | 10.51 | B |
| ATOM | 2418 | CA | GLY | B | 62 | 51.653 | 54.847 | 32.930 | 1.00 | 11.84 | B |
| ATOM | 2419 | C | GLY | B | 62 | 50.340 | 54.109 | 32.768 | 1.00 | 10.79 | B |
| ATOM | 2420 | O | GLY | B | 62 | 49.416 | 54.599 | 32.116 | 1.00 | 10.82 | B |
| ATOM | 2421 | N | ALA | B | 63 | 50.264 | 52.927 | 33.370 | 1.00 | 12.75 | B |
| ATOM | 2422 | CA | ALA | B | 63 | 49.065 | 52.100 | 33.330 | 1.00 | 13.25 | B |
| ATOM | 2423 | CB | ALA | B | 63 | 49.122 | 51.145 | 32.147 | 1.00 | 13.85 | B |
| ATOM | 2424 | C | ALA | B | 63 | 49.002 | 51.321 | 34.637 | 1.00 | 14.70 | B |
| ATOM | 2425 | O | ALA | B | 63 | 50.030 | 51.085 | 35.276 | 1.00 | 14.41 | B |
| ATOM | 2426 | N | ARG | B | 64 | 47.796 | 50.926 | 35.032 | 1.00 | 15.95 | B |
| ATOM | 2427 | CA | ARG | B | 64 | 47.603 | 50.178 | 36.268 | 1.00 | 18.84 | B |
| ATOM | 2428 | CB | ARG | B | 64 | 46.555 | 50.896 | 37.129 | 1.00 | 20.04 | B |
| ATOM | 2429 | CG | ARG | B | 64 | 46.300 | 50.249 | 38.487 | 1.00 | 23.49 | B |
| ATOM | 2430 | CD | ARG | B | 64 | 44.957 | 49.522 | 38.541 | 1.00 | 27.55 | B |
| ATOM | 2431 | NE | ARG | B | 64 | 43.825 | 50.438 | 38.421 | 1.00 | 28.94 | B |
| ATOM | 2432 | CZ | ARG | B | 64 | 42.551 | 50.076 | 38.545 | 1.00 | 30.80 | B |
| ATOM | 2433 | NH1 | ARG | B | 64 | 42.238 | 48.812 | 38.797 | 1.00 | 29.54 | B |
| ATOM | 2434 | NH2 | ARG | B | 64 | 41.587 | 50.977 | 38.417 | 1.00 | 31.20 | B |
| ATOM | 2435 | C | ARG | B | 64 | 47.233 | 48.734 | 35.961 | 1.00 | 19.59 | B |
| ATOM | 2436 | O | ARG | B | 65 | 46.286 | 48.470 | 35.220 | 1.00 | 19.24 | B |
| ATOM | 2437 | N | ASP | B | 65 | 47.990 | 47.801 | 36.530 | 1.00 | 21.81 | B |
| ATOM | 2438 | CA | ASP | B | 65 | 47.723 | 46.382 | 36.327 | 1.00 | 23.78 | B |
| ATOM | 2439 | CB | ASP | B | 65 | 48.852 | 45.563 | 36.956 | 1.00 | 24.91 | B |
| ATOM | 2440 | CG | ASP | B | 65 | 48.744 | 44.084 | 36.636 | 1.00 | 26.39 | B |
| ATOM | 2441 | OD1 | ASP | B | 65 | 48.998 | 43.707 | 35.473 | 1.00 | 27.64 | B |
| ATOM | 2442 | OD2 | ASP | B | 65 | 48.397 | 43.302 | 37.544 | 1.00 | 28.63 | B |
| ATOM | 2443 | C | ASP | B | 65 | 46.356 | 46.098 | 36.941 | 1.00 | 24.92 | B |
| ATOM | 2444 | O | ASP | B | 65 | 46.096 | 46.465 | 38.086 | 1.00 | 25.04 | B |
| ATOM | 2445 | N | ALA | B | 66 | 45.483 | 45.444 | 36.181 | 1.00 | 26.95 | B |
| ATOM | 2446 | CA | ALA | B | 66 | 44.140 | 45.136 | 36.659 | 1.00 | 28.23 | B |
| ATOM | 2447 | CB | ALA | B | 66 | 43.292 | 44.605 | 35.509 | 1.00 | 28.97 | B |
| ATOM | 2448 | C | ALA | B | 66 | 44.129 | 44.140 | 37.814 | 1.00 | 28.97 | B |
| ATOM | 2449 | O | ALA | B | 66 | 43.167 | 44.083 | 38.580 | 1.00 | 29.82 | B |
| ATOM | 2450 | N | ASN | B | 67 | 45.198 | 43.363 | 37.942 | 1.00 | 28.97 | B |
| ATOM | 2451 | CA | ASN | B | 67 | 45.289 | 42.365 | 39.000 | 1.00 | 29.76 | B |
| ATOM | 2452 | CB | ASN | B | 67 | 46.029 | 41.137 | 38.458 | 1.00 | 30.71 | B |
| ATOM | 2453 | CG | ASN | B | 67 | 45.317 | 40.501 | 37.279 | 1.00 | 32.34 | B |
| ATOM | 2454 | OD1 | ASN | B | 67 | 44.160 | 40.094 | 37.385 | 1.00 | 33.09 | B |
| ATOM | 2455 | ND2 | ASN | B | 67 | 46.007 | 40.411 | 36.146 | 1.00 | 33.17 | B |
| ATOM | 2456 | C | ASN | B | 67 | 45.917 | 42.922 | 40.274 | 1.00 | 28.95 | B |
| ATOM | 2457 | O | ASN | B | 67 | 45.293 | 42.916 | 41.336 | 1.00 | 29.70 | B |
| ATOM | 2458 | N | THR | B | 68 | 47.151 | 43.405 | 40.163 | 1.00 | 27.58 | B |
| ATOM | 2459 | CA | THR | B | 68 | 47.876 | 43.945 | 41.309 | 1.00 | 25.51 | B |
| ATOM | 2460 | CB | THR | B | 68 | 49.394 | 43.894 | 41.073 | 1.00 | 25.12 | B |
| ATOM | 2461 | OG1 | THR | B | 68 | 49.745 | 44.801 | 41.019 | 1.00 | 20.95 | B |
| ATOM | 2462 | CG2 | THR | B | 68 | 49.822 | 42.489 | 40.687 | 1.00 | 24.96 | B |
| ATOM | 2463 | C | THR | B | 68 | 47.505 | 45.384 | 41.647 | 1.00 | 24.97 | B |
| ATOM | 2464 | O | THR | B | 68 | 47.760 | 45.849 | 42.758 | 1.00 | 25.29 | B |
| ATOM | 2465 | N | LYS | B | 69 | 46.909 | 46.083 | 40.687 | 1.00 | 24.16 | B |
| ATOM | 2466 | CA | LYS | B | 69 | 46.514 | 47.475 | 40.868 | 1.00 | 23.37 | B |
| ATOM | 2467 | CB | LYS | B | 69 | 45.496 | 47.579 | 42.010 | 1.00 | 26.52 | B |
| ATOM | 2468 | CG | LYS | B | 69 | 44.139 | 46.975 | 41.679 | 1.00 | 29.62 | B |
| ATOM | 2469 | CD | LYS | B | 69 | 43.086 | 47.390 | 42.694 | 1.00 | 32.54 | B |
| ATOM | 2470 | CE | LYS | B | 69 | 41.702 | 46.906 | 42.287 | 1.00 | 34.99 | B |
| ATOM | 2471 | NZ | LYS | B | 69 | 40.648 | 47.378 | 43.230 | 1.00 | 35.23 | B |
| ATOM | 2472 | C | LYS | B | 69 | 47.732 | 48.372 | 41.062 | 1.00 | 21.74 | B |
| ATOM | 2473 | O | LYS | B | 69 | 47.623 | 49.479 | 41.593 | 1.00 | 20.74 | B |
| ATOM | 2474 | N | ILE | B | 70 | 48.891 | 47.890 | 40.628 | 1.00 | 18.80 | B |
| ATOM | 2475 | CA | ILE | B | 70 | 50.126 | 48.657 | 40.735 | 1.00 | 17.97 | B |
| ATOM | 2476 | CB | ILE | B | 70 | 51.352 | 47.729 | 40.900 | 1.00 | 18.88 | B |
| ATOM | 2477 | CG2 | ILE | B | 70 | 52.640 | 48.538 | 40.806 | 1.00 | 20.23 | B |
| ATOM | 2478 | CG1 | ILE | B | 70 | 51.269 | 46.995 | 42.241 | 1.00 | 20.76 | B |
| ATOM | 2479 | CD1 | ILE | B | 70 | 52.423 | 46.045 | 42.499 | 1.00 | 22.88 | B |
| ATOM | 2480 | C | ILE | B | 70 | 50.309 | 49.492 | 39.471 | 1.00 | 15.47 | B |
| ATOM | 2481 | O | ILE | B | 70 | 50.047 | 49.019 | 38.365 | 1.00 | 15.13 | B |
| ATOM | 2482 | N | TRP | B | 71 | 50.742 | 50.737 | 39.644 | 1.00 | 13.13 | B |
| ATOM | 2483 | CA | TRP | B | 71 | 50.975 | 51.636 | 38.520 | 1.00 | 12.67 | B |
| ATOM | 2484 | CB | TRP | B | 71 | 50.674 | 53.079 | 38.937 | 1.00 | 12.72 | B |
| ATOM | 2485 | CG | TRP | B | 71 | 49.224 | 53.410 | 38.968 | 1.00 | 13.58 | B |
| ATOM | 2486 | CD2 | TRP | B | 71 | 48.438 | 53.888 | 37.873 | 1.00 | 14.46 | B |
| ATOM | 2487 | CE2 | TRP | B | 71 | 47.116 | 54.046 | 38.345 | 1.00 | 15.69 | B |
| ATOM | 2488 | CE3 | TRP | B | 71 | 48.722 | 54.198 | 36.536 | 1.00 | 13.52 | B |
| ATOM | 2489 | CD1 | TRP | B | 71 | 48.373 | 53.297 | 40.033 | 1.00 | 14.51 | B |
| ATOM | 2490 | NE1 | TRP | B | 71 | 47.106 | 53.679 | 39.665 | 1.00 | 16.14 | B |
| ATOM | 2491 | CZ2 | TRP | B | 71 | 46.077 | 54.500 | 37.526 | 1.00 | 14.84 | B |
| ATOM | 2492 | CZ3 | TRP | B | 71 | 47.687 | 54.651 | 35.721 | 1.00 | 15.20 | B |
| ATOM | 2493 | CH2 | TRP | B | 71 | 46.381 | 54.796 | 36.222 | 1.00 | 14.88 | B |
| ATOM | 2494 | C | TRP | B | 71 | 52.402 | 51.491 | 38.026 | 1.00 | 11.67 | B |
| ATOM | 2495 | O | TRP | B | 71 | 53.335 | 51.458 | 38.826 | 1.00 | 13.19 | B |
| ATOM | 2496 | N | ASN | B | 72 | 52.574 | 51.412 | 36.708 | 1.00 | 10.89 | B |
| ATOM | 2497 | CA | ASN | B | 72 | 53.909 | 51.305 | 36.136 | 1.00 | 10.92 | B |
| ATOM | 2498 | CB | ASN | B | 72 | 53.971 | 50.121 | 35.155 | 1.00 | 11.32 | B |
| ATOM | 2499 | CG | ASN | B | 72 | 53.110 | 50.331 | 33.920 | 1.00 | 11.78 | B |
| ATOM | 2500 | OD1 | ASN | B | 72 | 52.720 | 51.456 | 33.596 | 1.00 | 12.64 | B |
| ATOM | 2501 | ND2 | ASN | B | 72 | 52.826 | 49.245 | 33.213 | 1.00 | 15.85 | B |
| ATOM | 2502 | C | ASN | B | 72 | 54.284 | 52.625 | 35.471 | 1.00 | 9.96 | B |
| ATOM | 2503 | O | ASN | B | 72 | 53.560 | 53.616 | 35.562 | 1.00 | 10.53 | B |
| ATOM | 2504 | N | GLY | B | 73 | 55.440 | 52.619 | 34.819 | 1.00 | 10.42 | B |
| ATOM | 2505 | CA | GLY | B | 73 | 55.915 | 53.781 | 34.089 | 1.00 | 9.99 | B |
| ATOM | 2506 | C | GLY | B | 73 | 56.175 | 55.046 | 34.877 | 1.00 | 9.20 | B |
| ATOM | 2507 | O | GLY | B | 73 | 56.366 | 55.017 | 36.093 | 1.00 | 9.61 | B |
| ATOM | 2508 | N | MET | B | 74 | 56.195 | 56.170 | 34.161 | 1.00 | 9.33 | B |
| ATOM | 2509 | CA | MET | B | 74 | 56.438 | 57.468 | 34.775 | 1.00 | 9.66 | B |
| ATOM | 2510 | CB | MET | B | 74 | 56.612 | 58.526 | 33.683 | 1.00 | 9.48 | B |
| ATOM | 2511 | CG | MET | B | 74 | 57.870 | 58.331 | 32.857 | 1.00 | 10.36 | B |
| ATOM | 2512 | SD | MET | B | 74 | 58.035 | 59.534 | 31.542 | 1.00 | 9.40 | B |
| ATOM | 2513 | CE | MET | B | 74 | 56.941 | 58.818 | 30.315 | 1.00 | 10.99 | B |
| ATOM | 2514 | C | MET | B | 74 | 55.345 | 57.817 | 35.767 | 1.00 | 8.74 | B |
| ATOM | 2515 | O | MET | B | 74 | 55.615 | 58.424 | 36.802 | 1.00 | 9.06 | B |
| ATOM | 2516 | N | VAL | B | 75 | 54.107 | 57.448 | 35.454 | 1.00 | 9.57 | B |
| ATOM | 2517 | CA | VAL | B | 75 | 53.012 | 57.722 | 36.372 | 1.00 | 10.23 | B |
| ATOM | 2518 | CB | VAL | B | 75 | 51.650 | 57.289 | 35.774 | 1.00 | 10.32 | B |
| ATOM | 2519 | CG1 | VAL | B | 75 | 50.555 | 57.392 | 36.832 | 1.00 | 11.55 | B |
| ATOM | 2520 | CG2 | VAL | B | 75 | 51.305 | 58.187 | 34.582 | 1.00 | 11.56 | B |
| ATOM | 2521 | C | VAL | B | 75 | 53.298 | 56.973 | 37.675 | 1.00 | 9.02 | B |
| ATOM | 2522 | O | VAL | B | 75 | 53.161 | 57.532 | 38.761 | 1.00 | 9.52 | B |
| ATOM | 2523 | N | GLY | B | 76 | 53.719 | 55.716 | 37.564 | 1.00 | 9.20 | B |
| ATOM | 2524 | CA | GLY | B | 76 | 54.035 | 54.948 | 38.756 | 1.00 | 10.49 | B |
| ATOM | 2525 | C | GLY | B | 76 | 55.137 | 55.590 | 39.585 | 1.00 | 10.06 | B |
| ATOM | 2526 | O | GLY | B | 76 | 55.057 | 55.627 | 40.812 | 1.00 | 10.24 | B |
| ATOM | 2527 | N | GLU | B | 77 | 56.172 | 56.106 | 38.927 | 1.00 | 10.03 | B |
| ATOM | 2528 | CA | GLU | B | 77 | 57.262 | 56.739 | 39.656 | 1.00 | 11.26 | B |
| ATOM | 2529 | CB | GLU | B | 77 | 58.387 | 57.115 | 38.688 | 1.00 | 12.58 | B |
| ATOM | 2530 | CG | GLU | B | 77 | 59.025 | 55.921 | 37.999 | 1.00 | 14.21 | B |
| ATOM | 2531 | CD | GLU | B | 77 | 60.416 | 56.222 | 37.479 | 1.00 | 16.09 | B |
| ATOM | 2532 | OE1 | GLU | B | 77 | 60.720 | 57.408 | 37.234 | 1.00 | 19.00 | B |
| ATOM | 2533 | OE2 | GLU | B | 77 | 61.204 | 55.270 | 37.307 | 1.00 | 16.26 | B |
| ATOM | 2534 | C | GLU | B | 77 | 56.742 | 57.918 | 40.466 | 1.00 | 10.75 | B |
| ATOM | 2535 | O | GLU | B | 77 | 57.215 | 58.169 | 41.569 | 1.00 | 11.53 | B |
| ATOM | 2536 | N | LEU | B | 78 | 55.770 | 58.644 | 39.924 | 1.00 | 9.82 | B |
| ATOM | 2537 | CA | LEU | B | 78 | 55.192 | 59.781 | 40.639 | 1.00 | 9.75 | B |
| ATOM | 2538 | CB | LEU | B | 78 | 54.402 | 60.650 | 39.664 | 1.00 | 10.18 | B |
| ATOM | 2539 | CG | LEU | B | 78 | 55.183 | 61.318 | 38.532 | 1.00 | 11.06 | B |
| ATOM | 2540 | CD1 | LEU | B | 78 | 54.201 | 62.002 | 37.607 | 1.00 | 12.43 | B |
| ATOM | 2541 | CD2 | LEU | B | 78 | 56.190 | 62.320 | 39.088 | 1.00 | 10.65 | B |
| ATOM | 2542 | C | LEU | B | 78 | 54.323 | 59.283 | 41.787 | 1.00 | 10.67 | B |
| ATOM | 2543 | O | LEU | B | 78 | 54.421 | 59.768 | 42.913 | 1.00 | 11.04 | B |
| ATOM | 2544 | N | VAL | B | 79 | 53.465 | 58.319 | 41.485 | 1.00 | 10.25 | B |
| ATOM | 2545 | CA | VAL | B | 79 | 52.557 | 57.754 | 42.470 | 1.00 | 11.27 | B |
| ATOM | 2546 | CB | VAL | B | 79 | 51.688 | 56.649 | 41.821 | 1.00 | 11.30 | B |
| ATOM | 2547 | CG1 | VAL | B | 79 | 50.928 | 55.865 | 42.888 | 1.00 | 12.72 | B |
| ATOM | 2548 | CG2 | VAL | B | 79 | 50.715 | 57.282 | 40.832 | 1.00 | 13.10 | B |
| ATOM | 2549 | C | VAL | B | 79 | 53.288 | 57.184 | 43.683 | 1.00 | 11.56 | B |
| ATOM | 2550 | O | VAL | B | 79 | 52.852 | 57.375 | 44.822 | 1.00 | 11.77 | B |
| ATOM | 2551 | N | TYR | B | 80 | 54.408 | 56.508 | 43.444 | 1.00 | 10.46 | B |
| ATOM | 2552 | CA | TYR | B | 80 | 55.167 | 55.895 | 44.532 | 1.00 | 11.28 | B |
| ATOM | 2553 | CB | TYR | B | 80 | 55.659 | 54.517 | 44.069 | 1.00 | 11.09 | B |
| ATOM | 2554 | CG | TYR | B | 80 | 54.513 | 53.621 | 43.670 | 1.00 | 10.97 | B |
| ATOM | 2555 | CD1 | TYR | B | 80 | 53.496 | 53.321 | 44.575 | 1.00 | 12.84 | B |
| ATOM | 2556 | CE1 | TYR | B | 80 | 52.384 | 52.571 | 44.191 | 1.00 | 12.75 | B |
| ATOM | 2557 | CD2 | TYR | B | 80 | 54.397 | 53.138 | 42.368 | 1.00 | 11.00 | B |
| ATOM | 2558 | CE2 | TYR | B | 80 | 53.291 | 52.388 | 41.973 | 1.00 | 12.20 | B |
| ATOM | 2559 | CZ | TYR | B | 80 | 52.286 | 52.112 | 42.887 | 1.00 | 11.32 | B |
| ATOM | 2560 | OH | TYR | B | 80 | 51.168 | 51.408 | 42.495 | 1.00 | 12.36 | B |
| ATOM | 2561 | C | TYR | B | 80 | 56.269 | 56.779 | 45.104 | 1.00 | 11.47 | B |
| ATOM | 2562 | O | TYR | B | 80 | 57.025 | 56.341 | 45.975 | 1.00 | 12.62 | B |
| ATOM | 2563 | N | GLY | B | 81 | 56.354 | 58.016 | 44.618 | 1.00 | 11.77 | B |
| ATOM | 2564 | CA | GLY | B | 81 | 57.343 | 58.963 | 45.114 | 1.00 | 13.08 | B |
| ATOM | 2565 | C | GLY | B | 81 | 58.785 | 58.763 | 44.684 | 1.00 | 14.28 | B |
| ATOM | 2566 | O | GLY | B | 81 | 59.702 | 59.201 | 45.381 | 1.00 | 16.40 | B |
| ATOM | 2567 | N | LYS | B | 82 | 58.995 | 58.121 | 43.540 | 1.00 | 12.68 | B |
| ATOM | 2568 | CA | LYS | B | 82 | 60.344 | 57.867 | 43.036 | 1.00 | 13.74 | B |
| ATOM | 2569 | CB | LYS | B | 82 | 60.338 | 56.557 | 42.236 | 1.00 | 14.63 | B |
| ATOM | 2570 | CG | LYS | B | 82 | 59.824 | 55.346 | 43.003 | 1.00 | 18.22 | B |
| ATOM | 2571 | CD | LYS | B | 82 | 60.734 | 54.978 | 44.159 | 1.00 | 21.14 | B |
| ATOM | 2572 | CE | LYS | B | 82 | 60.223 | 53.738 | 44.882 | 1.00 | 23.62 | B |
| ATOM | 2573 | NZ | LYS | B | 82 | 61.113 | 53.339 | 46.009 | 1.00 | 25.94 | B |
| ATOM | 2574 | C | LYS | B | 82 | 60.849 | 59.056 | 42.224 | 1.00 | 12.88 | B |
| ATOM | 2575 | O | LYS | B | 82 | 62.050 | 59.190 | 41.982 | 1.00 | 14.84 | B |
| ATOM | 2576 | N | ALA | B | 83 | 59.924 | 59.907 | 41.794 | 1.00 | 12.38 | B |
| ATOM | 2577 | CA | ALA | B | 83 | 60.263 | 61.096 | 41.016 | 1.00 | 11.64 | B |
| ATOM | 2578 | CB | ALA | B | 83 | 60.123 | 60.805 | 39.527 | 1.00 | 12.37 | B |
| ATOM | 2579 | C | ALA | B | 83 | 59.327 | 62.232 | 41.421 | 1.00 | 10.70 | B |
| ATOM | 2580 | O | ALA | B | 83 | 58.227 | 61.985 | 41.920 | 1.00 | 11.07 | B |
| ATOM | 2581 | N | ASP | B | 84 | 59.761 | 63.470 | 41.198 | 1.00 | 10.03 | B |
| ATOM | 2582 | CA | ASP | B | 84 | 58.968 | 64.643 | 41.560 | 1.00 | 9.72 | B |
| ATOM | 2583 | CB | ASP | B | 84 | 59.898 | 65.733 | 42.102 | 1.00 | 12.11 | B |
| ATOM | 2584 | CG | ASP | B | 84 | 60.716 | 65.272 | 43.284 | 1.00 | 14.44 | B |
| ATOM | 2585 | OD1 | ASP | B | 84 | 60.120 | 64.803 | 44.275 | 1.00 | 17.95 | B |
| ATOM | 2586 | OD2 | ASP | B | 84 | 61.958 | 65.384 | 43.221 | 1.00 | 15.95 | B |
| ATOM | 2587 | C | ASP | B | 84 | 58.118 | 65.171 | 40.418 | 1.00 | 10.31 | B |
| ATOM | 2588 | O | ASP | B | 84 | 57.076 | 65.793 | 40.643 | 1.00 | 10.64 | B |
| ATOM | 2589 | N | ILE | B | 85 | 58.563 | 64.936 | 39.191 | 1.00 | 9.52 | B |
| ATOM | 2590 | CA | ILE | B | 85 | 57.849 | 65.443 | 38.030 | 1.00 | 9.74 | B |
| ATOM | 2591 | CB | ILE | B | 85 | 58.257 | 66.925 | 37.781 | 1.00 | 11.00 | B |
| ATOM | 2592 | CG2 | ILE | B | 85 | 59.734 | 66.996 | 37.445 | 1.00 | 13.21 | B |
| ATOM | 2593 | CG1 | ILE | B | 85 | 57.403 | 67.561 | 36.683 | 1.00 | 11.45 | B |
| ATOM | 2594 | CD1 | ILE | B | 85 | 57.490 | 69.082 | 36.677 | 1.00 | 14.00 | B |
| ATOM | 2595 | C | ILE | B | 85 | 58.191 | 64.599 | 36.812 | 1.00 | 9.21 | B |
| ATOM | 2596 | O | ILE | B | 85 | 59.292 | 64.060 | 36.719 | 1.00 | 9.90 | B |
| ATOM | 2597 | N | ALA | B | 86 | 57.234 | 64.468 | 35.900 | 1.00 | 8.46 | B |
| ATOM | 2598 | CA | ALA | B | 86 | 57.446 | 63.718 | 34.664 | 1.00 | 8.54 | B |
| ATOM | 2599 | CB | ALA | B | 86 | 56.422 | 62.582 | 34.529 | 1.00 | 8.59 | B |
| ATOM | 2600 | C | ALA | B | 86 | 57.291 | 64.702 | 33.515 | 1.00 | 8.28 | B |
| ATOM | 2601 | O | ALA | B | 86 | 56.272 | 65.388 | 33.414 | 1.00 | 9.05 | B |
| ATOM | 2602 | N | ILE | B | 87 | 58.318 | 64.795 | 32.676 | 1.00 | 8.75 | B |
| ATOM | 2603 | CA | ILE | B | 87 | 58.300 | 65.686 | 31.520 | 1.00 | 9.37 | B |
| ATOM | 2604 | CB | ILE | B | 87 | 59.341 | 66.823 | 31.675 | 1.00 | 9.87 | B |
| ATOM | 2605 | CG2 | ILE | B | 87 | 59.286 | 67.753 | 30.470 | 1.00 | 11.56 | B |
| ATOM | 2606 | CG1 | ILE | B | 87 | 59.052 | 67.609 | 32.960 | 1.00 | 9.11 | B |
| ATOM | 2607 | CD1 | ILE | B | 87 | 60.022 | 68.736 | 33.238 | 1.00 | 11.51 | B |
| ATOM | 2608 | C | ILE | B | 87 | 58.612 | 64.810 | 30.311 | 1.00 | 9.80 | B |
| ATOM | 2609 | O | ILE | B | 87 | 59.768 | 64.458 | 30.053 | 1.00 | 9.23 | B |
| ATOM | 2610 | N | ALA | B | 88 | 57.563 | 64.462 | 29.572 | 1.00 | 9.10 | B |
| ATOM | 2611 | CA | ALA | B | 88 | 57.681 | 63.569 | 28.427 | 1.00 | 9.13 | B |
| ATOM | 2612 | CB | ALA | B | 88 | 57.847 | 62.140 | 28.934 | 1.00 | 11.65 | B |
| ATOM | 2613 | C | ALA | B | 88 | 56.408 | 63.667 | 27.594 | 1.00 | 8.94 | B |
| ATOM | 2614 | O | ALA | B | 88 | 55.499 | 64.424 | 27.930 | 1.00 | 8.74 | B |
| ATOM | 2615 | N | PRO | B | 89 | 56.336 | 62.920 | 26.480 | 1.00 | 8.93 | B |
| ATOM | 2616 | CD | PRO | B | 89 | 57.448 | 62.251 | 25.779 | 1.00 | 9.53 | B |
| ATOM | 2617 | CA | PRO | B | 89 | 55.127 | 62.960 | 25.649 | 1.00 | 9.58 | B |
| ATOM | 2618 | CB | PRO | B | 89 | 55.642 | 62.481 | 24.287 | 1.00 | 10.07 | B |
| ATOM | 2619 | CG | PRO | B | 89 | 56.712 | 61.472 | 24.635 | 1.00 | 9.31 | B |
| ATOM | 2620 | C | PRO | B | 89 | 54.080 | 62.072 | 26.339 | 1.00 | 8.71 | B |
| ATOM | 2621 | O | PRO | B | 89 | 53.659 | 61.038 | 25.812 | 1.00 | 9.89 | B |
| ATOM | 2622 | N | LEU | B | 90 | 53.682 | 62.494 | 27.536 | 1.00 | 8.48 | B |
| ATOM | 2623 | CA | LEU | B | 90 | 52.721 | 61.763 | 28.357 | 1.00 | 8.00 | B |
| ATOM | 2624 | CB | LEU | B | 90 | 53.138 | 61.902 | 29.822 | 1.00 | 8.24 | B |
| ATOM | 2625 | CG | LEU | B | 90 | 52.294 | 61.143 | 30.850 | 1.00 | 7.30 | B |
| ATOM | 2626 | CD1 | LEU | B | 90 | 52.467 | 59.638 | 30.677 | 1.00 | 8.93 | B |
| ATOM | 2627 | CD2 | LEU | B | 90 | 52.718 | 61.562 | 32.243 | 1.00 | 10.45 | B |
| ATOM | 2628 | C | LEU | B | 90 | 51.311 | 62.259 | 28.092 | 1.00 | 7.45 | B |
| ATOM | 2629 | O | LEU | B | 90 | 50.937 | 63.364 | 28.495 | 1.00 | 8.57 | B |
| ATOM | 2630 | N | THR | B | 91 | 50.519 | 61.427 | 27.433 | 1.00 | 7.15 | B |
| ATOM | 2631 | CA | THR | B | 91 | 49.161 | 61.794 | 27.089 | 1.00 | 8.89 | B |
| ATOM | 2632 | CB | THR | B | 91 | 48.597 | 60.775 | 26.103 | 1.00 | 9.01 | B |
| ATOM | 2633 | OG1 | THR | B | 91 | 49.485 | 60.701 | 24.981 | 1.00 | 9.51 | B |
| ATOM | 2634 | CG2 | THR | B | 91 | 47.210 | 61.182 | 25.620 | 1.00 | 10.88 | B |
| ATOM | 2635 | C | THR | B | 91 | 48.250 | 61.931 | 28.298 | 1.00 | 9.07 | B |
| ATOM | 2636 | O | THR | B | 91 | 48.247 | 61.096 | 29.204 | 1.00 | 8.09 | B |
| ATOM | 2637 | N | ILE | B | 92 | 47.489 | 63.016 | 28.312 | 1.00 | 9.29 | B |
| ATOM | 2638 | CA | ILE | B | 92 | 46.552 | 63.276 | 29.392 | 1.00 | 8.36 | B |
| ATOM | 2639 | CB | ILE | B | 92 | 46.109 | 64.758 | 29.386 | 1.00 | 8.94 | B |
| ATOM | 2640 | CG2 | ILE | B | 92 | 44.990 | 64.984 | 30.399 | 1.00 | 9.48 | B |
| ATOM | 2641 | CG1 | ILE | B | 92 | 47.314 | 65.654 | 29.682 | 1.00 | 9.04 | B |
| ATOM | 2642 | CD1 | ILE | B | 92 | 47.030 | 67.131 | 29.538 | 1.00 | 11.48 | B |
| ATOM | 2643 | C | ILE | B | 92 | 45.344 | 62.371 | 29.186 | 1.00 | 10.25 | B |
| ATOM | 2644 | O | ILE | B | 92 | 44.636 | 62.484 | 28.183 | 1.00 | 10.43 | B |
| ATOM | 2645 | N | THR | B | 93 | 45.136 | 61.447 | 30.118 | 1.00 | 9.09 | B |
| ATOM | 2646 | CA | THR | B | 93 | 44.001 | 60.532 | 30.039 | 1.00 | 10.64 | B |
| ATOM | 2647 | CB | THR | B | 93 | 44.434 | 59.090 | 29.707 | 1.00 | 10.99 | B |
| ATOM | 2648 | OG1 | THR | B | 93 | 45.168 | 58.543 | 30.810 | 1.00 | 12.28 | B |
| ATOM | 2649 | CG2 | THR | B | 93 | 45.305 | 59.065 | 28.457 | 1.00 | 11.50 | B |
| ATOM | 2650 | C | THR | B | 93 | 43.288 | 60.513 | 31.382 | 1.00 | 9.94 | B |
| ATOM | 2651 | O | THR | B | 93 | 43.864 | 60.876 | 32.407 | 1.00 | 10.71 | B |
| ATOM | 2652 | N | LEU | B | 94 | 42.034 | 60.075 | 31.368 | 1.00 | 10.44 | B |
| ATOM | 2653 | CA | LEU | B | 94 | 41.226 | 60.019 | 32.576 | 1.00 | 11.79 | B |
| ATOM | 2654 | CB | LEU | B | 94 | 39.814 | 59.549 | 32.215 | 1.00 | 12.07 | B |
| ATOM | 2655 | CG | LEU | B | 94 | 38.891 | 59.243 | 33.400 | 1.00 | 12.92 | B |
| ATOM | 2656 | CD1 | LEU | B | 94 | 38.623 | 60.506 | 34.198 | 1.00 | 14.90 | B |
| ATOM | 2657 | CD2 | LEU | B | 94 | 37.591 | 58.650 | 32.881 | 1.00 | 16.53 | B |
| ATOM | 2658 | C | LEU | B | 94 | 41.848 | 59.168 | 33.674 | 1.00 | 10.90 | B |
| ATOM | 2659 | O | LEU | B | 94 | 41.979 | 59.617 | 64.809 | 1.00 | 11.19 | B |
| ATOM | 2660 | N | VAL | B | 95 | 42.230 | 57.939 | 33.344 | 1.00 | 11.42 | B |
| ATOM | 2661 | CA | VAL | B | 95 | 42.791 | 57.059 | 34.360 | 1.00 | 12.28 | B |
| ATOM | 2662 | CB | VAL | B | 95 | 43.048 | 55.630 | 33.813 | 1.00 | 12.96 | B |
| ATOM | 2663 | CG1 | VAL | B | 95 | 41.729 | 54.989 | 33.409 | 1.00 | 16.92 | B |
| ATOM | 2664 | CG2 | VAL | B | 95 | 44.007 | 55.673 | 32.640 | 1.00 | 14.82 | B |
| ATOM | 2665 | C | VAL | B | 95 | 44.064 | 57.613 | 34.984 | 1.00 | 11.38 | B |
| ATOM | 2666 | O | VAL | B | 95 | 44.313 | 57.404 | 36.168 | 1.00 | 12.07 | B |
| ATOM | 2667 | N | ARG | B | 96 | 44.868 | 58.326 | 34.199 | 1.00 | 10.76 | B |
| ATOM | 2668 | CA | ARG | B | 96 | 46.097 | 58.910 | 34.730 | 1.00 | 10.29 | B |
| ATOM | 2669 | CB | ARG | B | 96 | 47.046 | 59.248 | 33.572 | 1.00 | 9.54 | B |
| ATOM | 2670 | CG | ARG | B | 96 | 47.619 | 58.041 | 32.856 | 1.00 | 10.00 | B |
| ATOM | 2671 | CD | ARG | B | 96 | 48.438 | 58.469 | 31.655 | 1.00 | 9.38 | B |
| ATOM | 2672 | NE | ARG | B | 96 | 49.028 | 57.323 | 30.974 | 1.00 | 9.28 | B |
| ATOM | 2673 | CZ | ARG | B | 96 | 49.552 | 57.376 | 29.757 | 1.00 | 8.53 | B |
| ATOM | 2674 | NH1 | ARG | B | 96 | 49.556 | 58.519 | 29.087 | 1.00 | 9.89 | B |
| ATOM | 2675 | NH2 | ARG | B | 96 | 50.077 | 56.290 | 29.210 | 1.00 | 9.10 | B |
| ATOM | 2676 | C | ARG | B | 96 | 45.761 | 60.121 | 35.598 | 1.00 | 9.41 | B |
| ATOM | 2677 | O | ARG | B | 96 | 46.335 | 60.308 | 36.668 | 1.00 | 10.66 | B |
| ATOM | 2678 | N | GLU | B | 97 | 44.825 | 60.939 | 35.124 | 1.00 | 9.82 | B |
| ATOM | 2679 | CA | GLU | B | 97 | 44.404 | 62.141 | 35.842 | 1.00 | 10.05 | B |
| ATOM | 2680 | CB | GLU | B | 97 | 43.397 | 62.910 | 34.981 | 1.00 | 10.93 | B |
| ATOM | 2681 | CG | GLU | B | 97 | 42.881 | 64.202 | 35.611 | 1.00 | 12.01 | B |
| ATOM | 2682 | CD | GLU | B | 97 | 43.933 | 65.292 | 35.683 | 1.00 | 11.95 | B |
| ATOM | 2683 | OE1 | GLU | B | 97 | 44.845 | 65.194 | 36.530 | 1.00 | 12.88 | B |
| ATOM | 2684 | OE2 | GLU | B | 97 | 43.846 | 66.250 | 34.885 | 1.00 | 14.79 | B |
| ATOM | 2685 | C | GLU | B | 97 | 43.841 | 61.791 | 37.213 | 1.00 | 11.65 | B |
| ATOM | 2686 | O | GLU | B | 97 | 43.883 | 62.602 | 38.138 | 1.00 | 11.62 | B |
| ATOM | 2687 | N | GLU | B | 98 | 43.305 | 60.583 | 37.335 | 1.00 | 10.80 | B |
| ATOM | 2688 | CA | GLU | B | 98 | 42.726 | 60.137 | 38.594 | 1.00 | 12.25 | B |
| ATOM | 2689 | CB | GLU | B | 98 | 41.868 | 58.892 | 38.334 | 1.00 | 13.12 | B |
| ATOM | 2690 | CG | GLU | B | 98 | 40.541 | 59.198 | 37.643 | 1.00 | 14.07 | B |
| ATOM | 2691 | CD | GLU | B | 98 | 39.787 | 57.953 | 37.208 | 1.00 | 15.79 | B |
| ATOM | 2692 | OE1 | GLU | B | 98 | 38.618 | 58.090 | 36.789 | 1.00 | 18.87 | B |
| ATOM | 2693 | OE2 | GLU | B | 98 | 40.356 | 56.841 | 37.269 | 1.00 | 17.43 | B |
| ATOM | 2694 | C | GLU | B | 98 | 43.775 | 59.913 | 39.676 | 1.00 | 12.93 | B |
| ATOM | 2695 | O | GLU | B | 98 | 43.468 | 60.027 | 40.866 | 1.00 | 13.27 | B |
| ATOM | 2696 | N | VAL | B | 99 | 45.012 | 59.617 | 39.282 | 1.00 | 11.95 | B |
| ATOM | 2697 | CA | VAL | B | 99 | 46.048 | 59.346 | 40.276 | 1.00 | 11.57 | B |
| ATOM | 2698 | CB | VAL | B | 99 | 46.627 | 57.925 | 40.091 | 1.00 | 12.29 | B |
| ATOM | 2699 | CG1 | VAL | B | 99 | 45.506 | 56.601 | 40.222 | 1.00 | 14.41 | B |
| ATOM | 2700 | CG2 | VAL | B | 99 | 47.318 | 57.800 | 38.736 | 1.00 | 11.21 | B |
| ATOM | 2701 | C | VAL | B | 99 | 47.198 | 60.345 | 40.404 | 1.00 | 11.85 | B |
| ATOM | 2702 | O | VAL | B | 99 | 47.912 | 60.334 | 41.402 | 1.00 | 11.93 | B |
| ATOM | 2703 | N | ILE | B | 100 | 47.394 | 61.195 | 39.401 | 1.00 | 10.79 | B |
| ATOM | 2704 | CA | ILE | B | 100 | 48.441 | 62.213 | 39.469 | 1.00 | 9.94 | B |
| ATOM | 2705 | CB | ILE | B | 100 | 49.712 | 61.820 | 38.640 | 1.00 | 9.74 | B |
| ATOM | 2706 | CG2 | ILE | B | 100 | 50.484 | 60.712 | 39.362 | 1.00 | 11.41 | B |
| ATOM | 2707 | CG1 | ILE | B | 100 | 49.336 | 61.392 | 37.217 | 1.00 | 10.34 | B |
| ATOM | 2708 | CD1 | ILE | B | 100 | 48.894 | 62.519 | 36.298 | 1.00 | 12.72 | B |
| ATOM | 2709 | C | ILE | B | 100 | 47.877 | 63.535 | 38.964 | 1.00 | 10.66 | B |
| ATOM | 2710 | O | ILE | B | 100 | 46.751 | 63.575 | 38.466 | 1.00 | 10.93 | B |
| ATOM | 2711 | N | ASP | B | 101 | 48.638 | 64.616 | 39.124 | 1.00 | 10.24 | B |
| ATOM | 2712 | CA | ASP | B | 101 | 48.210 | 65.934 | 38.650 | 1.00 | 11.76 | B |
| ATOM | 2713 | CB | ASP | B | 101 | 48.604 | 67.031 | 39.655 | 1.00 | 12.35 | B |
| ATOM | 2714 | CG | ASP | B | 101 | 47.795 | 66.994 | 40.940 | 1.00 | 11.56 | B |
| ATOM | 2715 | OD1 | ASP | B | 101 | 46.712 | 66.373 | 40.967 | 1.00 | 13.87 | B |
| ATOM | 2716 | OD2 | ASP | B | 101 | 48.255 | 67.614 | 41.925 | 1.00 | 14.17 | B |
| ATOM | 2717 | C | ASP | B | 101 | 48.845 | 66.195 | 37.285 | 1.00 | 10.88 | B |
| ATOM | 2718 | O | ASP | B | 101 | 50.017 | 65.872 | 37.068 | 1.00 | 13.39 | B |
| ATOM | 2719 | N | PHE | B | 102 | 48.070 | 66.775 | 36.371 | 1.00 | 10.57 | B |
| ATOM | 2720 | CA | PHE | B | 102 | 48.546 | 67.128 | 35.034 | 1.00 | 10.95 | B |
| ATOM | 2721 | CB | PHE | B | 102 | 47.659 | 66.498 | 33.956 | 1.00 | 10.59 | B |
| ATOM | 2722 | CG | PHE | B | 102 | 48.068 | 65.123 | 33.520 | 1.00 | 10.43 | B |
| ATOM | 2723 | CD1 | PHE | B | 102 | 49.236 | 64.916 | 32.991 | 1.00 | 10.86 | B |
| ATOM | 2724 | CD2 | PHE | B | 102 | 47.214 | 64.157 | 33.603 | 1.00 | 11.54 | B |
| ATOM | 2725 | CE1 | PHE | B | 102 | 49.609 | 63.664 | 32.488 | 1.00 | 10.96 | B |
| ATOM | 2726 | CE2 | PHE | B | 102 | 47.490 | 62.875 | 33.132 | 1.00 | 12.28 | B |
| ATOM | 2727 | CZ | PHE | B | 102 | 48.712 | 62.626 | 32.562 | 1.00 | 10.89 | B |
| ATOM | 2728 | C | PHE | B | 102 | 48.503 | 68.638 | 34.881 | 1.00 | 11.31 | B |
| ATOM | 2729 | O | PHE | B | 102 | 47.583 | 69.294 | 35.373 | 1.00 | 11.38 | B |
| ATOM | 2730 | N | SER | B | 103 | 49.488 | 69.185 | 34.182 | 1.00 | 10.15 | B |
| ATOM | 2731 | CA | SER | B | 103 | 49.504 | 70.606 | 33.882 | 1.00 | 10.78 | B |
| ATOM | 2732 | CB | SER | B | 103 | 50.916 | 71.025 | 33.479 | 1.00 | 10.95 | B |
| ATOM | 2733 | OG | SER | B | 103 | 51.238 | 70.473 | 32.201 | 1.00 | 11.20 | B |
| ATOM | 2734 | C | SER | B | 103 | 48.559 | 70.713 | 32.694 | 1.00 | 11.47 | B |
| ATOM | 2735 | O | SER | B | 103 | 48.115 | 69.701 | 32.145 | 1.00 | 11.65 | B |
| ATOM | 2736 | N | LYS | B | 104 | 48.244 | 71.941 | 32.298 | 1.00 | 12.25 | B |
| ATOM | 2737 | CA | LYS | B | 104 | 47.439 | 72.141 | 31.111 | 1.00 | 12.94 | B |
| ATOM | 2738 | CB | LYS | B | 104 | 47.125 | 73.633 | 30.960 | 1.00 | 14.44 | B |
| ATOM | 2739 | CG | LYS | B | 104 | 46.200 | 74.190 | 32.033 | 1.00 | 16.62 | B |
| ATOM | 2740 | CD | LYS | B | 104 | 44.876 | 73.439 | 32.048 | 1.00 | 15.36 | B |
| ATOM | 2741 | CE | LYS | B | 104 | 43.868 | 74.061 | 33.019 | 1.00 | 15.44 | B |
| ATOM | 2742 | NZ | LYS | B | 104 | 44.262 | 73.945 | 34.457 | 1.00 | 15.24 | B |
| ATOM | 2743 | C | LYS | B | 104 | 48.357 | 71.583 | 30.015 | 1.00 | 11.94 | B |
| ATOM | 2744 | O | LYS | B | 104 | 49.576 | 71.535 | 30.192 | 1.00 | 11.20 | B |
| ATOM | 2745 | N | PRO | B | 105 | 47.796 | 71.169 | 28.872 | 1.00 | 11.15 | B |
| ATOM | 2746 | CD | PRO | B | 105 | 46.369 | 71.191 | 28.518 | 1.00 | 13.19 | B |
| ATOM | 2747 | CA | PRO | B | 105 | 48.604 | 70.611 | 27.777 | 1.00 | 11.83 | B |
| ATOM | 2748 | CB | PRO | B | 105 | 47.554 | 70.259 | 26.718 | 1.00 | 14.56 | B |
| ATOM | 2749 | CG | PRO | B | 105 | 46.275 | 70.014 | 27.488 | 1.00 | 16.32 | B |
| ATOM | 2750 | C | PRO | B | 105 | 49.698 | 71.534 | 27.261 | 1.00 | 9.71 | B |
| ATOM | 2751 | O | PRO | B | 105 | 49.480 | 72.734 | 27.112 | 1.00 | 10.01 | B |
| ATOM | 2752 | N | PHE | B | 106 | 50.872 | 70.969 | 26.978 | 1.00 | 9.28 | B |
| ATOM | 2753 | CA | PHE | B | 106 | 51.961 | 71.764 | 26.423 | 1.00 | 9.25 | B |
| ATOM | 2754 | CB | PHE | B | 106 | 53.271 | 71.499 | 27.189 | 1.00 | 8.75 | B |
| ATOM | 2755 | CG | PHE | B | 106 | 53.781 | 70.080 | 27.104 | 1.00 | 8.48 | B |
| ATOM | 2756 | CD1 | PHE | B | 106 | 54.389 | 69.661 | 26.034 | 1.00 | 8.53 | B |
| ATOM | 2757 | CD2 | PHE | B | 106 | 53.661 | 69.290 | 28.130 | 1.00 | 8.74 | B |
| ATOM | 2758 | CE1 | PHE | B | 106 | 54.941 | 68.365 | 25.944 | 1.00 | 8.18 | B |
| ATOM | 2759 | CE2 | PHE | B | 106 | 54.176 | 67.980 | 28.138 | 1.00 | 9.15 | B |
| ATOM | 2760 | CZ | PHE | B | 106 | 54.830 | 67.513 | 27.021 | 1.00 | 8.81 | B |
| ATOM | 2761 | C | PHE | B | 106 | 52.060 | 71.513 | 24.920 | 1.00 | 8.84 | B |
| ATOM | 2762 | O | PHE | B | 106 | 52.751 | 72.238 | 24.207 | 1.00 | 10.20 | B |
| ATOM | 2763 | N | MET | B | 107 | 51.349 | 70.499 | 24.437 | 1.00 | 8.41 | B |
| ATOM | 2764 | CA | MET | B | 107 | 51.357 | 70.156 | 23.019 | 1.00 | 9.63 | B |
| ATOM | 2765 | CB | MET | B | 107 | 52.612 | 69.329 | 22.702 | 1.00 | 12.06 | B |
| ATOM | 2766 | CG | MET | B | 107 | 52.735 | 68.894 | 21.240 | 1.00 | 13.02 | B |
| ATOM | 2767 | SD | MET | B | 107 | 54.220 | 67.912 | 20.886 | 1.00 | 14.69 | B |
| ATOM | 2768 | CE | MET | B | 107 | 55.453 | 69.181 | 20.827 | 1.00 | 16.10 | B |
| ATOM | 2769 | C | MET | B | 107 | 50.074 | 69.407 | 22.689 | 1.00 | 9.16 | B |
| ATOM | 2770 | O | MET | B | 107 | 49.612 | 68.580 | 23.477 | 1.00 | 8.38 | B |
| ATOM | 2771 | N | SER | B | 108 | 49.509 | 69.688 | 21.522 | 1.00 | 9.03 | B |
| ATOM | 2772 | CA | SER | B | 108 | 48.289 | 69.020 | 21.085 | 1.00 | 11.10 | B |
| ATOM | 2773 | CB | SER | B | 108 | 47.425 | 70.011 | 20.314 | 1.00 | 13.63 | B |
| ATOM | 2774 | OG | SER | B | 108 | 48.083 | 70.392 | 19.120 | 1.00 | 14.86 | B |
| ATOM | 2775 | C | SER | B | 108 | 48.640 | 67.805 | 20.234 | 1.00 | 10.23 | B |
| ATOM | 2776 | O | SER | B | 108 | 49.702 | 67.744 | 19.614 | 1.00 | 10.94 | B |
| ATOM | 2777 | N | LEU | B | 109 | 47.739 | 66.832 | 20.215 | 1.00 | 11.38 | B |
| ATOM | 2778 | CA | LEU | B | 109 | 47.932 | 65.645 | 19.397 | 1.00 | 10.10 | B |
| ATOM | 2779 | CB | LEU | B | 109 | 49.071 | 64.789 | 19.953 | 1.00 | 11.95 | B |
| ATOM | 2780 | CG | LEU | B | 109 | 48.706 | 63.945 | 21.173 | 1.00 | 15.12 | B |
| ATOM | 2781 | CD1 | LEU | B | 109 | 49.597 | 62.739 | 21.219 | 1.00 | 19.71 | B |
| ATOM | 2782 | CD2 | LEU | B | 109 | 48.824 | 64.759 | 22.437 | 1.00 | 14.94 | B |
| ATOM | 2783 | C | LEU | B | 109 | 46.631 | 64.872 | 19.325 | 1.00 | 9.73 | B |
| ATOM | 2784 | O | LEU | B | 109 | 45.696 | 65.114 | 20.088 | 1.00 | 10.33 | B |
| ATOM | 2785 | N | GLY | B | 110 | 46.593 | 63.932 | 18.393 | 1.00 | 9.90 | B |
| ATOM | 2786 | CA | GLY | B | 110 | 45.441 | 63.075 | 18.220 | 1.00 | 9.38 | B |
| ATOM | 2787 | C | GLY | B | 110 | 45.945 | 61.804 | 17.577 | 1.00 | 9.48 | B |
| ATOM | 2788 | O | GLY | B | 110 | 46.981 | 61.824 | 16.912 | 1.00 | 9.14 | B |
| ATOM | 2789 | N | ILE | B | 111 | 45.246 | 60.694 | 17.787 | 1.00 | 9.65 | B |
| ATOM | 2790 | CA | ILE | B | 111 | 45.655 | 59.443 | 17.166 | 1.00 | 9.35 | B |
| ATOM | 2791 | CB | ILE | B | 111 | 44.767 | 58.279 | 17.643 | 1.00 | 9.94 | B |
| ATOM | 2792 | CG2 | ILE | B | 111 | 45.057 | 57.020 | 16.831 | 1.00 | 10.66 | B |
| ATOM | 2793 | CG1 | ILE | B | 111 | 45.028 | 58.033 | 19.130 | 1.00 | 10.22 | B |
| ATOM | 2794 | CD1 | ILE | B | 111 | 44.019 | 57.120 | 19.792 | 1.00 | 10.21 | B |
| ATOM | 2795 | C | ILE | B | 111 | 45.512 | 59.671 | 15.666 | 1.00 | 9.48 | B |
| ATOM | 2796 | O | ILE | B | 111 | 44.561 | 60.313 | 15.212 | 1.00 | 9.88 | B |
| ATOM | 2797 | N | SER | B | 112 | 46.475 | 59.164 | 14.905 | 1.00 | 9.02 | B |
| ATOM | 2798 | CA | SER | B | 112 | 46.490 | 59.360 | 13.462 | 1.00 | 9.29 | B |
| ATOM | 2799 | CB | SER | B | 112 | 47.377 | 60.566 | 13.145 | 1.00 | 9.53 | B |
| ATOM | 2800 | OG | SER | B | 112 | 48.730 | 60.281 | 13.466 | 1.00 | 10.83 | B |
| ATOM | 2801 | C | SER | B | 112 | 46.965 | 58.115 | 12.745 | 1.00 | 9.10 | B |
| ATOM | 2802 | O | SER | B | 112 | 47.467 | 57.177 | 13.360 | 1.00 | 9.87 | B |
| ATOM | 2803 | N | ILE | B | 113 | 46.831 | 58.143 | 11.425 | 1.00 | 9.61 | B |
| ATOM | 2804 | CA | ILE | B | 113 | 47.237 | 57.036 | 10.573 | 1.00 | 10.86 | B |
| ATOM | 2805 | CB | ILE | B | 113 | 46.128 | 56.682 | 9.556 | 1.00 | 10.47 | B |
| ATOM | 2806 | CG2 | ILE | B | 113 | 46.583 | 55.521 | 8.678 | 1.00 | 12.50 | B |
| ATOM | 2807 | CG1 | ILE | B | 113 | 44.829 | 56.346 | 10.290 | 1.00 | 10.99 | B |
| ATOM | 2808 | CD1 | ILE | B | 113 | 43.621 | 56.260 | 9.385 | 1.00 | 11.97 | B |
| ATOM | 2809 | C | ILE | B | 113 | 48.500 | 57.360 | 9.779 | 1.00 | 9.78 | B |
| ATOM | 2810 | O | ILE | B | 113 | 48.582 | 58.403 | 9.126 | 1.00 | 10.59 | B |
| ATOM | 2811 | N | MET | B | 114 | 49.483 | 56.468 | 9.852 | 1.00 | 10.31 | B |
| ATOM | 2812 | CA | MET | B | 114 | 50.712 | 56.611 | 9.079 | 1.00 | 10.19 | B |
| ATOM | 2813 | CB | MET | B | 114 | 51.952 | 56.472 | 9.968 | 1.00 | 10.87 | B |
| ATOM | 2814 | CG | MET | B | 114 | 53.248 | 56.420 | 9.141 | 1.00 | 10.87 | B |
| ATOM | 2815 | SD | MET | B | 114 | 54.716 | 55.935 | 10.065 | 1.00 | 12.20 | B |
| ATOM | 2816 | CE | MET | B | 114 | 54.960 | 57.401 | 11.082 | 1.00 | 13.18 | B |
| ATOM | 2817 | C | MET | B | 114 | 50.679 | 55.522 | 8.021 | 1.00 | 11.79 | B |
| ATOM | 2818 | O | MET | B | 114 | 50.500 | 54.345 | 8.341 | 1.00 | 10.71 | B |
| ATOM | 2819 | N | ILE | B | 115 | 50.848 | 55.916 | 6.764 | 1.00 | 11.46 | B |
| ATOM | 2820 | CA | ILE | B | 115 | 50.869 | 54.964 | 5.664 | 1.00 | 11.48 | B |
| ATOM | 2821 | CB | ILE | B | 115 | 49.708 | 55.182 | 4.658 | 1.00 | 12.30 | B |
| ATOM | 2822 | CG2 | ILE | B | 115 | 48.376 | 54.889 | 5.317 | 1.00 | 12.90 | B |
| ATOM | 2823 | CG1 | ILE | B | 115 | 49.768 | 56.606 | 4.090 | 1.00 | 13.44 | B |
| ATOM | 2824 | CD1 | ILE | B | 115 | 48.838 | 56.840 | 2.906 | 1.00 | 17.65 | B |
| ATOM | 2825 | C | ILE | B | 115 | 52.150 | 55.147 | 4.873 | 1.00 | 11.42 | B |
| ATOM | 2826 | O | ILE | B | 115 | 52.814 | 56.183 | 4.966 | 1.00 | 12.08 | B |
| ATOM | 2827 | N | LYS | B | 116 | 52.502 | 54.125 | 4.104 | 1.00 | 13.24 | B |
| ATOM | 2828 | CA | LYS | B | 116 | 53.655 | 54.213 | 3.227 | 1.00 | 10.86 | B |
| ATOM | 2829 | CB | LYS | B | 116 | 54.161 | 52.812 | 2.879 | 1.00 | 12.45 | B |
| ATOM | 2830 | CG | LYS | B | 116 | 55.410 | 52.821 | 2.009 | 1.00 | 13.34 | B |
| ATOM | 2831 | CD | LYS | B | 116 | 55.911 | 51.416 | 1.723 | 1.00 | 16.54 | B |
| ATOM | 2832 | CE | LYS | B | 116 | 57.216 | 51.446 | 0.938 | 1.00 | 18.02 | B |
| ATOM | 2833 | NZ | LYS | B | 116 | 57.032 | 51.944 | −0.453 | 1.00 | 19.21 | B |
| ATOM | 2834 | C | LYS | B | 116 | 53.041 | 54.945 | 2.040 | 1.00 | 11.09 | B |
| ATOM | 2835 | O | LYS | B | 116 | 51.917 | 54.638 | 1.644 | 1.00 | 11.68 | B |
| ATOM | 2836 | N | LYS | B | 117 | 53.755 | 55.914 | 1.482 | 1.00 | 11.23 | B |
| ATOM | 2837 | CA | LYS | B | 117 | 53.224 | 56.665 | 0.349 | 1.00 | 11.74 | B |
| ATOM | 2838 | CB | LYS | B | 117 | 54.313 | 57.586 | −0.209 | 1.00 | 12.60 | B |
| ATOM | 2839 | CG | LYS | B | 117 | 54.610 | 58.789 | 0.672 | 1.00 | 13.79 | B |
| ATOM | 2840 | CD | LYS | B | 117 | 55.636 | 59.715 | 0.031 | 1.00 | 13.40 | B |
| ATOM | 2841 | CE | LYS | B | 117 | 55.903 | 60.935 | 0.904 | 1.00 | 15.47 | B |
| ATOM | 2842 | NZ | LYS | B | 117 | 56.912 | 61.846 | 0.294 | 1.00 | 15.56 | B |
| ATOM | 2843 | C | LYS | B | 117 | 52.633 | 55.741 | −0.705 | 1.00 | 11.47 | B |
| ATOM | 2844 | O | LYS | B | 117 | 53.233 | 54.725 | −1.067 | 1.00 | 11.50 | B |
| ATOM | 2845 | N | GLY | B | 118 | 51.447 | 56.095 | −1.187 | 1.00 | 12.24 | B |
| ATOM | 2846 | CA | GLY | B | 118 | 50.801 | 55.294 | −2.212 | 1.00 | 13.17 | B |
| ATOM | 2847 | C | GLY | B | 118 | 49.736 | 54.336 | −1.709 | 1.00 | 14.01 | B |
| ATOM | 2848 | O | GLY | B | 118 | 48.956 | 53.802 | −2.499 | 1.00 | 15.21 | B |
| ATOM | 2849 | N | THR | B | 119 | 49.698 | 54.108 | −0.400 | 1.00 | 13.30 | B |
| ATOM | 2850 | CA | THR | B | 119 | 48.702 | 53.210 | 0.176 | 1.00 | 13.36 | B |
| ATOM | 2851 | CB | THR | B | 119 | 48.958 | 52.999 | 1.676 | 1.00 | 13.62 | B |
| ATOM | 2852 | OG1 | THR | B | 119 | 50.257 | 52.426 | 1.854 | 1.00 | 13.79 | B |
| ATOM | 2853 | CG2 | THR | B | 119 | 47.920 | 52.059 | 2.272 | 1.00 | 14.41 | B |
| ATOM | 2854 | C | THR | B | 119 | 47.308 | 53.801 | −0.022 | 1.00 | 13.25 | B |
| ATOM | 2855 | O | THR | B | 119 | 47.061 | 54.957 | 0.315 | 1.00 | 13.63 | B |
| ATOM | 2856 | N | PRO | B | 120 | 46.376 | 53.008 | −0.578 | 1.00 | 15.18 | B |
| ATOM | 2857 | CD | PRO | B | 120 | 46.588 | 51.676 | −1.171 | 1.00 | 14.18 | B |
| ATOM | 2858 | CA | PRO | B | 120 | 45.008 | 53.472 | −0.820 | 1.00 | 15.67 | B |
| ATOM | 2859 | CB | PRO | B | 120 | 44.527 | 52.508 | −1.904 | 1.00 | 16.05 | B |
| ATOM | 2860 | CG | PRO | B | 120 | 45.117 | 51.200 | −1.471 | 1.00 | 16.26 | B |
| ATOM | 2861 | C | PRO | B | 120 | 44.141 | 53.538 | 0.435 | 1.00 | 15.82 | B |
| ATOM | 2862 | O | PRO | B | 120 | 43.130 | 52.843 | 0.541 | 1.00 | 17.43 | B |
| ATOM | 2863 | N | ILE | B | 121 | 44.544 | 54.391 | 1.372 | 1.00 | 15.76 | B |
| ATOM | 2864 | CA | ILE | B | 121 | 43.828 | 54.587 | 2.632 | 1.00 | 14.92 | B |
| ATOM | 2865 | CB | ILE | B | 121 | 44.529 | 53.848 | 3.796 | 1.00 | 14.97 | B |
| ATOM | 2866 | CG2 | ILE | B | 121 | 43.962 | 54.318 | 5.131 | 1.00 | 12.68 | B |
| ATOM | 2867 | CG1 | ILE | B | 121 | 44.370 | 52.334 | 3.634 | 1.00 | 14.33 | B |
| ATOM | 2868 | CD1 | ILE | B | 121 | 42.945 | 51.832 | 3.803 | 1.00 | 15.40 | B |
| ATOM | 2869 | C | ILE | B | 121 | 43.800 | 56.082 | 2.945 | 1.00 | 15.76 | B |
| ATOM | 2870 | O | ILE | B | 121 | 44.832 | 56.750 | 2.885 | 1.00 | 16.49 | B |
| ATOM | 2871 | N | GLU | B | 122 | 42.625 | 56.604 | 3.284 | 1.00 | 18.35 | B |
| ATOM | 2872 | CA | GLU | B | 122 | 42.489 | 58.025 | 3.581 | 1.00 | 20.17 | B |
| ATOM | 2873 | CB | GLU | B | 122 | 41.780 | 58.710 | 2.406 | 1.00 | 23.24 | B |
| ATOM | 2874 | CG | GLU | B | 122 | 42.475 | 58.553 | 1.066 | 1.00 | 29.97 | B |
| ATOM | 2875 | CD | GLU | B | 122 | 41.732 | 59.255 | −0.054 | 1.00 | 34.16 | B |
| ATOM | 2876 | OE1 | GLU | B | 122 | 40.546 | 58.930 | −0.277 | 1.00 | 37.06 | B |
| ATOM | 2877 | OE2 | GLU | B | 122 | 42.332 | 60.131 | −0.712 | 1.00 | 37.63 | B |
| ATOM | 2878 | C | GLU | B | 122 | 41.787 | 58.303 | 4.905 | 1.00 | 19.62 | B |
| ATOM | 2879 | O | GLU | B | 122 | 41.633 | 59.463 | 5.287 | 1.00 | 20.59 | B |
| ATOM | 2880 | N | SER | B | 123 | 41.362 | 57.257 | 5.609 | 1.00 | 17.88 | B |
| ATOM | 2881 | CA | SER | B | 123 | 40.658 | 57.453 | 6.874 | 1.00 | 16.36 | B |
| ATOM | 2882 | CB | SER | B | 123 | 39.255 | 57.981 | 6.575 | 1.00 | 16.51 | B |
| ATOM | 2883 | OG | SER | B | 123 | 38.469 | 56.965 | 5.970 | 1.00 | 19.10 | B |
| ATOM | 2884 | C | SER | B | 123 | 40.604 | 56.182 | 7.707 | 1.00 | 14.92 | B |
| ATOM | 2885 | O | SER | B | 123 | 40.928 | 55.096 | 7.230 | 1.00 | 13.22 | B |
| ATOM | 2886 | N | ALA | B | 124 | 40.183 | 56.330 | 8.960 | 1.00 | 14.58 | B |
| ATOM | 2887 | CA | ALA | B | 124 | 40.047 | 55.186 | 9.852 | 1.00 | 14.74 | B |
| ATOM | 2888 | CB | ALA | B | 124 | 39.681 | 55.653 | 11.253 | 1.00 | 14.54 | B |
| ATOM | 2889 | C | ALA | B | 124 | 38.948 | 54.287 | 9.289 | 1.00 | 14.66 | B |
| ATOM | 2890 | O | ALA | B | 124 | 39.046 | 53.063 | 9.342 | 1.00 | 15.39 | B |
| ATOM | 2891 | N | ALA | B | 125 | 37.900 | 54.903 | 8.749 | 1.00 | 14.87 | B |
| ATOM | 2892 | CA | ALA | B | 125 | 36.803 | 54.146 | 8.160 | 1.00 | 14.63 | B |
| ATOM | 2893 | CB | ALA | B | 125 | 35.747 | 55.092 | 7.603 | 1.00 | 16.21 | B |
| ATOM | 2894 | C | ALA | B | 125 | 37.353 | 53.249 | 7.053 | 1.00 | 14.66 | B |
| ATOM | 2895 | O | ALA | B | 125 | 36.984 | 52.077 | 6.951 | 1.00 | 15.81 | B |
| ATOM | 2896 | N | ASP | B | 126 | 38.239 | 53.798 | 6.224 | 1.00 | 15.21 | B |
| ATOM | 2897 | CA | ASP | B | 126 | 38.841 | 53.022 | 5.142 | 1.00 | 14.84 | B |
| ATOM | 2898 | CB | ASP | B | 126 | 39.836 | 53.890 | 4.359 | 1.00 | 16.41 | B |
| ATOM | 2899 | CG | ASP | B | 126 | 39.157 | 54.897 | 3.449 | 1.00 | 19.30 | B |
| ATOM | 2900 | OD1 | ASP | B | 126 | 37.951 | 54.740 | 3.157 | 1.00 | 19.38 | B |
| ATOM | 2901 | OD2 | ASP | B | 126 | 39.845 | 55.841 | 3.012 | 1.00 | 20.31 | B |
| ATOM | 2902 | C | ASP | B | 126 | 39.502 | 51.767 | 5.707 | 1.00 | 14.18 | B |
| ATOM | 2903 | O | ASP | B | 126 | 39.317 | 50.667 | 5.188 | 1.00 | 16.38 | B |
| ATOM | 2904 | N | LEU | B | 127 | 40.283 | 51.939 | 6.768 | 1.00 | 14.10 | B |
| ATOM | 2905 | CA | LEU | B | 127 | 40.971 | 50.816 | 7.392 | 1.00 | 14.07 | B |
| ATOM | 2906 | CB | LEU | B | 127 | 41.920 | 51.336 | 8.478 | 1.00 | 13.27 | B |
| ATOM | 2907 | CG | LEU | B | 127 | 43.217 | 52.037 | 8.074 | 1.00 | 13.45 | B |
| ATOM | 2908 | CD1 | LEU | B | 127 | 43.886 | 52.575 | 9.329 | 1.00 | 13.91 | B |
| ATOM | 2909 | CD2 | LEU | B | 127 | 44.141 | 51.071 | 7.345 | 1.00 | 12.03 | B |
| ATOM | 2910 | C | LEU | B | 127 | 40.001 | 49.782 | 7.944 | 1.00 | 14.19 | B |
| ATOM | 2911 | O | LEU | B | 127 | 40.156 | 48.588 | 7.704 | 1.00 | 14.70 | B |
| ATOM | 2912 | N | SER | B | 128 | 39.005 | 50.250 | 8.689 | 1.00 | 15.92 | B |
| ATOM | 2913 | CA | SER | B | 128 | 38.028 | 49.369 | 9.320 | 1.00 | 16.44 | B |
| ATOM | 2914 | CB | SER | B | 128 | 37.075 | 50.218 | 10.169 | 1.00 | 16.30 | B |
| ATOM | 2915 | OG | SER | B | 128 | 36.163 | 50.936 | 9.353 | 1.00 | 17.54 | B |
| ATOM | 2916 | C | SER | B | 128 | 37.275 | 48.459 | 8.359 | 1.00 | 17.64 | B |
| ATOM | 2917 | O | SER | B | 128 | 36.909 | 47.340 | 8.723 | 1.00 | 17.84 | B |
| ATOM | 2918 | N | LYS | B | 129 | 37.045 | 48.933 | 7.140 | 1.00 | 18.64 | B |
| ATOM | 2919 | CA | LYS | B | 129 | 36.291 | 48.172 | 6.147 | 1.00 | 21.45 | B |
| ATOM | 2920 | CB | LYS | B | 129 | 35.591 | 49.161 | 5.210 | 1.00 | 23.01 | B |
| ATOM | 2921 | CG | LYS | B | 129 | 34.696 | 50.170 | 5.905 | 1.00 | 26.51 | B |
| ATOM | 2922 | CD | LYS | B | 129 | 34.148 | 51.180 | 4.909 | 1.00 | 30.10 | B |
| ATOM | 2923 | CE | LYS | B | 129 | 33.342 | 52.266 | 5.602 | 1.00 | 32.04 | B |
| ATOM | 2924 | NZ | LYS | B | 129 | 32.858 | 53.291 | 4.635 | 1.00 | 32.54 | B |
| ATOM | 2925 | C | LYS | B | 129 | 37.096 | 47.135 | 5.374 | 1.00 | 22.41 | B |
| ATOM | 2926 | O | LYS | B | 129 | 36.567 | 46.508 | 4.456 | 1.00 | 24.11 | B |
| ATOM | 2927 | N | GLN | B | 130 | 38.361 | 46.938 | 5.731 | 1.00 | 21.03 | B |
| ATOM | 2928 | CA | GLN | B | 130 | 39.176 | 45.976 | 5.000 | 1.00 | 18.74 | B |
| ATOM | 2929 | CB | GLN | B | 130 | 39.796 | 46.699 | 3.801 | 1.00 | 18.95 | B |
| ATOM | 2930 | CG | GLN | B | 130 | 40.709 | 47.855 | 4.180 | 1.00 | 17.18 | B |
| ATOM | 2931 | CD | GLN | B | 130 | 42.095 | 47.392 | 4.572 | 1.00 | 16.68 | B |
| ATOM | 2932 | OE1 | GLN | B | 130 | 42.830 | 46.846 | 3.753 | 1.00 | 15.68 | B |
| ATOM | 2933 | NE2 | GLN | B | 130 | 42.459 | 47.605 | 5.831 | 1.00 | 14.42 | B |
| ATOM | 2934 | C | GLN | B | 130 | 40.203 | 45.264 | 5.873 | 1.00 | 17.60 | B |
| ATOM | 2935 | O | GLN | B | 130 | 40.355 | 45.572 | 7.057 | 1.00 | 18.74 | B |
| ATOM | 2936 | N | THR | B | 131 | 40.908 | 44.309 | 5.269 | 1.00 | 18.69 | B |
| ATOM | 2937 | CA | THR | B | 131 | 41.926 | 43.533 | 5.968 | 1.00 | 18.81 | B |
| ATOM | 2938 | CB | THR | B | 131 | 41.385 | 42.152 | 6.393 | 1.00 | 19.46 | B |
| ATOM | 2939 | OG1 | THR | B | 131 | 40.921 | 41.444 | 5.237 | 1.00 | 19.99 | B |
| ATOM | 2940 | CG2 | THR | B | 131 | 40.239 | 42.310 | 7.384 | 1.00 | 18.92 | B |
| ATOM | 2941 | C | THR | B | 131 | 43.178 | 43.317 | 5.118 | 1.00 | 19.13 | B |
| ATOM | 2942 | O | THR | B | 131 | 44.192 | 42.835 | 5.619 | 1.00 | 19.35 | B |
| ATOM | 2943 | N | GLU | B | 132 | 43.101 | 43.667 | 3.836 | 1.00 | 19.48 | B |
| ATOM | 2944 | CA | GLU | B | 132 | 44.236 | 43.516 | 2.923 | 1.00 | 19.54 | B |
| ATOM | 2945 | CB | GLU | B | 132 | 43.817 | 43.982 | 1.524 | 1.00 | 21.53 | B |
| ATOM | 2946 | CG | GLU | B | 132 | 44.962 | 44.077 | 0.523 | 1.00 | 29.35 | B |
| ATOM | 2947 | CD | GLU | B | 132 | 45.722 | 42.774 | 0.370 | 1.00 | 33.86 | B |
| ATOM | 2948 | OE1 | GLU | B | 132 | 45.088 | 41.748 | 0.047 | 1.00 | 36.49 | B |
| ATOM | 2949 | OE2 | GLU | B | 132 | 46.956 | 42.776 | 0.570 | 1.00 | 37.14 | B |
| ATOM | 2950 | C | GLU | B | 132 | 45.430 | 44.285 | 3.476 | 1.00 | 17.61 | B |
| ATOM | 2951 | O | GLU | B | 132 | 46.560 | 43.791 | 3.494 | 1.00 | 17.21 | B |
| ATOM | 2952 | N | ILE | B | 133 | 45.166 | 45.508 | 3.913 | 1.00 | 15.23 | B |
| ATOM | 2953 | CA | ILE | B | 133 | 46.191 | 46.355 | 4.493 | 1.00 | 14.15 | B |
| ATOM | 2954 | CB | ILE | B | 133 | 45.970 | 47.829 | 4.103 | 1.00 | 14.40 | B |
| ATOM | 2955 | CG2 | ILE | B | 133 | 47.019 | 48.710 | 4.762 | 1.00 | 14.60 | B |
| ATOM | 2956 | CG1 | ILE | B | 133 | 46.020 | 47.970 | 2.580 | 1.00 | 16.04 | B |
| ATOM | 2957 | CD1 | ILE | B | 133 | 45.624 | 49.339 | 2.071 | 1.00 | 16.35 | B |
| ATOM | 2958 | C | ILE | B | 133 | 46.052 | 46.193 | 6.002 | 1.00 | 12.86 | B |
| ATOM | 2959 | O | ILE | B | 133 | 45.075 | 46.649 | 6.596 | 1.00 | 14.16 | B |
| ATOM | 2960 | N | ALA | B | 134 | 47.014 | 45.515 | 6.614 | 1.00 | 11.59 | B |
| ATOM | 2961 | CA | ALA | B | 134 | 46.981 | 45.294 | 8.055 | 1.00 | 12.05 | B |
| ATOM | 2962 | CB | ALA | B | 134 | 48.019 | 44.256 | 8.447 | 1.00 | 11.73 | B |
| ATOM | 2963 | C | ALA | B | 134 | 47.262 | 46.608 | 8.762 | 1.00 | 11.57 | B |
| ATOM | 2964 | O | ALA | B | 134 | 47.892 | 47.500 | 8.195 | 1.00 | 11.94 | B |
| ATOM | 2965 | N | TYR | B | 135 | 46.799 | 46.734 | 10.000 | 1.00 | 11.30 | B |
| ATOM | 2966 | CA | TYR | B | 135 | 47.032 | 47.959 | 10.750 | 1.00 | 10.32 | B |
| ATOM | 2967 | CB | TYR | B | 135 | 45.987 | 49.012 | 10.353 | 1.00 | 10.24 | B |
| ATOM | 2968 | CG | TYR | B | 135 | 44.547 | 48.604 | 10.580 | 1.00 | 10.46 | B |
| ATOM | 2969 | CD1 | TYR | B | 135 | 43.901 | 48.892 | 11.782 | 1.00 | 12.70 | B |
| ATOM | 2970 | CE1 | TYR | B | 135 | 42.569 | 48.538 | 11.985 | 1.00 | 11.96 | B |
| ATOM | 2971 | CD2 | TYR | B | 135 | 43.825 | 47.947 | 9.585 | 1.00 | 11.79 | B |
| ATOM | 2972 | CE2 | TYR | B | 135 | 42.493 | 47.588 | 9.779 | 1.00 | 11.82 | B |
| ATOM | 2973 | CZ | TYR | B | 135 | 41.873 | 47.889 | 10.979 | 1.00 | 11.81 | B |
| ATOM | 2974 | OH | TYR | B | 135 | 40.556 | 47.545 | 11.174 | 1.00 | 11.82 | B |
| ATOM | 2975 | C | TYR | B | 135 | 47.071 | 47.660 | 12.236 | 1.00 | 10.77 | B |
| ATOM | 2976 | O | TYR | B | 135 | 46.371 | 46.769 | 12.711 | 1.00 | 10.80 | B |
| ATOM | 2977 | N | GLY | B | 136 | 47.894 | 48.407 | 12.966 | 1.00 | 10.64 | B |
| ATOM | 2978 | CA | GLY | B | 136 | 48.011 | 48.187 | 14.393 | 1.00 | 10.44 | B |
| ATOM | 2979 | C | GLY | B | 136 | 48.539 | 49.378 | 15.170 | 1.00 | 11.38 | B |
| ATOM | 2980 | O | GLY | B | 136 | 48.744 | 50.469 | 14.623 | 1.00 | 10.73 | B |
| ATOM | 2981 | N | THR | B | 137 | 48.771 | 49.150 | 16.457 | 1.00 | 11.87 | B |
| ATOM | 2982 | CA | THR | B | 137 | 49.256 | 50.173 | 17.376 | 1.00 | 12.05 | B |
| ATOM | 2983 | CB | THR | B | 137 | 48.162 | 50.568 | 18.389 | 1.00 | 14.38 | B |
| ATOM | 2984 | OG1 | THR | B | 137 | 47.931 | 49.465 | 19.279 | 1.00 | 14.95 | B |
| ATOM | 2985 | CG2 | THR | B | 137 | 46.855 | 50.908 | 17.685 | 1.00 | 14.15 | B |
| ATOM | 2986 | C | THR | B | 137 | 50.414 | 49.623 | 18.201 | 1.00 | 12.31 | B |
| ATOM | 2987 | O | THR | B | 137 | 50.849 | 48.483 | 18.015 | 1.00 | 11.64 | B |
| ATOM | 2988 | N | LEU | B | 138 | 50.911 | 50.446 | 19.118 | 1.00 | 12.97 | B |
| ATOM | 2989 | CA | LEU | B | 138 | 51.967 | 50.018 | 20.019 | 1.00 | 13.12 | B |
| ATOM | 2990 | CB | LEU | B | 138 | 52.521 | 51.230 | 20.768 | 1.00 | 13.27 | B |
| ATOM | 2991 | CG | LEU | B | 138 | 53.491 | 52.127 | 20.004 | 1.00 | 14.13 | B |
| ATOM | 2992 | CD1 | LEU | B | 138 | 53.699 | 53.428 | 20.771 | 1.00 | 14.57 | B |
| ATOM | 2993 | CD2 | LEU | B | 138 | 54.806 | 51.390 | 19.798 | 1.00 | 15.37 | B |
| ATOM | 2994 | C | LEU | B | 138 | 51.309 | 48.994 | 20.938 | 1.00 | 15.45 | B |
| ATOM | 2995 | O | LEU | B | 138 | 50.087 | 49.008 | 21.116 | 1.00 | 15.27 | B |
| ATOM | 2996 | N | ASP | B | 139 | 52.115 | 48.113 | 21.523 | 1.00 | 16.69 | B |
| ATOM | 2997 | CA | ASP | B | 139 | 51.596 | 47.074 | 22.410 | 1.00 | 20.52 | B |
| ATOM | 2998 | CB | ASP | B | 139 | 52.621 | 45.943 | 22.503 | 1.00 | 24.45 | B |
| ATOM | 2999 | CG | ASP | B | 139 | 53.920 | 46.390 | 23.137 | 1.00 | 30.93 | B |
| ATOM | 3000 | OD1 | ASP | B | 139 | 54.543 | 47.337 | 22.611 | 1.00 | 35.31 | B |
| ATOM | 3001 | OD2 | ASP | B | 139 | 54.318 | 45.794 | 24.160 | 1.00 | 36.08 | B |
| ATOM | 3002 | C | ASP | B | 139 | 51.200 | 47.629 | 23.773 | 1.00 | 20.31 | B |
| ATOM | 3003 | O | ASP | B | 139 | 50.639 | 46.912 | 24.604 | 1.00 | 20.48 | B |
| ATOM | 3004 | N | SER | B | 140 | 51.505 | 48.900 | 24.008 | 1.00 | 18.89 | B |
| ATOM | 3005 | CA | SER | B | 140 | 51.163 | 49.553 | 25.266 | 1.00 | 18.19 | B |
| ATOM | 3006 | CB | SER | B | 140 | 52.215 | 49.208 | 26.325 | 1.00 | 20.09 | B |
| ATOM | 3007 | OG | SER | B | 140 | 53.475 | 49.758 | 25.990 | 1.00 | 24.92 | B |
| ATOM | 3008 | C | SER | B | 140 | 51.048 | 51.046 | 24.996 | 1.00 | 17.29 | B |
| ATOM | 3009 | O | SER | B | 140 | 51.628 | 51.551 | 24.038 | 1.00 | 18.70 | B |
| ATOM | 3010 | N | GLY | B | 141 | 50.299 | 51.752 | 25.835 | 1.00 | 15.40 | B |
| ATOM | 3011 | CA | GLY | B | 141 | 50.142 | 53.180 | 25.638 | 1.00 | 13.91 | B |
| ATOM | 3012 | C | GLY | B | 141 | 48.694 | 53.598 | 25.478 | 1.00 | 13.36 | B |
| ATOM | 3013 | O | GLY | B | 141 | 47.800 | 52.758 | 25.365 | 1.00 | 13.55 | B |
| ATOM | 3014 | N | SER | B | 142 | 48.468 | 54.905 | 25.437 | 1.00 | 10.87 | B |
| ATOM | 3015 | CA | SER | B | 142 | 47.123 | 55.452 | 25.328 | 1.00 | 10.24 | B |
| ATOM | 3016 | CB | SER | B | 142 | 47.177 | 56.953 | 25.615 | 1.00 | 12.05 | B |
| ATOM | 3017 | OG | SER | B | 142 | 47.796 | 57.203 | 26.862 | 1.00 | 18.50 | B |
| ATOM | 3018 | C | SER | B | 142 | 46.408 | 55.141 | 24.021 | 1.00 | 10.17 | B |
| ATOM | 3019 | O | SER | B | 142 | 45.182 | 55.122 | 23.980 | 1.00 | 10.97 | B |
| ATOM | 3020 | N | THR | B | 143 | 47.152 | 54.911 | 22.946 | 1.00 | 9.32 | B |
| ATOM | 3021 | CA | THR | B | 143 | 46.499 | 54.606 | 21.684 | 1.00 | 9.97 | B |
| ATOM | 3022 | CB | THR | B | 143 | 47.488 | 54.685 | 20.518 | 1.00 | 9.07 | B |
| ATOM | 3023 | OG1 | THR | B | 143 | 47.987 | 56.024 | 20.423 | 1.00 | 10.01 | B |
| ATOM | 3024 | CG2 | THR | B | 143 | 46.807 | 54.318 | 19.213 | 1.00 | 10.12 | B |
| ATOM | 3025 | C | THR | B | 143 | 45.858 | 53.222 | 21.743 | 1.00 | 10.20 | B |
| ATOM | 3026 | O | THR | B | 143 | 44.717 | 53.046 | 21.324 | 1.00 | 11.59 | B |
| ATOM | 3027 | N | LYS | B | 144 | 46.589 | 52.244 | 22.268 | 1.00 | 10.97 | B |
| ATOM | 3028 | CA | LYS | B | 144 | 46.046 | 50.899 | 22.382 | 1.00 | 12.59 | B |
| ATOM | 3029 | CB | LYS | B | 144 | 47.135 | 49.953 | 22.892 | 1.00 | 14.35 | B |
| ATOM | 3030 | CG | LYS | B | 144 | 46.688 | 48.500 | 22.991 | 1.00 | 17.17 | B |
| ATOM | 3031 | CD | LYS | B | 144 | 47.786 | 47.635 | 23.588 | 1.00 | 21.73 | B |
| ATOM | 3032 | CE | LYS | B | 144 | 47.327 | 46.200 | 23.777 | 1.00 | 23.63 | B |
| ATOM | 3033 | NZ | LYS | B | 144 | 48.380 | 45.367 | 24.418 | 1.00 | 25.69 | B |
| ATOM | 3034 | C | LYS | B | 144 | 44.824 | 50.955 | 23.293 | 1.00 | 12.47 | B |
| ATOM | 3035 | O | LYS | B | 144 | 43.800 | 50.339 | 23.005 | 1.00 | 13.35 | B |
| ATOM | 3036 | N | GLU | B | 145 | 44.934 | 51.703 | 24.388 | 1.00 | 13.84 | B |
| ATOM | 3037 | CA | GLU | B | 145 | 43.829 | 51.833 | 25.334 | 1.00 | 13.52 | B |
| ATOM | 3038 | CB | GLU | B | 145 | 44.314 | 52.574 | 26.584 | 1.00 | 17.01 | B |
| ATOM | 3039 | CG | GLU | B | 145 | 45.335 | 51.804 | 27.403 | 1.00 | 22.59 | B |
| ATOM | 3040 | CD | GLU | B | 145 | 44.878 | 50.393 | 27.716 | 1.00 | 26.96 | B |
| ATOM | 3041 | OE1 | GLU | B | 145 | 43.740 | 50.229 | 28.203 | 1.00 | 28.77 | B |
| ATOM | 3042 | OE2 | GLU | B | 145 | 45.661 | 49.447 | 27.480 | 1.00 | 30.33 | B |
| ATOM | 3043 | C | GLU | B | 145 | 42.624 | 52.498 | 24.683 | 1.00 | 12.56 | B |
| ATOM | 3044 | O | GLU | B | 145 | 41.481 | 52.136 | 24.969 | 1.00 | 12.58 | B |
| ATOM | 3045 | N | PHE | B | 146 | 42.877 | 53.474 | 23.815 | 1.00 | 12.47 | B |
| ATOM | 3046 | CA | PHE | B | 146 | 41.799 | 54.171 | 23.124 | 1.00 | 12.35 | B |
| ATOM | 3047 | CB | PHE | B | 146 | 42.381 | 55.212 | 22.160 | 1.00 | 11.86 | B |
| ATOM | 3048 | CG | PHE | B | 146 | 41.356 | 55.817 | 21.234 | 1.00 | 12.49 | B |
| ATOM | 3049 | CD1 | PHE | B | 146 | 40.678 | 56.851 | 21.611 | 1.00 | 13.03 | B |
| ATOM | 3050 | CD2 | PHE | B | 146 | 41.148 | 55.295 | 20.067 | 1.00 | 13.25 | B |
| ATOM | 3051 | CE1 | PHE | B | 146 | 39.709 | 57.446 | 20.797 | 1.00 | 14.59 | B |
| ATOM | 3052 | CE2 | PHE | B | 146 | 40.198 | 55.816 | 19.175 | 1.00 | 14.19 | B |
| ATOM | 3053 | CZ | PHE | B | 146 | 39.466 | 56.917 | 19.554 | 1.00 | 14.18 | B |
| ATOM | 3054 | C | PHE | B | 146 | 40.944 | 53.148 | 22.399 | 1.00 | 11.89 | B |
| ATOM | 3055 | O | PHE | B | 146 | 39.721 | 53.133 | 22.533 | 1.00 | 13.17 | B |
| ATOM | 3056 | N | PHE | B | 147 | 41.602 | 52.292 | 21.626 | 1.00 | 10.83 | B |
| ATOM | 3057 | CA | PHE | B | 147 | 40.898 | 51.277 | 20.861 | 1.00 | 12.64 | B |
| ATOM | 3058 | CB | PHE | B | 147 | 41.865 | 50.681 | 19.831 | 1.00 | 10.75 | B |
| ATOM | 3059 | CG | PHE | B | 147 | 42.100 | 51.573 | 18.643 | 1.00 | 10.57 | B |
| ATOM | 3060 | CD1 | PHE | B | 147 | 41.169 | 51.718 | 17.754 | 1.00 | 10.12 | B |
| ATOM | 3061 | CD2 | PHE | B | 147 | 43.223 | 52.209 | 18.518 | 1.00 | 9.16 | B |
| ATOM | 3062 | CE1 | PHE | B | 147 | 41.320 | 52.549 | 16.641 | 1.00 | 11.01 | B |
| ATOM | 3063 | CE2 | PHE | B | 147 | 43.473 | 53.069 | 17.438 | 1.00 | 10.93 | B |
| ATOM | 3064 | CZ | PHE | B | 147 | 42.500 | 53.239 | 16.483 | 1.00 | 10.78 | B |
| ATOM | 3065 | C | PHE | B | 147 | 40.233 | 50.239 | 21.752 | 1.00 | 13.03 | B |
| ATOM | 3066 | O | PHE | B | 147 | 39.075 | 49.893 | 21.538 | 1.00 | 13.65 | B |
| ATOM | 3067 | N | ARG | B | 148 | 40.952 | 49.761 | 22.760 | 1.00 | 13.08 | B |
| ATOM | 3068 | CA | ARG | B | 148 | 40.400 | 48.755 | 23.658 | 1.00 | 14.29 | B |
| ATOM | 3069 | CB | ARG | B | 148 | 41.491 | 48.312 | 24.641 | 1.00 | 14.69 | B |
| ATOM | 3070 | CG | ARG | B | 148 | 41.017 | 47.284 | 25.665 | 1.00 | 18.17 | B |
| ATOM | 3071 | CD | ARG | B | 148 | 42.129 | 46.894 | 26.629 | 1.00 | 22.36 | B |
| ATOM | 3072 | NE | ARG | B | 148 | 43.229 | 46.208 | 25.957 | 1.00 | 25.91 | B |
| ATOM | 3073 | CZ | ARG | B | 148 | 44.326 | 45.772 | 26.570 | 1.00 | 27.70 | B |
| ATOM | 3074 | NH1 | ARG | B | 148 | 44.475 | 45.949 | 27.876 | 1.00 | 27.74 | B |
| ATOM | 3075 | NH2 | ARG | B | 148 | 45.275 | 45.154 | 25.878 | 1.00 | 26.51 | B |
| ATOM | 3076 | C | ARG | B | 148 | 39.126 | 49.217 | 24.366 | 1.00 | 15.76 | B |
| ATOM | 3077 | O | ARG | B | 148 | 38.210 | 48.419 | 24.566 | 1.00 | 16.77 | B |
| ATOM | 3078 | N | ARG | B | 149 | 39.052 | 50.494 | 24.736 | 1.00 | 15.90 | B |
| ATOM | 3079 | CA | ARG | B | 149 | 37.880 | 50.992 | 25.458 | 1.00 | 17.70 | B |
| ATOM | 3080 | CB | ARG | B | 149 | 38.350 | 51.867 | 26.625 | 1.00 | 20.41 | B |
| ATOM | 3081 | CG | ARG | B | 149 | 39.513 | 51.316 | 27.425 | 1.00 | 25.96 | B |
| ATOM | 3082 | CD | ARG | B | 149 | 39.611 | 52.011 | 28.777 | 1.00 | 30.05 | B |
| ATOM | 3083 | NE | ARG | B | 149 | 39.203 | 53.414 | 28.720 | 1.00 | 33.23 | B |
| ATOM | 3084 | CZ | ARG | B | 149 | 39.840 | 54.363 | 28.039 | 1.00 | 34.57 | B |
| ATOM | 3085 | NH1 | ARG | B | 149 | 40.933 | 54.072 | 27.344 | 1.00 | 35.30 | B |
| ATOM | 3086 | NH2 | ARG | B | 149 | 39.379 | 55.608 | 28.052 | 1.00 | 34.31 | B |
| ATOM | 3087 | C | ARG | B | 149 | 36.837 | 51.717 | 24.614 | 1.00 | 16.79 | B |
| ATOM | 3088 | O | ARG | B | 149 | 35.791 | 52.110 | 25.135 | 1.00 | 17.75 | B |
| ATOM | 3089 | N | SER | B | 150 | 37.093 | 51.898 | 23.325 | 1.00 | 16.03 | B |
| ATOM | 3090 | CA | SER | B | 150 | 36.143 | 52.625 | 22.491 | 1.00 | 16.26 | B |
| ATOM | 3091 | CB | SER | B | 150 | 36.792 | 52.953 | 21.145 | 1.00 | 16.56 | B |
| ATOM | 3092 | OG | SER | B | 150 | 35.916 | 53.744 | 20.358 | 1.00 | 16.88 | B |
| ATOM | 3093 | C | SER | B | 150 | 34.797 | 51.932 | 22.318 | 1.00 | 17.00 | B |
| ATOM | 3094 | O | SER | B | 150 | 34.717 | 50.705 | 22.226 | 1.00 | 17.43 | B |
| ATOM | 3095 | N | ALA | B | 151 | 33.744 | 52.746 | 22.270 | 1.00 | 16.82 | B |
| ATOM | 3096 | CA | ALA | B | 151 | 32.379 | 52.272 | 22.073 | 1.00 | 17.70 | B |
| ATOM | 3097 | CB | ALA | B | 151 | 31.475 | 52.764 | 23.209 | 1.00 | 18.36 | B |
| ATOM | 3098 | C | ALA | B | 151 | 31.890 | 52.816 | 20.730 | 1.00 | 18.27 | B |
| ATOM | 3099 | O | ALA | B | 151 | 30.736 | 52.618 | 20.345 | 1.00 | 20.02 | B |
| ATOM | 3100 | N | ILE | B | 152 | 32.780 | 53.518 | 20.029 | 1.00 | 17.82 | B |
| ATOM | 3101 | CA | ILE | B | 152 | 32.472 | 54.083 | 18.716 | 1.00 | 17.05 | B |
| ATOM | 3102 | CB | ILE | B | 152 | 33.450 | 55.229 | 18.363 | 1.00 | 17.75 | B |
| ATOM | 3103 | CG2 | ILE | B | 152 | 33.186 | 55.730 | 16.949 | 1.00 | 17.27 | B |
| ATOM | 3104 | CG1 | ILE | B | 152 | 33.301 | 56.367 | 19.379 | 1.00 | 19.71 | B |
| ATOM | 3105 | CD1 | ILE | B | 152 | 34.204 | 57.559 | 19.120 | 1.00 | 23.70 | B |
| ATOM | 3106 | C | ILE | B | 152 | 32.599 | 52.960 | 17.683 | 1.00 | 16.26 | B |
| ATOM | 3107 | O | ILE | B | 152 | 33.642 | 52.317 | 17.582 | 1.00 | 16.10 | B |
| ATOM | 3108 | N | ALA | B | 153 | 31.537 | 52.732 | 16.918 | 1.00 | 15.92 | B |
| ATOM | 3109 | CA | ALA | B | 153 | 31.502 | 51.659 | 15.924 | 1.00 | 15.10 | B |
| ATOM | 3110 | CB | ALA | B | 153 | 30.302 | 51.848 | 15.004 | 1.00 | 15.55 | B |
| ATOM | 3111 | C | ALA | B | 153 | 32.763 | 51.445 | 15.088 | 1.00 | 13.13 | B |
| ATOM | 3112 | O | ALA | B | 153 | 33.301 | 50.334 | 15.037 | 1.00 | 12.87 | B |
| ATOM | 3113 | N | VAL | B | 154 | 33.237 | 52.491 | 14.422 | 1.00 | 13.30 | B |
| ATOM | 3114 | CA | VAL | B | 154 | 34.427 | 52.346 | 13.593 | 1.00 | 13.40 | B |
| ATOM | 3115 | CB | VAL | B | 154 | 34.762 | 53.660 | 12.843 | 1.00 | 14.79 | B |
| ATOM | 3116 | CG1 | VAL | B | 154 | 36.114 | 53.540 | 12.150 | 1.00 | 15.68 | B |
| ATOM | 3117 | CG2 | VAL | B | 154 | 33.682 | 53.955 | 11.818 | 1.00 | 16.59 | B |
| ATOM | 3118 | C | VAL | B | 154 | 35.646 | 51.914 | 14.402 | 1.00 | 11.56 | B |
| ATOM | 3119 | O | VAL | B | 154 | 36.395 | 51.027 | 13.984 | 1.00 | 12.09 | B |
| ATOM | 3120 | N | PHE | B | 155 | 35.831 | 52.519 | 15.572 | 1.00 | 11.82 | B |
| ATOM | 3121 | CA | PHE | B | 155 | 36.986 | 52.203 | 16.403 | 1.00 | 12.53 | B |
| ATOM | 3122 | CB | PHE | B | 155 | 37.251 | 53.361 | 17.377 | 1.00 | 12.92 | B |
| ATOM | 3123 | CG | PHE | B | 155 | 37.352 | 54.714 | 16.714 | 1.00 | 13.23 | B |
| ATOM | 3124 | CD1 | PHE | B | 155 | 37.914 | 54.839 | 15.552 | 1.00 | 13.45 | B |
| ATOM | 3125 | CD2 | PHE | B | 155 | 36.884 | 55.756 | 17.320 | 1.00 | 13.49 | B |
| ATOM | 3126 | CE1 | PHE | B | 155 | 38.038 | 56.083 | 14.914 | 1.00 | 13.54 | B |
| ATOM | 3127 | CE2 | PHE | B | 155 | 36.959 | 57.038 | 16.770 | 1.00 | 14.37 | B |
| ATOM | 3128 | CZ | PHE | B | 155 | 37.547 | 57.201 | 15.543 | 1.00 | 13.87 | B |
| ATOM | 3129 | C | PHE | B | 155 | 36.894 | 50.850 | 17.102 | 1.00 | 13.04 | B |
| ATOM | 3130 | O | PHE | B | 155 | 37.905 | 50.151 | 17.219 | 1.00 | 12.48 | B |
| ATOM | 3131 | N | ASP | B | 156 | 35.712 | 50.467 | 17.586 | 1.00 | 13.73 | B |
| ATOM | 3132 | CA | ASP | B | 156 | 35.623 | 49.170 | 18.249 | 1.00 | 14.18 | B |
| ATOM | 3133 | CB | ASP | B | 156 | 34.350 | 49.070 | 19.118 | 1.00 | 17.34 | B |
| ATOM | 3134 | CG | ASP | B | 156 | 33.076 | 49.316 | 18.349 | 1.00 | 20.27 | B |
| ATOM | 3135 | OD1 | ASP | B | 156 | 32.889 | 48.711 | 17.276 | 1.00 | 21.62 | B |
| ATOM | 3136 | OD2 | ASP | B | 156 | 32.243 | 50.109 | 18.841 | 1.00 | 22.52 | B |
| ATOM | 3137 | C | ASP | B | 156 | 35.788 | 48.054 | 17.221 | 1.00 | 13.30 | B |
| ATOM | 3138 | O | ASP | B | 156 | 36.299 | 46.983 | 17.549 | 1.00 | 13.86 | B |
| ATOM | 3139 | N | LYS | B | 157 | 35.382 | 48.299 | 15.978 | 1.00 | 12.32 | B |
| ATOM | 3140 | CA | LYS | B | 157 | 35.564 | 47.294 | 14.938 | 1.00 | 13.15 | B |
| ATOM | 3141 | CB | LYS | B | 157 | 34.775 | 47.679 | 13.684 | 1.00 | 14.71 | B |
| ATOM | 3142 | CG | LYS | B | 157 | 34.974 | 46.697 | 12.541 | 1.00 | 16.66 | B |
| ATOM | 3143 | CD | LYS | B | 157 | 33.986 | 46.924 | 11.409 | 1.00 | 20.89 | B |
| ATOM | 3144 | CE | LYS | B | 157 | 34.159 | 45.872 | 10.321 | 1.00 | 22.42 | B |
| ATOM | 3145 | NZ | LYS | B | 157 | 33.183 | 46.041 | 9.211 | 1.00 | 26.26 | B |
| ATOM | 3146 | C | LYS | B | 157 | 37.067 | 47.166 | 14.681 | 1.00 | 11.75 | B |
| ATOM | 3147 | O | LYS | B | 157 | 37.583 | 46.070 | 14.477 | 1.00 | 12.82 | B |
| ATOM | 3148 | N | MET | B | 158 | 37.769 | 48.298 | 14.692 | 1.00 | 11.60 | B |
| ATOM | 3149 | CA | MET | B | 158 | 39.209 | 48.291 | 14.475 | 1.00 | 11.34 | B |
| ATOM | 3150 | CB | MET | B | 158 | 39.707 | 49.734 | 14.344 | 1.00 | 10.92 | B |
| ATOM | 3151 | CG | MET | B | 158 | 39.355 | 50.389 | 13.013 | 1.00 | 10.61 | B |
| ATOM | 3152 | SD | MET | B | 158 | 39.778 | 52.145 | 12.967 | 1.00 | 11.68 | B |
| ATOM | 3153 | CE | MET | B | 158 | 41.562 | 52.071 | 12.823 | 1.00 | 11.70 | B |
| ATOM | 3154 | C | MET | B | 158 | 39.907 | 47.506 | 15.579 | 1.00 | 11.19 | B |
| ATOM | 3155 | O | MET | B | 158 | 40.836 | 46.749 | 15.309 | 1.00 | 10.94 | B |
| ATOM | 3156 | N | TRP | B | 159 | 39.455 | 47.675 | 16.817 | 1.00 | 11.18 | B |
| ATOM | 3157 | CA | TRP | B | 159 | 40.056 | 46.966 | 17.943 | 1.00 | 11.28 | B |
| ATOM | 3158 | CB | TRP | B | 159 | 39.478 | 47.492 | 19.259 | 1.00 | 10.97 | B |
| ATOM | 3159 | CG | TRP | B | 159 | 39.968 | 46.698 | 20.436 | 1.00 | 12.16 | B |
| ATOM | 3160 | CD2 | TRP | B | 159 | 41.328 | 46.562 | 20.866 | 1.00 | 13.67 | B |
| ATOM | 3161 | CE2 | TRP | B | 159 | 41.332 | 45.679 | 21.968 | 1.00 | 14.30 | B |
| ATOM | 3162 | CE3 | TRP | B | 159 | 42.545 | 47.101 | 20.425 | 1.00 | 14.40 | B |
| ATOM | 3163 | CD1 | TRP | B | 159 | 39.221 | 45.915 | 21.274 | 1.00 | 13.44 | B |
| ATOM | 3164 | NE1 | TRP | B | 159 | 40.035 | 45.300 | 22.197 | 1.00 | 14.53 | B |
| ATOM | 3165 | CZ2 | TRP | B | 159 | 42.508 | 45.320 | 22.637 | 1.00 | 16.70 | B |
| ATOM | 3166 | CZ3 | TRP | B | 159 | 43.716 | 46.744 | 21.092 | 1.00 | 15.71 | B |
| ATOM | 3167 | CH2 | TRP | B | 159 | 43.687 | 45.861 | 22.185 | 1.00 | 16.50 | B |
| ATOM | 3168 | C | TRP | B | 159 | 39.865 | 45.465 | 17.806 | 1.00 | 11.68 | B |
| ATOM | 3169 | O | TRP | B | 159 | 40.800 | 44.688 | 17.998 | 1.00 | 11.26 | B |
| ATOM | 3170 | N | THR | B | 160 | 38.642 | 45.062 | 17.487 | 1.00 | 12.22 | B |
| ATOM | 3171 | CA | THR | B | 160 | 38.339 | 43.646 | 17.334 | 1.00 | 11.26 | B |
| ATOM | 3172 | CB | THR | B | 160 | 36.858 | 43.441 | 16.993 | 1.00 | 13.83 | B |
| ATOM | 3173 | OG1 | THR | B | 160 | 36.063 | 43.862 | 18.110 | 1.00 | 16.23 | B |
| ATOM | 3174 | CG2 | THR | B | 160 | 36.571 | 41.978 | 16.688 | 1.00 | 15.16 | B |
| ATOM | 3175 | C | THR | B | 160 | 39.223 | 43.048 | 16.246 | 1.00 | 10.15 | B |
| ATOM | 3176 | O | THR | B | 160 | 39.730 | 41.937 | 16.394 | 1.00 | 10.71 | B |
| ATOM | 3177 | N | TYR | B | 161 | 39.408 | 43.790 | 15.157 | 1.00 | 11.18 | B |
| ATOM | 3178 | CA | TYR | B | 161 | 40.261 | 43.332 | 14.067 | 1.00 | 11.24 | B |
| ATOM | 3179 | CB | TYR | B | 161 | 40.176 | 44.301 | 12.880 | 1.00 | 10.21 | B |
| ATOM | 3180 | CG | TYR | B | 161 | 41.426 | 44.244 | 12.029 | 1.00 | 11.28 | B |
| ATOM | 3181 | CD1 | TYR | B | 161 | 41.574 | 43.286 | 11.027 | 1.00 | 11.96 | B |
| ATOM | 3182 | CE1 | TYR | B | 161 | 42.774 | 43.152 | 10.330 | 1.00 | 12.51 | B |
| ATOM | 3183 | CD2 | TYR | B | 161 | 42.511 | 45.076 | 12.306 | 1.00 | 10.43 | B |
| ATOM | 3184 | CE2 | TYR | B | 161 | 43.710 | 44.950 | 11.619 | 1.00 | 12.75 | B |
| ATOM | 3185 | CZ | TYR | B | 161 | 43.839 | 43.984 | 10.635 | 1.00 | 12.24 | B |
| ATOM | 3186 | OH | TYR | B | 161 | 45.046 | 43.836 | 9.993 | 1.00 | 15.00 | B |
| ATOM | 3187 | C | TYR | B | 161 | 41.696 | 43.195 | 14.567 | 1.00 | 11.23 | B |
| ATOM | 3188 | O | TYR | B | 161 | 42.354 | 42.180 | 14.334 | 1.00 | 11.92 | B |
| ATOM | 3189 | N | MET | B | 162 | 42.182 | 44.229 | 15.249 | 1.00 | 11.29 | B |
| ATOM | 3190 | CA | MET | B | 162 | 43.552 | 44.231 | 15.754 | 1.00 | 11.30 | B |
| ATOM | 3191 | CB | MET | B | 162 | 43.849 | 45.563 | 16.442 | 1.00 | 13.62 | B |
| ATOM | 3192 | CG | MET | B | 162 | 44.043 | 46.742 | 15.531 | 1.00 | 12.58 | B |
| ATOM | 3193 | SD | MET | B | 162 | 44.548 | 48.154 | 16.533 | 1.00 | 18.19 | B |
| ATOM | 3194 | CE | MET | B | 162 | 43.054 | 49.101 | 16.546 | 1.00 | 20.27 | B |
| ATOM | 3195 | C | MET | B | 162 | 43.851 | 43.072 | 16.679 | 1.00 | 12.13 | B |
| ATOM | 3196 | O | MET | B | 162 | 44.889 | 42.423 | 16.560 | 1.00 | 12.09 | B |
| ATOM | 3197 | N | ARG | B | 163 | 42.943 | 42.820 | 17.614 | 1.00 | 11.82 | B |
| ATOM | 3198 | CA | ARG | B | 163 | 43.153 | 41.759 | 18.582 | 1.00 | 13.21 | B |
| ATOM | 3199 | CB | ARG | B | 163 | 42.128 | 41.911 | 19.710 | 1.00 | 16.98 | B |
| ATOM | 3200 | CG | ARG | B | 163 | 42.556 | 41.238 | 20.999 | 1.00 | 20.62 | B |
| ATOM | 3201 | CD | ARG | B | 163 | 41.505 | 41.347 | 22.087 | 1.00 | 24.44 | B |
| ATOM | 3202 | NE | ARG | B | 163 | 42.003 | 40.782 | 23.337 | 1.00 | 27.50 | B |
| ATOM | 3203 | CZ | ARG | B | 163 | 41.245 | 40.491 | 24.387 | 1.00 | 28.64 | B |
| ATOM | 3204 | NH1 | ARG | B | 163 | 39.938 | 40.711 | 24.348 | 1.00 | 28.24 | B |
| ATOM | 3205 | NH2 | ARG | B | 163 | 41.797 | 39.977 | 25.478 | 1.00 | 29.63 | B |
| ATOM | 3206 | C | ARG | B | 163 | 43.142 | 40.376 | 17.947 | 1.00 | 12.18 | B |
| ATOM | 3207 | O | ARG | B | 163 | 43.786 | 39.459 | 18.454 | 1.00 | 12.88 | B |
| ATOM | 3208 | N | SER | B | 164 | 42.435 | 40.226 | 16.831 | 1.00 | 10.89 | B |
| ATOM | 3209 | CA | SER | B | 164 | 42.333 | 38.925 | 16.173 | 1.00 | 11.71 | B |
| ATOM | 3210 | CB | SER | B | 164 | 40.853 | 38.625 | 15.918 | 1.00 | 12.69 | B |
| ATOM | 3211 | OG | SER | B | 164 | 40.312 | 39.503 | 14.945 | 1.00 | 14.22 | B |
| ATOM | 3212 | C | SER | B | 164 | 43.176 | 38.762 | 14.907 | 1.00 | 11.81 | B |
| ATOM | 3213 | O | SER | B | 164 | 43.220 | 37.674 | 14.330 | 1.00 | 12.97 | B |
| ATOM | 3214 | N | ALA | B | 165 | 43.849 | 39.824 | 14.478 | 1.00 | 12.37 | B |
| ATOM | 3215 | CA | ALA | B | 165 | 44.662 | 39.768 | 13.267 | 1.00 | 11.61 | B |
| ATOM | 3216 | CB | ALA | B | 165 | 45.026 | 41.184 | 12.826 | 1.00 | 12.88 | B |
| ATOM | 3217 | C | ALA | B | 165 | 45.931 | 38.923 | 13.391 | 1.00 | 12.82 | B |
| ATOM | 3218 | O | ALA | B | 165 | 46.588 | 38.921 | 14.428 | 1.00 | 12.59 | B |
| ATOM | 3219 | N | GLU | B | 166 | 46.267 | 38.213 | 12.314 | 1.00 | 13.40 | B |
| ATOM | 3220 | CA | GLU | B | 166 | 47.460 | 37.370 | 12.265 | 1.00 | 14.47 | B |
| ATOM | 3221 | CB | GLU | B | 166 | 47.061 | 35.898 | 12.407 | 1.00 | 13.75 | B |
| ATOM | 3222 | CG | GLU | B | 166 | 46.338 | 35.546 | 13.692 | 1.00 | 13.66 | B |
| ATOM | 3223 | CD | GLU | B | 166 | 46.229 | 34.045 | 13.885 | 1.00 | 12.43 | B |
| ATOM | 3224 | OE1 | GLU | B | 166 | 45.542 | 33.383 | 13.078 | 1.00 | 14.72 | B |
| ATOM | 3225 | OE2 | GLU | B | 166 | 46.844 | 33.530 | 14.838 | 1.00 | 15.12 | B |
| ATOM | 3226 | C | GLU | B | 166 | 48.198 | 37.628 | 10.955 | 1.00 | 15.00 | B |
| ATOM | 3227 | O | GLU | B | 166 | 47.583 | 37.638 | 9.887 | 1.00 | 17.00 | B |
| ATOM | 3228 | N | PRO | B | 167 | 49.523 | 37.850 | 11.017 | 1.00 | 14.83 | B |
| ATOM | 3229 | CD | PRO | B | 167 | 50.360 | 37.853 | 9.807 | 1.00 | 16.26 | B |
| ATOM | 3230 | CA | PRO | B | 167 | 50.364 | 37.879 | 12.220 | 1.00 | 13.45 | B |
| ATOM | 3231 | CB | PRO | B | 167 | 51.789 | 37.889 | 11.643 | 1.00 | 14.40 | B |
| ATOM | 3232 | CG | PRO | B | 167 | 51.666 | 37.159 | 10.327 | 1.00 | 18.88 | B |
| ATOM | 3233 | C | PRO | B | 167 | 50.008 | 39.094 | 13.071 | 1.00 | 13.13 | B |
| ATOM | 3234 | O | PRO | B | 167 | 49.294 | 39.990 | 12.614 | 1.00 | 13.35 | B |
| ATOM | 3235 | N | SER | B | 168 | 50.511 | 39.129 | 14.301 | 1.00 | 13.08 | B |
| ATOM | 3236 | CA | SER | B | 168 | 50.230 | 40.240 | 15.203 | 1.00 | 12.23 | B |
| ATOM | 3237 | CB | SER | B | 168 | 51.048 | 40.081 | 16.481 | 1.00 | 14.03 | B |
| ATOM | 3238 | OG | SER | B | 168 | 50.885 | 41.230 | 17.293 | 1.00 | 14.91 | B |
| ATOM | 3239 | C | SER | B | 168 | 50.487 | 41.578 | 14.528 | 1.00 | 11.20 | B |
| ATOM | 3240 | O | SER | B | 168 | 51.514 | 41.770 | 13.873 | 1.00 | 12.75 | B |
| ATOM | 3241 | N | VAL | B | 169 | 49.554 | 42.506 | 14.708 | 1.00 | 10.70 | B |
| ATOM | 3242 | CA | VAL | B | 169 | 49.676 | 43.839 | 14.128 | 1.00 | 11.80 | B |
| ATOM | 3243 | CB | VAL | B | 169 | 48.309 | 44.362 | 13.619 | 1.00 | 12.79 | B |
| ATOM | 3244 | CG1 | VAL | B | 169 | 47.828 | 43.496 | 12.459 | 1.00 | 13.33 | B |
| ATOM | 3245 | CG2 | VAL | B | 169 | 47.289 | 44.371 | 14.755 | 1.00 | 12.38 | B |
| ATOM | 3246 | C | VAL | B | 169 | 50.244 | 44.841 | 15.126 | 1.00 | 11.83 | B |
| ATOM | 3247 | O | VAL | B | 169 | 50.406 | 46.020 | 14.809 | 1.00 | 12.00 | B |
| ATOM | 3248 | N | PHE | B | 170 | 50.558 | 44.370 | 16.328 | 1.00 | 10.85 | B |
| ATOM | 3249 | CA | PHE | B | 170 | 51.108 | 45.237 | 17.363 | 1.00 | 12.02 | B |
| ATOM | 3250 | CB | PHE | B | 170 | 50.653 | 44.728 | 18.737 | 1.00 | 11.90 | B |
| ATOM | 3251 | CG | PHE | B | 170 | 49.163 | 44.770 | 18.935 | 1.00 | 12.25 | B |
| ATOM | 3252 | CD1 | PHE | B | 170 | 48.561 | 45.905 | 19.094 | 1.00 | 12.64 | B |
| ATOM | 3253 | CD2 | PHE | B | 170 | 48.480 | 43.669 | 18.920 | 1.00 | 12.85 | B |
| ATOM | 3254 | CE1 | PHE | B | 170 | 47.169 | 45.996 | 19.251 | 1.00 | 12.78 | B |
| ATOM | 3255 | CE2 | PHE | B | 170 | 47.088 | 43.652 | 19.068 | 1.00 | 14.94 | B |
| ATOM | 3256 | CZ | PHE | B | 170 | 46.423 | 44.843 | 19.236 | 1.00 | 14.35 | B |
| ATOM | 3257 | C | PHE | B | 170 | 52.623 | 45.335 | 17.238 | 1.00 | 12.05 | B |
| ATOM | 3258 | O | PHE | B | 170 | 53.282 | 44.936 | 16.787 | 1.00 | 14.89 | B |
| ATOM | 3259 | N | VAL | B | 171 | 53.168 | 46.482 | 17.633 | 1.00 | 12.21 | B |
| ATOM | 3260 | CA | VAL | B | 171 | 54.610 | 46.715 | 17.597 | 1.00 | 13.01 | B |
| ATOM | 3261 | CB | VAL | B | 171 | 54.996 | 47.764 | 16.522 | 1.00 | 13.51 | B |
| ATOM | 3262 | CG1 | VAL | B | 171 | 54.606 | 47.252 | 15.143 | 1.00 | 14.10 | B |
| ATOM | 3263 | CG2 | VAL | B | 171 | 54.317 | 49.093 | 16.808 | 1.00 | 14.18 | B |
| ATOM | 3264 | C | VAL | B | 171 | 55.065 | 47.178 | 18.979 | 1.00 | 13.79 | B |
| ATOM | 3265 | O | VAL | B | 171 | 54.265 | 47.699 | 19.754 | 1.00 | 13.72 | B |
| ATOM | 3266 | N | ALA | B | 172 | 56.345 | 46.986 | 19.283 | 1.00 | 15.08 | B |
| ATOM | 3267 | CA | ALA | B | 172 | 56.881 | 47.343 | 20.592 | 1.00 | 14.94 | B |
| ATOM | 3268 | CB | ALA | B | 172 | 58.007 | 46.384 | 20.960 | 1.00 | 16.72 | B |
| ATOM | 3269 | C | ALA | B | 172 | 57.366 | 48.776 | 20.744 | 1.00 | 15.27 | B |
| ATOM | 3270 | O | ALA | B | 172 | 57.327 | 49.332 | 21.842 | 1.00 | 14.95 | B |
| ATOM | 3271 | N | THR | B | 173 | 57.826 | 49.373 | 19.651 | 1.00 | 14.44 | B |
| ATOM | 3272 | CA | THR | B | 173 | 58.341 | 50.736 | 19.698 | 1.00 | 14.47 | B |
| ATOM | 3273 | CB | THR | B | 173 | 59.871 | 50.743 | 19.713 | 1.00 | 14.68 | B |
| ATOM | 3274 | OG1 | THR | B | 173 | 60.345 | 50.242 | 18.457 | 1.00 | 15.72 | B |
| ATOM | 3275 | CG2 | THR | B | 173 | 60.404 | 49.868 | 20.835 | 1.00 | 14.59 | B |
| ATOM | 3276 | C | THR | B | 173 | 57.909 | 51.529 | 18.476 | 1.00 | 13.61 | B |
| ATOM | 3277 | O | THR | B | 173 | 57.500 | 50.960 | 17.463 | 1.00 | 13.63 | B |
| ATOM | 3278 | N | THR | B | 174 | 58.023 | 52.849 | 18.570 | 1.00 | 12.92 | B |
| ATOM | 3279 | CA | THR | B | 174 | 57.667 | 53.716 | 17.461 | 1.00 | 13.32 | B |
| ATOM | 3280 | CB | THR | B | 174 | 57.910 | 55.192 | 17.821 | 1.00 | 12.58 | B |
| ATOM | 3281 | OG1 | THR | B | 174 | 57.062 | 55.556 | 18.916 | 1.00 | 13.66 | B |
| ATOM | 3282 | CG2 | THR | B | 174 | 57.601 | 56.095 | 16.632 | 1.00 | 12.16 | B |
| ATOM | 3283 | C | THR | B | 174 | 58.507 | 53.342 | 16.244 | 1.00 | 13.99 | B |
| ATOM | 3284 | O | THR | B | 174 | 57.984 | 53.199 | 15.140 | 1.00 | 12.99 | B |
| ATOM | 3285 | N | ALA | B | 175 | 59.808 | 53.165 | 16.456 | 1.00 | 13.92 | B |
| ATOM | 3286 | CA | ALA | B | 175 | 60.710 | 52.803 | 15.368 | 1.00 | 15.41 | B |
| ATOM | 3287 | CB | ALA | B | 175 | 62.135 | 52.681 | 15.894 | 1.00 | 15.77 | B |
| ATOM | 3288 | C | ALA | B | 175 | 60.284 | 51.499 | 14.696 | 1.00 | 14.93 | B |
| ATOM | 3289 | O | ALA | B | 175 | 60.398 | 51.354 | 13.481 | 1.00 | 15.03 | B |
| ATOM | 3290 | N | GLU | B | 176 | 59.789 | 50.552 | 15.488 | 1.00 | 13.48 | B |
| ATOM | 3291 | CA | GLU | B | 176 | 59.345 | 49.267 | 14.953 | 1.00 | 14.93 | B |
| ATOM | 3292 | CB | GLU | B | 176 | 59.061 | 48.314 | 16.117 | 1.00 | 16.39 | B |
| ATOM | 3293 | CG | GLU | B | 176 | 58.774 | 46.884 | 15.701 | 1.00 | 21.24 | B |
| ATOM | 3294 | CD | GLU | B | 176 | 58.344 | 46.020 | 16.870 | 1.00 | 23.16 | B |
| ATOM | 3295 | OE1 | GLU | B | 176 | 58.988 | 46.099 | 17.938 | 1.00 | 26.24 | B |
| ATOM | 3296 | OE2 | GLU | B | 176 | 57.368 | 45.256 | 16.721 | 1.00 | 25.11 | B |
| ATOM | 3297 | C | GLU | B | 176 | 58.131 | 49.476 | 14.051 | 1.00 | 13.37 | B |
| ATOM | 3298 | O | GLU | B | 176 | 58.021 | 48.867 | 12.984 | 1.00 | 13.90 | B |
| ATOM | 3299 | N | GLY | B | 177 | 57.210 | 50.327 | 14.494 | 1.00 | 11.77 | B |
| ATOM | 3300 | CA | GLY | B | 177 | 56.023 | 50.604 | 13.707 | 1.00 | 11.87 | B |
| ATOM | 3301 | C | GLY | B | 177 | 56.386 | 51.275 | 12.397 | 1.00 | 11.77 | B |
| ATOM | 3302 | O | GLY | B | 177 | 55.859 | 50.920 | 11.342 | 1.00 | 11.59 | B |
| ATOM | 3303 | N | VAL | B | 178 | 57.298 | 52.240 | 12.467 | 1.00 | 12.02 | B |
| ATOM | 3304 | CA | VAL | B | 178 | 57.734 | 52.967 | 11.280 | 1.00 | 11.85 | B |
| ATOM | 3305 | CB | VAL | B | 178 | 58.676 | 54.134 | 11.661 | 1.00 | 13.33 | B |
| ATOM | 3306 | CG1 | VAL | B | 178 | 59.278 | 54.754 | 10.408 | 1.00 | 12.69 | B |
| ATOM | 3307 | CG2 | VAL | B | 178 | 57.908 | 55.184 | 12.449 | 1.00 | 14.50 | B |
| ATOM | 3308 | C | VAL | B | 178 | 58.448 | 52.028 | 10.308 | 1.00 | 11.37 | B |
| ATOM | 3309 | O | VAL | B | 178 | 58.199 | 52.068 | 9.104 | 1.00 | 11.74 | B |
| ATOM | 3310 | N | ALA | B | 179 | 59.329 | 51.179 | 10.831 | 1.00 | 11.93 | B |
| ATOM | 3311 | CA | ALA | B | 179 | 60.050 | 50.231 | 9.987 | 1.00 | 12.51 | B |
| ATOM | 3312 | CB | ALA | B | 179 | 61.048 | 49.435 | 10.824 | 1.00 | 13.53 | B |
| ATOM | 3313 | C | ALA | B | 179 | 59.070 | 49.283 | 9.304 | 1.00 | 13.12 | B |
| ATOM | 3314 | O | ALA | B | 179 | 59.230 | 48.952 | 8.129 | 1.00 | 13.52 | B |
| ATOM | 3315 | N | ARG | B | 180 | 58.047 | 48.850 | 10.038 | 1.00 | 12.34 | B |
| ATOM | 3316 | CA | ARG | B | 180 | 57.060 | 47.936 | 9.477 | 1.00 | 11.65 | B |
| ATOM | 3317 | CB | ARG | B | 180 | 56.143 | 47.429 | 10.590 | 1.00 | 12.34 | B |
| ATOM | 3318 | CG | ARG | B | 180 | 55.216 | 46.324 | 10.142 | 1.00 | 12.89 | B |
| ATOM | 3319 | CD | ARG | B | 180 | 54.600 | 45.637 | 11.340 | 1.00 | 13.42 | B |
| ATOM | 3320 | NE | ARG | B | 180 | 53.642 | 44.618 | 10.932 | 1.00 | 14.57 | B |
| ATOM | 3321 | CZ | ARG | B | 180 | 52.972 | 43.849 | 11.781 | 1.00 | 13.41 | B |
| ATOM | 3322 | NH1 | ARG | B | 180 | 53.167 | 43.981 | 13.086 | 1.00 | 14.58 | B |
| ATOM | 3323 | NH2 | ARG | B | 180 | 52.092 | 42.967 | 11.326 | 1.00 | 15.45 | B |
| ATOM | 3324 | C | ARG | B | 180 | 56.313 | 48.602 | 8.333 | 1.00 | 11.18 | B |
| ATOM | 3325 | O | ARG | B | 180 | 56.050 | 47.981 | 7.305 | 1.00 | 12.37 | B |
| ATOM | 3326 | N | VAL | B | 181 | 55.962 | 49.870 | 8.510 | 1.00 | 10.42 | B |
| ATOM | 3327 | CA | VAL | B | 181 | 55.274 | 50.597 | 7.450 | 1.00 | 11.21 | B |
| ATOM | 3328 | CB | VAL | B | 181 | 54.931 | 52.040 | 7.895 | 1.00 | 11.89 | B |
| ATOM | 3329 | CG1 | VAL | B | 181 | 54.495 | 52.876 | 6.696 | 1.00 | 12.20 | B |
| ATOM | 3330 | CG2 | VAL | B | 181 | 53.826 | 52.009 | 8.946 | 1.00 | 12.54 | B |
| ATOM | 3331 | C | VAL | B | 181 | 56.176 | 50.663 | 6.211 | 1.00 | 11.72 | B |
| ATOM | 3332 | O | VAL | B | 181 | 55.755 | 50.346 | 5.098 | 1.00 | 11.91 | B |
| ATOM | 3333 | N | ARG | B | 182 | 57.427 | 51.051 | 6.426 | 1.00 | 12.96 | B |
| ATOM | 3334 | CA | ARG | B | 182 | 58.391 | 51.198 | 5.342 | 1.00 | 13.16 | B |
| ATOM | 3335 | CB | ARG | B | 182 | 59.645 | 51.882 | 5.889 | 1.00 | 14.59 | B |
| ATOM | 3336 | CG | ARG | B | 182 | 59.443 | 53.341 | 6.247 | 1.00 | 13.75 | B |
| ATOM | 3337 | CD | ARG | B | 182 | 60.707 | 53.964 | 6.811 | 1.00 | 14.39 | B |
| ATOM | 3338 | NE | ARG | B | 182 | 60.590 | 55.145 | 6.901 | 1.00 | 14.11 | B |
| ATOM | 3339 | CZ | ARG | B | 182 | 61.274 | 56.170 | 7.756 | 1.00 | 14.96 | B |
| ATOM | 3340 | NH1 | ARG | B | 182 | 62.126 | 55.613 | 8.604 | 1.00 | 14.64 | B |
| ATOM | 3341 | NH2 | ARG | B | 182 | 61.105 | 57.484 | 7.762 | 1.00 | 16.39 | B |
| ATOM | 3342 | C | ARG | B | 182 | 58.728 | 49.920 | 4.593 | 1.00 | 13.56 | B |
| ATOM | 3343 | O | ARG | B | 182 | 59.000 | 49.966 | 3.394 | 1.00 | 15.12 | B |
| ATOM | 3344 | N | ALA | B | 183 | 58.710 | 48.786 | 5.285 | 1.00 | 13.23 | B |
| ATOM | 3345 | CA | ALA | B | 183 | 59.071 | 47.524 | 4.650 | 1.00 | 14.60 | B |
| ATOM | 3346 | CB | ALA | B | 183 | 60.033 | 46.766 | 5.559 | 1.00 | 15.58 | B |
| ATOM | 3347 | C | ALA | B | 183 | 57.923 | 46.609 | 4.246 | 1.00 | 15.34 | B |
| ATOM | 3348 | O | ALA | B | 183 | 58.162 | 45.524 | 3.718 | 1.00 | 17.94 | B |
| ATOM | 3349 | N | SER | B | 184 | 56.684 | 47.038 | 4.461 | 1.00 | 15.42 | B |
| ATOM | 3350 | CA | SER | B | 184 | 55.542 | 46.187 | 4.134 | 1.00 | 15.22 | B |
| ATOM | 3351 | CB | SER | B | 184 | 54.556 | 46.222 | 5.307 | 1.00 | 15.25 | B |
| ATOM | 3352 | OG | SER | B | 184 | 55.158 | 45.671 | 6.468 | 1.00 | 15.24 | B |
| ATOM | 3353 | C | SER | B | 184 | 54.886 | 46.457 | 2.785 | 1.00 | 14.80 | B |
| ATOM | 3354 | O | SER | B | 184 | 53.788 | 45.972 | 2.520 | 1.00 | 14.65 | B |
| ATOM | 3355 | N | ALA | B | 185 | 55.561 | 47.225 | 1.935 | 1.00 | 15.93 | B |
| ATOM | 3356 | CA | ALA | B | 185 | 55.062 | 47.533 | 0.597 | 1.00 | 16.07 | B |
| ATOM | 3357 | CB | ALA | B | 185 | 55.203 | 46.307 | −0.302 | 1.00 | 16.51 | B |
| ATOM | 3358 | C | ALA | B | 185 | 53.619 | 48.022 | 0.579 | 1.00 | 16.96 | B |
| ATOM | 3359 | O | ALA | B | 185 | 52.847 | 47.673 | −0.320 | 1.00 | 18.07 | B |
| ATOM | 3360 | N | GLY | B | 186 | 53.262 | 48.831 | 1.572 | 1.00 | 16.20 | B |
| ATOM | 3361 | CA | GLY | B | 186 | 51.918 | 49.370 | 1.645 | 1.00 | 15.49 | B |
| ATOM | 3362 | C | GLY | B | 186 | 50.886 | 48.450 | 2.269 | 1.00 | 13.47 | B |
| ATOM | 3363 | O | GLY | B | 186 | 49.697 | 48.771 | 2.260 | 1.00 | 15.31 | B |
| ATOM | 3364 | N | LYS | B | 187 | 51.325 | 47.317 | 2.814 | 1.00 | 13.66 | B |
| ATOM | 3365 | CA | LYS | B | 187 | 50.403 | 46.366 | 3.428 | 1.00 | 13.11 | B |
| ATOM | 3366 | CB | LYS | B | 187 | 50.801 | 44.941 | 3.025 | 1.00 | 15.45 | B |
| ATOM | 3367 | CG | LYS | B | 187 | 50.827 | 44.701 | 1.525 | 1.00 | 16.91 | B |
| ATOM | 3368 | CD | LYS | B | 187 | 49.471 | 44.942 | 0.885 | 1.00 | 19.00 | B |
| ATOM | 3369 | CE | LYS | B | 187 | 49.525 | 44.665 | −0.611 | 1.00 | 21.36 | B |
| ATOM | 3370 | NZ | LYS | B | 187 | 48.215 | 44.906 | −1.274 | 1.00 | 22.80 | B |
| ATOM | 3371 | C | LYS | B | 187 | 50.281 | 46.538 | 4.939 | 1.00 | 12.81 | B |
| ATOM | 3372 | O | LYS | B | 187 | 49.689 | 45.698 | 5.608 | 1.00 | 12.65 | B |
| ATOM | 3373 | N | TYR | B | 188 | 50.853 | 47.609 | 5.483 | 1.00 | 12.17 | B |
| ATOM | 3374 | CA | TYR | B | 188 | 50.750 | 47.878 | 6.914 | 1.00 | 11.57 | B |
| ATOM | 3375 | CB | TYR | B | 188 | 51.961 | 47.319 | 7.676 | 1.00 | 11.09 | B |
| ATOM | 3376 | CG | TYR | B | 188 | 51.841 | 47.487 | 9.186 | 1.00 | 10.63 | B |
| ATOM | 3377 | CD1 | TYR | B | 188 | 51.028 | 46.636 | 9.943 | 1.00 | 10.72 | B |
| ATOM | 3378 | CE1 | TYR | B | 188 | 50.867 | 46.818 | 11.321 | 1.00 | 11.82 | B |
| ATOM | 3379 | CD2 | TYR | B | 188 | 52.495 | 48.527 | 9.852 | 1.00 | 10.42 | B |
| ATOM | 3380 | CE2 | TYR | B | 188 | 52.337 | 48.718 | 11.233 | 1.00 | 10.53 | B |
| ATOM | 3381 | CZ | TYR | B | 188 | 51.523 | 47.860 | 11.958 | 1.00 | 9.62 | B |
| ATOM | 3382 | OH | TYR | B | 188 | 51.377 | 48.051 | 13.316 | 1.00 | 10.96 | B |
| ATOM | 3383 | C | TYR | B | 188 | 50.600 | 49.371 | 7.141 | 1.00 | 10.17 | B |
| ATOM | 3384 | O | TYR | B | 188 | 51.349 | 50.169 | 6.577 | 1.00 | 12.88 | B |
| ATOM | 3385 | N | ALA | B | 189 | 49.623 | 49.737 | 7.962 | 1.00 | 10.30 | B |
| ATOM | 3386 | CA | ALA | B | 189 | 49.384 | 51.130 | 8.327 | 1.00 | 11.13 | B |
| ATOM | 3387 | CB | ALA | B | 189 | 47.984 | 51.562 | 7.928 | 1.00 | 12.49 | B |
| ATOM | 3388 | C | ALA | B | 189 | 49.550 | 51.164 | 9.844 | 1.00 | 10.34 | B |
| ATOM | 3389 | O | ALA | B | 189 | 49.112 | 50.245 | 10.545 | 1.00 | 11.00 | B |
| ATOM | 3390 | N | TYR | B | 190 | 50.175 | 52.219 | 10.352 | 1.00 | 9.97 | B |
| ATOM | 3391 | CA | TYR | B | 190 | 50.445 | 52.334 | 11.777 | 1.00 | 9.16 | B |
| ATOM | 3392 | CB | TYR | B | 190 | 51.970 | 52.483 | 11.932 | 1.00 | 10.28 | B |
| ATOM | 3393 | CG | TYR | B | 190 | 52.483 | 52.705 | 13.336 | 1.00 | 9.88 | B |
| ATOM | 3394 | CD1 | TYR | B | 190 | 51.959 | 51.999 | 14.415 | 1.00 | 10.57 | B |
| ATOM | 3395 | CE1 | TYR | B | 190 | 52.433 | 52.209 | 15.712 | 1.00 | 10.96 | B |
| ATOM | 3396 | CD2 | TYR | B | 190 | 53.503 | 53.625 | 13.581 | 1.00 | 11.85 | B |
| ATOM | 3397 | CE2 | TYR | B | 190 | 53.984 | 53.842 | 14.867 | 1.00 | 12.34 | B |
| ATOM | 3398 | CZ | TYR | B | 190 | 53.443 | 53.137 | 15.927 | 1.00 | 12.44 | B |
| ATOM | 3399 | OH | TYR | B | 190 | 53.887 | 53.392 | 17.206 | 1.00 | 15.74 | B |
| ATOM | 3400 | C | TYR | B | 190 | 49.635 | 53.443 | 12.442 | 1.00 | 9.62 | B |
| ATOM | 3401 | O | TYR | B | 190 | 49.608 | 54.576 | 11.963 | 1.00 | 10.03 | B |
| ATOM | 3402 | N | LEU | B | 191 | 48.965 | 53.105 | 13.543 | 1.00 | 9.49 | B |
| ATOM | 3403 | CA | LEU | B | 191 | 48.167 | 54.070 | 14.296 | 1.00 | 9.49 | B |
| ATOM | 3404 | CB | LEU | B | 191 | 46.900 | 53.382 | 14.815 | 1.00 | 10.49 | B |
| ATOM | 3405 | CG | LEU | B | 191 | 45.817 | 53.104 | 13.771 | 1.00 | 13.15 | B |
| ATOM | 3406 | CD1 | LEU | B | 191 | 44.943 | 51.951 | 14.230 | 1.00 | 16.03 | B |
| ATOM | 3407 | CD2 | LEU | B | 191 | 44.996 | 54.368 | 13.532 | 1.00 | 13.37 | B |
| ATOM | 3408 | C | LEU | B | 191 | 49.049 | 54.615 | 15.405 | 1.00 | 9.27 | B |
| ATOM | 3409 | O | LEU | B | 191 | 49.453 | 53.873 | 16.303 | 1.00 | 9.67 | B |
| ATOM | 3410 | N | LEU | B | 192 | 49.345 | 55.911 | 15.339 | 1.00 | 9.51 | B |
| ATOM | 3411 | CA | LEU | B | 192 | 50.213 | 56.544 | 16.326 | 1.00 | 8.61 | B |
| ATOM | 3412 | CB | LEU | B | 192 | 51.672 | 56.423 | 15.866 | 1.00 | 9.70 | B |
| ATOM | 3413 | CG | LEU | B | 192 | 52.141 | 57.440 | 14.815 | 1.00 | 10.24 | B |
| ATOM | 3414 | CD1 | LEU | B | 192 | 53.640 | 57.295 | 14.607 | 1.00 | 10.76 | B |
| ATOM | 3415 | CD2 | LEU | B | 192 | 51.398 | 57.241 | 13.497 | 1.00 | 10.44 | B |
| ATOM | 3416 | C | LEU | B | 192 | 49.792 | 57.994 | 16.532 | 1.00 | 8.70 | B |
| ATOM | 3417 | O | LEU | B | 192 | 48.979 | 58.535 | 15.779 | 1.00 | 8.53 | B |
| ATOM | 3418 | N | GLU | B | 193 | 50.364 | 58.633 | 17.543 | 1.00 | 9.07 | B |
| ATOM | 3419 | CA | GLU | B | 193 | 50.020 | 60.018 | 17.817 | 1.00 | 8.43 | B |
| ATOM | 3420 | CB | GLU | B | 193 | 50.518 | 60.365 | 19.221 | 1.00 | 8.06 | B |
| ATOM | 3421 | CG | GLU | B | 193 | 49.851 | 59.448 | 20.244 | 1.00 | 8.82 | B |
| ATOM | 3422 | CD | GLU | B | 193 | 50.226 | 59.724 | 21.680 | 1.00 | 8.07 | B |
| ATOM | 3423 | OE1 | GLU | B | 193 | 51.381 | 60.107 | 21.946 | 1.00 | 8.83 | B |
| ATOM | 3424 | OE2 | GLU | B | 193 | 49.350 | 59.529 | 22.546 | 1.00 | 9.84 | B |
| ATOM | 3425 | C | GLU | B | 193 | 50.519 | 60.919 | 16.688 | 1.00 | 8.17 | B |
| ATOM | 3426 | O | GLU | B | 193 | 51.614 | 60.729 | 16.151 | 1.00 | 8.73 | B |
| ATOM | 3427 | N | SER | B | 194 | 49.687 | 61.893 | 16.328 | 1.00 | 8.44 | B |
| ATOM | 3428 | CA | SER | B | 194 | 49.968 | 62.821 | 15.232 | 1.00 | 9.11 | B |
| ATOM | 3429 | CB | SER | B | 194 | 48.824 | 63.834 | 15.147 | 1.00 | 9.75 | B |
| ATOM | 3430 | OG | SER | B | 194 | 48.684 | 64.552 | 16.361 | 1.00 | 9.27 | B |
| ATOM | 3431 | C | SER | B | 194 | 51.334 | 63.477 | 15.333 | 1.00 | 8.86 | B |
| ATOM | 3432 | O | SER | B | 194 | 51.987 | 63.743 | 14.318 | 1.00 | 9.10 | B |
| ATOM | 3433 | N | THR | B | 195 | 51.754 | 63.753 | 16.560 | 1.00 | 8.85 | B |
| ATOM | 3434 | CA | THR | B | 195 | 53.055 | 64.353 | 16.816 | 1.00 | 9.23 | B |
| ATOM | 3435 | CB | THR | B | 195 | 53.224 | 64.564 | 18.314 | 1.00 | 10.50 | B |
| ATOM | 3436 | OG1 | THR | B | 195 | 52.689 | 63.420 | 18.990 | 1.00 | 10.30 | B |
| ATOM | 3437 | CG2 | THR | B | 195 | 52.474 | 65.813 | 18.766 | 1.00 | 11.47 | B |
| ATOM | 3438 | C | THR | B | 195 | 54.185 | 63.468 | 16.278 | 1.00 | 10.40 | B |
| ATOM | 3439 | O | THR | B | 195 | 55.065 | 63.953 | 15.577 | 1.00 | 9.85 | B |
| ATOM | 3440 | N | MET | B | 196 | 54.174 | 62.176 | 16.604 | 1.00 | 10.06 | B |
| ATOM | 3441 | CA | MET | B | 196 | 55.215 | 61.276 | 16.101 | 1.00 | 10.48 | B |
| ATOM | 3442 | CB | MET | B | 196 | 55.136 | 59.909 | 16.796 | 1.00 | 10.02 | B |
| ATOM | 3443 | CG | MET | B | 196 | 55.727 | 59.866 | 18.184 | 1.00 | 11.31 | B |
| ATOM | 3444 | SD | MET | B | 196 | 54.694 | 60.705 | 19.367 | 1.00 | 11.81 | B |
| ATOM | 3445 | CE | MET | B | 196 | 55.074 | 59.782 | 20.855 | 1.00 | 13.94 | B |
| ATOM | 3446 | C | MET | B | 196 | 55.084 | 61.135 | 14.595 | 1.00 | 9.86 | B |
| ATOM | 3447 | O | MET | B | 196 | 56.087 | 61.070 | 13.881 | 1.00 | 10.95 | B |
| ATOM | 3448 | N | ASN | B | 197 | 53.844 | 61.075 | 14.119 | 1.00 | 9.10 | B |
| ATOM | 3449 | CA | ASN | B | 197 | 53.567 | 60.924 | 12.694 | 1.00 | 9.87 | B |
| ATOM | 3450 | CB | ASN | B | 197 | 52.046 | 60.840 | 12.500 | 1.00 | 9.27 | B |
| ATOM | 3451 | CG | ASN | B | 197 | 51.647 | 60.155 | 11.201 | 1.00 | 12.08 | B |
| ATOM | 3452 | OD1 | ASN | B | 197 | 52.465 | 59.953 | 10.304 | 1.00 | 11.55 | B |
| ATOM | 3453 | ND2 | ASN | B | 197 | 50.375 | 59.802 | 11.095 | 1.00 | 9.98 | B |
| ATOM | 3454 | C | ASN | B | 197 | 54.209 | 62.081 | 11.927 | 1.00 | 10.21 | B |
| ATOM | 3455 | O | ASN | B | 197 | 54.915 | 61.869 | 10.934 | 1.00 | 10.46 | B |
| ATOM | 3456 | N | GLU | B | 198 | 53.960 | 63.303 | 12.393 | 1.00 | 10.61 | B |
| ATOM | 3457 | CA | GLU | B | 198 | 54.495 | 64.515 | 11.763 | 1.00 | 10.80 | B |
| ATOM | 3458 | CB | GLU | B | 198 | 53.880 | 65.740 | 12.447 | 1.00 | 13.24 | B |
| ATOM | 3459 | CG | GLU | B | 198 | 52.385 | 65.875 | 12.258 | 1.00 | 15.36 | B |
| ATOM | 3460 | CD | GLU | B | 198 | 51.732 | 66.735 | 13.327 | 1.00 | 18.47 | B |
| ATOM | 3461 | OE1 | GLU | B | 198 | 50.503 | 66.941 | 13.247 | 1.00 | 21.12 | B |
| ATOM | 3462 | OE2 | GLU | B | 198 | 52.440 | 67.200 | 14.249 | 1.00 | 19.03 | B |
| ATOM | 3463 | C | GLU | B | 198 | 56.016 | 64.538 | 11.787 | 1.00 | 10.81 | B |
| ATOM | 3464 | O | GLU | B | 198 | 56.656 | 64.981 | 10.828 | 1.00 | 12.73 | B |
| ATOM | 3465 | N | TYR | B | 199 | 56.596 | 64.078 | 12.889 | 1.00 | 11.33 | B |
| ATOM | 3466 | CA | TYR | B | 199 | 58.044 | 64.048 | 13.032 | 1.00 | 12.01 | B |
| ATOM | 3467 | CB | TYR | B | 199 | 58.391 | 63.624 | 14.457 | 1.00 | 12.78 | B |
| ATOM | 3468 | CG | TYR | B | 199 | 59.826 | 63.191 | 14.619 | 1.00 | 12.16 | B |
| ATOM | 3469 | CD1 | TYR | B | 199 | 60.868 | 64.117 | 14.563 | 1.00 | 14.08 | B |
| ATOM | 3470 | CE1 | TYR | B | 199 | 62.199 | 63.710 | 14.694 | 1.00 | 14.94 | B |
| ATOM | 3471 | CD2 | TYR | B | 199 | 60.145 | 61.848 | 14.812 | 1.00 | 13.76 | B |
| ATOM | 3472 | CE2 | TYR | B | 199 | 61.467 | 61.432 | 14.943 | 1.00 | 15.28 | B |
| ATOM | 3473 | CZ | TYR | B | 199 | 62.486 | 62.367 | 14.883 | 1.00 | 15.02 | B |
| ATOM | 3474 | OH | TYR | B | 199 | 63.790 | 61.949 | 15.007 | 1.00 | 17.71 | B |
| ATOM | 3475 | C | TYR | B | 199 | 58.657 | 63.129 | 11.980 | 1.00 | 11.96 | B |
| ATOM | 3476 | O | TYR | B | 199 | 59.571 | 63.519 | 11.254 | 1.00 | 12.45 | B |
| ATOM | 3477 | N | ILE | B | 200 | 58.148 | 61.905 | 11.910 | 1.00 | 11.56 | B |
| ATOM | 3478 | CA | ILE | B | 200 | 58.638 | 60.909 | 10.962 | 1.00 | 11.82 | B |
| ATOM | 3479 | CB | ILE | B | 200 | 57.940 | 59.547 | 11.205 | 1.00 | 12.48 | B |
| ATOM | 3480 | CG2 | ILE | B | 200 | 58.368 | 58.541 | 10.147 | 1.00 | 12.77 | B |
| ATOM | 3481 | CG1 | ILE | B | 200 | 58.273 | 59.021 | 12.609 | 1.00 | 13.25 | B |
| ATOM | 3482 | CD1 | ILE | B | 200 | 59.733 | 58.644 | 12.819 | 1.00 | 13.40 | B |
| ATOM | 3483 | C | ILE | B | 200 | 58.428 | 61.329 | 9.502 | 1.00 | 12.47 | B |
| ATOM | 3484 | O | ILE | B | 200 | 59.240 | 61.005 | 8.632 | 1.00 | 13.01 | B |
| ATOM | 3485 | N | GLU | B | 201 | 57.344 | 62.051 | 9.240 | 1.00 | 11.53 | B |
| ATOM | 3486 | CA | GLU | B | 201 | 57.036 | 62.508 | 7.890 | 1.00 | 14.28 | B |
| ATOM | 3487 | CB | GLU | B | 201 | 55.673 | 63.197 | 7.905 | 1.00 | 14.24 | B |
| ATOM | 3488 | CG | GLU | B | 201 | 55.102 | 63.502 | 6.535 | 1.00 | 19.66 | B |
| ATOM | 3489 | CD | GLU | B | 201 | 53.701 | 64.075 | 6.616 | 1.00 | 21.29 | B |
| ATOM | 3490 | OE1 | GLU | B | 201 | 53.544 | 65.193 | 7.151 | 1.00 | 25.24 | B |
| ATOM | 3491 | OE2 | GLU | B | 201 | 52.756 | 63.406 | 6.152 | 1.00 | 25.00 | B |
| ATOM | 3492 | C | GLU | B | 201 | 58.144 | 63.403 | 7.348 | 1.00 | 15.97 | B |
| ATOM | 3493 | O | GLU | B | 201 | 58.273 | 63.570 | 6.133 | 1.00 | 16.88 | B |
| ATOM | 3494 | N | GLN | B | 202 | 58.939 | 63.980 | 8.245 | 1.00 | 15.22 | B |
| ATOM | 3495 | CA | GLN | B | 202 | 60.029 | 64.864 | 7.844 | 1.00 | 18.83 | B |
| ATOM | 3496 | CB | GLN | B | 202 | 59.961 | 66.150 | 8.677 | 1.00 | 19.32 | B |
| ATOM | 3497 | CG | GLN | B | 202 | 58.627 | 66.882 | 8.602 | 1.00 | 24.25 | B |
| ATOM | 3498 | CD | GLN | B | 202 | 58.205 | 67.193 | 7.178 | 1.00 | 27.58 | B |
| ATOM | 3499 | OE1 | GLN | B | 202 | 58.957 | 67.795 | 6.411 | 1.00 | 31.06 | B |
| ATOM | 3500 | NE2 | GLN | B | 202 | 56.992 | 66.787 | 6.819 | 1.00 | 29.66 | B |
| ATOM | 3501 | C | GLN | B | 202 | 61.379 | 64.164 | 7.952 | 1.00 | 18.85 | B |
| ATOM | 3502 | O | GLN | B | 202 | 62.423 | 64.816 | 7.925 | 1.00 | 21.64 | B |
| ATOM | 3503 | N | ARG | B | 203 | 61.361 | 62.841 | 8.070 | 1.00 | 19.16 | B |
| ATOM | 3504 | CA | ARG | B | 203 | 62.594 | 62.069 | 8.183 | 1.00 | 19.65 | B |
| ATOM | 3505 | CB | ARG | B | 203 | 62.526 | 61.193 | 9.441 | 1.00 | 20.67 | B |
| ATOM | 3506 | CG | ARG | B | 203 | 62.292 | 61.956 | 10.734 | 1.00 | 22.05 | B |
| ATOM | 3507 | CD | ARG | B | 203 | 63.531 | 62.718 | 11.174 | 1.00 | 24.79 | B |
| ATOM | 3508 | NE | ARG | B | 203 | 64.605 | 61.819 | 11.589 | 1.00 | 25.67 | B |
| ATOM | 3509 | CZ | ARG | B | 203 | 65.771 | 62.226 | 12.079 | 1.00 | 26.71 | B |
| ATOM | 3510 | NH1 | ARG | B | 203 | 66.018 | 63.522 | 12.215 | 1.00 | 26.28 | B |
| ATOM | 3511 | NH2 | ARG | B | 203 | 66.691 | 61.338 | 12.435 | 1.00 | 26.10 | B |
| ATOM | 3512 | C | ARG | B | 203 | 62.822 | 61.249 | 6.917 | 1.00 | 19.60 | B |
| ATOM | 3513 | O | ARG | B | 203 | 61.880 | 60.713 | 6.335 | 1.00 | 17.46 | B |
| ATOM | 3514 | N | LYS | B | 204 | 64.077 | 61.156 | 6.491 | 1.00 | 20.33 | B |
| ATOM | 3515 | CA | LYS | B | 204 | 64.415 | 60.370 | 5.312 | 1.00 | 21.37 | B |
| ATOM | 3516 | CB | LYS | B | 204 | 65.896 | 60.578 | 4.975 | 1.00 | 21.77 | B |
| ATOM | 3517 | CG | LYS | B | 204 | 66.243 | 61.991 | 4.525 | 1.00 | 25.46 | B |
| ATOM | 3518 | CD | LYS | B | 204 | 67.752 | 62.222 | 4.512 | 1.00 | 28.33 | B |
| ATOM | 3519 | CE | LYS | B | 204 | 68.468 | 61.239 | 3.600 | 1.00 | 29.88 | B |
| ATOM | 3520 | NZ | LYS | B | 204 | 69.950 | 61.384 | 3.686 | 1.00 | 31.44 | B |
| ATOM | 3521 | C | LYS | B | 204 | 64.068 | 58.928 | 5.664 | 1.00 | 21.04 | B |
| ATOM | 3522 | O | LYS | B | 204 | 64.127 | 58.541 | 6.831 | 1.00 | 22.46 | B |
| ATOM | 3523 | N | PRO | B | 205 | 63.735 | 58.101 | 4.662 | 1.00 | 20.47 | B |
| ATOM | 3524 | CD | PRO | B | 205 | 63.604 | 56.655 | 4.915 | 1.00 | 20.59 | B |
| ATOM | 3525 | CA | PRO | B | 205 | 63.652 | 58.378 | 3.225 | 1.00 | 20.46 | B |
| ATOM | 3526 | CB | PRO | B | 205 | 63.880 | 56.996 | 2.620 | 1.00 | 21.06 | B |
| ATOM | 3527 | CG | PRO | B | 205 | 63.090 | 56.109 | 3.530 | 1.00 | 22.08 | B |
| ATOM | 3528 | C | PRO | B | 205 | 62.396 | 59.099 | 2.725 | 1.00 | 19.52 | B |
| ATOM | 3529 | O | PRO | B | 205 | 62.064 | 59.011 | 1.543 | 1.00 | 19.08 | B |
| ATOM | 3530 | N | CYS | B | 206 | 61.690 | 59.799 | 3.608 | 1.00 | 17.99 | B |
| ATOM | 3531 | CA | CYS | B | 206 | 60.504 | 60.544 | 3.190 | 1.00 | 16.87 | B |
| ATOM | 3532 | C | CYS | B | 206 | 59.509 | 59.659 | 2.453 | 1.00 | 15.84 | B |
| ATOM | 3533 | O | CYS | B | 206 | 58.973 | 60.055 | 1.418 | 1.00 | 15.47 | B |
| ATOM | 3534 | CB | CYS | B | 206 | 60.955 | 61.721 | 2.326 | 1.00 | 19.04 | B |
| ATOM | 3535 | SG | CYS | B | 206 | 62.275 | 62.714 | 3.076 | 1.00 | 20.10 | B |
| ATOM | 3536 | N | ASP | B | 207 | 59.245 | 58.472 | 2.988 | 1.00 | 15.12 | B |
| ATOM | 3537 | CA | ASP | B | 207 | 58.332 | 57.545 | 2.336 | 1.00 | 14.66 | B |
| ATOM | 3538 | CB | ASP | B | 207 | 59.096 | 56.256 | 2.019 | 1.00 | 15.55 | B |
| ATOM | 3539 | CG | ASP | B | 207 | 59.615 | 55.567 | 3.266 | 1.00 | 15.30 | B |
| ATOM | 3540 | OD1 | ASP | B | 207 | 59.661 | 56.220 | 4.330 | 1.00 | 17.08 | B |
| ATOM | 3541 | OD2 | ASP | B | 207 | 59.989 | 54.377 | 3.182 | 1.00 | 18.19 | B |
| ATOM | 3542 | C | ASP | B | 207 | 57.061 | 57.298 | 3.135 | 1.00 | 14.35 | B |
| ATOM | 3543 | O | ASP | B | 207 | 56.287 | 56.400 | 2.811 | 1.00 | 13.73 | B |
| ATOM | 3544 | N | THR | B | 208 | 56.845 | 58.088 | 4.181 | 1.00 | 14.62 | B |
| ATOM | 3545 | CA | THR | B | 208 | 55.645 | 57.925 | 4.992 | 1.00 | 13.43 | B |
| ATOM | 3546 | CB | THR | B | 208 | 55.978 | 57.704 | 6.485 | 1.00 | 13.85 | B |
| ATOM | 3547 | OG1 | THR | B | 208 | 56.642 | 58.860 | 7.008 | 1.00 | 15.61 | B |
| ATOM | 3548 | CG2 | THR | B | 208 | 56.869 | 56.483 | 6.657 | 1.00 | 15.09 | B |
| ATOM | 3549 | C | THR | B | 208 | 54.765 | 59.159 | 4.871 | 1.00 | 13.60 | B |
| ATOM | 3550 | O | THR | B | 208 | 55.221 | 60.228 | 4.458 | 1.00 | 14.47 | B |
| ATOM | 3551 | N | MET | B | 209 | 53.498 | 59.009 | 5.234 | 1.00 | 13.97 | B |
| ATOM | 3552 | CA | MET | B | 209 | 52.569 | 60.118 | 5.156 | 1.00 | 14.24 | B |
| ATOM | 3553 | CB | MET | B | 209 | 52.053 | 60.224 | 3.720 | 1.00 | 15.73 | B |
| ATOM | 3554 | CG | MET | B | 209 | 50.998 | 61.291 | 3.526 | 1.00 | 18.55 | B |
| ATOM | 3555 | SD | MET | B | 209 | 50.531 | 61.480 | 1.800 | 1.00 | 24.13 | B |
| ATOM | 3556 | CE | MET | B | 209 | 49.421 | 60.119 | 1.585 | 1.00 | 24.81 | B |
| ATOM | 3557 | C | MET | B | 209 | 51.445 | 59.977 | 6.168 | 1.00 | 13.47 | B |
| ATOM | 3558 | O | MET | B | 209 | 50.973 | 58.873 | 6.439 | 1.00 | 13.39 | B |
| ATOM | 3559 | N | LYS | B | 210 | 51.029 | 61.107 | 6.728 | 1.00 | 14.58 | B |
| ATOM | 3560 | CA | LYS | B | 210 | 49.925 | 61.135 | 7.677 | 1.00 | 15.13 | B |
| ATOM | 3561 | CB | LYS | B | 210 | 50.163 | 62.239 | 8.713 | 1.00 | 15.35 | B |
| ATOM | 3562 | CG | LYS | B | 210 | 48.994 | 62.449 | 9.660 | 1.00 | 17.22 | B |
| ATOM | 3563 | CD | LYS | B | 210 | 49.267 | 63.588 | 10.630 | 1.00 | 20.64 | B |
| ATOM | 3564 | CE | LYS | B | 210 | 48.098 | 63.805 | 11.570 | 1.00 | 21.70 | B |
| ATOM | 3565 | NZ | LYS | B | 210 | 48.312 | 64.990 | 12.443 | 1.00 | 25.64 | B |
| ATOM | 3566 | C | LYS | B | 210 | 48.679 | 61.367 | 6.832 | 1.00 | 15.45 | B |
| ATOM | 3567 | O | LYS | B | 210 | 48.629 | 62.316 | 6.047 | 1.00 | 16.38 | B |
| ATOM | 3568 | N | VAL | B | 211 | 47.676 | 60.508 | 6.984 | 1.00 | 14.47 | B |
| ATOM | 3569 | CA | VAL | B | 211 | 46.447 | 60.646 | 6.209 | 1.00 | 16.16 | B |
| ATOM | 3570 | CB | VAL | B | 211 | 46.308 | 59.517 | 5.161 | 1.00 | 15.70 | B |
| ATOM | 3571 | CG1 | VAL | B | 211 | 47.420 | 59.623 | 4.134 | 1.00 | 18.13 | B |
| ATOM | 3572 | CG2 | VAL | B | 211 | 46.347 | 58.158 | 5.847 | 1.00 | 16.74 | B |
| ATOM | 3573 | C | VAL | B | 211 | 45.191 | 60.655 | 7.068 | 1.00 | 17.02 | B |
| ATOM | 3574 | O | VAL | B | 211 | 45.155 | 60.059 | 8.147 | 1.00 | 15.85 | B |
| ATOM | 3575 | N | GLY | B | 212 | 44.160 | 61.332 | 6.569 | 1.00 | 18.91 | B |
| ATOM | 3576 | CA | GLY | B | 212 | 42.902 | 61.420 | 7.285 | 1.00 | 19.63 | B |
| ATOM | 3577 | C | GLY | B | 212 | 42.989 | 62.341 | 8.483 | 1.00 | 18.48 | B |
| ATOM | 3578 | O | GLY | B | 212 | 44.077 | 62.764 | 8.877 | 1.00 | 20.50 | B |
| ATOM | 3579 | N | GLY | B | 213 | 41.839 | 62.670 | 9.059 | 1.00 | 18.28 | B |
| ATOM | 3580 | CA | GLY | B | 213 | 41.837 | 63.530 | 10.224 | 1.00 | 17.13 | B |
| ATOM | 3581 | C | GLY | B | 213 | 42.213 | 62.700 | 11.433 | 1.00 | 15.25 | B |
| ATOM | 3582 | O | GLY | B | 213 | 42.205 | 61.465 | 11.373 | 1.00 | 17.51 | B |
| ATOM | 3583 | N | ASN | B | 214 | 42.560 | 63.362 | 12.530 | 1.00 | 14.08 | B |
| ATOM | 3584 | CA | ASN | B | 214 | 42.914 | 62.635 | 13.738 | 1.00 | 12.59 | B |
| ATOM | 3585 | CB | ASN | B | 214 | 43.539 | 63.594 | 14.754 | 1.00 | 13.59 | B |
| ATOM | 3586 | CG | ASN | B | 214 | 44.882 | 64.128 | 14.304 | 1.00 | 14.04 | B |
| ATOM | 3587 | OD1 | ASN | B | 214 | 45.520 | 63.570 | 13.410 | 1.00 | 15.39 | B |
| ATOM | 3588 | ND2 | ASN | B | 214 | 45.328 | 65.205 | 14.938 | 1.00 | 15.44 | B |
| ATOM | 3589 | C | ASN | B | 214 | 41.662 | 61.945 | 14.260 | 1.00 | 12.51 | B |
| ATOM | 3590 | O | ASN | B | 214 | 40.545 | 62.434 | 14.070 | 1.00 | 13.98 | B |
| ATOM | 3591 | N | LEU | B | 215 | 41.853 | 60.811 | 14.922 | 1.00 | 11.43 | B |
| ATOM | 3592 | CA | LEU | B | 215 | 40.736 | 60.052 | 15.471 | 1.00 | 10.90 | B |
| ATOM | 3593 | CB | LEU | B | 215 | 41.146 | 58.586 | 15.635 | 1.00 | 11.99 | B |
| ATOM | 3594 | CG | LEU | B | 215 | 41.241 | 57.742 | 14.361 | 1.00 | 12.59 | B |
| ATOM | 3595 | CD1 | LEU | B | 215 | 42.292 | 58.306 | 13.416 | 1.00 | 16.18 | B |
| ATOM | 3596 | CD2 | LEU | B | 215 | 41.581 | 56.316 | 14.744 | 1.00 | 13.71 | B |
| ATOM | 3597 | C | LEU | B | 215 | 40.223 | 60.670 | 16.765 | 1.00 | 10.91 | B |
| ATOM | 3598 | O | LEU | B | 215 | 39.087 | 60.410 | 17.165 | 1.00 | 12.75 | B |
| ATOM | 3599 | N | ASP | B | 216 | 41.066 | 61.453 | 17.437 | 1.00 | 10.37 | B |
| ATOM | 3600 | CA | ASP | B | 216 | 40.678 | 62.123 | 18.673 | 1.00 | 9.90 | B |
| ATOM | 3601 | CB | ASP | B | 216 | 40.772 | 61.151 | 19.873 | 1.00 | 9.05 | B |
| ATOM | 3602 | CG | ASP | B | 216 | 42.194 | 60.733 | 20.221 | 1.00 | 9.75 | B |
| ATOM | 3603 | OD1 | ASP | B | 216 | 43.159 | 61.145 | 19.546 | 1.00 | 9.43 | B |
| ATOM | 3604 | OD2 | ASP | B | 216 | 42.342 | 59.975 | 21.208 | 1.00 | 9.63 | B |
| ATOM | 3605 | C | ASP | B | 216 | 41.484 | 63.405 | 18.826 | 1.00 | 9.20 | B |
| ATOM | 3606 | O | ASP | B | 216 | 42.306 | 63.728 | 17.968 | 1.00 | 10.98 | B |
| ATOM | 3607 | N | SER | B | 217 | 41.222 | 64.143 | 19.903 | 1.00 | 9.88 | B |
| ATOM | 3608 | CA | SER | B | 217 | 41.899 | 65.409 | 20.174 | 1.00 | 9.92 | B |
| ATOM | 3609 | CB | SER | B | 217 | 40.987 | 66.560 | 19.746 | 1.00 | 12.87 | B |
| ATOM | 3610 | OG | SER | B | 217 | 40.715 | 66.507 | 18.361 | 1.00 | 20.77 | B |
| ATOM | 3611 | C | SER | B | 217 | 42.271 | 65.483 | 21.645 | 1.00 | 10.98 | B |
| ATOM | 3612 | O | SER | B | 217 | 41.398 | 65.520 | 22.511 | 1.00 | 12.32 | B |
| ATOM | 3613 | N | LYS | B | 218 | 43.567 | 65.524 | 21.928 | 1.00 | 11.11 | B |
| ATOM | 3614 | CA | LYS | B | 218 | 44.037 | 65.574 | 23.302 | 1.00 | 9.81 | B |
| ATOM | 3615 | CB | LYS | B | 218 | 44.082 | 64.143 | 23.854 | 1.00 | 11.05 | B |
| ATOM | 3616 | CG | LYS | B | 218 | 44.895 | 63.171 | 23.013 | 1.00 | 11.12 | B |
| ATOM | 3617 | CD | LYS | B | 218 | 44.435 | 61.740 | 23.254 | 1.00 | 9.73 | B |
| ATOM | 3618 | CE | LYS | B | 218 | 45.266 | 60.748 | 22.453 | 1.00 | 9.17 | B |
| ATOM | 3619 | NZ | LYS | B | 218 | 44.647 | 59.391 | 22.476 | 1.00 | 8.65 | B |
| ATOM | 3620 | C | LYS | B | 218 | 45.366 | 66.306 | 22.376 | 1.00 | 9.32 | B |
| ATOM | 3621 | O | LYS | B | 218 | 45.705 | 67.084 | 22.486 | 1.00 | 8.84 | B |
| ATOM | 3622 | N | GLY | B | 219 | 46.116 | 66.069 | 24.445 | 1.00 | 9.27 | B |
| ATOM | 3623 | CA | GLY | B | 219 | 47.398 | 66.735 | 24.585 | 1.00 | 8.98 | B |
| ATOM | 3624 | C | GLY | B | 219 | 48.316 | 66.051 | 25.573 | 1.00 | 8.88 | B |
| ATOM | 3625 | O | GLY | B | 219 | 47.897 | 65.140 | 26.292 | 1.00 | 9.06 | B |
| ATOM | 3626 | N | TYR | B | 220 | 49.578 | 66.473 | 25.574 | 1.00 | 7.83 | B |
| ATOM | 3627 | CA | TYR | B | 220 | 50.574 | 65.962 | 26.507 | 1.00 | 8.61 | B |
| ATOM | 3628 | CB | TYR | B | 220 | 51.961 | 65.899 | 25.871 | 1.00 | 8.83 | B |
| ATOM | 3629 | CG | TYR | B | 220 | 52.107 | 64.983 | 24.697 | 1.00 | 9.25 | B |
| ATOM | 3630 | CD1 | TYR | B | 220 | 51.520 | 63.719 | 24.690 | 1.00 | 9.06 | B |
| ATOM | 3631 | CE1 | TYR | B | 220 | 51.708 | 62.850 | 23.625 | 1.00 | 8.63 | B |
| ATOM | 3632 | CD2 | TYR | B | 220 | 52.883 | 65.359 | 23.607 | 1.00 | 8.80 | B |
| ATOM | 3633 | CE2 | TYR | B | 220 | 53.079 | 64.501 | 22.540 | 1.00 | 8.78 | B |
| ATOM | 3634 | CZ | TYR | B | 220 | 52.491 | 63.251 | 22.554 | 1.00 | 8.54 | B |
| ATOM | 3635 | OH | TYR | B | 220 | 52.691 | 62.403 | 21.491 | 1.00 | 9.33 | B |
| ATOM | 3636 | C | TYR | B | 220 | 50.597 | 66.928 | 27.670 | 1.00 | 7.92 | B |
| ATOM | 3637 | O | TYR | B | 220 | 50.487 | 68.145 | 27.478 | 1.00 | 8.32 | B |
| ATOM | 3638 | N | GLY | B | 221 | 50.770 | 66.391 | 28.869 | 1.00 | 8.36 | B |
| ATOM | 3639 | CA | GLY | B | 221 | 50.840 | 67.239 | 30.039 | 1.00 | 9.29 | B |
| ATOM | 3640 | C | GLY | B | 221 | 52.001 | 66.867 | 30.936 | 1.00 | 8.00 | B |
| ATOM | 3641 | O | GLY | B | 221 | 52.469 | 65.727 | 30.916 | 1.00 | 8.77 | B |
| ATOM | 3642 | N | ILE | B | 222 | 52.495 | 67.840 | 31.693 | 1.00 | 8.57 | B |
| ATOM | 3643 | CA | ILE | B | 222 | 53.565 | 67.590 | 32.646 | 1.00 | 8.46 | B |
| ATOM | 3644 | CB | ILE | B | 222 | 54.310 | 68.895 | 33.010 | 1.00 | 8.80 | B |
| ATOM | 3645 | CG2 | ILE | B | 222 | 55.246 | 68.652 | 34.190 | 1.00 | 11.10 | B |
| ATOM | 3646 | CG1 | ILE | B | 222 | 55.082 | 69.402 | 31.785 | 1.00 | 12.64 | B |
| ATOM | 3647 | CD1 | ILE | B | 222 | 55.892 | 70.668 | 32.024 | 1.00 | 14.32 | B |
| ATOM | 3648 | C | ILE | B | 222 | 52.848 | 67.016 | 33.872 | 1.00 | 8.77 | B |
| ATOM | 3649 | O | ILE | B | 222 | 51.804 | 67.531 | 34.285 | 1.00 | 10.76 | B |
| ATOM | 3650 | N | ALA | B | 223 | 53.390 | 65.951 | 34.450 | 1.00 | 9.70 | B |
| ATOM | 3651 | CA | ALA | B | 223 | 52.745 | 65.321 | 35.596 | 1.00 | 9.37 | B |
| ATOM | 3652 | CB | ALA | B | 223 | 52.454 | 63.852 | 35.284 | 1.00 | 9.67 | B |
| ATOM | 3653 | C | ALA | B | 223 | 53.536 | 65.428 | 36.889 | 1.00 | 9.44 | B |
| ATOM | 3654 | O | ALA | B | 223 | 54.764 | 65.406 | 36.890 | 1.00 | 8.84 | B |
| ATOM | 3655 | N | THR | B | 224 | 52.802 | 65.546 | 37.990 | 1.00 | 9.86 | B |
| ATOM | 3656 | CA | THR | B | 224 | 53.384 | 65.630 | 39.322 | 1.00 | 10.14 | B |
| ATOM | 3657 | CB | THR | B | 224 | 53.408 | 67.088 | 39.846 | 1.00 | 10.69 | B |
| ATOM | 3658 | OG1 | THR | B | 224 | 52.133 | 67.698 | 39.623 | 1.00 | 12.13 | B |
| ATOM | 3659 | CG2 | THR | B | 224 | 54.491 | 67.895 | 39.153 | 1.00 | 10.75 | B |
| ATOM | 3660 | C | THR | B | 224 | 52.507 | 64.796 | 40.252 | 1.00 | 10.82 | B |
| ATOM | 3661 | O | THR | B | 224 | 51.354 | 64.513 | 39.937 | 1.00 | 11.22 | B |
| ATOM | 3662 | N | PRO | B | 225 | 53.049 | 64.370 | 41.403 | 1.00 | 11.54 | B |
| ATOM | 3663 | CD | PRO | B | 225 | 54.399 | 64.568 | 41.953 | 1.00 | 12.42 | B |
| ATOM | 3664 | CA | PRO | B | 225 | 52.216 | 63.576 | 42.312 | 1.00 | 12.84 | B |
| ATOM | 3665 | CB | PRO | B | 225 | 53.217 | 63.129 | 43.380 | 1.00 | 13.64 | B |
| ATOM | 3666 | CG | PRO | B | 225 | 54.171 | 64.288 | 43.486 | 1.00 | 17.80 | B |
| ATOM | 3667 | C | PRO | B | 225 | 51.063 | 64.440 | 42.812 | 1.00 | 14.09 | B |
| ATOM | 3668 | O | PRO | B | 225 | 51.195 | 65.658 | 42.906 | 1.00 | 14.20 | B |
| ATOM | 3669 | N | LYS | B | 226 | 49.931 | 63.816 | 43.119 | 1.00 | 15.70 | B |
| ATOM | 3670 | CA | LYS | B | 226 | 48.783 | 64.565 | 43.612 | 1.00 | 19.44 | B |
| ATOM | 3671 | CB | LYS | B | 226 | 47.619 | 63.607 | 43.874 | 1.00 | 20.33 | B |
| ATOM | 3672 | CG | LYS | B | 226 | 46.650 | 63.471 | 42.723 | 1.00 | 22.92 | B |
| ATOM | 3673 | CD | LYS | B | 226 | 45.386 | 62.769 | 43.184 | 1.00 | 24.41 | B |
| ATOM | 3674 | CE | LYS | B | 226 | 44.326 | 62.765 | 42.102 | 1.00 | 25.95 | B |
| ATOM | 3675 | NZ | LYS | B | 226 | 43.062 | 62.157 | 42.594 | 1.00 | 27.19 | B |
| ATOM | 3676 | C | LYS | B | 226 | 49.154 | 65.371 | 44.845 | 1.00 | 20.40 | B |
| ATOM | 3677 | O | LYS | B | 226 | 49.795 | 64.861 | 45.765 | 1.00 | 20.12 | B |
| ATOM | 3678 | N | GLY | B | 227 | 48.751 | 66.637 | 44.856 | 1.00 | 21.76 | B |
| ATOM | 3679 | CA | GLY | B | 227 | 49.034 | 67.493 | 45.992 | 1.00 | 22.87 | B |
| ATOM | 3680 | C | GLY | B | 227 | 50.411 | 68.126 | 45.994 | 1.00 | 23.69 | B |
| ATOM | 3681 | O | GLY | B | 227 | 50.720 | 68.932 | 46.871 | 1.00 | 24.90 | B |
| ATOM | 3682 | N | SER | B | 228 | 51.243 | 67.767 | 45.020 | 1.00 | 23.21 | B |
| ATOM | 3683 | CA | SER | B | 228 | 52.591 | 68.318 | 44.926 | 1.00 | 24.09 | B |
| ATOM | 3684 | CB | SER | B | 228 | 53.279 | 67.758 | 43.678 | 1.00 | 23.78 | B |
| ATOM | 3685 | OG | SER | B | 228 | 54.504 | 68.431 | 43.442 | 1.00 | 24.30 | B |
| ATOM | 3686 | C | SER | B | 228 | 52.570 | 69.839 | 44.942 | 1.00 | 24.66 | B |
| ATOM | 3687 | O | SER | B | 228 | 51.767 | 70.466 | 44.254 | 1.00 | 24.48 | B |
| ATOM | 3688 | N | SER | B | 229 | 53.470 | 70.428 | 45.721 | 1.00 | 25.30 | B |
| ATOM | 3689 | CA | SER | B | 229 | 53.561 | 71.879 | 45.828 | 1.00 | 25.59 | B |
| ATOM | 3690 | CB | SER | B | 229 | 54.252 | 72.236 | 47.146 | 1.00 | 25.94 | B |
| ATOM | 3691 | OG | SER | B | 229 | 55.577 | 71.737 | 47.170 | 1.00 | 28.01 | B |
| ATOM | 3692 | C | SER | B | 229 | 54.278 | 72.456 | 44.613 | 1.00 | 24.69 | B |
| ATOM | 3693 | O | SER | B | 229 | 54.496 | 73.664 | 44.528 | 1.00 | 25.48 | B |
| ATOM | 3694 | N | LEU | B | 230 | 54.651 | 71.587 | 43.678 | 1.00 | 22.34 | B |
| ATOM | 3695 | CA | LEU | B | 230 | 55.346 | 72.009 | 42.468 | 1.00 | 21.30 | B |
| ATOM | 3696 | CB | LEU | B | 230 | 56.461 | 71.002 | 42.157 | 1.00 | 23.11 | B |
| ATOM | 3697 | CG | LEU | B | 230 | 57.896 | 71.524 | 42.050 | 1.00 | 25.25 | B |
| ATOM | 3698 | CD1 | LEU | B | 230 | 57.978 | 72.580 | 40.962 | 1.00 | 24.39 | B |
| ATOM | 3699 | CD2 | LEU | B | 230 | 58.338 | 72.097 | 43.387 | 1.00 | 27.73 | B |
| ATOM | 3700 | C | LEU | B | 230 | 54.340 | 72.143 | 41.333 | 1.00 | 19.83 | B |
| ATOM | 3701 | O | LEU | B | 230 | 54.577 | 72.858 | 40.359 | 1.00 | 19.58 | B |
| ATOM | 3702 | N | GLY | B | 231 | 53.212 | 71.455 | 41.479 | 1.00 | 18.69 | B |
| ATOM | 3703 | CA | GLY | B | 231 | 52.177 | 71.473 | 40.461 | 1.00 | 18.22 | B |
| ATOM | 3704 | C | GLY | B | 231 | 51.704 | 72.832 | 39.981 | 1.00 | 17.36 | B |
| ATOM | 3705 | O | GLY | B | 231 | 51.672 | 73.084 | 38.778 | 1.00 | 16.54 | B |
| ATOM | 3706 | N | ASN | B | 232 | 51.332 | 73.711 | 40.907 | 1.00 | 17.32 | B |
| ATOM | 3707 | CA | ASN | B | 232 | 50.844 | 75.033 | 40.534 | 1.00 | 18.12 | B |
| ATOM | 3708 | CB | ASN | B | 232 | 50.409 | 75.789 | 41.792 | 1.00 | 19.85 | B |
| ATOM | 3709 | CG | ASN | B | 232 | 49.897 | 77.181 | 41.479 | 1.00 | 21.52 | B |
| ATOM | 3710 | OD1 | ASN | B | 232 | 48.916 | 77.345 | 40.751 | 1.00 | 22.55 | B |
| ATOM | 3711 | ND2 | ASN | B | 232 | 50.561 | 78.194 | 42.025 | 1.00 | 22.41 | B |
| ATOM | 3712 | C | ASN | B | 232 | 51.861 | 75.814 | 39.710 | 1.00 | 16.03 | B |
| ATOM | 3713 | O | ASN | B | 232 | 51.540 | 76.321 | 38.635 | 1.00 | 14.82 | B |
| ATOM | 3714 | N | ALA | B | 233 | 53.083 | 75.917 | 40.223 | 1.00 | 16.42 | B |
| ATOM | 3715 | CA | ALA | B | 233 | 54.139 | 76.650 | 39.536 | 1.00 | 15.32 | B |
| ATOM | 3716 | CB | ALA | B | 233 | 55.423 | 76.610 | 40.355 | 1.00 | 16.83 | B |
| ATOM | 3717 | C | ALA | B | 233 | 54.391 | 76.089 | 38.140 | 1.00 | 13.41 | B |
| ATOM | 3718 | O | ALA | B | 233 | 54.495 | 76.838 | 37.171 | 1.00 | 13.67 | B |
| ATOM | 3719 | N | VAL | B | 234 | 54.489 | 74.769 | 38.043 | 1.00 | 13.56 | B |
| ATOM | 3720 | CA | VAL | B | 234 | 54.732 | 74.124 | 36.760 | 1.00 | 13.50 | B |
| ATOM | 3721 | CB | VAL | B | 234 | 54.857 | 72.597 | 36.933 | 1.00 | 15.11 | B |
| ATOM | 3722 | CG1 | VAL | B | 234 | 54.762 | 71.899 | 35.585 | 1.00 | 15.97 | B |
| ATOM | 3723 | CG2 | VAL | B | 234 | 56.181 | 72.272 | 37.602 | 1.00 | 15.87 | B |
| ATOM | 3724 | C | VAL | B | 234 | 53.635 | 74.445 | 35.753 | 1.00 | 13.01 | B |
| ATOM | 3725 | O | VAL | B | 234 | 53.917 | 74.715 | 34.586 | 1.00 | 12.10 | B |
| ATOM | 3726 | N | ASN | B | 235 | 52.382 | 74.427 | 36.199 | 1.00 | 12.91 | B |
| ATOM | 3727 | CA | ASN | B | 235 | 51.275 | 74.729 | 35.303 | 1.00 | 12.22 | B |
| ATOM | 3728 | CB | ASN | B | 235 | 49.948 | 74.416 | 35.998 | 1.00 | 12.94 | B |
| ATOM | 3729 | CG | ASN | B | 235 | 48.758 | 74.628 | 35.086 | 1.00 | 12.76 | B |
| ATOM | 3730 | OD1 | ASN | B | 235 | 48.660 | 74.013 | 34.025 | 1.00 | 12.94 | B |
| ATOM | 3731 | ND2 | ASN | B | 235 | 47.849 | 75.512 | 35.488 | 1.00 | 13.11 | B |
| ATOM | 3732 | C | ASN | B | 235 | 51.356 | 76.175 | 34.820 | 1.00 | 12.14 | B |
| ATOM | 3733 | O | ASN | B | 235 | 51.184 | 76.449 | 33.634 | 1.00 | 12.71 | B |
| ATOM | 3734 | N | LEU | B | 236 | 51.607 | 77.102 | 35.741 | 1.00 | 12.49 | B |
| ATOM | 3735 | CA | LEU | B | 236 | 51.708 | 78.505 | 35.364 | 1.00 | 11.62 | B |
| ATOM | 3736 | CB | LEU | B | 236 | 51.878 | 79.369 | 36.618 | 1.00 | 13.06 | B |
| ATOM | 3737 | CG | LEU | B | 236 | 50.698 | 79.361 | 37.594 | 1.00 | 14.67 | B |
| ATOM | 3738 | CD1 | LEU | B | 236 | 50.983 | 80.306 | 38.747 | 1.00 | 16.14 | B |
| ATOM | 3739 | CD2 | LEU | B | 236 | 49.426 | 79.778 | 36.872 | 1.00 | 16.50 | B |
| ATOM | 3740 | C | LEU | B | 236 | 52.841 | 78.678 | 34.367 | 1.00 | 11.05 | B |
| ATOM | 3741 | O | LEU | B | 236 | 52.729 | 79.461 | 33.433 | 1.00 | 10.82 | B |
| ATOM | 3742 | N | ALA | B | 237 | 53.931 | 77.942 | 34.563 | 1.00 | 10.62 | B |
| ATOM | 3743 | CA | ALA | B | 237 | 55.071 | 78.039 | 33.658 | 1.00 | 11.05 | B |
| ATOM | 3744 | CB | ALA | B | 237 | 56.226 | 77.198 | 34.174 | 1.00 | 11.84 | B |
| ATOM | 3745 | C | ALA | B | 237 | 54.670 | 77.585 | 32.257 | 1.00 | 8.78 | B |
| ATOM | 3746 | O | ALA | B | 237 | 55.036 | 78.210 | 31.261 | 1.00 | 10.25 | B |
| ATOM | 3747 | N | VAL | B | 238 | 53.916 | 76.493 | 32.175 | 1.00 | 8.92 | B |
| ATOM | 3748 | CA | VAL | B | 238 | 53.468 | 75.998 | 30.882 | 1.00 | 9.79 | B |
| ATOM | 3749 | CB | VAL | B | 238 | 52.646 | 74.697 | 31.038 | 1.00 | 8.65 | B |
| ATOM | 3750 | CG1 | VAL | B | 238 | 51.961 | 74.350 | 29.726 | 1.00 | 11.53 | B |
| ATOM | 3751 | CG2 | VAL | B | 238 | 53.563 | 73.557 | 31.469 | 1.00 | 10.18 | B |
| ATOM | 3752 | C | VAL | B | 238 | 52.615 | 77.059 | 30.188 | 1.00 | 9.93 | B |
| ATOM | 3753 | O | VAL | B | 238 | 52.772 | 77.318 | 28.992 | 1.00 | 9.81 | B |
| ATOM | 3754 | N | LEU | B | 239 | 51.713 | 77.684 | 30.940 | 1.00 | 10.23 | B |
| ATOM | 3755 | CA | LEU | B | 239 | 50.861 | 78.704 | 30.351 | 1.00 | 9.82 | B |
| ATOM | 3756 | CB | LEU | B | 239 | 49.767 | 79.092 | 31.349 | 1.00 | 12.39 | B |
| ATOM | 3757 | CG | LEU | B | 239 | 48.681 | 78.022 | 31.517 | 1.00 | 13.67 | B |
| ATOM | 3758 | CD1 | LEU | B | 239 | 47.624 | 78.512 | 32.492 | 1.00 | 14.95 | B |
| ATOM | 3759 | CD2 | LEU | B | 239 | 48.046 | 77.710 | 30.169 | 1.00 | 16.99 | B |
| ATOM | 3760 | C | LEU | B | 239 | 51.683 | 79.887 | 29.853 | 1.00 | 9.15 | B |
| ATOM | 3761 | O | LEU | B | 239 | 51.403 | 80.427 | 28.786 | 1.00 | 9.90 | B |
| ATOM | 3762 | N | LYS | B | 240 | 52.700 | 80.287 | 30.611 | 1.00 | 11.16 | B |
| ATOM | 3763 | CA | LYS | B | 240 | 53.535 | 81.405 | 30.184 | 1.00 | 10.48 | B |
| ATOM | 3764 | CB | LYS | B | 240 | 54.473 | 81.823 | 31.317 | 1.00 | 12.71 | B |
| ATOM | 3765 | CG | LYS | B | 240 | 55.399 | 82.972 | 30.928 | 1.00 | 14.68 | B |
| ATOM | 3766 | CD | LYS | B | 240 | 56.197 | 83.484 | 32.115 | 1.00 | 20.09 | B |
| ATOM | 3767 | CE | LYS | B | 240 | 55.297 | 84.164 | 33.136 | 1.00 | 22.68 | B |
| ATOM | 3768 | NZ | LYS | B | 240 | 56.076 | 84.727 | 34.276 | 1.00 | 24.21 | B |
| ATOM | 3769 | C | LYS | B | 240 | 54.279 | 81.045 | 28.905 | 1.00 | 10.44 | B |
| ATOM | 3770 | O | LYS | B | 240 | 54.281 | 81.819 | 27.949 | 1.00 | 11.84 | B |
| ATOM | 3771 | N | LEU | B | 241 | 54.909 | 79.871 | 28.884 | 1.00 | 10.42 | B |
| ATOM | 3772 | CA | LEU | B | 241 | 55.649 | 79.440 | 27.698 | 1.00 | 10.58 | B |
| ATOM | 3773 | CB | LEU | B | 241 | 56.324 | 78.091 | 27.976 | 1.00 | 10.58 | B |
| ATOM | 3774 | CG | LEU | B | 241 | 57.391 | 78.072 | 29.078 | 1.00 | 10.95 | B |
| ATOM | 3775 | CD1 | LEU | B | 241 | 57.857 | 76.646 | 29.303 | 1.00 | 11.46 | B |
| ATOM | 3776 | CD2 | LEU | B | 241 | 58.559 | 78.971 | 28.691 | 1.00 | 12.35 | B |
| ATOM | 3777 | C | LEU | B | 241 | 54.717 | 79.409 | 26.493 | 1.00 | 10.01 | B |
| ATOM | 3778 | O | LEU | B | 241 | 55.105 | 79.781 | 25.387 | 1.00 | 11.26 | B |
| ATOM | 3779 | N | SER | B | 242 | 53.485 | 78.958 | 26.712 | 1.00 | 9.61 | B |
| ATOM | 3780 | CA | SER | B | 242 | 52.489 | 78.907 | 25.652 | 1.00 | 10.02 | B |
| ATOM | 3781 | CB | SER | B | 242 | 51.216 | 78.249 | 26.186 | 1.00 | 9.83 | B |
| ATOM | 3782 | OG | SER | B | 242 | 50.149 | 78.410 | 25.262 | 1.00 | 11.46 | B |
| ATOM | 3783 | C | SER | B | 242 | 52.217 | 80.307 | 25.121 | 1.00 | 9.68 | B |
| ATOM | 3784 | O | SER | B | 242 | 52.314 | 80.558 | 23.921 | 1.00 | 10.65 | B |
| ATOM | 3785 | N | GLU | B | 243 | 51.870 | 81.212 | 26.032 | 1.00 | 10.68 | B |
| ATOM | 3786 | CA | GLU | B | 243 | 51.541 | 82.582 | 25.666 | 1.00 | 12.37 | B |
| ATOM | 3787 | CB | GLU | B | 243 | 50.899 | 83.274 | 26.876 | 1.00 | 12.07 | B |
| ATOM | 3788 | CG | GLU | B | 243 | 49.549 | 82.660 | 27.240 | 1.00 | 12.41 | B |
| ATOM | 3789 | CD | GLU | B | 243 | 48.811 | 83.390 | 28.345 | 1.00 | 12.64 | B |
| ATOM | 3790 | OE1 | GLU | B | 243 | 49.314 | 84.415 | 28.855 | 1.00 | 14.32 | B |
| ATOM | 3791 | OE2 | GLU | B | 243 | 47.710 | 82.925 | 28.704 | 1.00 | 13.44 | B |
| ATOM | 3792 | C | GLU | B | 243 | 52.694 | 83.364 | 25.054 | 1.00 | 11.58 | B |
| ATOM | 3793 | O | GLU | B | 243 | 52.460 | 84.308 | 24.295 | 1.00 | 13.14 | B |
| ATOM | 3794 | N | GLN | B | 244 | 53.927 | 82.974 | 25.367 | 1.00 | 12.52 | B |
| ATOM | 3795 | CA | GLN | B | 244 | 55.107 | 83.650 | 24.830 | 1.00 | 12.54 | B |
| ATOM | 3796 | CB | GLN | B | 244 | 56.275 | 83.506 | 25.811 | 1.00 | 15.35 | B |
| ATOM | 3797 | CG | GLN | B | 244 | 56.078 | 84.191 | 27.149 | 1.00 | 19.99 | B |
| ATOM | 3798 | CD | GLN | B | 244 | 57.242 | 83.953 | 28.095 | 1.00 | 24.17 | B |
| ATOM | 3799 | OE1 | GLN | B | 244 | 57.579 | 82.809 | 28.409 | 1.00 | 28.41 | B |
| ATOM | 3800 | NE2 | GLN | B | 244 | 57.860 | 85.032 | 28.555 | 1.00 | 26.83 | B |
| ATOM | 3801 | C | GLN | B | 244 | 55.457 | 83.116 | 23.450 | 1.00 | 12.24 | B |
| ATOM | 3802 | O | GLN | B | 244 | 56.320 | 83.672 | 22.769 | 1.00 | 13.81 | B |
| ATOM | 3803 | N | GLY | B | 245 | 54.795 | 82.038 | 23.043 | 1.00 | 12.05 | B |
| ATOM | 3804 | CA | GLY | B | 245 | 55.061 | 81.440 | 21.745 | 1.00 | 12.13 | B |
| ATOM | 3805 | C | GLY | B | 245 | 56.273 | 80.523 | 21.751 | 1.00 | 10.48 | B |
| ATOM | 3806 | O | GLY | B | 245 | 56.667 | 79.992 | 20.710 | 1.00 | 11.40 | B |
| ATOM | 3807 | N | LEU | B | 246 | 56.850 | 80.316 | 22.929 | 1.00 | 10.40 | B |
| ATOM | 3808 | CA | LEU | B | 246 | 58.035 | 79.473 | 23.072 | 1.00 | 11.35 | B |
| ATOM | 3809 | CB | LEU | B | 246 | 58.634 | 79.705 | 24.461 | 1.00 | 12.45 | B |
| ATOM | 3810 | CG | LEU | B | 246 | 59.258 | 81.086 | 24.690 | 1.00 | 15.77 | B |
| ATOM | 3811 | CD1 | LEU | B | 246 | 59.700 | 81.221 | 26.139 | 1.00 | 19.66 | B |
| ATOM | 3812 | CD2 | LEU | B | 246 | 60.440 | 81.273 | 23.750 | 1.00 | 17.98 | B |
| ATOM | 3813 | C | LEU | B | 246 | 57.770 | 77.999 | 22.771 | 1.00 | 10.58 | B |
| ATOM | 3814 | O | LEU | B | 246 | 58.653 | 77.292 | 22.283 | 1.00 | 11.68 | B |
| ATOM | 3815 | N | LEU | B | 247 | 56.566 | 77.521 | 23.063 | 1.00 | 10.70 | B |
| ATOM | 3816 | CA | LEU | B | 247 | 56.247 | 76.127 | 22.787 | 1.00 | 10.56 | B |
| ATOM | 3817 | CB | LEU | B | 247 | 54.971 | 75.736 | 23.539 | 1.00 | 12.11 | B |
| ATOM | 3818 | CG | LEU | B | 247 | 55.059 | 75.852 | 25.063 | 1.00 | 11.27 | B |
| ATOM | 3819 | CD1 | LEU | B | 247 | 53.732 | 75.415 | 25.671 | 1.00 | 11.53 | B |
| ATOM | 3820 | CD2 | LEU | B | 247 | 56.198 | 74.997 | 25.601 | 1.00 | 13.61 | B |
| ATOM | 3821 | C | LEU | B | 247 | 56.161 | 75.934 | 21.277 | 1.00 | 10.81 | B |
| ATOM | 3822 | O | LEU | B | 247 | 56.552 | 74.891 | 20.760 | 1.00 | 10.76 | B |
| ATOM | 3823 | N | ASP | B | 248 | 55.654 | 76.938 | 20.565 | 1.00 | 10.50 | B |
| ATOM | 3824 | CA | ASP | B | 248 | 55.564 | 76.843 | 19.112 | 1.00 | 12.17 | B |
| ATOM | 3825 | CB | ASP | B | 248 | 54.736 | 78.007 | 18.552 | 1.00 | 12.81 | B |
| ATOM | 3826 | CG | ASP | B | 248 | 53.288 | 77.971 | 18.998 | 1.00 | 14.09 | B |
| ATOM | 3827 | OD1 | ASP | B | 248 | 52.693 | 76.873 | 19.027 | 1.00 | 16.17 | B |
| ATOM | 3828 | OD2 | ASP | B | 248 | 52.737 | 79.050 | 19.302 | 1.00 | 17.55 | B |
| ATOM | 3829 | C | ASP | B | 248 | 56.979 | 76.806 | 18.537 | 1.00 | 12.32 | B |
| ATOM | 3830 | O | ASP | B | 248 | 57.263 | 76.046 | 17.608 | 1.00 | 13.88 | B |
| ATOM | 3831 | N | LYS | B | 249 | 57.862 | 77.631 | 19.091 | 1.00 | 12.62 | B |
| ATOM | 3832 | CA | LYS | B | 249 | 59.243 | 77.686 | 18.628 | 1.00 | 13.25 | B |
| ATOM | 3833 | CB | LYS | B | 249 | 59.971 | 78.828 | 19.343 | 1.00 | 15.24 | B |
| ATOM | 3834 | CG | LYS | B | 249 | 61.042 | 79.036 | 18.878 | 1.00 | 21.15 | B |
| ATOM | 3835 | CD | LYS | B | 249 | 62.062 | 80.194 | 19.609 | 1.00 | 23.46 | B |
| ATOM | 3836 | CE | LYS | B | 249 | 63.487 | 80.412 | 19.123 | 1.00 | 25.24 | B |
| ATOM | 3837 | NZ | LYS | B | 249 | 63.529 | 80.703 | 17.659 | 1.00 | 27.50 | B |
| ATOM | 3838 | C | LYS | B | 249 | 59.922 | 76.337 | 18.830 | 1.00 | 11.89 | B |
| ATOM | 3839 | O | LYS | B | 249 | 60.638 | 75.860 | 17.948 | 1.00 | 11.35 | B |
| ATOM | 3840 | N | LEU | B | 250 | 59.696 | 75.727 | 19.990 | 1.00 | 11.05 | B |
| ATOM | 3841 | CA | LEU | B | 250 | 60.292 | 74.434 | 20.296 | 1.00 | 12.56 | B |
| ATOM | 3842 | CB | LEU | B | 250 | 60.072 | 74.106 | 21.776 | 1.00 | 14.40 | B |
| ATOM | 3843 | CG | LEU | B | 250 | 60.913 | 74.904 | 22.777 | 1.00 | 15.38 | B |
| ATOM | 3844 | CD1 | LEU | B | 250 | 60.628 | 74.408 | 24.188 | 1.00 | 18.75 | B |
| ATOM | 3845 | CD2 | LEU | B | 250 | 62.391 | 74.745 | 22.451 | 1.00 | 19.61 | B |
| ATOM | 3846 | C | LEU | B | 250 | 59.791 | 73.343 | 19.358 | 1.00 | 10.20 | B |
| ATOM | 3847 | O | LEU | B | 250 | 60.569 | 72.483 | 18.944 | 1.00 | 11.44 | B |
| ATOM | 3848 | N | LYS | B | 251 | 58.504 | 73.360 | 19.009 | 1.00 | 10.95 | B |
| ATOM | 3849 | CA | LYS | B | 251 | 58.017 | 72.330 | 18.097 | 1.00 | 12.43 | B |
| ATOM | 3850 | CB | LYS | B | 251 | 56.485 | 72.312 | 18.023 | 1.00 | 13.29 | B |
| ATOM | 3851 | CG | LYS | B | 251 | 55.996 | 71.181 | 17.113 | 1.00 | 16.55 | B |
| ATOM | 3852 | CD | LYS | B | 251 | 54.490 | 71.004 | 17.085 | 1.00 | 17.86 | B |
| ATOM | 3853 | CE | LYS | B | 251 | 54.135 | 69.736 | 16.308 | 1.00 | 19.60 | B |
| ATOM | 3854 | NZ | LYS | B | 251 | 52.675 | 69.544 | 16.140 | 1.00 | 20.85 | B |
| ATOM | 3855 | C | LYS | B | 251 | 58.654 | 72.517 | 16.726 | 1.00 | 11.79 | B |
| ATOM | 3856 | O | LYS | B | 251 | 59.086 | 71.549 | 16.105 | 1.00 | 11.81 | B |
| ATOM | 3857 | N | ASN | B | 252 | 58.713 | 73.759 | 16.250 | 1.00 | 11.81 | B |
| ATOM | 3858 | CA | ASN | B | 252 | 59.316 | 74.021 | 14.946 | 1.00 | 13.39 | B |
| ATOM | 3859 | CB | ASN | B | 252 | 59.170 | 75.502 | 14.585 | 1.00 | 16.26 | B |
| ATOM | 3860 | CG | ASN | B | 252 | 57.746 | 75.882 | 14.262 | 1.00 | 19.99 | B |
| ATOM | 3861 | OD1 | ASN | B | 252 | 57.000 | 75.092 | 13.684 | 1.00 | 23.83 | B |
| ATOM | 3862 | ND2 | ASN | B | 252 | 57.363 | 77.103 | 14.614 | 1.00 | 22.55 | B |
| ATOM | 3863 | C | ASN | B | 252 | 60.764 | 73.564 | 14.941 | 1.00 | 13.53 | B |
| ATOM | 3864 | O | ASN | B | 252 | 61.230 | 72.966 | 13.974 | 1.00 | 13.33 | B |
| ATOM | 3865 | N | LYS | B | 253 | 61.471 | 73.844 | 16.029 | 1.00 | 12.13 | B |
| ATOM | 3866 | CA | LYS | B | 253 | 62.872 | 73.461 | 16.147 | 1.00 | 13.00 | B |
| ATOM | 3867 | CB | LYS | B | 253 | 63.413 | 73.951 | 17.495 | 1.00 | 14.25 | B |
| ATOM | 3868 | CG | LYS | B | 253 | 64.812 | 73.439 | 17.823 | 1.00 | 13.83 | B |
| ATOM | 3869 | CD | LYS | B | 253 | 65.278 | 73.913 | 19.192 | 1.00 | 14.51 | B |
| ATOM | 3870 | CE | LYS | B | 253 | 66.663 | 73.377 | 19.522 | 1.00 | 17.96 | B |
| ATOM | 3871 | NZ | LYS | B | 253 | 67.083 | 73.685 | 20.922 | 1.00 | 20.12 | B |
| ATOM | 3872 | C | LYS | B | 253 | 63.098 | 71.966 | 15.954 | 1.00 | 12.93 | B |
| ATOM | 3873 | O | LYS | B | 253 | 63.931 | 71.555 | 15.139 | 1.00 | 13.54 | B |
| ATOM | 3874 | N | TRP | B | 254 | 62.350 | 71.154 | 16.696 | 1.00 | 11.94 | B |
| ATOM | 3875 | CA | TRP | B | 254 | 62.516 | 69.709 | 16.654 | 1.00 | 12.21 | B |
| ATOM | 3876 | CB | TRP | B | 254 | 62.240 | 69.165 | 18.063 | 1.00 | 12.24 | B |
| ATOM | 3877 | CG | TRP | B | 254 | 63.252 | 69.634 | 19.062 | 1.00 | 10.70 | B |
| ATOM | 3878 | CD2 | TRP | B | 254 | 64.591 | 69.148 | 19.204 | 1.00 | 9.88 | B |
| ATOM | 3879 | CE2 | TRP | B | 254 | 65.213 | 69.920 | 20.210 | 1.00 | 10.80 | B |
| ATOM | 3880 | CE3 | TRP | B | 254 | 65.327 | 68.137 | 18.573 | 1.00 | 10.93 | B |
| ATOM | 3881 | CD1 | TRP | B | 254 | 63.116 | 70.653 | 19.965 | 1.00 | 10.24 | B |
| ATOM | 3882 | NE1 | TRP | B | 254 | 64.292 | 70.831 | 20.659 | 1.00 | 10.60 | B |
| ATOM | 3883 | CZ2 | TRP | B | 254 | 66.540 | 69.711 | 20.600 | 1.00 | 10.82 | B |
| ATOM | 3884 | CZ3 | TRP | B | 254 | 66.647 | 67.929 | 18.960 | 1.00 | 11.90 | B |
| ATOM | 3885 | CH2 | TRP | B | 254 | 67.238 | 68.713 | 19.965 | 1.00 | 10.73 | B |
| ATOM | 3886 | C | TRP | B | 254 | 61.752 | 68.957 | 15.571 | 1.00 | 10.95 | B |
| ATOM | 3887 | O | TRP | B | 254 | 62.073 | 67.801 | 15.287 | 1.00 | 12.40 | B |
| ATOM | 3888 | N | TRP | B | 255 | 60.765 | 69.598 | 14.957 | 1.00 | 11.38 | B |
| ATOM | 3889 | CA | TRP | B | 255 | 59.962 | 68.952 | 13.923 | 1.00 | 13.10 | B |
| ATOM | 3890 | CB | TRP | B | 255 | 58.475 | 69.182 | 14.224 | 1.00 | 12.80 | B |
| ATOM | 3891 | CG | TRP | B | 255 | 57.813 | 68.115 | 15.019 | 1.00 | 10.68 | B |
| ATOM | 3892 | CD2 | TRP | B | 255 | 57.957 | 67.882 | 16.420 | 1.00 | 9.63 | B |
| ATOM | 3893 | CE2 | TRP | B | 255 | 57.149 | 66.771 | 16.742 | 1.00 | 10.00 | B |
| ATOM | 3894 | CE3 | TRP | B | 255 | 58.693 | 68.505 | 17.437 | 1.00 | 10.25 | B |
| ATOM | 3895 | CD1 | TRP | B | 255 | 56.947 | 67.167 | 14.556 | 1.00 | 11.48 | B |
| ATOM | 3896 | NE1 | TRP | B | 255 | 56.543 | 66.355 | 15.586 | 1.00 | 10.11 | B |
| ATOM | 3897 | CZ2 | TRP | B | 255 | 57.054 | 66.265 | 18.041 | 1.00 | 10.61 | B |
| ATOM | 3898 | CZ3 | TRP | B | 255 | 58.598 | 68.002 | 18.732 | 1.00 | 10.77 | B |
| ATOM | 3899 | CH2 | TRP | B | 255 | 57.783 | 66.890 | 19.021 | 1.00 | 11.15 | B |
| ATOM | 3900 | C | TRP | B | 255 | 60.289 | 69.392 | 12.505 | 1.00 | 14.72 | B |
| ATOM | 3901 | O | TRP | B | 255 | 60.491 | 68.560 | 11.620 | 1.00 | 15.12 | B |
| ATOM | 3902 | N | TYR | B | 256 | 60.349 | 70.702 | 12.295 | 1.00 | 17.10 | B |
| ATOM | 3903 | CA | TYR | B | 256 | 60.544 | 71.233 | 10.954 | 1.00 | 19.22 | B |
| ATOM | 3904 | CB | TYR | B | 256 | 59.340 | 72.129 | 10.666 | 1.00 | 19.05 | B |
| ATOM | 3905 | CG | TYR | B | 256 | 58.030 | 71.468 | 11.035 | 1.00 | 19.92 | B |
| ATOM | 3906 | CD1 | TYR | B | 256 | 57.599 | 70.317 | 10.378 | 1.00 | 22.52 | B |
| ATOM | 3907 | CE1 | TYR | B | 256 | 56.415 | 69.677 | 10.746 | 1.00 | 21.25 | B |
| ATOM | 3908 | CD2 | TYR | B | 256 | 57.242 | 71.968 | 12.071 | 1.00 | 20.19 | B |
| ATOM | 3909 | CE2 | TYR | B | 256 | 56.059 | 71.336 | 12.446 | 1.00 | 22.92 | B |
| ATOM | 3910 | CZ | TYR | B | 256 | 55.653 | 70.191 | 11.779 | 1.00 | 22.06 | B |
| ATOM | 3911 | OH | TYR | B | 256 | 54.491 | 69.554 | 12.153 | 1.00 | 24.67 | B |
| ATOM | 3912 | C | TYR | B | 256 | 61.871 | 71.894 | 10.609 | 1.00 | 21.40 | B |
| ATOM | 3913 | O | TYR | B | 256 | 62.350 | 71.749 | 9.482 | 1.00 | 22.61 | B |
| ATOM | 3914 | N | ASP | B | 257 | 62.464 | 72.623 | 11.547 | 1.00 | 23.95 | B |
| ATOM | 3915 | CA | ASP | B | 257 | 63.732 | 73.290 | 11.266 | 1.00 | 27.44 | B |
| ATOM | 3916 | CB | ASP | B | 257 | 64.165 | 74.112 | 12.487 | 1.00 | 27.78 | B |
| ATOM | 3917 | CG | ASP | B | 257 | 63.211 | 75.254 | 12.789 | 1.00 | 29.85 | B |
| ATOM | 3918 | OD1 | ASP | B | 257 | 62.565 | 75.759 | 11.846 | 1.00 | 32.11 | B |
| ATOM | 3919 | OD2 | ASP | B | 257 | 63.118 | 75.659 | 13.966 | 1.00 | 31.30 | B |
| ATOM | 3920 | C | ASP | B | 257 | 64.784 | 72.285 | 10.812 | 1.00 | 29.29 | B |
| ATOM | 3921 | O | ASP | B | 257 | 65.688 | 72.622 | 10.047 | 1.00 | 30.78 | B |
| ATOM | 3922 | N | GLY | B | 258 | 64.659 | 71.050 | 11.283 | 1.00 | 31.43 | B |
| ATOM | 3923 | CA | GLY | B | 258 | 65.605 | 70.017 | 10.904 | 1.00 | 32.34 | B |
| ATOM | 3924 | C | GLY | B | 258 | 65.005 | 69.063 | 9.891 | 1.00 | 33.02 | B |
| ATOM | 3925 | O | GLY | B | 258 | 65.565 | 68.000 | 9.619 | 1.00 | 33.65 | B |
| ATOM | 3926 | N | GLY | B | 259 | 63.860 | 69.445 | 9.332 | 1.00 | 33.63 | B |
| ATOM | 3927 | CA | GLY | B | 259 | 63.195 | 68.610 | 8.348 | 1.00 | 33.47 | B |
| ATOM | 3928 | C | GLY | B | 259 | 64.149 | 68.089 | 7.291 | 1.00 | 34.04 | B |
| ATOM | 3929 | O | GLY | B | 259 | 65.116 | 68.761 | 6.933 | 1.00 | 34.51 | B |
| ATOM | 3930 | N | GLU | B | 260 | 63.878 | 66.888 | 6.792 | 1.00 | 33.23 | B |
| ATOM | 3931 | CA | GLU | B | 260 | 64.722 | 66.272 | 5.775 | 1.00 | 32.24 | B |
| ATOM | 3932 | CB | GLU | B | 260 | 65.348 | 65.000 | 6.355 | 1.00 | 31.96 | B |
| ATOM | 3933 | CG | GLU | B | 260 | 66.243 | 65.236 | 7.560 | 1.00 | 32.09 | B |
| ATOM | 3934 | CD | GLU | B | 260 | 66.683 | 63.943 | 8.220 | 1.00 | 32.53 | B |
| ATOM | 3935 | OE1 | GLU | B | 260 | 67.511 | 64.000 | 9.154 | 1.00 | 33.55 | B |
| ATOM | 3936 | OE2 | GLU | B | 260 | 66.196 | 62.868 | 7.808 | 1.00 | 32.24 | B |
| ATOM | 3937 | C | GLU | B | 260 | 63.916 | 66.009 | 4.512 | 1.00 | 31.67 | B |
| ATOM | 3938 | O | GLU | B | 260 | 64.439 | 65.473 | 3.534 | 1.00 | 32.39 | B |
| ATOM | 3939 | N | CYS | B | 261 | 62.643 | 66.388 | 4.534 | 1.00 | 31.23 | B |
| ATOM | 3940 | CA | CYS | B | 261 | 61.765 | 66.175 | 3.391 | 1.00 | 31.10 | B |
| ATOM | 3941 | CB | CYS | B | 261 | 60.671 | 65.181 | 3.789 | 1.00 | 28.33 | B |
| ATOM | 3942 | SG | CYS | B | 261 | 61.295 | 63.662 | 4.576 | 1.00 | 22.63 | B |
| ATOM | 3943 | C | CYS | B | 261 | 61.212 | 67.499 | 2.882 | 1.00 | 33.06 | B |
| ATOM | 3944 | OT1 | CYS | B | 261 | 61.395 | 67.786 | 1.682 | 1.00 | 34.09 | B |
| ATOM | 3945 | OT2 | CYS | B | 261 | 60.596 | 68.229 | 3.687 | 1.00 | 34.77 | B |
| ATOM | 3946 | N | KAI | C | 999 | 36.732 | 83.699 | 24.381 | 1.00 | 8.97 | C |
| ATOM | 3947 | CD | KAI | C | 999 | 35.356 | 84.162 | 23.978 | 1.00 | 11.13 | C |
| ATOM | 3948 | CD1 | KAI | C | 999 | 33.448 | 86.124 | 23.049 | 1.00 | 11.15 | C |
| ATOM | 3949 | CD2 | KAI | C | 999 | 34.433 | 88.083 | 24.099 | 1.00 | 10.37 | C |
| ATOM | 3950 | CA | KAI | C | 999 | 37.571 | 84.834 | 24.580 | 1.00 | 9.63 | C |
| ATOM | 3951 | CB | KAI | C | 999 | 36.895 | 86.049 | 23.967 | 1.00 | 9.40 | C |
| ATOM | 3952 | CB1 | KAI | C | 999 | 37.007 | 86.149 | 22.439 | 1.00 | 9.89 | C |
| ATOM | 3953 | CG | KAI | C | 999 | 35.496 | 85.730 | 24.227 | 1.00 | 10.71 | C |
| ATOM | 3954 | CG1 | KAI | C | 999 | 38.226 | 86.898 | 21.921 | 1.00 | 11.01 | C |
| ATOM | 3955 | CG2 | KAI | C | 999 | 34.361 | 86.658 | 23.754 | 1.00 | 9.12 | C |
| ATOM | 3956 | C | KAI | C | 999 | 37.920 | 85.004 | 26.067 | 1.00 | 9.43 | C |
| ATOM | 3957 | O | KAI | C | 999 | 37.684 | 84.018 | 26.801 | 1.00 | 9.25 | C |
| ATOM | 3958 | OD1 | KAI | C | 999 | 38.397 | 86.868 | 20.684 | 1.00 | 11.02 | C |
| ATOM | 3959 | OD2 | KAI | C | 999 | 38.955 | 87.487 | 22.751 | 1.00 | 11.20 | C |
| ATOM | 3960 | OT | KAI | C | 999 | 38.417 | 86.102 | 26.400 | 1.00 | 9.41 | C |
| ATOM | 3961 | N1 | LY3 | E | 888 | 50.824 | 75.300 | 24.218 | 1.00 | 14.69 | E |
| ATOM | 3962 | C2 | LY3 | E | 888 | 49.837 | 74.767 | 24.101 | 1.00 | 12.99 | E |
| ATOM | 3963 | C3 | LY3 | E | 888 | 48.551 | 74.064 | 23.955 | 1.00 | 14.06 | E |
| ATOM | 3964 | C4 | LY3 | E | 888 | 47.446 | 74.389 | 24.833 | 1.00 | 11.93 | E |
| ATOM | 3965 | C5 | LY3 | E | 888 | 46.201 | 73.657 | 24.724 | 1.00 | 11.26 | E |
| ATOM | 3966 | C6 | LY3 | E | 888 | 46.065 | 72.599 | 23.727 | 1.00 | 9.88 | E |
| ATOM | 3967 | C7 | LY3 | E | 888 | 47.174 | 72.297 | 22.862 | 1.00 | 12.86 | E |
| ATOM | 3968 | C8 | LY3 | E | 888 | 48.410 | 73.022 | 22.968 | 1.00 | 13.14 | E |
| ATOM | 3969 | C9 | LY3 | E | 888 | 44.688 | 71.853 | 23.704 | 1.00 | 9.90 | E |
| ATOM | 3970 | C10 | LY3 | E | 888 | 43.409 | 72.534 | 23.750 | 1.00 | 8.29 | E |
| ATOM | 3971 | C11 | LY3 | E | 888 | 42.177 | 71.761 | 23.749 | 1.00 | 6.63 | E |
| ATOM | 3972 | C12 | LY3 | E | 888 | 42.220 | 70.314 | 23.697 | 1.00 | 8.27 | E |
| ATOM | 3973 | C13 | LY3 | E | 888 | 43.501 | 69.642 | 23.639 | 1.00 | 9.35 | E |
| ATOM | 3974 | C14 | LY3 | E | 888 | 44.721 | 70.401 | 23.646 | 1.00 | 9.41 | E |
| ATOM | 3975 | C15 | LY3 | E | 888 | 40.937 | 69.538 | 23.732 | 1.00 | 6.94 | E |
| ATOM | 3976 | C16 | LY3 | E | 888 | 40.519 | 69.153 | 22.327 | 1.00 | 9.61 | E |
| ATOM | 3977 | C17 | LY3 | E | 888 | 41.080 | 68.279 | 24.574 | 1.00 | 9.87 | E |
| ATOM | 3978 | N18 | LY3 | E | 888 | 41.562 | 68.515 | 25.961 | 1.00 | 9.17 | E |
| ATOM | 3979 | S19 | LY3 | E | 888 | 42.499 | 67.367 | 26.745 | 1.00 | 8.97 | E |
| ATOM | 3980 | O20 | LY3 | E | 888 | 43.612 | 67.035 | 25.887 | 1.00 | 10.09 | E |
| ATOM | 3981 | O21 | LY3 | E | 888 | 42.912 | 68.015 | 27.994 | 1.00 | 10.41 | E |
| ATOM | 3982 | C22 | LY3 | E | 888 | 41.536 | 66.117 | 26.870 | 1.00 | 9.66 | E |
| ATOM | 3983 | C23 | LY3 | E | 888 | 42.386 | 65.070 | 27.545 | 1.00 | 14.10 | E |
| ATOM | 3984 | C24 | LY3 | E | 888 | 40.409 | 66.529 | 27.730 | 1.00 | 11.22 | E |
| ATOM | 3985 | OH2 | WAT | W | 1 | 47.102 | 58.092 | 22.068 | 1.00 | 9.42 | W |
| ATOM | 3986 | OH2 | WAT | W | 2 | 41.892 | 84.589 | 22.087 | 1.00 | 9.65 | W |
| ATOM | 3987 | OH2 | WAT | W | 3 | 36.678 | 85.311 | 19.219 | 1.00 | 9.95 | W |
| ATOM | 3988 | OH2 | WAT | W | 4 | 52.289 | 57.357 | 19.194 | 1.00 | 10.29 | W |
| ATOM | 3989 | OH2 | WAT | W | 5 | 46.379 | 81.533 | 26.710 | 1.00 | 10.86 | W |
| ATOM | 3990 | OH2 | WAT | W | 6 | 33.845 | 77.899 | 30.965 | 1.00 | 11.49 | W |
| ATOM | 3991 | OH2 | WAT | W | 7 | 50.870 | 71.891 | 20.044 | 1.00 | 11.56 | W |
| ATOM | 3992 | OH2 | WAT | W | 8 | 55.114 | 64.760 | 30.992 | 1.00 | 11.41 | W |
| ATOM | 3993 | OH2 | WAT | W | 9 | 55.092 | 72.606 | 21.327 | 1.00 | 12.00 | W |
| ATOM | 3994 | OH2 | WAT | W | 10 | 48.599 | 82.653 | 23.121 | 1.00 | 12.64 | W |
| ATOM | 3995 | OH2 | WAT | W | 11 | 38.122 | 70.791 | 20.060 | 1.00 | 12.16 | W |
| ATOM | 3996 | OH2 | WAT | W | 12 | 40.419 | 60.055 | 23.138 | 1.00 | 12.75 | W |
| ATOM | 3997 | OH2 | WAT | W | 13 | 42.595 | 61.145 | 26.695 | 1.00 | 11.43 | W |
| ATOM | 3998 | OH2 | WAT | W | 14 | 37.945 | 60.776 | 21.876 | 1.00 | 11.88 | W |
| ATOM | 3999 | OH2 | WAT | W | 15 | 68.135 | 69.797 | 23.658 | 1.00 | 12.94 | W |
| ATOM | 4000 | OH2 | WAT | W | 16 | 33.881 | 70.066 | 21.295 | 1.00 | 12.65 | W |
| ATOM | 4001 | OH2 | WAT | W | 17 | 39.517 | 90.174 | 22.369 | 1.00 | 11.67 | W |
| ATOM | 4002 | OH2 | WAT | W | 18 | 20.860 | 72.869 | 23.649 | 1.00 | 14.37 | W |
| ATOM | 4003 | OH2 | WAT | W | 19 | 40.816 | 62.182 | 24.797 | 1.00 | 12.94 | W |
| ATOM | 4004 | OH2 | WAT | W | 20 | 50.159 | 61.009 | 42.996 | 1.00 | 14.37 | W |
| ATOM | 4005 | OH2 | WAT | W | 21 | 49.483 | 52.474 | 22.361 | 1.00 | 12.62 | W |
| ATOM | 4006 | OH2 | WAT | W | 22 | 41.787 | 101.520 | 16.042 | 1.00 | 12.71 | W |
| ATOM | 4007 | OH2 | WAT | W | 23 | 66.949 | 69.772 | 30.438 | 1.00 | 13.34 | W |
| ATOM | 4008 | OH2 | WAT | W | 24 | 47.186 | 41.155 | 16.033 | 1.00 | 12.63 | W |
| ATOM | 4009 | OH2 | WAT | W | 25 | 22.033 | 72.872 | 30.430 | 1.00 | 13.42 | W |
| ATOM | 4010 | OH2 | WAT | W | 26 | 38.836 | 81.698 | 42.991 | 1.00 | 14.92 | W |
| ATOM | 4011 | OH2 | WAT | W | 27 | 38.981 | 40.210 | 18.711 | 1.00 | 13.28 | W |
| ATOM | 4012 | OH2 | WAT | W | 28 | 45.202 | 83.684 | 25.166 | 1.00 | 13.63 | W |
| ATOM | 4013 | OH2 | WAT | W | 29 | 50.995 | 81.871 | 21.899 | 1.00 | 12.75 | W |
| ATOM | 4014 | OH2 | WAT | W | 30 | 51.886 | 68.752 | 36.988 | 1.00 | 14.14 | W |
| ATOM | 4015 | OH2 | WAT | W | 31 | 53.804 | 78.573 | 22.322 | 1.00 | 13.21 | W |
| ATOM | 4016 | OH2 | WAT | W | 32 | 37.114 | 73.904 | 36.993 | 1.00 | 13.79 | W |
| ATOM | 4017 | OH2 | WAT | W | 33 | 50.186 | 77.511 | 22.712 | 1.00 | 13.81 | W |
| ATOM | 4018 | OH2 | WAT | W | 34 | 49.758 | 96.872 | 9.527 | 1.00 | 14.63 | W |
| ATOM | 4019 | OH2 | WAT | W | 35 | 39.170 | 45.814 | 9.527 | 1.00 | 15.33 | W |
| ATOM | 4020 | OH2 | WAT | W | 36 | 35.210 | 64.071 | 22.298 | 1.00 | 13.53 | W |
| ATOM | 4021 | OH2 | WAT | W | 37 | 43.727 | 58.990 | 25.122 | 1.00 | 14.75 | W |
| ATOM | 4022 | OH2 | WAT | W | 38 | 38.820 | 65.158 | 22.768 | 1.00 | 13.75 | W |
| ATOM | 4023 | OH2 | WAT | W | 39 | 70.814 | 63.704 | 34.074 | 1.00 | 13.03 | W |
| ATOM | 4024 | OH2 | WAT | W | 40 | 46.180 | 87.108 | 37.618 | 1.00 | 15.72 | W |
| ATOM | 4025 | OH2 | WAT | W | 41 | 64.124 | 55.179 | 27.857 | 1.00 | 13.51 | W |
| ATOM | 4026 | OH2 | WAT | W | 42 | 48.163 | 80.493 | 24.804 | 1.00 | 13.14 | W |
| ATOM | 4027 | OH2 | WAT | W | 43 | 24.858 | 87.455 | 27.843 | 1.00 | 13.24 | W |
| ATOM | 4028 | OH2 | WAT | W | 44 | 42.829 | 55.548 | 37.593 | 1.00 | 15.05 | W |
| ATOM | 4029 | OH2 | WAT | W | 45 | 36.635 | 48.648 | 21.536 | 1.00 | 14.77 | W |
| ATOM | 4030 | OH2 | WAT | W | 46 | 43.414 | 105.324 | 17.763 | 1.00 | 15.06 | W |
| ATOM | 4031 | OH2 | WAT | W | 47 | 41.508 | 56.892 | 30.417 | 1.00 | 14.57 | W |
| ATOM | 4032 | OH2 | WAT | W | 48 | 49.989 | 102.506 | 18.712 | 1.00 | 14.52 | W |
| ATOM | 4033 | OH2 | WAT | W | 49 | 25.983 | 88.936 | 29.899 | 1.00 | 14.36 | W |
| ATOM | 4034 | OH2 | WAT | W | 50 | 54.492 | 56.093 | 18.081 | 1.00 | 12.38 | W |
| ATOM | 4035 | OH2 | WAT | W | 51 | 38.480 | 63.295 | 20.839 | 1.00 | 13.21 | W |
| ATOM | 4036 | OH2 | WAT | W | 52 | 34.506 | 86.536 | 18.104 | 1.00 | 13.07 | W |
| ATOM | 4037 | OH2 | WAT | W | 53 | 62.976 | 53.766 | 29.878 | 1.00 | 15.14 | W |
| ATOM | 4038 | OH2 | WAT | W | 54 | 52.374 | 94.064 | 21.522 | 1.00 | 15.10 | W |
| ATOM | 4039 | OH2 | WAT | W | 55 | 32.437 | 75.719 | 43.086 | 1.00 | 18.07 | W |
| ATOM | 4040 | OH2 | WAT | W | 56 | 45.572 | 37.362 | 17.727 | 1.00 | 14.20 | W |
| ATOM | 4041 | OH2 | WAT | W | 57 | 18.811 | 85.476 | 29.350 | 1.00 | 15.28 | W |
| ATOM | 4042 | OH2 | WAT | W | 58 | 44.689 | 74.110 | 20.571 | 1.00 | 13.59 | W |
| ATOM | 4043 | OH2 | WAT | W | 59 | 70.181 | 57.203 | 29.405 | 1.00 | 15.34 | W |
| ATOM | 4044 | OH2 | WAT | W | 60 | 50.535 | 79.365 | 20.810 | 1.00 | 14.25 | W |
| ATOM | 4045 | OH2 | WAT | W | 61 | 59.364 | 59.220 | 6.177 | 1.00 | 13.93 | W |
| ATOM | 4046 | OH2 | WAT | W | 62 | 18.152 | 78.966 | 34.116 | 1.00 | 13.75 | W |
| ATOM | 4047 | OH2 | WAT | W | 63 | 47.480 | 85.774 | 30.437 | 1.00 | 15.69 | W |
| ATOM | 4048 | OH2 | WAT | W | 64 | 52.509 | 73.385 | 21.803 | 1.00 | 12.40 | W |
| ATOM | 4049 | OH2 | WAT | W | 65 | 36.499 | 69.305 | 21.796 | 1.00 | 13.29 | W |
| ATOM | 4050 | OH2 | WAT | W | 66 | 56.541 | 66.956 | 43.061 | 1.00 | 19.69 | W |
| ATOM | 4051 | OH2 | WAT | W | 67 | 44.592 | 65.330 | 39.631 | 1.00 | 15.23 | W |
| ATOM | 4052 | OH2 | WAT | W | 68 | 43.422 | 66.065 | 16.840 | 1.00 | 16.31 | W |
| ATOM | 4053 | OH2 | WAT | W | 69 | 45.595 | 76.658 | 16.838 | 1.00 | 15.43 | W |
| ATOM | 4054 | OH2 | WAT | W | 70 | 52.117 | 76.180 | 21.515 | 1.00 | 15.25 | W |
| ATOM | 4055 | OH2 | WAT | W | 71 | 34.389 | 82.628 | 8.611 | 1.00 | 15.39 | W |
| ATOM | 4056 | OH2 | WAT | W | 72 | 43.502 | 56.313 | 25.895 | 1.00 | 13.93 | W |
| ATOM | 4057 | OH2 | WAT | W | 73 | 44.383 | 77.298 | 39.674 | 1.00 | 15.09 | W |
| ATOM | 4058 | OH2 | WAT | W | 74 | 31.402 | 80.991 | 4.150 | 1.00 | 16.58 | W |
| ATOM | 4059 | OH2 | WAT | W | 75 | 45.484 | 86.340 | 25.866 | 1.00 | 13.07 | W |
| ATOM | 4060 | OH2 | WAT | W | 76 | 29.658 | 83.471 | 6.193 | 1.00 | 16.11 | W |
| ATOM | 4061 | OH2 | WAT | W | 77 | 50.842 | 53.536 | 18.587 | 1.00 | 16.64 | W |
| ATOM | 4062 | OH2 | WAT | W | 78 | 52.363 | 98.944 | 13.085 | 1.00 | 16.61 | W |
| ATOM | 4063 | OH2 | WAT | W | 79 | 38.066 | 89.045 | 18.567 | 1.00 | 16.20 | W |
| ATOM | 4064 | OH2 | WAT | W | 80 | 44.290 | 68.562 | 20.537 | 1.00 | 13.98 | W |
| ATOM | 4065 | OH2 | WAT | W | 81 | 54.651 | 59.977 | 8.635 | 1.00 | 16.03 | W |
| ATOM | 4066 | OH2 | WAT | W | 82 | 36.867 | 66.495 | 21.576 | 1.00 | 15.68 | W |
| ATOM | 4067 | OH2 | WAT | W | 83 | 23.603 | 84.009 | 21.786 | 1.00 | 15.33 | W |
| ATOM | 4068 | OH2 | WAT | W | 84 | 54.729 | 50.430 | −1.548 | 1.00 | 19.38 | W |
| ATOM | 4069 | OH2 | WAT | W | 85 | 57.530 | 61.744 | 4.120 | 1.00 | 16.87 | W |
| ATOM | 4070 | OH2 | WAT | W | 86 | 41.088 | 105.022 | 16.290 | 1.00 | 15.69 | W |
| ATOM | 4071 | OH2 | WAT | W | 87 | 38.319 | 71.451 | 36.984 | 1.00 | 17.14 | W |
| ATOM | 4072 | OH2 | WAT | W | 88 | 31.517 | 89.824 | 37.467 | 1.00 | 16.58 | W |
| ATOM | 4073 | OH2 | WAT | W | 89 | 50.641 | 71.222 | 36.997 | 1.00 | 18.14 | W |
| ATOM | 4074 | OH2 | WAT | W | 90 | 55.999 | 54.399 | −1.005 | 1.00 | 14.41 | W |
| ATOM | 4075 | OH2 | WAT | W | 91 | 48.673 | 35.111 | 15.911 | 1.00 | 17.63 | W |
| ATOM | 4076 | OH2 | WAT | W | 92 | 36.357 | 90.765 | −0.686 | 1.00 | 15.56 | W |
| ATOM | 4077 | OH2 | WAT | W | 93 | 47.926 | 37.673 | 16.359 | 1.00 | 15.70 | W |
| ATOM | 4078 | OH2 | WAT | W | 94 | 61.370 | 53.955 | 18.871 | 1.00 | 17.39 | W |
| ATOM | 4079 | OH2 | WAT | W | 95 | 48.563 | 49.341 | −0.111 | 1.00 | 19.51 | W |
| ATOM | 4080 | OH2 | WAT | W | 96 | 57.430 | 52.827 | 37.437 | 1.00 | 15.87 | W |
| ATOM | 4081 | OH2 | WAT | W | 97 | 32.940 | 88.241 | −0.945 | 1.00 | 16.26 | W |
| ATOM | 4082 | OH2 | WAT | W | 98 | 65.341 | 58.661 | 21.797 | 1.00 | 17.64 | W |
| ATOM | 4083 | OH2 | WAT | W | 99 | 59.363 | 46.482 | 12.477 | 1.00 | 20.25 | W |
| ATOM | 4084 | OH2 | WAT | W | 100 | 59.009 | 55.962 | 24.448 | 1.00 | 17.12 | W |
| ATOM | 4085 | OH2 | WAT | W | 101 | 27.659 | 88.655 | 18.900 | 1.00 | 19.39 | W |
| ATOM | 4086 | OH2 | WAT | W | 102 | 19.552 | 70.631 | 37.180 | 1.00 | 19.27 | W |
| ATOM | 4087 | OH2 | WAT | W | 103 | 62.732 | 52.896 | 9.518 | 1.00 | 18.07 | W |
| ATOM | 4088 | OH2 | WAT | W | 104 | 45.507 | 67.690 | 37.224 | 1.00 | 17.53 | W |
| ATOM | 4089 | OH2 | WAT | W | 105 | 38.378 | 83.608 | 45.067 | 1.00 | 17.12 | W |
| ATOM | 4090 | OH2 | WAT | W | 106 | 50.624 | 59.091 | 45.021 | 1.00 | 17.16 | W |
| ATOM | 4091 | OH2 | WAT | W | 107 | 36.693 | 43.685 | 13.087 | 1.00 | 17.34 | W |
| ATOM | 4092 | OH2 | WAT | W | 108 | 30.022 | 86.690 | 24.483 | 1.00 | 16.79 | W |
| ATOM | 4093 | OH2 | WAT | W | 109 | 26.705 | 89.770 | 12.172 | 1.00 | 19.72 | W |
| ATOM | 4094 | OH2 | WAT | W | 110 | 78.751 | 67.589 | 30.438 | 1.00 | 16.39 | W |
| ATOM | 4095 | OH2 | WAT | W | 111 | 43.442 | 74.935 | 37.288 | 1.00 | 18.43 | W |
| ATOM | 4096 | OH2 | WAT | W | 112 | 62.328 | 52.870 | 12.127 | 1.00 | 18.43 | W |
| ATOM | 4097 | OH2 | WAT | W | 113 | 52.635 | 51.855 | −0.691 | 1.00 | 15.44 | W |
| ATOM | 4098 | OH2 | WAT | W | 114 | 40.454 | 93.344 | −0.107 | 1.00 | 20.15 | W |
| ATOM | 4099 | OH2 | WAT | W | 115 | 40.297 | 107.578 | 15.942 | 1.00 | 17.59 | W |
| ATOM | 4100 | OH2 | WAT | W | 116 | 53.414 | 49.405 | 4.296 | 1.00 | 16.45 | W |
| ATOM | 4101 | OH2 | WAT | W | 117 | 69.415 | 72.026 | 37.172 | 1.00 | 18.90 | W |
| ATOM | 4102 | OH2 | WAT | W | 118 | 38.129 | 90.784 | 4.520 | 1.00 | 17.56 | W |
| ATOM | 4103 | OH2 | WAT | W | 119 | 38.146 | 55.292 | 23.299 | 1.00 | 16.51 | W |
| ATOM | 4104 | OH2 | WAT | W | 120 | 18.017 | 73.683 | 27.997 | 1.00 | 17.00 | W |
| ATOM | 4105 | OH2 | WAT | W | 121 | 53.528 | 43.974 | 7.984 | 1.00 | 16.88 | W |
| ATOM | 4106 | OH2 | WAT | W | 122 | 70.985 | 68.949 | 27.995 | 1.00 | 17.79 | W |
| ATOM | 4107 | OH2 | WAT | W | 123 | 35.576 | 93.322 | 4.318 | 1.00 | 16.95 | W |
| ATOM | 4108 | OH2 | WAT | W | 124 | 43.104 | 86.584 | 29.818 | 1.00 | 19.05 | W |
| ATOM | 4109 | OH2 | WAT | W | 125 | 48.853 | 41.039 | 10.115 | 1.00 | 18.07 | W |
| ATOM | 4110 | OH2 | WAT | W | 126 | 29.628 | 96.265 | 12.429 | 1.00 | 20.43 | W |
| ATOM | 4111 | OH2 | WAT | W | 127 | 37.667 | 89.314 | 29.451 | 1.00 | 18.21 | W |
| ATOM | 4112 | OH2 | WAT | W | 128 | 10.197 | 75.072 | 30.468 | 1.00 | 17.84 | W |
| ATOM | 4113 | OH2 | WAT | W | 129 | 27.695 | 76.838 | 11.844 | 1.00 | 17.47 | W |
| ATOM | 4114 | OH2 | WAT | W | 130 | 51.352 | 53.281 | 29.447 | 1.00 | 17.26 | W |
| ATOM | 4115 | OH2 | WAT | W | 131 | 46.024 | 56.163 | 29.682 | 1.00 | 18.32 | W |
| ATOM | 4116 | OH2 | WAT | W | 132 | 50.807 | 87.407 | 23.278 | 1.00 | 17.70 | W |
| ATOM | 4117 | OH2 | WAT | W | 133 | 29.565 | 83.957 | 17.933 | 1.00 | 19.40 | W |
| ATOM | 4118 | OH2 | WAT | W | 134 | 40.142 | 101.601 | 10.131 | 1.00 | 17.69 | W |
| ATOM | 4119 | OH2 | WAT | W | 135 | 53.886 | 52.579 | 28.038 | 1.00 | 16.20 | W |
| ATOM | 4120 | OH2 | WAT | W | 136 | 30.858 | 94.458 | 1.501 | 1.00 | 19.15 | W |
| ATOM | 4121 | OH2 | WAT | W | 137 | 48.663 | 73.041 | 18.714 | 1.00 | 17.83 | W |
| ATOM | 4122 | OH2 | WAT | W | 138 | 59.527 | 58.698 | 17.871 | 1.00 | 19.95 | W |
| ATOM | 4123 | OH2 | WAT | W | 139 | 58.111 | 48.183 | 1.475 | 1.00 | 18.47 | W |
| ATOM | 4124 | OH2 | WAT | W | 140 | 26.262 | 89.710 | 9.524 | 1.00 | 18.76 | W |
| ATOM | 4125 | OH2 | WAT | W | 141 | 61.241 | 65.789 | 11.822 | 1.00 | 18.51 | W |
| ATOM | 4126 | OH2 | WAT | W | 142 | 35.455 | 98.631 | 8.012 | 1.00 | 18.57 | W |
| ATOM | 4127 | OH2 | WAT | W | 143 | 35.097 | 90.126 | 28.102 | 1.00 | 15.69 | W |
| ATOM | 4128 | OH2 | WAT | W | 144 | 40.256 | 69.610 | 18.629 | 1.00 | 18.94 | W |
| ATOM | 4129 | OH2 | WAT | W | 145 | 24.207 | 86.822 | 36.953 | 1.00 | 21.47 | W |
| ATOM | 4130 | OH2 | WAT | W | 146 | 50.943 | 51.838 | 4.464 | 1.00 | 18.39 | W |
| ATOM | 4131 | OH2 | WAT | W | 147 | 31.576 | 56.567 | 23.098 | 1.00 | 19.24 | W |
| ATOM | 4132 | OH2 | WAT | W | 148 | 64.776 | 55.837 | 37.034 | 1.00 | 20.18 | W |
| ATOM | 4133 | OH2 | WAT | W | 149 | 41.752 | 88.655 | 30.593 | 1.00 | 19.15 | W |
| ATOM | 4134 | OH2 | WAT | W | 150 | 28.943 | 90.064 | 36.740 | 1.00 | 18.17 | W |
| ATOM | 4135 | OH2 | WAT | W | 151 | 53.126 | 74.689 | 17.462 | 1.00 | 21.71 | W |
| ATOM | 4136 | OH2 | WAT | W | 152 | 57.481 | 86.100 | 23.079 | 1.00 | 19.09 | W |
| ATOM | 4137 | OH2 | WAT | W | 153 | 35.919 | 68.039 | 17.377 | 1.00 | 22.31 | W |
| ATOM | 4138 | OH2 | WAT | W | 154 | 51.862 | 84.976 | 9.501 | 1.00 | 19.42 | W |
| ATOM | 4139 | OH2 | WAT | W | 155 | 34.813 | 82.161 | 45.754 | 1.00 | 19.06 | W |
| ATOM | 4140 | OH2 | WAT | W | 156 | 60.087 | 52.558 | 36.734 | 1.00 | 18.62 | W |
| ATOM | 4141 | OH2 | WAT | W | 157 | 41.492 | 74.254 | 17.305 | 1.00 | 22.53 | W |
| ATOM | 4142 | OH2 | WAT | W | 158 | 51.400 | 69.620 | 18.407 | 1.00 | 19.54 | W |
| ATOM | 4143 | OH2 | WAT | W | 159 | 43.474 | 90.722 | 33.704 | 1.00 | 22.13 | W |
| ATOM | 4144 | OH2 | WAT | W | 160 | 55.691 | 62.546 | −2.287 | 1.00 | 19.40 | W |
| ATOM | 4145 | OH2 | WAT | W | 161 | 33.174 | 60.145 | −2.336 | 1.00 | 17.53 | W |
| ATOM | 4146 | OH2 | WAT | W | 162 | 54.074 | 60.472 | 45.738 | 1.00 | 20.40 | W |
| ATOM | 4147 | OH2 | WAT | W | 163 | 45.519 | 51.887 | 33.616 | 1.00 | 24.73 | W |
| ATOM | 4148 | OH2 | WAT | W | 164 | 32.388 | 61.060 | 18.277 | 1.00 | 17.09 | W |
| ATOM | 4149 | OH2 | WAT | W | 165 | 56.542 | 81.596 | 18.358 | 1.00 | 18.68 | W |
| ATOM | 4150 | OH2 | WAT | W | 166 | 50.758 | 68.823 | 41.601 | 1.00 | 18.56 | W |
| ATOM | 4151 | OH2 | WAT | W | 167 | 10.404 | 82.696 | 30.006 | 1.00 | 17.07 | W |
| ATOM | 4152 | OH2 | WAT | W | 168 | 39.208 | 57.693 | 24.249 | 1.00 | 15.79 | W |
| ATOM | 4153 | OH2 | WAT | W | 169 | 47.497 | 68.384 | 17.369 | 1.00 | 22.69 | W |
| ATOM | 4154 | OH2 | WAT | W | 170 | 20.230 | 68.989 | 32.778 | 1.00 | 19.32 | W |
| ATOM | 4155 | OH2 | WAT | W | 171 | 38.240 | 73.855 | 41.614 | 1.00 | 19.18 | W |
| ATOM | 4156 | OH2 | WAT | W | 172 | 52.104 | 36.869 | 15.182 | 1.00 | 23.40 | W |
| ATOM | 4157 | OH2 | WAT | W | 173 | 47.160 | 53.954 | 30.617 | 1.00 | 20.86 | W |
| ATOM | 4158 | OH2 | WAT | W | 174 | 29.888 | 88.727 | 21.062 | 1.00 | 19.28 | W |
| ATOM | 4159 | OH2 | WAT | W | 175 | 32.681 | 90.508 | 26.429 | 1.00 | 23.03 | W |
| ATOM | 4160 | OH2 | WAT | W | 176 | 57.504 | 94.320 | 14.819 | 1.00 | 19.43 | W |
| ATOM | 4161 | OH2 | WAT | W | 177 | 31.443 | 48.305 | 14.849 | 1.00 | 18.59 | W |
| ATOM | 4162 | OH2 | WAT | W | 178 | 57.549 | 87.754 | 14.045 | 1.00 | 20.94 | W |
| ATOM | 4163 | OH2 | WAT | W | 179 | 45.086 | 60.385 | 10.733 | 1.00 | 23.96 | W |
| ATOM | 4164 | OH2 | WAT | W | 180 | 68.770 | 73.686 | 32.727 | 1.00 | 18.69 | W |
| ATOM | 4165 | OH2 | WAT | W | 181 | 31.593 | 54.992 | 13.997 | 1.00 | 21.30 | W |
| ATOM | 4166 | OH2 | WAT | W | 182 | 37.645 | 73.062 | 18.423 | 1.00 | 20.42 | W |
| ATOM | 4167 | OH2 | WAT | W | 183 | 76.394 | 58.932 | 28.770 | 1.00 | 18.62 | W |
| ATOM | 4168 | OH2 | WAT | W | 184 | 37.029 | 105.750 | 15.195 | 1.00 | 25.05 | W |
| ATOM | 4169 | OH2 | WAT | W | 185 | 49.799 | 84.965 | 24.229 | 1.00 | 16.64 | W |
| ATOM | 4170 | OH2 | WAT | W | 186 | 29.729 | 49.663 | 19.033 | 1.00 | 24.99 | W |
| ATOM | 4171 | OH2 | WAT | W | 187 | 78.577 | 60.024 | 29.973 | 1.00 | 18.33 | W |
| ATOM | 4172 | OH2 | WAT | W | 188 | 73.129 | 67.863 | 35.211 | 1.00 | 19.22 | W |
| ATOM | 4173 | OH2 | WAT | W | 189 | 56.199 | 52.141 | 26.472 | 1.00 | 21.85 | W |
| ATOM | 4174 | OH2 | WAT | W | 190 | 51.216 | 48.189 | 35.676 | 1.00 | 22.68 | W |
| ATOM | 4175 | OH2 | WAT | W | 191 | 38.496 | 37.885 | 13.551 | 1.00 | 20.85 | W |
| ATOM | 4176 | OH2 | WAT | W | 192 | 42.144 | 83.131 | 43.841 | 1.00 | 22.20 | W |
| ATOM | 4177 | OH2 | WAT | W | 193 | 37.800 | 99.161 | 6.541 | 1.00 | 20.77 | W |
| ATOM | 4178 | OH2 | WAT | W | 194 | 79.202 | 59.134 | 23.199 | 1.00 | 22.25 | W |
| ATOM | 4179 | OH2 | WAT | W | 195 | 43.464 | 84.043 | 0.966 | 1.00 | 22.99 | W |
| ATOM | 4180 | OH2 | WAT | W | 196 | 9.774 | 83.521 | 23.123 | 1.00 | 21.99 | W |
| ATOM | 4181 | OH2 | WAT | W | 197 | 40.774 | 56.321 | 25.940 | 1.00 | 24.08 | W |
| ATOM | 4182 | OH2 | WAT | W | 198 | 36.174 | 56.191 | 21.648 | 1.00 | 20.43 | W |
| ATOM | 4183 | OH2 | WAT | W | 199 | 37.100 | 57.779 | 9.382 | 1.00 | 21.73 | W |
| ATOM | 4184 | OH2 | WAT | W | 200 | 67.040 | 54.065 | 35.139 | 1.00 | 19.42 | W |
| ATOM | 4185 | OH2 | WAT | W | 201 | 54.990 | 87.263 | 23.288 | 1.00 | 18.23 | W |
| ATOM | 4186 | OH2 | WAT | W | 202 | 29.566 | 81.588 | −1.127 | 1.00 | 24.32 | W |
| ATOM | 4187 | OH2 | WAT | W | 203 | 50.482 | 104.877 | 13.543 | 1.00 | 21.51 | W |
| ATOM | 4188 | OH2 | WAT | W | 204 | 33.809 | 75.981 | 8.987 | 1.00 | 21.22 | W |
| ATOM | 4189 | OH2 | WAT | W | 205 | 58.446 | 82.817 | 21.143 | 1.00 | 24.77 | W |
| ATOM | 4190 | OH2 | WAT | W | 206 | 33.990 | 55.358 | 23.381 | 1.00 | 19.17 | W |
| ATOM | 4191 | OH2 | WAT | W | 207 | 59.100 | 53.954 | 21.096 | 1.00 | 21.68 | W |
| ATOM | 4192 | OH2 | WAT | W | 208 | 43.863 | 75.725 | 43.193 | 1.00 | 25.02 | W |
| ATOM | 4193 | OH2 | WAT | W | 209 | 59.257 | 92.897 | 18.903 | 1.00 | 24.44 | W |
| ATOM | 4194 | OH2 | WAT | W | 210 | 15.872 | 74.869 | 35.220 | 1.00 | 18.53 | W |
| ATOM | 4195 | OH2 | WAT | W | 211 | 37.990 | 100.485 | 8.868 | 1.00 | 19.08 | W |
| ATOM | 4196 | OH2 | WAT | W | 212 | 44.528 | 104.608 | 10.069 | 1.00 | 28.45 | W |
| ATOM | 4197 | OH2 | WAT | W | 213 | 22.020 | 67.110 | 33.698 | 1.00 | 22.25 | W |
| ATOM | 4198 | OH2 | WAT | W | 214 | 45.524 | 58.541 | 0.865 | 1.00 | 24.20 | W |
| ATOM | 4199 | OH2 | WAT | W | 215 | 46.880 | 59.443 | 43.760 | 1.00 | 21.51 | W |
| ATOM | 4200 | OH2 | WAT | W | 216 | 44.398 | 37.868 | 10.230 | 1.00 | 29.45 | W |
| ATOM | 4201 | OH2 | WAT | W | 217 | 42.403 | 47.455 | 1.037 | 1.00 | 22.17 | W |
| ATOM | 4202 | OH2 | WAT | W | 218 | 21.877 | 88.573 | 35.113 | 1.00 | 20.88 | W |
| ATOM | 4203 | OH2 | WAT | W | 219 | 55.162 | 66.763 | 8.963 | 1.00 | 22.51 | W |
| ATOM | 4204 | OH2 | WAT | W | 220 | 52.791 | 86.448 | 21.601 | 1.00 | 21.18 | W |
| ATOM | 4205 | OH2 | WAT | W | 221 | 51.147 | 43.483 | 6.559 | 1.00 | 20.91 | W |
| ATOM | 4206 | OH2 | WAT | W | 222 | 43.808 | 82.212 | 10.720 | 1.00 | 26.33 | W |
| ATOM | 4207 | OH2 | WAT | W | 223 | 12.617 | 83.802 | 28.751 | 1.00 | 20.30 | W |
| ATOM | 4208 | OH2 | WAT | W | 224 | 6.078 | 75.280 | 26.850 | 1.00 | 22.38 | W |
| ATOM | 4209 | OH2 | WAT | W | 225 | 47.881 | 57.392 | −0.792 | 1.00 | 22.09 | W |
| ATOM | 4210 | OH2 | WAT | W | 226 | 50.980 | 42.104 | 8.831 | 1.00 | 19.78 | W |
| ATOM | 4211 | OH2 | WAT | W | 227 | 41.154 | 85.258 | −0.774 | 1.00 | 22.59 | W |
| ATOM | 4212 | OH2 | WAT | W | 228 | 25.986 | 84.606 | 36.424 | 1.00 | 19.93 | W |
| ATOM | 4213 | OH2 | WAT | W | 229 | 37.760 | 94.424 | 35.734 | 1.00 | 24.94 | W |
| ATOM | 4214 | OH2 | WAT | W | 230 | 53.942 | 75.516 | 42.827 | 1.00 | 21.96 | W |
| ATOM | 4215 | OH2 | WAT | W | 231 | 62.956 | 58.032 | 36.430 | 1.00 | 20.68 | W |
| ATOM | 4216 | OH2 | WAT | W | 232 | 48.172 | 76.513 | 38.212 | 1.00 | 24.35 | W |
| ATOM | 4217 | OH2 | WAT | W | 233 | 47.611 | 102.405 | 12.413 | 1.00 | 18.05 | W |
| ATOM | 4218 | OH2 | WAT | W | 234 | 66.970 | 75.519 | 33.669 | 1.00 | 22.17 | W |
| ATOM | 4219 | OH2 | WAT | W | 235 | 42.530 | 53.001 | 37.024 | 1.00 | 25.29 | W |
| ATOM | 4220 | OH2 | WAT | W | 236 | 46.593 | 95.318 | 0.982 | 1.00 | 22.70 | W |
| ATOM | 4221 | OH2 | WAT | W | 237 | 59.434 | 61.102 | −1.153 | 1.00 | 23.03 | W |
| ATOM | 4222 | OH2 | WAT | W | 238 | 41.428 | 40.251 | 12.333 | 1.00 | 19.70 | W |
| ATOM | 4223 | OH2 | WAT | W | 239 | 30.566 | 59.844 | 21.226 | 1.00 | 24.21 | W |
| ATOM | 4224 | OH2 | WAT | W | 240 | 40.963 | 66.286 | 38.250 | 1.00 | 25.23 | W |
| ATOM | 4225 | OH2 | WAT | W | 241 | 35.100 | 102.269 | 13.651 | 1.00 | 23.54 | W |
| ATOM | 4226 | OH2 | WAT | W | 242 | 46.138 | 72.116 | 35.284 | 1.00 | 22.17 | W |
| ATOM | 4227 | OH2 | WAT | W | 243 | 59.052 | 47.058 | 31.892 | 1.00 | 23.41 | W |
| ATOM | 4228 | OH2 | WAT | W | 244 | 27.083 | 65.207 | 15.974 | 1.00 | 24.57 | W |
| ATOM | 4229 | OH2 | WAT | W | 245 | 49.228 | 51.393 | 44.483 | 1.00 | 25.93 | W |
| ATOM | 4230 | OH2 | WAT | W | 246 | 53.883 | 40.364 | 13.609 | 1.00 | 20.63 | W |
| ATOM | 4231 | OH2 | WAT | W | 247 | 19.324 | 82.747 | 33.510 | 1.00 | 19.13 | W |
| ATOM | 4232 | OH2 | WAT | W | 248 | 26.043 | 85.533 | 19.642 | 1.00 | 23.45 | W |
| ATOM | 4233 | OH2 | WAT | W | 249 | 35.021 | 67.121 | 42.825 | 1.00 | 22.49 | W |
| ATOM | 4234 | OH2 | WAT | W | 250 | 46.735 | 76.516 | 12.252 | 1.00 | 24.05 | W |
| ATOM | 4235 | OH2 | WAT | W | 251 | 82.969 | 67.303 | 26.717 | 1.00 | 22.57 | W |
| ATOM | 4236 | OH2 | WAT | W | 252 | 45.209 | 67.018 | 43.177 | 1.00 | 27.94 | W |
| ATOM | 4237 | OH2 | WAT | W | 253 | 55.602 | 79.331 | 37.534 | 1.00 | 24.03 | W |
| ATOM | 4238 | OH2 | WAT | W | 254 | 69.676 | 59.919 | 33.683 | 1.00 | 20.99 | W |
| ATOM | 4239 | OH2 | WAT | W | 255 | 64.125 | 59.002 | 14.884 | 1.00 | 25.33 | W |
| ATOM | 4240 | OH2 | WAT | W | 256 | 42.314 | 66.230 | 12.402 | 1.00 | 23.88 | W |
| ATOM | 4241 | OH2 | WAT | W | 257 | 33.279 | 63.349 | 37.448 | 1.00 | 22.10 | W |
| ATOM | 4242 | OH2 | WAT | W | 258 | 39.649 | 91.675 | 44.503 | 1.00 | 27.13 | W |
| ATOM | 4243 | OH2 | WAT | W | 259 | 50.183 | 77.624 | 17.514 | 1.00 | 28.33 | W |
| ATOM | 4244 | OH2 | WAT | W | 260 | 61.881 | 77.494 | 15.876 | 1.00 | 23.81 | W |
| ATOM | 4245 | OH2 | WAT | W | 261 | 62.925 | 57.128 | 19.612 | 1.00 | 21.49 | W |
| ATOM | 4246 | OH2 | WAT | W | 262 | 78.761 | 69.710 | 28.537 | 1.00 | 21.54 | W |
| ATOM | 4247 | OH2 | WAT | W | 263 | 43.443 | 78.531 | 26.450 | 1.00 | 51.57 | W |
| ATOM | 4248 | OH2 | WAT | W | 264 | 64.436 | 66.462 | 15.185 | 1.00 | 24.50 | W |
| ATOM | 4249 | OH2 | WAT | W | 265 | 29.863 | 95.634 | 31.799 | 1.00 | 22.57 | W |
| ATOM | 4250 | OH2 | WAT | W | 266 | 38.733 | 65.028 | 17.513 | 1.00 | 22.79 | W |
| ATOM | 4251 | OH2 | WAT | W | 267 | 42.862 | 70.588 | 35.156 | 1.00 | 23.17 | W |
| ATOM | 4252 | OH2 | WAT | W | 268 | 18.722 | 72.838 | 25.393 | 1.00 | 22.19 | W |
| ATOM | 4253 | OH2 | WAT | W | 269 | 73.723 | 64.597 | 19.000 | 1.00 | 31.96 | W |
| ATOM | 4254 | OH2 | WAT | W | 270 | 62.723 | 57.662 | 10.545 | 1.00 | 24.92 | W |
| ATOM | 4255 | OH2 | WAT | W | 271 | 57.874 | 62.395 | 44.609 | 1.00 | 21.51 | W |
| ATOM | 4256 | OH2 | WAT | W | 272 | 48.768 | 50.303 | 27.819 | 1.00 | 19.31 | W |
| ATOM | 4257 | OH2 | WAT | W | 273 | 29.763 | 86.426 | 47.578 | 1.00 | 28.69 | W |
| ATOM | 4258 | OH2 | WAT | W | 274 | 31.100 | 59.737 | 35.193 | 1.00 | 31.03 | W |
| ATOM | 4259 | OH2 | WAT | W | 275 | 57.280 | 52.145 | 23.039 | 1.00 | 31.70 | W |
| ATOM | 4260 | OH2 | WAT | W | 276 | 39.141 | 75.601 | 16.301 | 1.00 | 19.15 | W |
| ATOM | 4261 | OH2 | WAT | W | 277 | 40.242 | 92.360 | 27.881 | 1.00 | 21.57 | W |
| ATOM | 4262 | OH2 | WAT | W | 278 | 45.848 | 39.924 | 20.297 | 1.00 | 19.61 | W |
| ATOM | 4263 | OH2 | WAT | W | 279 | 37.239 | 92.961 | 30.038 | 1.00 | 35.26 | W |
| ATOM | 4264 | OH2 | WAT | W | 280 | 34.443 | 91.256 | 24.199 | 1.00 | 23.25 | W |
| ATOM | 4265 | OH2 | WAT | W | 281 | 32.844 | 98.633 | 13.982 | 1.00 | 25.62 | W |
| ATOM | 4266 | OH2 | WAT | W | 282 | 27.691 | 84.905 | −0.900 | 1.00 | 31.39 | W |
| ATOM | 4267 | OH2 | WAT | W | 283 | 49.770 | 67.058 | 16.244 | 1.00 | 19.14 | W |
| ATOM | 4268 | OH2 | WAT | W | 284 | 7.549 | 84.030 | 30.735 | 1.00 | 23.79 | W |
| ATOM | 4269 | OH2 | WAT | W | 285 | 41.424 | 76.017 | 13.321 | 1.00 | 26.91 | W |
| ATOM | 4270 | OH2 | WAT | W | 286 | 26.035 | 92.828 | 18.463 | 1.00 | 27.45 | W |
| ATOM | 4271 | OH2 | WAT | W | 287 | 32.937 | 94.119 | 24.137 | 1.00 | 48.00 | W |
| ATOM | 4272 | OH2 | WAT | W | 288 | 26.166 | 88.282 | 25.556 | 1.00 | 20.51 | W |
| ATOM | 4273 | OH2 | WAT | W | 289 | 63.983 | 65.811 | 11.880 | 1.00 | 27.50 | W |
| ATOM | 4274 | OH2 | WAT | W | 290 | 22.109 | 58.615 | 30.718 | 1.00 | 23.84 | W |
| ATOM | 4275 | OH2 | WAT | W | 291 | 19.238 | 68.372 | 19.647 | 1.00 | 28.58 | W |
| ATOM | 4276 | OH2 | WAT | W | 292 | 56.100 | 44.047 | 14.035 | 1.00 | 24.23 | W |
| ATOM | 4277 | OH2 | WAT | W | 293 | 54.648 | 51.202 | 24.011 | 1.00 | 23.26 | W |
| ATOM | 4278 | OH2 | WAT | W | 294 | 31.678 | 90.398 | 23.056 | 1.00 | 26.00 | W |
| ATOM | 4279 | OH2 | WAT | W | 295 | 53.694 | 43.318 | 2.052 | 1.00 | 22.76 | W |
| ATOM | 4280 | OH2 | WAT | W | 296 | 26.404 | 85.036 | 10.533 | 1.00 | 25.60 | W |
| ATOM | 4281 | OH2 | WAT | W | 297 | 57.999 | 82.967 | 35.181 | 1.00 | 32.74 | W |
| ATOM | 4282 | OH2 | WAT | W | 298 | 24.844 | 83.608 | 14.725 | 1.00 | 32.65 | W |
| ATOM | 4283 | OH2 | WAT | W | 299 | 66.137 | 73.414 | 43.426 | 1.00 | 23.33 | W |
| ATOM | 4284 | OH2 | WAT | W | 300 | 60.389 | 52.123 | 26.401 | 1.00 | 27.82 | W |
| ATOM | 4285 | OH2 | WAT | W | 301 | 45.077 | 83.036 | 43.695 | 1.00 | 28.17 | W |
| ATOM | 4286 | OH2 | WAT | W | 302 | 63.568 | 69.731 | 13.225 | 1.00 | 40.57 | W |
| ATOM | 4287 | OH2 | WAT | W | 303 | 15.309 | 78.322 | 19.060 | 1.00 | 29.40 | W |
| ATOM | 4288 | OH2 | WAT | W | 304 | 6.803 | 75.844 | 23.827 | 1.00 | 27.70 | W |
| ATOM | 4289 | OH2 | WAT | W | 305 | 24.548 | 76.310 | 15.211 | 1.00 | 27.04 | W |
| ATOM | 4290 | OH2 | WAT | W | 306 | 21.315 | 69.478 | 26.567 | 1.00 | 25.89 | W |
| ATOM | 4291 | OH2 | WAT | W | 307 | 55.614 | 92.278 | 9.486 | 1.00 | 28.03 | W |
| ATOM | 4292 | OH2 | WAT | W | 308 | 33.401 | 50.305 | 9.283 | 1.00 | 26.50 | W |
| ATOM | 4293 | OH2 | WAT | W | 309 | 44.860 | 41.748 | 8.296 | 1.00 | 22.14 | W |
| ATOM | 4294 | OH2 | WAT | W | 310 | 36.981 | 98.022 | 38.919 | 1.00 | 27.75 | W |
| ATOM | 4295 | OH2 | WAT | W | 311 | 36.049 | 71.473 | 13.872 | 1.00 | 40.12 | W |
| ATOM | 4296 | OH2 | WAT | W | 312 | 62.799 | 54.411 | 25.492 | 1.00 | 20.27 | W |
| ATOM | 4297 | OH2 | WAT | W | 313 | 68.900 | 66.841 | 46.634 | 1.00 | 40.74 | W |
| ATOM | 4298 | OH2 | WAT | W | 314 | 52.771 | 46.326 | −2.783 | 1.00 | 27.19 | W |
| ATOM | 4299 | OH2 | WAT | W | 315 | 25.076 | 76.838 | 11.881 | 1.00 | 29.09 | W |
| ATOM | 4300 | OH2 | WAT | W | 316 | 37.345 | 55.941 | 35.837 | 1.00 | 31.26 | W |
| END | | | | | | | | | | | |

What is claimed:

1. A compound having formula 1:
R¹—R²R³R⁴—R⁵
where R¹ and R⁵ are the same and may be alkylaminosulfonyl, alkylaminosulfonylalkyl, alkylsulfonyl, alkylsulfonylalkyl, alkylaminocarbonyl, alkylaminocarbonylalkyl, alkylaminocarboxy, alkylaminocarboxyalkyl;
where R² and R⁴ are preferably, but not necessarily, the same, and are ring structures, optionally substituted, which may be aryl, cycloalkyl, or heterocyclyl; and
where R³ may be a bond between R² and R⁴ which either is shared by or joins the two rings without being shared in the rings themselves.

2. The compound of claim 1 wherein R²R³R⁴ is a naphthyl group.

3. The compound of claim 1 wherein R²R³R⁴ is a bi-aryl group.

4. A compound having formula 2:
where R⁶ and R⁷ are the same or different and may be alkylaminosulfonyl, 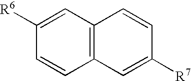
alkylaminosulfonylalkyl, alkylsulfonyl, alkylsulfonylalkyl, alkylaminocarbonyl, alkylaminocarbonylalkyl, alkylaminocarboxy, alkylaminocarboxyalkyl.

5. The compound of claim 4 wherein R⁶ and R⁷ are the same and are both alkylaminosulfonyl groups.

6. The compound of claim 4 wherein R⁶ and R⁷ are the same and are both alkylaminosulfonylalkyl groups.

7. The compound of claim 6 which has formula 3:

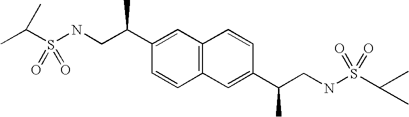

8. A compound having formula 4: 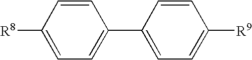
where R⁸ and R⁹ are the same and may be alkylaminosulfonyl, alkylaminosulfonylalkyl, alkylsulfonyl, alkylsulfonylalkyl, alkylaminocarbonyl, alkylaminocarbonylalkyl, alkylaminocarboxy, alkylaminocarboxyalkyl.

9. The compound of claim 8 wherein R⁸ and R⁹ are both alkylaminosulfonyl groups.

10. The compound of claim 8 wherein R⁸ and R⁹ are both alkylaminosulfonylalkyl groups.

11. The compound of claim 10 which has formula 5:

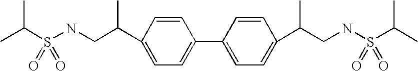

12. A compound of formula 6: 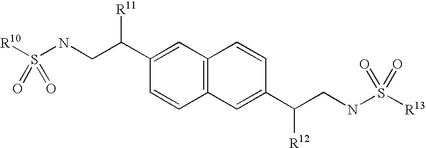
where R¹⁰ and R¹³ are the same, or, if they are not, then R¹¹ and R¹² are the same, and each of R¹⁰, R¹¹, R¹², and R¹³ may be H, methyl, ethyl, propyl, isopropyl or butyl.

13. A compound of formula 7: 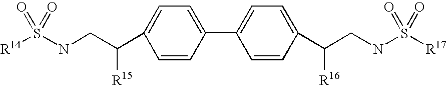
where R¹⁴ and R¹⁷ are the same, or, if they are not, then R¹⁵ and R¹⁶ are the same, and each of R¹⁴, R¹⁵, R¹⁶, and R¹⁷ individually may be H, methyl, ethyl, propyl, isopropyl or butyl.

14. A method of treating a disorder selected from the group consisting of anxiety and depression, comprising administering an effective amount of a compound of claim 1.

15. A method of treating a disorder selected from the group consisting of anxiety and depression, comprising administering an effective amount of a compound of claim 2.

16. A method of treating a disorder selected from the group consisting of anxiety and depression, comprising administering an effective amount of a compound of claim 3.

17. A method of treating a disorder selected from the group consisting of anxiety and depression, comprising administering an effective amount of a compound of claim 4.

18. A method of treating a disorder selected from the group consisting of anxiety and depression, comprising administering an effective amount of a compound of claim 5.

19. A method of treating a disorder selected from the group consisting of anxiety and depression, comprising administering an effective amount of a compound of claim 6.

20. A method of treating a disorder selected from the group consisting of anxiety and depression, comprising administering an effective amount of a compound of claim 7.

21. A method of treating a disorder selected from the group consisting of anxiety and depression, comprising administering an effective amount of a compound of claim 8.

22. A method of treating a disorder selected from the group consisting of anxiety and depression, comprising administering an effective amount of a compound of claim 9.

23. A method of treating a disorder selected from the group consisting of anxiety and depression, comprising administering an effective amount of a compound of claim 10.

24. A method of treating a disorder selected from the group consisting of anxiety and depression, comprising administering an effective amount of a compound of claim 11.

25. A method of treating a disorder selected from the group consisting of anxiety and depression, comprising administering an effective amount of a compound of claim 12.

26. A method of treating a disorder selected from the group consisting of anxiety and depression, comprising administering an effective amount of a compound of claim 13.

27. Use of a compound of any of claims 1-13 for preparing a pharmaceutical for use in treating anxiety or depression.
